(19) United States
(12) Patent Application Publication
(10) Pub. No.: US 2007/0213340 A1
(43) Pub. Date: Sep. 13, 2007

(54) Farnesyl protein transferase inhibitors

(76) Inventors: Joseph M. Kelly, Parlin, NJ (US); Adriano Afonso, West Caldwell, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/654,160

(22) Filed: Jan. 17, 2007

Related U.S. Application Data
(60) Provisional application No. 60/760,317, filed on Jan. 19, 2006.

(51) Int. Cl. A61K 31/496; C07D 403/02

(52) U.S. Cl. 514/253.03; 544/361

(57) ABSTRACT

Disclosed are compounds of the formula: 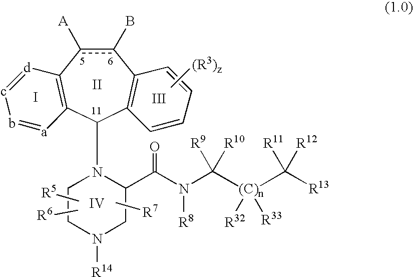
wherein R¹³ represents an imidazole ring; R¹⁴ represents a carbamate, urea, amide or sulfonamide group, and the remaining substituents are as defined herein. Also disclosed is a method of treating cancer and a method of inhibiting farnesyl protein transferase using the disclosed compounds.

### REFERENCE TO RELATED APPLICATION

This application claims the benefit of U.S. Provisional Application Ser. No. 60/760,317 filed Jan. 19, 2006, the disclosure of which is incorporated herein by reference thereto.

### BACKGROUND

U.S. Pat. No. 6,372,747B1, issued Apr. 16, 2002 discloses compounds of the formula: 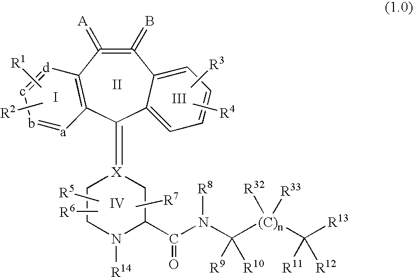
such as, for example, compounds of the formula:

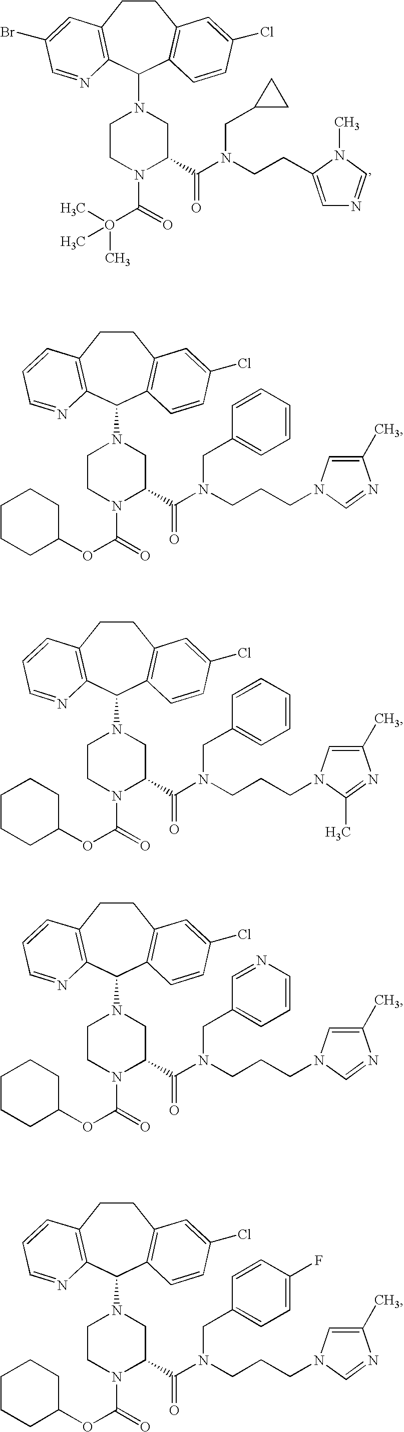

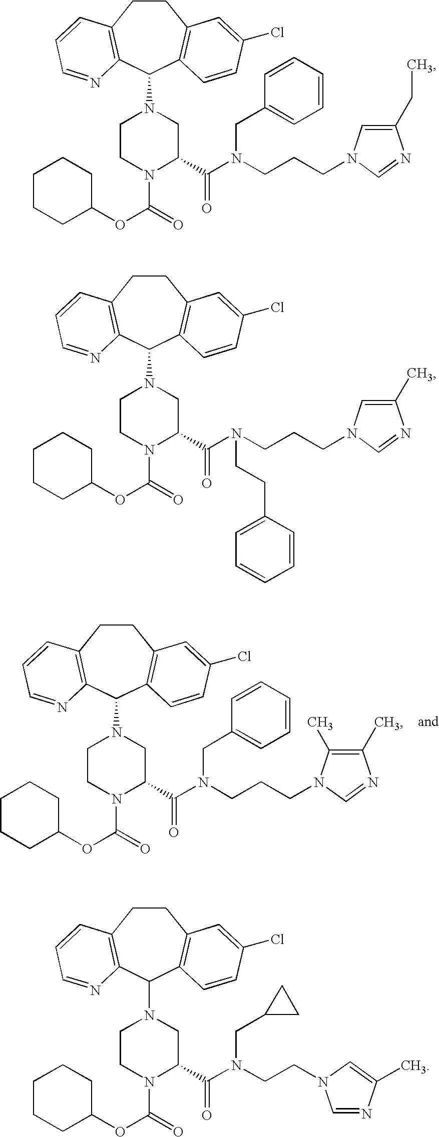

WO 95/10516, published Apr. 20, 1995, WO96/31478, published Oct. 10, 1996, and copending application Ser. No. 09/094,687 filed Jun. 15, 1998 discloses tricyclic compounds useful for inhibiting farnesyl protein transferase.

In view of the current interest in inhibitors of farnesyl protein transferase, a welcome contribution to the art would be compounds useful for the inhibition of farnesyl protein transferase. Such a contribution is provided by this invention.

### SUMMARY OF THE INVENTION

This invention provides compounds useful for the inhibition of farnesyl protein transferase (FPT). The compounds of this invention are represented by the formula: 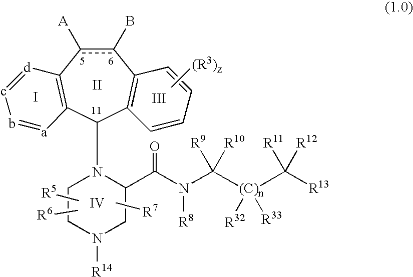
or a pharmaceutically acceptable salt or thereof, wherein the substituents are as defined below.

This invention is also directed to a pharmaceutical composition comprising an effective amount of at least one (e.g., 1, 2 or 3, or 1 or 2, or 1, and usually 1) compound of formula 1.0, or a pharmaceutically acceptable salt thereof, and a pharmaceutically acceptable carrier.

This invention is also directed to a pharmaceutical composition comprising an effective amount of at least one (e.g., 1, 2 or 3, or 1 or 2, or 1, and usually 1) compound of formula 1.0, or a pharmaceutically acceptable salt thereof, and at least one other pharmaceutically active ingredient (e.g., 1, 2 or 3, or 1 or 2, or 1, and usually 1), and a pharmaceutically acceptable carrier.

This invention is also directed to a method of treating cancer in a patient in need of such treatment, said method comprising administering to said patient an effective amount of at least one (e.g., 1, 2 or 3, or 1 or 2, or 1, and usually 1) compound of formula 1.0, or a pharmaceutically acceptable salt thereof. Radiation therapy can optionally be used in this method.

This invention is also directed to a method of treating cancer in a patient in need of such treatment, said method comprising administering to said patient an effective amount of at least one (e.g., 1, 2 or 3, or 1 or 2, or 1, and usually 1) compound of formula 1.0, or a pharmaceutically acceptable salt thereof, in combination with at least one (e.g., 1, 2 or 3, or 1 or 2, or 1) chemotherapeutic agent.

This invention is also directed to a method of treating cancer in a patient in need of such treatment, said method comprising administering to said patient an effective amount of a pharmaceutical composition comprising at least one (e.g., 1, 2 or 3, or 1 or 2, or 1, and usually 1) compound of formula 1.0, or a pharmaceutically acceptable salt thereof. Radiation therapy can optionally be used in this method.

This invention is also directed to a method of treating cancer in a patient in need of such treatment, said method comprising administering to said patient an effective amount of a pharmaceutical composition comprising at least one (e.g., 1, 2 or 3, or 1 or 2, or 1, and usually 1) compound of formula 1.0, or a pharmaceutically acceptable salt thereof, in combination with at least one (e.g., 1, 2 or 3, or 1 or 2, or 1) chemotherapeutic agent.

This invention is also directed to a method of inhibiting farnesyl protein transferase in a patient in need of such treatment, said method comprising administering to said patient an effective amount of at least one (e.g., 1, 2 or 3, or 1 or 2, or 1, and usually 1) compound of formula 1.0, or a pharmaceutically acceptable salt thereof.

This invention is also directed to a method of inhibiting farnesyl protein transferase in a patient in need of such treatment, said method comprising administering to said patient an effective amount of a pharmaceutical composition comprising at least one (e.g., 1, 2 or 3, or 1 or 2, or 1, and usually 1) compound of formula 1.0, or a pharmaceutically acceptable salt thereof.

This invention is also directed to a method of inhibiting farnesyl protein transferase in a patient in need of such treatment, said method comprising administering to said patient a pharmaceutical composition comprising an effective amount of at least one (e.g., 1, 2 or 3, or 1 or 2, or 1, and usually 1) compound of formula 1.0, or a pharmaceutically acceptable salt thereof.

This invention also provides a method of treating cancer in a patient in need of such treatment, said method comprising administering to said patient an effective amount of at least one (e.g., 1, 2 or 3, 1 or 2, and usually 1) compound of formula 1.0 in combination with at least one (e.g., 1, 2 or 3, 1 or 2, and usually 1) signal transduction inhibitor.

This invention also provides a method of treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 in combination with at least one (e.g., 1, 2 or 3, 1 or 2, and usually 1) signal transduction inhibitor.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma, in a patient in need of such treatment, said method comprising administering to said patient an effective amount of at least one (e.g., 1, 2 or 3, 1 or 2, and usually 1) compound of formula 1.0.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating cancer in a patient in need of such treatment, said method comprising administering to said patient an effective amount of at least one (e.g., 1, 2 or 3, 1 or 2, and usually 1) compound of formula 1.0 wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent wherein said cancer is selected from the group consisting of: melanoma, pancreatic cancer, thyroid cancer, colorectal cancer, lung cancer, breast cancer, and ovarian cancer.

This invention also provides a method for treating melanoma in a patient in need of such treatment, said method comprising administering to said patient an effective amount of at least one (e.g., 1, 2 or 3, 1 or 2, and usually 1) compound of formula 1.0.

This invention also provides a method for treating melanoma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating melanoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating melanoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating pancreatic cancer in a patient in need of such treatment, said method comprising administering to said patient an effective amount of at least one (e.g., 1, 2 or 3, 1 or 2, and usually 1) compound of formula 1.0.

This invention also provides a method for treating pancreatic cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating pancreatic cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating pancreatic cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating thyroid cancer in a patient in need of such treatment, said method comprising administering to said patient an effective amount of at least one (e.g., 1, 2 or 3, 1 or 2, and usually 1) compound of formula 1.0.

This invention also provides a method for treating thyroid cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating thyroid cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating thyroid cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating colorectal cancer in a patient in need of such treatment, said method comprising administering to said patient an effective amount of at least one (e.g., 1, 2 or 3, 1 or 2, and usually 1) compound of formula 1.0.

This invention also provides a method for treating colorectal cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating colorectal cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating colorectal cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating lung cancer in a patient in need of such treatment, said method comprising administering to said patient an effective amount of at least one (e.g., 1, 2 or 3, 1 or 2, and usually 1) compound of formula 1.0.

This invention also provides a method for treating lung cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating lung cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating lung cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating breast cancer in a patient in need of such treatment, said method comprising administering to said patient an effective amount of at least one (e.g., 1, 2 or 3, 1 or 2, and usually 1) compound of formula 1.0.

This invention also provides a method for treating breast cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating breast cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating breast cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating ovarian cancer in a patient in need of such treatment, said method comprising administering to said patient an effective amount of at least one (e.g., 1, 2 or 3, 1 or 2, and usually 1) compound of formula 1.0.

This invention also provides a method for treating ovarian cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating ovarian cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating ovarian cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides methods of treating breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents).

This invention also provides methods of treating breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents).

This invention also provides methods of treating breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

This invention also provides methods of treating breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

The methods of treating breast cancer described herein include the treatment of hormone-dependent metastatic and advanced breast cancer, adjuvant therapy for hormone-dependent primary and early breast cancer, the treatment of ductal carcinoma in situ, and the treatment of inflammatory breast cancer in situ.

The methods of treating hormone-dependent breast cancer can also be used to prevent breast cancer in patients having a high risk of developing breast cancer.

Thus, this invention also provides methods of preventing breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents).

This invention also provides methods of preventing breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents).

This invention also provides methods of preventing breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

This invention also provides methods of preventing breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 in combination with hormonal therapies (i.e., antihormonal agents), and in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) a in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating brain cancer (e.g., glioma, such as glioma blastoma multiforme) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0, in combination with an effective amount of a chemotherapeutic agent wherein said chemotherapeutic agent is temozolomide.

This invention also provides a method for treating brain cancer (e.g., glioma, such as glioma blastoma multiforme) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of a chemotherapeutic agent, wherein said chemotherapeutic agent is temozolomide.

This invention also provides a method for treating prostate cancer in a patient in need of such treatment, said method comprising administering to said patient an effective amount of at least one (e.g., 1, 2 or 3, 1 or 2, and usually 1) compound of formula 1.0.

This invention also provides a method for treating prostate cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating prostate cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating prostate cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myelodysplastic syndrome in a patient in need of such treatment, said method comprising administering to said patient an effective amount of at least one (e.g., 1, 2 or 3, 1 or 2, and usually 1) compound of formula 1.0.

This invention also provides a method for treating myelodysplastic syndrome in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myelodysplastic syndrome in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating myelodysplastic syndrome in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating acute myelogenous leukemia (AML) in a patient in need of such treatment, said method comprising administering to said patient an effective amount of at least one (e.g., 1, 2 or 3, 1 or 2, and usually 1) compound of formula 1.0.

This invention also provides a method for treating acute myelogenous leukemia (AML) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating acute myelogenous leukemia (AML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating acute myelogenous leukemia (AML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelomonocytic leukemia (CMML) in a patient in need of such treatment, said method comprising administering to said patient an effective amount of at least one (e.g., 1, 2 or 3, 1 or 2, and usually 1) compound of formula 1.0.

This invention also provides a method for treating chronic myelomonocytic leukemia (CMML) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelomonocytic leukemia (CMML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating chronic myelomonocytic leukemia (CMML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelogenous leukemia (chronic myeloid leukemia, CML) in a patient in need of such treatment, said method comprising administering to said patient an effective amount of at least one (e.g., 1, 2 or 3, 1 or 2, and usually 1) compound of formula 1.0.

This invention also provides a method for treating chronic myelogenous leukemia (chronic myeloid leukemia, CML) in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating chronic myelogenous leukemia (chronic myeloid leukemia, CML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating chronic myelogenous leukemia (chronic myeloid leukemia, CML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating bladder cancer in a patient in need of such treatment, said method comprising administering to said patient an effective amount of at least one (e.g., 1, 2 or 3, 1 or 2, and usually 1) compound of formula 1.0.

This invention also provides a method for treating bladder cancer in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating bladder cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating bladder cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating non-Hodgkin's lymphoma in a patient in need of such treatment, said method comprising administering to said patient an effective amount of at least one (e.g., 1, 2 or 3, 1 or 2, and usually 1) compound of formula 1.0.

This invention also provides a method for treating non-Hodgkin's lymphoma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating non-Hodgkin's lymphoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating non-Hodgkin's lymphoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating multiple myeloma in a patient in need of such treatment, said method comprising administering to said patient an effective amount of at least one (e.g., 1, 2 or 3, 1 or 2, and usually 1) compound of formula 1.0.

This invention also provides a method for treating multiple myeloma in a patient in need of such treatment, said method comprising administering to said patient an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

This invention also provides a method for treating multiple myeloma in a patient in need of such treatment, said method comprising administering to said patient an effective amount of a pharmaceutical composition comprising an effective amount of at least one (e.g., 1, 2 or 3, 1 or 2, and usually 1) compound of formula 1.0.

This invention also provides a method for treating multiple myeloma in a patient in need of such treatment, said method comprising administering to said patient an effective amount of a pharmaceutical composition comprising an effective amount of at least one (e.g., 1, 2 or 3, 1 or 2, and usually 1) compound of formula 1.0, in combination with an effective amount of at least one (e.g., 1, 2 or 3, 1 or 2, or 1) chemotherapeutic agent.

In the methods of this invention the compounds of this invention can be administered concurrently or sequentially (i.e., consecutively) with the chemotherapeutic agents or the signal transduction inhibitor.

The methods of treating cancers described herein can optionally include the administration of an effective amount of radiation (i.e., the methods of treating cancers described herein optionally include the administration of radiation therapy).

The compounds of this invention: (i) potently inhibit farnesyl protein transferase, but not geranylgeranyl protein transferase I, in vitro; (ii) block the phenotypic change induced by a form of transforming Ras which is a farnesyl acceptor but not by a form of transforming Ras engineered to be a geranylgeranyl acceptor; (iii) block intracellular processing of Ras which is a farnesyl acceptor but not of Ras engineered to be a geranylgeranyl acceptor; and (iv) block abnormal cell growth in culture induced by transforming Ras.

The compounds of this invention inhibit farnesyl protein transferase and the farnesylation of the oncogene protein Ras. Thus, this invention further provides a method of inhibiting farnesyl protein transferase, (e.g., ras farnesyl protein transferase) in mammals, especially humans, by the administration of an effective amount of the compounds of formula 1.0. The administration of the compounds of formula 1.0 to patients, to inhibit farnesyl protein transferase, is useful in the treatment of the cancers described below.

This invention provides a method for inhibiting or treating the abnormal growth of cells, including transformed cells, by administering an effective amount of a compound of formula 1.0. Abnormal growth of cells refers to cell growth independent of normal regulatory mechanisms (e.g., loss of contact inhibition). This includes the abnormal growth of: (1) tumor cells (tumors) expressing an activated Ras oncogene; (2) tumor cells in which the Ras protein is activated as a result of oncogenic mutation in another gene; and (3) benign and malignant cells of other proliferative diseases in which aberrant Ras activation occurs.

This invention also provides a method for inhibiting or treating tumor growth (i.e., cancer) by administering an effective amount of the compounds of formula 1.0 to a mammal (e.g., a human) in need of such treatment. In particular, this invention provides a method for inhibiting or treating the growth of tumors (cancers) expressing an activated Ras oncogene by the administration of an effective amount of the compounds of formula 1.0.

Examples of tumors (cancers) which may be inhibited or treated include, but are not limited to: lung cancer (e.g., lung adenocarcinoma), pancreatic cancers (e.g., pancreatic carcinoma such as, for example, exocrine pancreatic carcinoma), colon cancers (e.g., colorectal carcinomas, such as, for example, colon adenocarcinoma and colon adenoma), myeloid leukemias (for example, acute myelogenous leukemia (AML)), thyroid follicular cancer, myelodysplastic syndrome (MDS), chronic myeloid leukemia (chronic myelogenous leukemia (CML)), chronic myelomonocytic leukemia (CMML), bladder carcinoma, epidermal carcinoma, melanoma, brain cancer (e.g., glioma and blastoglioma multiforme), ovarian cancer, breast cancer and prostate cancer.

It is believed that this invention also provides a method for inhibiting or treating proliferative diseases, both benign and malignant, wherein Ras proteins are aberrantly activated as a result of oncogenic mutation in other genes—i.e., the Ras gene itself is not activated by mutation to an oncogenic form—with said inhibition or treatment being accomplished by the administration of an effective amount of the tricyclic compounds described herein, to a mammal (e.g., a human) in need of such treatment. For example, the benign proliferative disorder neurofibromatosis, or tumors in which Ras is activated due to mutation or overexpression of tyrosine kinase oncogenes (e.g., neu, src, abl, lck, and fyn), may be inhibited or treated by the tricyclic compounds described herein.

The compounds of formula 1.0 useful in the methods of this invention inhibit or treat the abnormal growth of cells. Without wishing to be bound by theory, it is believed that these compounds may function through the inhibition of G-protein function, such as ras p21, by blocking G-protein isoprenylation, thus making them useful in the treatment of proliferative diseases such as tumor growth and cancer. Without wishing to be bound by theory, it is believed that these compounds inhibit ras farnesyl protein transferase, and thus show antiproliferative activity against ras transformed cells.

### DETAILED DESCRIPTION OF THE INVENTION

As used herein, the following terms are used as defined below unless otherwise indicated:
MH⁺—represents the molecular ion plus hydrogen of the molecule in the mass spectrum;
BOC—represents tert-butyloxycarbonyl;
CBZ—represents —C(O)OCH₂C₆H₅ (i.e., benzyloxycarbonyl);
CH₂Cl₂—represents dichloromethane;
CIMS—represents chemical ionization mass spectrum;
DEAD—represents diethylazodicarboxylate;
DEC—represents EDCl which represents 1-(3-dimethyl-aminopropyl)-3-ethylcarbodiimide hydrochloride;
DMF—represents N,N-dimethylformamide;
Et—represents ethyl;
EtOAc—represents ethyl acetate;
EtOH—represents ethanol;
HOBT—represents 1-hydroxybenzotriazole hydrate;
IPA—represents isopropanol;
iPrOH—represents isopropanol;
Me—represents methyl;
MeOH—represents methanol;
MS—represents mass spectroscopy;
NMM—represents N-methylmorpholine;
Ph—represents phenyl;
Pr—represents propyl;
TBDMS—represents tert-butyldimethylsilyl;
TEA—represents triethylamine;
TFA—represents trifluoroacetic acid;
THF—represents tetrahydrofuran;
Tr—represents trityl;

Also, as described herein, unless otherwise indicated, the use of a drug or compound in a specified period is per treatment cycle. For example, once a day means once per day of each day of the treatment cycle. Twice a day means twice per day each day of the treatment cycle. Once a week means one time per week during the treatment cycle. Once every three weeks means once per three weeks during the treatment cycle.

As used herein, unless otherwise specified, the following terms have the following meanings:
“anti-cancer agent” means a drug (medicament or pharmaceutically active ingredient) for treating cancer;
“antineoplastic agent” means a drug (medicament or pharmaceutically active ingredient) for treating cancer (i.e., a chemotherapeutic agent);
“at least one”, as used in reference to the number of compounds of this invention means for example 1-6, generally 1-4, more generally 1, 2 or 3, and usually one or two, and more usually one;
“at least one”, as used in reference to the number of chemotherapeutic agents used, means for example 1-6, generally 1-4, more generally 1, 2 or 3, and usually one or two, or one;
“chemotherapeutic agent” means a drug (medicament or pharmaceutically active ingredient) for treating cancer (i.e., and antineeoplastic agent);
“compound” with reference to the antineoplastic agents, includes the agents that are antibodies;
“concurrently” means (1) simultaneously in time (e.g., at the same time); or (2) at different times during the course of a common treatment schedule;
“consecutively” means one following the other;
“different” as used in the phrase “different antineoplastic agents” means that the agents are not the same compound or structure; preferably, “different” as used in the phrase “different antineoplastic agents” means not from the same class of antineoplastic agents; for example, one antineoplastic agent is a taxane, and another antineoplastic agent is a platinum coordinator compound;
effective amount-represents a therapeutically effective amount; for example, the amount of the compound (or drug), or radiation, that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor; for example, in the treatment of lung cancer (e.g., non small cell lung cancer) a therapeutically effective amount is that amount that alleviates or eliminates cough, shortness of breath and/or pain; also, for example, a therapeutically effective amount of the FPT inhibitor is that amount which results in the reduction of farnesylation; the reduction in farnesylation may be determined by the analysis of pharmacodynamic markers such as Prelamin A and HDJ-2 (DNAJ-2) using techniques well known in the art;
“one or more” has the same meaning as “at least one”;
“patient” means an animal, such as a mammal (e.g., a human being, and preferably a human being);
“prodrug” means compounds that are rapidly transformed, for example, by hydrolysis in blood, in vivo to the parent compound, i.e., to the compounds of formula 1.0 or to a salt and/or to a solvate thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes Prodrugs of the novel compounds of this invention;
sequentially-represents (1) administration of one component of the method ((a) compound of the invention, or (b) chemotherapeutic agent, signal transduction inhibitor and/or radiation therapy) followed by administration of the other component or components; after administration of one component, the next component can be administered substantially immediately after the first component, or the next component can be administered after an effective time period after the first component; the effective time period is the amount of time given for realization of maximum benefit from the administration of the first component; and
“solvate” means a physical association of a compound of this invention with one or more solvent molecules; this physical association involves varying degrees of ionic and covalent bonding, including hydrogen bonding; in certain instances the solvate will be capable of isolation, for example when one or more solvent molecules are incorporated in the crystal lattice of the crystalline solid; “solvate” encompasses both solution-phase and isolatable solvates; non-limiting examples of suitable solvates include ethanolates, methanolates, and the like; “hydrate” is a solvate wherein the solvent molecule is H₂O.

As used herein, unless otherwise specified, the following terms have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl, and the like):
“acyl” means an H—C(O)—, alkyl-C(O)—, alkenyl-C(O)—, Alkynyl-C(O)—, cycloalkyl-C(O)—, cycloalkenyl-C(O)—, or cycloalkynyl-C(O)— group in which the various groups are as defined below (and as defined below, the alkyl, alkenyl, alkynyl, cycloalkyl, cycloalkenyl and cycloalkynyl moieties can be substituted); the bond to the parent moiety is through the carbonyl; preferred acyls contain a lower alkyl; Non-limiting examples of suitable acyl groups include formyl, acetyl, propanoyl, 2-methylpropanoyl, butanoyl and cyclohexanoyl;
“alkenyl” means an aliphatic hydrocarbon group (chain) comprising at least one carbon to carbon double bond, wherein the chain can be straight or branched, and wherein said group comprises about 2 to about 15 carbon atoms; Preferred alkenyl groups comprise about 2 to about 12 carbon atoms in the chain; and more preferably about 2 to about 6 carbon atoms in the chain; branched means that one or more lower alkyl groups, such as methyl, ethyl or propyl, or alkenyl groups are attached to a linear alkenyl chain; “lower alkenyl” means an alkenyl group comprising about 2 to about 6 carbon atoms in the chain, and the chain can be straight or branched; the term “substituted alkenyl” means that the alkenyl group is substituted by one or more independently selected substituents, and each substituent is independently selected from the group consisting of: halo, alkyl, aryl, cycloalkyl, cyano, alkoxy and —S(alkyl); non-limiting examples of suitable alkenyl groups include ethenyl, propenyl, n-butenyl, 3-methylbut-2-phenyl, n-pentenyl, octenyl and decenyl;
“alkoxy” means an alkyl-O— group (i.e., the bond to the parent moiety is through the ether oxygen) in which the alkyl group is unsubstituted or substituted as described below; non-limiting examples of suitable alkoxy groups include methoxy, ethoxy, n-propoxy, isopropoxy, n-butoxy and heptoxy;
“alkoxycarbonyl” means an alkyl-O—CO— group (i.e., the bond to the parent moiety is through the carbonyl) wherein the alkyl group is unsubstituted or substituted as previously defined; non-limiting examples of suitable alkoxycarbonyl groups include methoxycarbonyl and ethoxycarbonyl;
“alkyl” (including the alkyl portions of other moieties, such as trifluoroalkyl and alkyloxy) means an aliphatic hydrocarbon group (chain) that can be straight or branched wherein said group comprises about 1 to about 20 carbon atoms in the chain; preferred alkyl groups comprise about 1 to about 12 carbon atoms in the chain; more preferred alkyl groups comprise about 1 to about 6 carbon atoms in the chain; branched means that one or more lower alkyl groups, such as methyl, ethyl or propyl, are attached to a linear alkyl chain; “lower alkyl” means a group comprising about 1 to about 6 carbon atoms in the chain, and said chain can be straight or branched; the term “substituted alkyl” means that the alkyl group is substituted by one or more independently selected substituents, and wherein each substituent is independently selected from the group consisting of: halo, aryl, cycloalkyl, cyano, hydroxy, alkoxy, alkylthio, amino, —NH(alkyl), —NH(cycloalkyl), —N(alkyl)₂, carboxy, —C(O)O-alkyl and —S(alkyl); non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl;
“alkylaryl” (or alkaryl) means an alkyl-aryl-group (i.e., the bond to the parent moiety is through the aryl group) wherein the alkyl group is unsubstituted or substituted as defined above, and the aryl group is unsubstituted or substituted as defined below; preferred alkylaryls comprise a lower alkyl group; non-limiting examples of suitable alkylaryl groups include o-tolyl, p-tolyl and xylyl;
“alkylheteroaryl” means an alkyl-heteroaryl-group (i.e., the bond to the parent moiety is through the heteroaryl group) wherein the alkyl is unsubstituted or substituted as defined above and the heteroaryl group is unsubstituted or substituted as defined below;
“alkylsulfinyl” means an alkyl-S(O)— group (i.e., the bond to the parent moiety is through the sulfinyl) wherein the alkyl group is unsubstituted or substituted as previously defined; preferred groups are those in which the alkyl group is lower alkyl;
“alkylsulfonyl” means an alkyl-S(O₂)— group (i.e., the bond to the parent moiety is through the sulfonyl) wherein the alkyl group is unsubstituted or substituted as previously defined; preferred groups are those in which the alkyl group is lower alkyl;
“alkylthio” means an alkyl-S— group (i.e., the bond to the parent moiety is through the sulfur) wherein the alkyl group is unsubstituted or substituted as previously described; non-limiting examples of suitable alkylthio groups include methylthio, ethylthio, i-propylthio and heptylthio;
“alkynyl” means an aliphatic hydrocarbon group (chain) comprising at least one carbon to carbon triple bond, wherein the chain can be straight or branched, and wherein the group comprises about 2 to about 15 carbon atoms in the; preferred alkynyl groups comprise about 2 to about 12 carbon atoms in the chain; and more preferably about 2 to about 4 carbon atoms in the chain; Branched means that one or more lower alkyl groups, such as methyl, ethyl or propyl, are attached to a linear alkynyl chain; “lower alkynyl” means an alkynyl group comprising about 2 to about 6 carbon atoms in the chain, and the chain can be straight or branched; non-limiting examples of suitable alkynyl groups include ethynyl, propynyl, 2-butynyl, 3-methylbutynyl, n-pentynyl, and decynyl; the term “substituted alkynyl” means that the alkynyl group is substituted by one or more independently selected, and each substituent is independently selected from the group consisting of alkyl; aryl and cycloalkyl;
“amino means a —NH₂ group;
“aralkenyl” (or arylalkenyl) means an aryl-alkenyl-group (i.e., the bond to the parent moiety is through the alkenyl group) wherein the aryl group is unsubstituted or substituted as defined below, and the alkenyl group is unsubstituted or substituted as defined above; preferred aralkenyls contain a lower alkenyl group; non-limiting examples of suitable aralkenyl groups include 2-phenethenyl and 2-naphthylethenyl;
“aralkyl” (or arylalkyl) means an aryl-alkyl-group (i.e., the bond to the parent moiety is through the alkyl group) wherein the aryl is unsubstituted or substituted as defined below and the alkyl is unsubstituted or substituted as defined above; preferred aralkyls comprise a lower alkyl group; non-limiting examples of suitable aralkyl groups include benzyl, 2-phenethyl and naphthalenylmethyl;
“aralkyloxy” (or arylalkyloxy) means an aralkyl-O— group (i.e., the bond to the parent moiety is through the ether oxygen) wherein the aralkyl group is unsubstituted or substituted as previously described; non-limiting examples of suitable aralkyloxy groups include benzyloxy and 1- or 2-naphthalenemethoxy;
“aralkoxycarbonyl” means an aralkyl-O—C(O)— group (i.e., the bond to the parent moiety is through the carbonyl) wherein the aralkyl group is unsubstituted or substituted as previously defined; a non-limiting example of a suitable aralkoxycarbonyl group is benzyloxycarbonyl;
“aralkylthio” means an aralkyl-S— group (i.e., the bond to the parent moiety is through the sulfur) wherein the aralkyl group is unsubstituted or substituted as previously described; a non-limiting example of a suitable aralkylthio group is benzylthio;
“aroyl” means an aryl-C(O)— group (i.e., the bond to the parent moiety is through the carbonyl) wherein the aryl group is unsubstituted or substituted as defined below; non-limiting examples of suitable groups include benzoyl and 1- and 2-naphthoyl;
“aryl” (sometimes abbreviated “ar”) means an aromatic monocyclic or multicyclic ring system comprising about 6 to about 14 carbon atoms, preferably about 6 to about 10 carbon atoms; the aryl group can be optionally substituted with one or more independently selected “ring system substituents” (defined below). Non-limiting examples of suitable aryl groups include phenyl and naphthyl;
“arylalkynyl” means an aryl-alkynyl-group (i.e., the bond to the parent moiety is through the alkynyl group) wherein the aryl group is unsubstituted or substituted as defined above, and the alkynyl group is unsubstituted or substituted as defined above;
“arylaminoheteroaryl” means an aryl-amino-heteroaryl group (i.e., the bond to the parent moiety is through the heteroaryl group) wherein the aryl group is unsubstituted or substituted as defined above, the amino group is as defined above (i.e., a —NH— here), and the heteroaryl group is unsubstituted or substituted as defined below;
“arylheteroaryl” means an aryl-heteroarylgroup- (i.e., the bond to the parent moiety is through the heteroaryl group) wherein the aryl group is unsubstituted or substituted as defined above, and the heteroaryl group is unsubstituted or substituted as defined below;
“aryloxy” means an aryl-O— group (i.e., the bond to the parent moiety is through the ether oxygen) wherein the aryl group is unsubstituted or substituted as defined above; non-limiting examples of suitable aryloxy groups include phenoxy and naphthoxy;
“aryloxycarbonyl” means an aryl-O—C(O)— group (i.e., the bond to the parent moiety is through the carbonyl) wherein the aryl group is unsubstituted or substituted as previously defined; non-limiting examples of suitable aryloxycarbonyl groups include phenoxycarbonyl and naphthoxycarbonyl;
“arylsulfinyl” means an aryl-S(O)— group (i.e., the bond to the parent moiety is through the sulfinyl) wherein aryl is unsubstituted or substituted as previously defined;
“arylsulfonyl” means an aryl-S(O₂)— group (i.e., the bond to the parent moiety is through the sulfonyl) wherein aryl is unsubstituted or substituted as previously defined;
“arylthio” means an aryl-S— group (i.e., the bond to the parent moiety is through the sulfur) wherein the aryl group is unsubstituted or substituted as previously described; non-limiting examples of suitable arylthio groups include phenylthio and naphthylthio;
“cycloalkenyl” means a non-aromatic mono or multicyclic ring system comprising about 3 to about 10 carbon atoms, preferably about 5 to about 10 carbon atoms that contains at least one carbon-carbon double bond; preferred cycloalkenyl rings contain about 5 to about 7 ring atoms; the cycloalkenyl can be optionally substituted with one or more independently selected “ring system substituents” (defined below); Non-limiting examples of suitable monocyclic cycloalkenyls include cyclopentenyl, cyclohexenyl, cycloheptenyl, and the like; a non-limiting example of a suitable multicyclic cycloalkenyl is norbornylenyl;
“cycloalkyl” means a non-aromatic mono- or multicyclic ring system comprising about 3 to about 7 carbon atoms, preferably about 3 to about 6 carbon atoms; the cycloalkyl can be optionally substituted with one or more independently selected “ring system substituents” (defined below); non-limiting examples of suitable monocyclic cycloalkyls include cyclopropyl, cyclopentyl, cyclohexyl, cycloheptyl and the like; non-limiting examples of suitable multicyclic cycloalkyls include 1-decalin, norbornyl, adamantyl and the like;
“cycloalkylalkyl” means a cycloalkyl-alkyl-group (i.e., the bond to the parent moiety is through the alkyl group) wherein the cycloalkyl moiety is unsubstituted or substituted as defined above, and the alkyl moiety is unsubstituted or substituted as defined above;
“halo” means fluoro, chloro, bromo, or iodo groups; preferred halos are fluoro, chloro or bromo, and more preferred are fluoro and chloro;
“halogen” means fluorine, chlorine, bromine, or iodine; preferred halogens are fluorine, chlorine and bromine;
“haloalkyl” means an alkyl, as defined above, wherein one or more hydrogen atoms on the alkyl is replaced by a halo group, as defined above;
“heteroaralkenyl” means a heteroaryl-alkenyl-group (i.e., the bond to the parent moiety is through the alkenyl group) wherein the heteroaryl group is unsubstituted or substituted as defined below, and the alkenyl group is unsubstituted or substituted as defined above;
“heteroaralkyl” (or heteroarylalkyl) means a heteroaryl-alkyl-group (i.e., the bond to the parent moiety is through the alkyl group) in which the heteroaryl is unsubstituted or substituted as defined below, and the alkyl group is unsubstituted or substituted as defined above; preferred heteroaralkyls comprise an alkyl group that is a lower alkyl group; non-limiting examples of suitable aralkyl groups include pyridylmethyl, 2-(furan-3-yl)ethyl and quinolin-3-ylmethyl;
“heteroaralkylthio” means a heteroaralkyl-S— group wherein the heteroaralkyl group is unsubstituted or substituted as defined above;
“heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination; preferred heteroaryls comprise about 5 to about 6 ring atoms; the “heteroaryl” can be optionally substituted by one or more independently selected “ring system substituents” (defined below); the prefix aza, oxa or thia before the heteroaryl root name means that at least a nitrogen, oxygen or sulfur atom, respectively, is present as a ring atom; a nitrogen atom of a heteroaryl can be optionally oxidized to the corresponding N-oxide; non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like;
“heteroarylalkynyl” (or heteroaralkynyl) means a heteroaryl-alkynyl-group (i.e., the bond to the parent moiety is through the alkynyl group) wherein the heteroaryl group is unsubstituted or substituted as defined above, and the alkynyl group is unsubstituted or substituted as defined above;
“heteroarylaryl” (or heteroararyl) means a heteroaryl-aryl-group (i.e., the bond to the parent moiety is through the aryl group) wherein the heteroaryl group is unsubstituted or substituted as defined above, and the aryl group is unsubstituted or substituted as defined above;
“heteroarylheteroarylaryl” means a heteroaryl-heteroaryl-group (i.e., the bond to the parent moiety is through the last heteroaryl group) wherein each heteroaryl group is independently unsubstituted or substituted as defined above;
“heteroarylsulfinyl” means a heteroaryl-SO— group wherein the heteroaryl group is unsubstituted or substituted as defined above;
“heteroarylsulfonyl” means a heteroaryl-SO₂— group wherein the heteroaryl group is unsubstituted or substituted as defined above;
“heteroarylthio” means a heteroaryl-S— group wherein the heteroaryl group is unsubstituted or substituted as defined above;
“heterocyclenyl” (or heterocycloalkenyl) means a non-aromatic monocyclic or multicyclic ring system comprising about 3 to about 10 ring atoms, preferably about 5 to about 10 ring atoms, in which one or more of the atoms in the ring system is an element other than carbon (for example one or more heteroatoms independently selected from the group consisting of nitrogen, oxygen and sulfur atom), and which contains at least one carbon-carbon double bond or carbon-nitrogen double bond; there are no adjacent oxygen and/or sulfur atoms present in the ring system; Preferred heterocyclenyl rings contain about 5 to about 6 ring atoms; the prefix aza, oxa or thia before the heterocyclenyl root name means that at least a nitrogen, oxygen or sulfur atom, respectively, is present as a ring atom; the heterocyclenyl can be optionally substituted by one or more independently selected “Ring system substituents” (defined below); the nitrogen or sulfur atom of the heterocyclenyl can be optionally oxidized to the corresponding N-oxide, S-oxide or S,S-dioxide; non-limiting examples of suitable monocyclic azaheterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like; Non-limiting examples of suitable oxaheterocyclenyl groups include 3,4-dihydro-2H-pyran, dihydrofuranyl, fluorodihydrofuranyl, and the like; A non-limiting example of a suitable multicyclic oxaheterocyclenyl group is 7-oxabicyclo[2.2.1]heptenyl; non-limiting examples of suitable monocyclic thiaheterocyclenyl rings include dihydrothiophenyl, dihydrothiopyranyl, and the like;
“heterocycloalkylalkyl” (or heterocyclylalkyl) means a heterocycloalkyl-alkyl-group (i.e., the bond to the parent moiety is through the alkyl group) wherein the heterocycloalkyl group (i.e., the heterocyclyl group) is unsubstituted or substituted as defined below, and the alkyl group is unsubstituted or substituted as defined above;
“heterocyclyl” (or heterocycloalkyl) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination; there are no adjacent oxygen and/or sulfur atoms present in the ring system; preferred heterocyclyls contain about 5 to about 6 ring atoms; the prefix aza, oxa or thia before the heterocyclyl root name means that at least a nitrogen, oxygen or sulfur atom respectively is present as a ring atom; the heterocyclyl can be optionally substituted by one or more independently selected “ring system substituents” (defined below); the nitrogen or sulfur atom of the heterocyclyl can be optionally oxidized to the corresponding N-oxide, S-oxide or S,S-dioxide; non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like;
“hydroxyalkyl” means a HO-alkyl-group wherein the alkyl group is substituted or unsubstituted as defined above; preferred hydroxyalkyls comprise a lower alkyl; Non-limiting examples of suitable hydroxyalkyl groups include hydroxymethyl and 2-hydroxyethyl; and
“ring system substituent” means a substituent attached to an aromatic or non-aromatic ring system that, for example, replaces an available hydrogen on the ring system; ring system substituents are each independently selected from the group consisting of: alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, R⁶⁰R⁶⁵N—, R⁶⁰R⁶⁵N-alkyl-, R⁶⁰R⁶⁵NC(O)— and R⁶⁰R⁶⁵NSO₂—, wherein R⁶⁰ and R⁶⁵ are each independently selected from the group consisting of: hydrogen, alkyl, aryl, and aralkyl; “Ring system substituent” also means a cyclic ring of 3 to 7 ring atoms, wherein 1-2 ring atoms can be heteroatoms, attached to an aryl, heteroaryl, heterocyclyl or heterocyclenyl ring by simultaneously substituting two ring hydrogen atoms on said aryl, heteroaryl, heterocyclyl or heterocyclenyl ring; Non-limiting examples include:

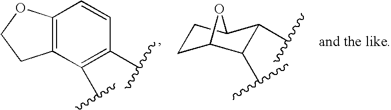

Lines drawn into a ring mean that the indicated bond may be attached to any of the substitutable ring carbon atoms.

Any carbon or heteroatom with unsatisfied valences in the text, schemes, examples, structural formulae, and any Tables herein is assumed to have the hydrogen atom or atoms to satisfy the valences.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, capsules, pills and the like. Similarly, the herein-described methods of treating a patient by administering a pharmaceutical composition of the present invention is also intended to encompass the administration of the afore-said bulk composition and individual dosage units.

Prodrugs of the compounds of the invention are also contemplated herein. The term “prodrug”, as employed herein, denotes a compound that is a drug precursor which, upon administration to a subject, undergoes chemical conversion by metabolic or chemical processes to yield a compound of formula 1.0 or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

For example, if a compound of formula 1.0, or a pharmaceutically acceptable salt, hydrate or solvate of the compound, contains a carboxylic acid functional group, a prodrug can comprise an ester formed by the replacement of the hydrogen atom of the acid group with a group such as, for example, (C₁-C₈)alkyl, (C₂-C₁₂)alkanoyloxy-methyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxy-carbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of formula 1.0 contains an alcohol functional group, a prodrug can be formed by the replacement of the hydrogen atom of the alcohol group with a group such as, for example, (C₁-C₆)alkanoyloxymethyl, 1-((C₁-C₆)alkanoyl-oxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of formula 1.0 incorporates an amine functional group, a prodrug can be formed by the replacement of a hydrogen atom in the amine group with a group such as, for example, R⁷⁰-carbonyl, R⁷⁰O-carbonyl, NR⁷⁰R⁷⁵-carbonyl where R⁷⁰ and R⁷⁵ are each independently (C₁-C₁₀)alkyl, (C₃-C₇) cycloalkyl, benzyl, or R⁷⁰-carbonyl is a natural α-aminoacyl or natural α-aminoacyl, —C(OH)C(O)OY⁸⁰ wherein Y⁸⁰ is H, (C₁-C₆)alkyl or benzyl, —C(OY⁸²)_{y84} wherein Y⁸² is (C₁-C₄) alkyl and Y⁸⁴ is (C₁-C₆)alkyl, carboxy (C₁-C₆)alkyl, amino(C₁-C₄)alkyl or mono-N— or di-N,N-(C₁-C₆)alkylaminoalkyl, —C(Y⁸⁶)Y⁸⁸ wherein Y⁸⁶ is H or methyl and Y⁸⁸ is mono-N— or di-N,N-(C₁-C₆)alkylamino morpholino, piperidin-1-yl or pyrrolidin-1-yl, and the like.

This invention also includes the compounds of this invention in isolated and purified form.

Polymorphic forms of the compounds of formula 1.0, and of the salts, solvates and prodrugs of the compounds of formula 1.0, are intended to be included in the present invention.

Certain compounds of the invention may exist in different isomeric (e.g., enantiomers, diastereoisomers, atropisomers) forms. The invention contemplates all such isomers both in pure form and in admixture, including racemic mixtures. Enol forms are also included.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prod rugs of the inventive compounds.

Diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

The compounds of formula 1.0 form salts that are also within the scope of this invention. Reference to a compound of formula 1.0 herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula 1.0 contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable salts) are preferred. Salts of the compounds of the formula 1.0 may be formed, for example, by reacting a compound of formula 1.0 with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiod ides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexyl-amine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid and base salts are intended to be pharmaceutically acceptable salts within the scope of the invention and all acid and base salts are considered equivalent to the free forms of the corresponding compounds for purposes of the invention.

Compounds of formula 1.0, and salts, solvates and prodrugs thereof, may exist in their tautomeric form (for example, as an amide or imino ether). All such tautomeric forms are contemplated herein as part of the present invention.

In hetero-atom containing ring systems of this invention, there are no hydroxyl groups on carbon atoms adjacent to a N, O or S, and there are no N or S groups on carbon adjacent to another heteroatom. Thus, for example, in the ring: 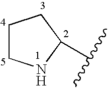
there is no —OH attached directly to carbons marked 2 and 5.

The compounds of formula 1.0 may exist in different tautomeric forms, and all such forms are embraced within the scope of the invention. Also, for example, all keto-enol and imine-enamine forms of the compounds are included in the invention.

Tautomeric forms such as, for example, the moieties: 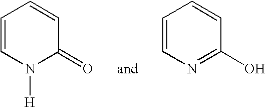
are considered equivalent in certain embodiments of this invention.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R³, etc.) occurs more than one time in any moiety or in any compound of formula 1.0, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of formula 1.0 (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of formula 1.0 can generally be prepared by following procedures analogous to those disclosed in the Schemes and/or in the Examples hereinbelow, by substituting an appropriate isotopically labelled reagent for a non-isotopically labelled reagent.

The positions in the tricyclic ring system are:

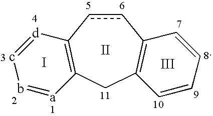

The compounds of this invention are represented by the formula: 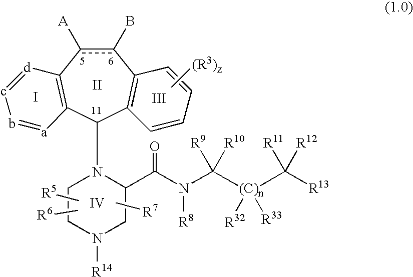
or a pharmaceutically acceptable salt or thereof, wherein:

one of a, b, c and d represents N or N⁺O⁻, and the remaining a, b, c and d groups represent CR¹ wherein each R¹ is independently selected; or

each of a, b, c, and d are CR¹ wherein each R¹ is independently selected;

the dotted line between carbon atoms 5 and 6 represents an optional bond;

when the optional bond is present between C5 and C6 (i.e., there is a double bond between C5 and C6), each A and B are each independently selected from the group consisting of: —R¹⁵, halo, —OR¹⁶, —OCO₂R¹⁶ and —OC(O)R¹⁵;

when the optional bond between C5 and C6 is not present (i.e., there is a single bond between carbon atoms 5 and 6), each A and B are each independently selected from the group consisting of: (a) H₂, (b) —(OR¹⁶)₂ wherein each R¹⁶ is independently selected, (c) H and halo, (d) dihalo wherein each halo is independently selected, (e) alkyl and H, (f) (alkyl)₂ wherein each alkyl is independently selected, (g) —H and —OC(O)R¹⁵, (h) H and —OR¹⁵, (i) ═O, (j) aryl and H, (k) ═NOR¹⁵ and (l) —O—(CH₂)ₚ—O— wherein p is 2, 3 or 4;

each R¹ is independently selected from the group consisting of: (a) H, (b) halo, (c) —CF₃, (d) —OR¹⁵ (e.g., —OCH₃), (e) —COR⁵, (f) —SR¹⁵ (e.g., —SCH₃ and —SCH₂C₆H₅), (g) —S(O)ₜR¹⁶ (wherein t is 0, 1 or 2, e.g., —SOCH₃ and —SO₂CH₃), (h) —N(R¹⁵)₂, (i) —NO₂, (j) —OC(O)R¹⁵, (k) —CO₂R¹⁵, (l) —OCO₂R¹⁶, (m) —CN, (n) —NR¹⁵COOR¹⁶, (o)—SR¹⁶C(O)OR¹⁶ (e.g., —SCH₂CO₂CH₃), (p) —SR¹⁶N(R¹⁷)₂ (provided that R¹⁶ in —SR¹⁶N(R¹⁷)₂ is not —CH₂—) wherein each R¹⁷ is independently selected from the group consisting of H and —C(O)OR¹⁶ (e.g., —S(CH₂)₂NHC(O)O-t-butyl and —S(CH₂)₂NH₂), (p) benzotriazol-1-yloxy, (q) tetrazol-5-ylthio, (r) substituted tetrazol-5-ylthio (e.g., alkyl substituted tetrazol-5-ylthio such as 1-methyl-tetrazol-5-ylthio), (s) alkynyl, (t) alkenyl and (u) alkyl, said alkyl or alkenyl group optionally being substituted with halo, —OR¹⁵ or —CO₂R¹⁵;

each R³ is independently selected from the group consisting of: (a) halo, (b) —CF₃, (c) —OR¹⁵ (e.g., —OCH₃), (d) —COR⁵, (e) —SR¹⁵ (e.g., —SCH₃ and —SCH₂C₆H₅), (f) —S(O)ₜR¹⁶ (wherein t is 0, 1 or 2, e.g., —SOCH₃ and —SO₂CH₃), (g) —N(R¹⁵)₂, (h) —NO₂, (i) —OC(O)R¹⁵, (j) —CO₂R¹⁵, (k) —OCO₂R¹⁶, (l) —CN, (m) —NR¹⁵COOR¹⁶, (n) —SR¹⁶C(O)OR¹⁶ (e.g., —SCH₂CO₂CH₃), —SR¹⁶N(R¹⁷)₂ (provided that R¹⁶ in —SR⁶N(R⁷)₂ is not —CH₂—) wherein each R¹⁷ is independently selected from the group consisting of H and —C(O)OR¹⁶ (e.g., —S(CH₂)₂NHC(O)O-t-butyl and —S(CH₂)₂NH₂), (o) benzotriazol-1-yloxy, (p) tetrazol-5-ylthio, (q) substituted tetrazol-5-ylthio (e.g., alkyl substituted tetrazol-5-ylthio such as 1-methyl-tetrazol-5-ylthio), (r) alkynyl, (s) alkenyl and (t) alkyl, said alkyl or alkenyl group optionally being substituted with halo, —OR¹⁵ or —CO₂R¹⁵; or

two R³ groups taken together with the carbon atoms to which they are bound form a saturated or unsaturated C₅-C₇ ring;

z is 0, 1, 2, or 3 (preferably 1 or 2, or 1);

R⁵, R⁶, and R⁷ are each independently selected from the group consisting of: H, —CF₃, —COR¹⁵, alkyl and aryl, wherein said alkyl or aryl is optionally substituted with —OR¹⁵, —SR¹⁵, —S(O)ₜR¹⁶, —NR¹⁵COOR¹⁶, —N(R¹⁵)₂, —NO₂, —COR¹⁵, —OCOR¹⁵, —OCO₂R¹⁶, —CO₂R¹⁵, and OPO₃R¹⁵, or R⁵ and R⁶ together represent ═O or ═S;

R⁸ is selected from the group consisting of: H, C₃ to C₇ alkyl (e.g., branched chain alkyl, for example, C₄ to C₇ branched chain alkyl), aryl, arylalkyl- (e.g., benzyl), heteroaryl, heteroarylalkyl-, cycloalkyl, cycloalkylalkyl-, substituted alkyl, substituted aryl, substituted arylalkyl-, substituted heteroaryl, substituted heteroarylalkyl-, substituted cycloalkyl, substituted cycloalkylalkyl-;

the substituents for the R⁸ substituted groups are independently selected from the group consisting of: alkyl, aryl, arylalkyl-, cycloalkyl, —N(R¹⁸)₂, —OR¹⁸, cycloalkyalkyl-, halo, CN, —C(O)N(R¹⁸)₂, —SO₂N(R¹⁸)₂ and —CO₂R¹⁸; provided that the —OR¹⁸ and —N(R¹⁸)₂ substituents are not bound to the carbon that is bound to the N of the —C(O)NR⁸— moiety;

R⁹ and R¹⁰ are independently selected from the group consisting of: H, alkyl, aryl, arylalkyl-, heteroaryl, heteroarylalkyl-, cycloalkyl or —CON(R¹⁸)₂ (wherein R¹⁸ is as defined above); and the substitutable R⁹ and R¹⁰ groups are optionally substituted with one or more (e.g., 1-3) substituents independently selected from the group consisting of: alkyl (e.g., methyl, ethyl, isopropyl, and the like), cycloalkyl, arylalkyl-, or heterarylalkyl- (i.e., the R⁹ and/or R¹⁰ groups can be unsubstituted, or the R⁹ and/or R¹⁰ groups (except when H) can be substituted with 1-3 of the substitutents described above); or

R⁹ and R¹⁰ together with the carbon atom to which they are bound, form a C₃ to C₆ cycloalkyl ring;

R¹¹ and R¹² are independently selected from the group consisting of: H, alkyl, aryl, arylalkyl-, heteroaryl, heteroarylalkyl-, cycloalkyl, —CON(R¹⁸)₂—OR¹⁸ or —N(R¹⁸)₂; wherein R¹⁸ is as defined above; provided that the —OR¹⁸ and —N(R¹⁸)₂ groups are not bound to a carbon atom that is adjacent to a nitrogen atom; and wherein said substitutable R¹¹ and R¹² groups are optionally substituted with one or more (e.g., 1-3) substituents selected from the group consisting of: alkyl (e.g., methyl, ethyl, isopropyl, and the like), cycloalkyl, arylalkyl-, or heterarylalkyl-; or

R¹¹ and R¹² together with the carbon atom to which they are bound, form a C₃ to C₆ cycloalkyl ring; or

R¹¹ and R¹² taken together with the carbon to which they are bound form a 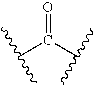
moiety, i.e., the moiety 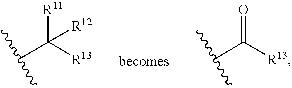
for example when R¹³ is —OR⁴⁰, R¹¹ and R¹² can be taken together with the carbon atom to which they are bound to form a —C(O)— group, that is, for example, the moiety

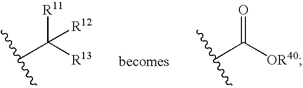

R¹³ is selected from the group consisting of: —OR⁴⁰ (wherein R⁴⁰ is an alkyl group, such as a C₁ to C₆ alkyl group, such as, for example, ethyl), —C(O)OR⁶⁰ and imidazolyl, wherein said imidazolyl is selected from the group consisting of: 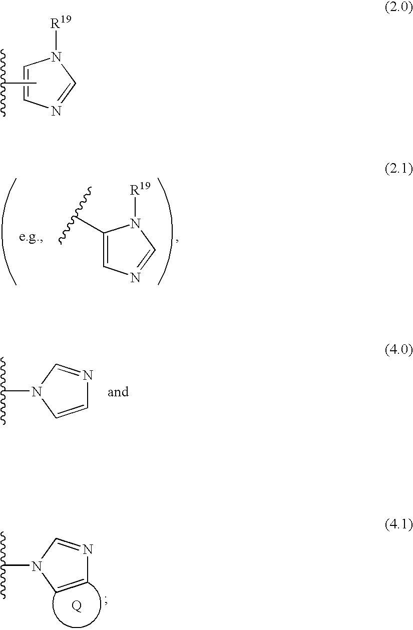
wherein said imidazolyl ring 2.0 or 2.1 is optionally substituted with one or two substituents, and said imidazole ring 4.0 is optionally substituted with 1-3 substituents, and said imidazole ring 4.1 is optionally substituted with one substituent, and wherein said optional substituents for said imidazolyl rings 2.0, 2.1, 4.0 and 4.1 are bound to the carbon atoms of said imidazolyl rings, and said optional substituents are independently selected from the group consisting of: —NHC(O)R¹⁸, —C(R³⁴)₂OR³⁵ (e.g., —CH₂OH, —CH₂OC(O)OR²⁰ and —CH₂OC(O)NHR²⁰), —OR¹⁸, —SR¹⁸, F, Cl, Br, alkyl, aryl, arylalkyl-, cycloalkyl, and —N(R¹⁸)₂ (wherein each R¹⁸ is independently selected);

Q represents an aryl ring (e.g., phenyl), a cycloalkyl ring (e.g., cyclopentyl or cyclohexyl), or a heteroaryl ring (e.g., furanyl, pyrrolyl, thienyl, oxazolyl or thiazolyl), said Q is optionally substituted with 1 to 4 substituents independently selected from the group consisting of: halo (e.g., F or Cl), alkyl, aryl, —OR¹⁸, —N(R¹⁸)₂ (wherein each R¹⁸ is independently selected), —OC(O)R¹⁸, and —C(O)N(R¹⁸)₂ (wherein each R¹⁸ is independently selected);

R¹⁴ is selected from the group consisting of:

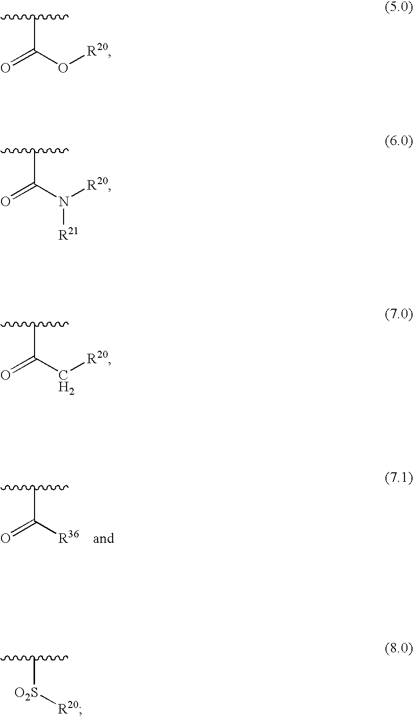

R¹⁵ is selected from the group consisting of: H, alkyl, aryl and arylalkyl-;

R¹⁶ is selected from the group consisting of: alkyl and aryl;

each R¹⁸ is independently selected from the group consisting of: H, alkyl, aryl, arylalkyl-, heteroaryl and cycloalkyl;

R¹⁹ is selected from the group consisting of: (1) H, (2) alkyl, (3) aryl, (4) arylalkyl-, (5) substituted arylalkyl-, (6) —C(aryl)₃ (e.g., —C(phenyl)₃, i.e., trityl) and (7) cycloalkyl; and wherein the substituents on said substituted arylalkyl- are selected from the group consisting of: halo (e.g., F and Cl) and CN;

R²⁰ is selected from the group consisting of: H, alkyl, alkoxy, aryl, arylalkyl-, cycloalkyl, heteroaryl, heteroarylalkyl- and heterocycloalkyl, provided that R²⁰ is not H when R¹⁴ is group 5.0 or 8.0;

when R²⁰ is other than H, then said R²⁰ group is optionally substituted with one or more (e.g., 1-3) substituents selected from the group consisting of: halo, alkyl, aryl, —OC(O)R¹⁸ (e.g., —OC(O)CH₃), —OR¹⁸ and —N(R¹⁸)₂, wherein each R¹⁸ group is the same or different, provided that said optional substituent is not bound to a carbon atom that is adjacent to an oxygen or nitrogen atom;

R²¹ is selected from the group consisting of: H, alkyl, aryl, arylalkyl-, cycloalkyl, heteroaryl, heteroarylalkyl- or heterocycloalkyl;

when R²¹ is other than H, then said R²¹ group is optionally substituted with one or more (e.g., 1-3) substituents selected from the group consisting of: halo, alkyl, aryl, —OR¹⁸ or —N(R¹⁸)₂, wherein each R¹⁸ group is the same or different, provided that said optional substituent is not bound to a carbon atom that is adjacent to an oxygen or nitrogen atom;

n is 0-5;

each R³² and each R³³ for each n (i.e., for each —C(R³²)(R³³)— group), is independently selected from the group consisting of: H, alkyl, aryl, arylalkyl-, heteroaryl, heteroarylalkyl-, cycloalkyl, —CON(R¹⁸)₂, —OR¹⁸ and —N(R¹⁸)₂; and wherein said substitutable R³² and R³³ groups are optionally substituted with one or more (e.g., 1-3) substituents selected from the group consisting of: alkyl (e.g., methyl, ethyl, isopropyl, and the like), cycloalkyl, arylalkyl-, and heterarylalkyl-; or

R³² and R³³ together with the carbon atom to which they are bound, form a C₃ to C₆ cycloalkyl ring;

each R³⁴ is independently selected from the group consisting of: H and alkyl (e.g. —CH₃), and R³⁴ is preferably H;

R³⁵ is selected from the group consisting of: H, —C(O)OR²⁰ and —C(O)NHR²⁰, (preferably R²⁰ is alkyl or cycloalkyl, most preferably cyclopentyl or cyclohexyl);

R³⁶ is selected from the group consisting of: branched alkyl, unbranched alkyl, cycloalkyl, heterocycloalkyl, and aryl (e.g., phenyl); and

R⁶⁰ is selected from the group consisting of: H and alkyl (e.g., C₁ to C₆ alkyl, such as ethyl).

This invention is also directed to the compounds of formula 1.0, wherein:

(1) when R¹⁴ is selected from: group 6.0, 7.0, 7.1 or 8.0, then R⁸ is selected from: C₃ to C₁₀ alkyl, substituted C₃ to C₁₀ alkyl, arylalkyl-, substituted arylalkyl-, heteroarylalkyl-, substituted heteroarylalkyl-, cycloalkylalkyl-, or substituted cycloalkylalkyl-; and

(2) when R¹⁴ is selected from: group 6.0, 7.0, 7.1 or 8.0, and R⁸ is H, then the alkyl chain between R¹³ and the amide moiety (i.e., the —C(O)NR⁸ group) is substituted, i.e.,: (a) at least one of R⁹, R¹⁰, R¹¹, R¹², R³², or R³³ is other than H, and/or (b) R⁹ and R¹⁰, and/or R¹¹ and R¹², are taken together to form a cycloalkyl ring.

This invention is also directed to the compounds of formula 1.0, wherein when R¹⁴ is group 5.0, and R⁸ is H, then the alkyl chain between R¹³, when R¹³ is the imidazolyl ring 2.0, 4.0 or 4.1), and the amide moiety (i.e., the —C(O)NR⁸ group) is substituted, i.e.: (a) at least one of R⁹, R¹⁰, R¹¹, R¹², R³², or R³³ is other than H, and/or (b) R⁹ and R¹⁰, and/or R¹¹ and R¹², are taken together to form a cycloalkyl ring.

The compounds of formula 1.0 include the 3S (formula 1.0A) and the 3R (formula 1.0B) isomers:

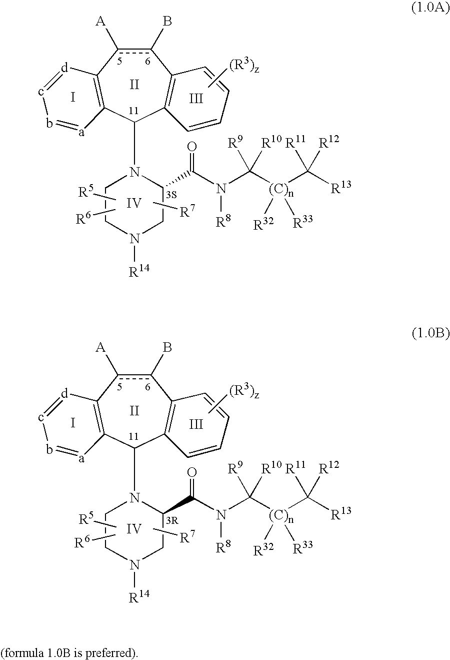

Examples of R⁸ substituents include: H and benzyl.

Other examples of R⁸ include: —CH₂C(CH₃)₂, —CH₂-cyclohexyl, —CH₂-cyclopropyl, —(CH₂)₂CH₃,

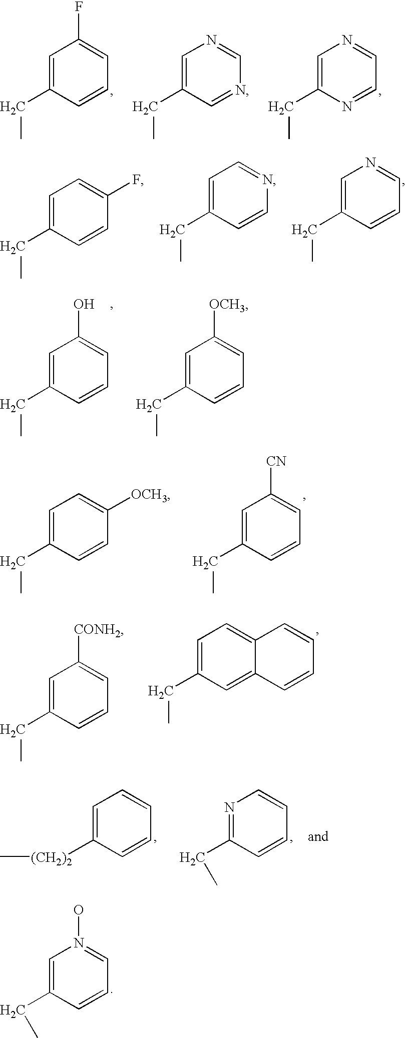

Examples of R⁹ and R¹⁰ groups include, but are not limited to: H and benzyl. In another example R⁹ and R¹⁰ are H.

Examples of R¹¹ and R¹² groups include: H, —CH₃, —CH₂CH(CH₃)₂, —(CH₂)₃CH₃, benzyl, ethyl, p-chlorophenyl, and —OH. In another example R¹¹ and R¹² are H.

Cyclopropyl is an Example of the R¹¹ and R¹² group being taken together with the carbon atom to which they are bound to form a cycloalkyl ring.

Examples of the optional substituents for the R¹³ imidazolyl moiety include: —CH₃, —CH₂OH, —CH₂OC(O)O-cyclohexyl, —CH₂OC(O)O-cyclopentyl, ethyl, isopropyl, NH₂, and —NHC(O)CF₃. In another example the optional substituent is —CH₃.

Examples of R¹⁹ include: —C(O)NH-cyclohexyl, —C(phenyl)₃, H, methyl or ethyl. In one example R¹⁹ is H. In another Example R¹⁹ is —CH₃.

Examples of R²⁰ for group 5.0 include: t-butyl, ethyl, benzyl, —CH(CH₃)₂, —CH₂CH(CH₃)₂, —(CH₂)₂CH₃, n-butyl, n-hexyl, n-octyl, p-chlorophenyl, cyclohexyl, cyclopentyl,

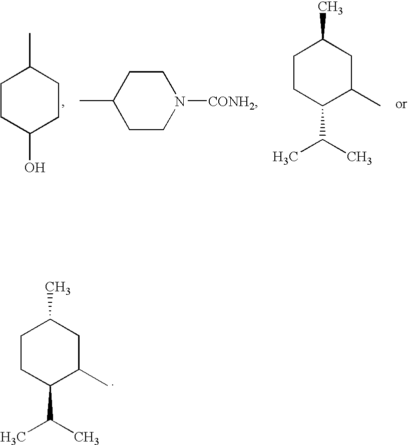

In another example, R²⁰ for group 5.0 is t-butyl.

Examples of R²⁰ and R²¹ for 6.0 include: cyclohexyl, t-butyl, H, —CH(CH₃)₂, ethyl, —(CH₂)₂CH₃, phenyl, benzyl, —(CH₂)₂-phenyl, and —CH₃.

Examples of R²⁰ for 7.0 include: 4-pyridylNO, —OCH₃, —CH(CH₃)₂, -t-butyl, H, propyl, cyclohexyl and

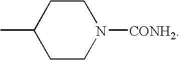

Examples for R³⁶ for 7.1 include: alkyl (such as, for example, t-butyl), cycloalkyl (such as, for example, cyclohexyl, cyclopentyl, cyclobutyl, and cyclopropyl), and heterocycloalkyl (such as, for example

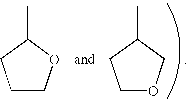

Examples for R²⁰ for 8.0 include: methyl, i-propyl and cyclohexylmethyl.

Examples of R³² and R³³ include: H, phenyl, —OH and benzyl. In one example, R³² and R³³ are H.

Compounds of this invention include compounds of formula 1.0 wherein R¹⁴ is selected from the group consisting of: 6.0, 7.0, 7.1 and 8.0, and R⁸ is selected from the group consisting of: arylalkyl-, substituted arylalkyl-, heteroarylalkyl-, substituted heteroarylalkyl-, cycloalkylalkyl-, and substituted cycloalkylalkyl-.

Compounds of this invention include compounds of formula 1.0 wherein R¹⁴ is 5.0, and R⁸ is selected from the group consisting of: arylalkyl-, substituted arylalkyl-, heteroarylalkyl-, substituted heteroarylalkyl-, cycloalkylalkyl-, and substituted cycloalkylalkyl-.

Compounds of this invention include compounds of formula 1.0 wherein R¹⁴ is 5.0, and R²⁰ is alkyl (e.g., t-butyl).

Compounds of this invention include compounds of formula 1.0 wherein R¹⁴ is 7.1, and R³⁶ is alkyl (e.g., t-butyl).

Compounds of this invention also include compounds wherein R⁸ is H.

Compounds of this invention also include compounds wherein R⁸ is benzyl.

Compounds of this invention also include compounds wherein R¹³ is —C(O)OR⁶⁰ and R⁶⁰ is H.

Compounds of this invention also include compounds wherein R¹³ is —C(O)OR⁶⁰ and R⁶⁰ is alkyl (e.g., ethyl).

Compounds of this invention also include compounds wherein R¹³ is 4.0.

Compounds of this invention also include compounds wherein R¹³ is 4.0, and said 4.0 is substituted.

Compounds of this invention also include compounds wherein R¹³ is 4.0, and said 4.0 is substituted with alkyl.

Compounds of this invention also include compounds wherein R¹³ is 4.0, and said 4.0 is substituted with one alkyl group (e.g., —CH₃).

Compounds of this invention also include compounds wherein R¹³ is 4.0, and said 4.0 is substituted with two independently selected alkyl groups.

Compounds of this invention also include compounds wherein R¹³ is 4.0, and said 4.0 is substituted with two alkyl groups (e.g., each alkyl group is —CH₃).

Compounds of this invention also include compounds wherein R¹³ is 4.0, and said 4.0 is substituted with three independently selected alkyl groups.

Compounds of this invention also include compounds wherein R¹³ is 4.0, and said 4.0 is substituted with three alkyl groups (e.g., each alkyl group is —CH₃).

Compounds of this invention also include compounds wherein: (a) R¹⁴ is 5.0 and R²⁰ is alkyl (e.g., t-butyl), or R¹⁴ is 7.1 wherein R³⁶ is alkyl (e.g. t-butyl), (b) R⁸ is H or benzyl, and (c) R¹³ is —C(O)OR⁶⁰ (e.g., R⁶⁰ is H or alkyl (e.g., ethyl)), or R¹³ is 4.0.

Compounds of this invention include compounds of formula 1.0 wherein R¹⁴ is 5.0, R²⁰ is alkyl (e.g., t-butyl), R⁹ is H, R¹⁰ is H, R³² is H, and R³³ is H.

Compounds of this invention include compounds of formula 1.0 wherein R¹⁴ is 7.1, R³⁶ is alkyl (e.g., t-butyl), R⁹ is H, R¹⁰ is H, R³² is H, and R³³ is H.

Compounds of this invention also include compounds wherein R⁸ is H, R⁹ is H R¹⁰ is H, R³² is H, and R³³ is H.

Compounds of this invention also include compounds wherein R⁸ is benzyl, R⁹ is H, R¹⁰ is H, R³² is H, and R³³ is H.

Compounds of this invention also include compounds wherein R¹³ is —C(O)OR⁶⁰, R⁶⁰ is H, R⁹ is H, R¹⁰ is H, R³² is H, and R³³ is H.

Compounds of this invention also include compounds wherein R¹³ is —C(O)OR⁶⁰, R⁶⁰ is alkyl (e.g., ethyl), R⁹ is H, R¹⁰ is H, R³² is H, and R³³ is H.

Compounds of this invention also include compounds wherein R¹³ is 4.0, R⁹ is H, R¹⁰ is H, R³² is H, and R³³ is H.

Compounds of this invention also include compounds wherein R¹³ is 4.0, said 4.0 is substituted, R⁹ is H, R¹⁰ is H, R³² is H, and R³³ is H.

Compounds of this invention also include compounds wherein R¹³ is 4.0, said 4.0 is substituted with alkyl, R⁹ is H, R¹⁰ is H, R³² is H, and R³³ is H.

Compounds of this invention also include compounds wherein R¹³ is 4.0, said 4.0 is substituted with one alkyl group (e.g., —CH₃), R⁹ is H, R¹⁰ is H, R³² is H, and R³³ is H.

Compounds of this invention also include compounds wherein R¹³ is 4.0, said 4.0 is substituted with two independently selected alkyl groups, R⁹ is H, R¹⁰ is H, R³² is H, and R³³ is H.

Compounds of this invention also include compounds wherein R¹³ is 4.0, said 4.0 is substituted with two alkyl groups (e.g., each alkyl group is —CH₃), R⁹ is H, R¹⁰ is H, R³² is H, and R³³ is H.

Compounds of this invention also include compounds wherein R¹³ is 4.0, said 4.0 is substituted with three independently selected alkyl groups, R⁹ is H, R¹⁰ is H, R³² is H, and R³³ is H.

Compounds of this invention also include compounds wherein R¹³ is 4.0, said 4.0 is substituted with three alkyl groups (e.g., each alkyl group is —CH₃), R⁹ is H, R¹⁰ is H, R³² is H, and R³³ is H.

Compounds of this invention also include compounds wherein: (a) R¹⁴ is 5.0 and R²⁰ is alkyl (e.g., t-butyl), or R¹⁴ is 7.1 wherein R³⁶ is alkyl (e.g. t-butyl), (b) R⁸ is H or benzyl, (c) R¹³ is —C(O)OR⁶⁰ (e.g., R⁶⁰ is H or alkyl (e.g., ethyl)), or R¹³ is 4.0, (d) R⁹ is H, (e) R¹⁰ is H, (f) R³² is H, and (g) R³³ is H.

Compounds of formula 1.0 include compounds described in the embodiments described below. The embodiments have been numbered for ease of reference. The term “as described in any one of Embodiment Numbers”, as used below, means that the particular embodiment using that term is intended to cover any one of the embodiments referred to as if any one of the referred to embodiments had been individually described.

Embodiment No. 1 is directed to compounds of formula 1.0 wherein R¹⁴ is 5.0.

Embodiment No. 2 is directed to compounds of formula 1.0 wherein R¹⁴ is 5.0 and R²⁰ is alkyl.

Embodiment No. 3 is directed to compounds of formula 1.0 wherein R¹⁴ is 5.0 and R²⁰ is t-butyl.

Embodiment No. 4 is directed to compounds of formula 1.0 wherein R⁸ is selected from the group consisting of: H and arylalkyl- (e.g., benzyl).

Embodiment No. 5 is directed to compounds of formula 1.0 wherein R¹³ is selected from the group consisting of —C(O)OR⁶⁰ and imidazolyl ring 4.0.

Embodiment No. 6 is directed to compounds of formula 1.0 wherein R¹³ is —C(O)OR⁶⁰.

Embodiment No. 7 is directed to compounds of formula 1.0 wherein R¹³ is —C(O)OR⁶⁰ and R⁶⁰ is H or ethyl.

Embodiment No. 8 is directed to compounds of formula 1.0 wherein R¹³ is —C(O)OH.

Embodiment No. 9 is directed to compounds of formula 1.0 wherein R¹³ is —C(O)OC₂H₅.

Embodiment No. 10 is directed to compounds of formula 1.0 wherein R⁹ and R¹⁰ are H.

Embodiment No. 11 is directed to compounds of formula 1.0 wherein R³² and R³³ are H, and n is 1 or 2.

Embodiment No. 12 is directed to compounds of formula 1.0 wherein a is N, and b, c and d are —CR¹.

Embodiment No. 13 is directed to compounds of formula 1.0 wherein a is N, and b, c and d are —CR¹ and R¹ is H.

Embodiment No. 14 is directed to compounds of formula 1.0 wherein a is N, c is —CR¹ wherein R¹ is halo (e.g., Br), and b and d are —CR¹ wherein R¹ is H.

Embodiment No. 15 is directed to compounds of formula 1.0 wherein the optional bond between C5 and C6 is absent (i.e., there is a single bond between C5 and C6), and A is H₂ and B is H₂.

Embodiment No. 16 is directed to compounds of formula 1.0 wherein R¹¹ and R¹² are H.

Embodiment No. 17 is directed to compounds of formula 1.0 wherein R¹⁴ is 5.0, and R⁸ is selected from the group consisting of: H and arylalkyl- (e.g., benzyl).

Embodiment No. 18 is directed to compounds of formula 1.0 wherein R¹⁴ is 5.0, R⁸ is selected from the group consisting of: H and arylalkyl- (e.g., benzyl), and R¹³ is selected from the group consisting of —C(O)OR⁶⁰ and imidazolyl ring 4.0.

Embodiment No. 19 is directed to compounds of formula 1.0 R¹⁴ is 5.0, R⁸ is selected from the group consisting of: H and arylalkyl- (e.g., benzyl), R¹³ is selected from the group consisting of —C(O)OR⁶⁰ and imidazolyl ring 4.0, R⁹ and R¹⁰ are H, R³² and R³³ are H, and n is 0 or 1.

Embodiment No. 20 is directed to compounds of formula 1.0 R¹⁴ is 5.0, R⁸ is selected from the group consisting of: H and arylalkyl- (e.g., benzyl), R¹³ is selected from the group consisting of —C(O)OR⁶⁰ and imidazolyl ring 4.0, R⁹ and R¹⁰ are H, R³² and R³³ are H, n is 0 or 1, and R¹¹ and R¹² are H.

Embodiment No. 21 is directed to compounds of formula 1.0 R¹⁴ is 5.0, R⁸ is selected from the group consisting of: H and arylalkyl- (e.g., benzyl), R¹³ is selected from the group consisting of —C(O)OR⁶⁰ and imidazolyl ring 4.0, R⁹ and R¹⁰ are H, R³² and R³³ are H, n is 0 or 1, R⁶⁰ is selected from the group consisting of H and ethyl, and R¹¹ and R¹² are H.

Embodiment No. 22 is directed to compounds of formula 1.0, as described in any one of Embodiment Numbers 17 to 21, wherein a is N, and b, c and d are —CR¹.

Embodiment No. 23 is directed to compounds of formula 1.0, as described in any one of Embodiment Numbers 17 to 21, wherein the optional bond between C5 and C6 is absent (i.e., there is a single bond between C5 and C6), and A is H₂ and B is H₂

Embodiment No. 24 is directed to compounds of formula 1.0, as described in any one of Embodiment Numbers 17 to 21, wherein a is N, and b, c and d are —CR¹, R¹ is H, and the optional bond between C5 and C6 is absent (i.e., there is a single bond between C5 and C6), and A is H₂ and B is H₂.

Embodiment No. 25 is directed to compounds of formula 1.0, as described in any one of Embodiment Numbers 17 to 24, wherein R¹³ is imidazolyl ring 4.0.

Embodiment No. 26 is directed to compounds of formula 1.0, as described in any one of Embodiment Numbers 17 to 24, wherein R¹³ is —C(O)OH.

Embodiment No. 27 is directed to compounds of formula 1.0, as described in any one of Embodiment Numbers 17 to 24, wherein R¹³ is —C(O)OC₂H₅.

Embodiment No. 28 is directed to compounds of formula 1.0, as described in any one of Embodiment Numbers 17 to 27, wherein R⁸ is H.

Embodiment No. 29 is directed to compounds of formula 1.0, as described in any one of Embodiment Numbers 17 to 27, wherein R⁸ is arylalkyl-.

Embodiment No. 30 is directed to compounds of formula 1.0, as described in any one of Embodiment Numbers 17 to 27, wherein R⁸ is benzyl.

Embodiment No. 31 is directed to compounds of formula 1.0, as described in any one of Embodiment Numbers 17 to 30, wherein R³ is halo, and z is 1 or 2.

Embodiment No. 32 is directed to compounds of formula 1.0, as described in any one of Embodiment Numbers 17 to 30, wherein R³ is halo, and z is 1.

Embodiment No. 33 is directed to compounds of formula 1.0, as described in any one of Embodiment Numbers 17 to 30, wherein z is 1 and R³ is Cl.

Embodiment No. 34 is directed to compounds of formula 1.0, as described in any one of Embodiment Numbers 17 to 30, wherein z is 1, and R³ is Cl at the C-8 position.

Embodiment No. 35 is directed to compounds of formula 1.0, as described in any one of Embodiment Numbers 1 to 34, wherein R¹⁴ is 6.0.

Embodiment No. 36 is directed to compounds of formula 1.0, as described in any one of Embodiment Numbers 1 to 34, wherein R⁴ is 7.0.

Embodiment No. 37 is directed to compounds of formula 1.0, as described in any one of Embodiment Numbers 1 to 34, wherein R¹⁴ is 7.1.

Embodiment No. 38 is directed to compounds of formula 1.0, as described in any one of Embodiment Numbers 1 to 34, wherein R¹⁴ is 8.0.

Embodiment No. 39 is directed to compounds of formula 1.0 wherein R¹⁴ is the carbamate group 5.0, R⁸ is cycloalkylalkyl or substituted cycloalkylalkyl (e.g., cycloalkylalkyl).

Embodiment No. 40 is directed to compounds of formula 1.0 wherein when R¹⁴ is group 5.0, and R⁸ is H, then the alkyl chain between R¹³ (i.e., imidazole ring 2.0, 4.0 or 4.1) and the amide moiety (i.e., the —C(O)NR⁸ group) is substituted, i.e.,: (a) at least one of R⁹, R¹⁰, R¹¹, R¹², R³², or R³³ is other than H, and/or (b) R⁹ and R¹⁰, and/or R¹¹ and R¹², are taken together to form a cycloalkyl ring, and the other substituents are as defined for formula 1.0.

Embodiment No. 41 is directed to compounds of formula 1.0 wherein R¹⁴ is a group selected from: 6.0, 7.0, 7.1 or 8.0, R⁸ is arylalkyl or substituted arylalkyl (e.g., arylalkyl) and the other substituents are as defined for formula 1.0.

Embodiment No. 42 is directed to compounds of formula 1.0 wherein R¹⁴ is a group selected from: 6.0, 7.0, 7.1 or 8.0, R⁸ is heteroarylalkyl or substituted heteroarylalkyl (preferably heteroarylalkyl) and the other substituents are as defined for formula 1.0.

Embodiment No. 43 is directed to compounds of formula 1.0 wherein R¹⁴ is a group selected from: 6.0, 7.0, 7.1 or 8.0, R⁸ is cycloalkylalkyl or substituted cycloalkylalkyl (e.g., cycloalkylalkyl) and the other substituents are as defined for formula 1.0.

Embodiment No. 44 is directed to compounds of formula 1.0, as described in any one of Embodiment Numbers 1 to 43, wherein the compound of formula 1.0 is a compound of formula 1.0A.

Embodiment No. 45 is directed to compounds of formula 1.0, as described in any one of Embodiment Numbers 1 to 43, wherein the compound of formula 1.0 is a compound of formula 1.0B.

Embodiment No. 46 is directed to compounds of formula 1.0, as described in any one of Embodiment Numbers 1 to 45, wherein R¹⁴ is 6.0.

Embodiment No. 47 is directed to compounds of formula 1.0, as described in any one of Embodiment Numbers 1 to 45, wherein R¹⁴ is 7.0.

Embodiment No. 48 is directed to compounds of formula 1.0, as described in any one of Embodiment Numbers 1 to 45, wherein R¹⁴ is 7.1.

Embodiment No. 49 is directed to compounds of formula 1.0, as described in any one of Embodiment Numbers 1 to 45, wherein R¹⁴ is 8.0.

Embodiment No. 50 is directed to a compound of formula 1.0 selected from the group consisting of the final compounds of Example Numbers 1, 2, 3, and 4.

Embodiment No. 51 is directed to a pharmaceutically acceptable salt of a compound of formula 1.0, as described in any one of Embodiment Numbers 1 to 50.

Embodiment No. 52 is directed to a pharmaceutical comprising an effective amount of at least one (e.g., 1, 2 or 3, or 1 or 2, or 1, and usually 1) compound as described in any one of Embodiment Numbers 1 to 51, and a pharmaceutically acceptable carrier.

Embodiment No. 53 is directed to a method of treating cancer in a patient in need of such treatment, said method comprising administering to said patient an effective amount of at least one (e.g., 1, 2 or 3, or 1 or 2, or 1, and usually 1) compound as described in any one of Embodiment Numbers 1 to 51.

Embodiment No. 54 is directed to a method of treating cancer in a patient in need of such treatment, said method comprising administering to said patient an effective amount of a pharmaceutical composition, as described in Embodiment No 52.

Embodiment No. 55 is directed to a method of treating cancer in a patient in need of such treatment, said method comprising administering to said patient an effective amount of at least one (e.g., 1, 2 or 3, or 1 or 2, or 1, and usually 1) compound as described in any one of Embodiment Numbers 1 to 51, in combination with an effective amount of at least one (e.g., 1, 2 or 3, or 1 or 2, or 1) chemotherapeutic agent.

Embodiment No. 56 is directed to a method of treating cancer in a patient in need of such treatment, said method comprising administering to said patient an effective amount of a pharmaceutical composition as described in Embodiment Numbers 52, in combination with an effective amount of at least one (e.g., 1, 2 or 3, or 1 or 2, or 1) chemotherapeutic agent.

Embodiment No. 57 is directed to a method of inhibiting farnesyl protein transferase in a patient in need of such treatment, said method comprising administering to said patient an effective amount of at least one (e.g., 1, 2 or 3, or 1 or 2, or 1, and usually 1) compound as described in any one of Embodiment Numbers 1 to 51.

Embodiment No. 58 is directed to a method of inhibiting farnesyl protein transferase in a patient in need of such treatment, said method comprising administering to said patient an effective amount of a pharmaceutical composition as described in Embodiment No. 52.

For the compounds of this invention, R¹, R², R³, and R⁴ are preferably selected from H or halo, and are more preferably selected from H, Br, F, or Cl, and are most preferably selected from H, Br or Cl. Representative compounds of formula 1.0 include trihalo, dihalo and monohalo substituted compounds, such as, for example: (1) 3,8,10-trihalo; (2) 3,7,8-trihalo; (3) 3,8-dihalo; (4) 8-halo; and (5) 10-halo substituted compounds; wherein each halo is independently selected. Compounds of formula 1.0 include: (1) 3-Br,8-Cl-10-Br-substituted compounds; (2) 3-Br,7-Br,8-Cl-substituted compounds; (3) 3-Br,8-Cl-substituted compounds; (4) 8-Cl-substituted compounds; and (5) 10-Cl-substituted compounds. Thus, for example, the compounds of formula 1.0 include 3,8-dihalo compounds. The 8-halo compounds of formula 1.0 are preferred. Thus, for example, 8-Cl substituted compounds are most preferred.

Compounds of formula 1.0 include compounds wherein substituent a is N or N⁺O⁻ with N being preferred.

For compounds of formula 1.0, A and B are preferably H₂, i.e., the optional bond is absent and the C₅-C₆ bridge is unsubstituted.

For compounds of formula 1.0, R⁵, R⁶, and R⁷ are preferably H.

Compounds of formula 1.0 include compounds wherein R⁸ is selected from the group consisting of: H, arylalkyl-, substituted arylalkyl-, heteroarylalkyl-, substituted heteroarylalkyl-, cycloalkylalkyl- and substituted cycloalkylalkyl-. Compounds of formula 1.0 also include compounds wherein R⁸ is selected from the group consisting of: aryl-(C₁-C₄)alkyl-, substituted aryl-(C₁-C₄)alkyl-, heteroaryl-(C₁-C₄)alkyl-, substituted heteroaryl-(C₁-C₄)alkyl-, cycloalkyl-(C₁-C₄)alkyl-, and substituted cycloalkyl-(C₁-C₄)alkyl-. Compounds of formula 1.0 also include compounds wherein R⁸ is selected from the group consisting of: aryl-CH₂—, substituted aryl-CH₂—, heteroaryl-CH₂—, substituted heteroaryl-CH₂, cycloalkyl-CH₂— and substituted cycloalkyl-CH₂—. Compounds of formula 1.0 also include compounds wherein R⁸ is selected from the group consisting of: benzyl, 3-pyridylmethyl, 4-fluoro-benzyl and cyclopropylmethyl. Compounds of formula 1.0 also include compounds wherein R⁸ is benzyl.

Compounds of formula 1.0 also include compounds wherein R¹³ is ring 2.0 or 4.0. When substituted on the substitutable carbon atoms of the imidazole ring, the substituents are generally selected from the group consisting of: —N(R¹⁸)₂, —NHC(O)R¹⁸, —C(R³⁴)₂OR³⁵, or alkyl, e.g., —CH₃, —CH₂OH, —CH₂OC(O)O-cyclohexyl, —CH₂OC(O)O-cyclopentyl, ethyl, isopropyl, NH₂, and —NHC(O)CF₃.

Compounds of formula 1.0 also include compounds wherein R¹⁹ is selected from the group consisting of: H and alkyl, (for example, R¹⁹ is H, methyl or ethyl, or R¹⁹ is methyl.

For compounds of formula 1.0, R¹⁴ is preferably a carbamate group represented by substituent 5.0 described above. Compounds of formula 1.0 include compounds wherein R²⁰ for substituent 5.0 is selected from the group consisting of: alkyl, substituted alkyl, aryl, cycloalkyl, or cycloalkyl substituted with —OH provided that said —OH substituent is not bound to a carbon that is adjacent to an oxygen atom. Compounds of formula 1.0 also include compounds wherein R²⁰ for substituent 5.0 is selected from the group consisting of: C₁ to C₄ alkyl and C₅ to C₇ cycloalkyl. Compounds of formula 1.0 also include compounds wherein R²⁰ for substituent 5.0 is selected from the group consisting of: t-butyl, i-propyl and cyclohexyl, with t-butyl being preferred.

Compounds of formula 1.0 also include compounds wherein R²⁰ in substituent 6.0 is selected from the group consisting of: alkyl and cycloalkyl (for example, t-butyl, isopropyl or cyclohexyl). Compounds of formula 1.0 also include compounds wherein R²¹ is selected from the group consisting of: H and alkyl (for example, H, methyl or isopropyl).

Compounds of formula 1.0 also include compounds wherein R²⁰ in substituent 7.0 is selected from the group consisting of: cycloalkyl and alkyl (for example, cyclohexyl, cyclopentyl, or isopropyl).

Compounds of formula 1.0 also include compounds wherein R³⁶ in substituent 7.1 is selected from the group consisting of: phenyl, cyclopropyl, cyclobutyl, cyclopentyl, cyclohexyl, 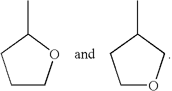
Compounds of formula 1.0 also include compounds wherein R³⁶ in substituent 7.1 is selected from the group consisting of: cyclopropyl, cyclobutyl, cyclopentyl or cyclohexyl.

Compounds of formula 1.0 also include compounds wherein R²⁰ in substituent 8.0 is selected from the group consisting of: alkyl and cycloalkylalkyl (for example, methyl, isopropyl or cyclohexylmethyl). Compounds of formula 1.0 also include compounds wherein R²⁰ in substituent 8.0 is selected from the group consisting of methyl and isopropyl. Compounds of formula 1.0 also include compounds wherein R²⁰ in substituent 8.0 is methyl.

Compounds of formula 1.0 also include compounds wherein R⁹, R¹⁰, R¹¹, and R¹² are selected from the group consisting of: H, C₁ to C₄ alkyl (e.g., methyl or isopropyl), and —CON(R¹⁸)₂ (e.g., —CONH₂), or when R⁹ and R¹⁰, and/or R¹¹ and R¹² are taken together to form a cycloalkyl ring, said ring is cyclopropyl cyclopentyl or cyclohexyl. Preferably, R⁹, R¹⁰, R¹¹, and R¹² are H.

Compounds of formula 1.0 also include compounds wherein R⁹, R¹⁰, R¹¹, and R¹² are H when R¹⁴ is the carbamate substituent 5.0 and R⁸ is not H.

Compounds of formula 1.0 also include compounds wherein when R⁴ is selected from substituents 6.0, 7.0, 7.1 and 8.0, and at least one of R⁹, R¹⁰, R¹¹, and R¹² is other than H, then at least one of R⁹, R¹⁰, R¹¹, and R¹² is:

(I) selected from the group consisting of: (1) C₁ to C₄ alkyl, (2) —CON(R¹⁸)₂ and (3) the cycloalkyl ring formed when R⁹ and R¹⁰, and/or R¹¹ and R², are taken together along with the carbon atom to which they are bound;

(II) selected from the group consisting of: (1) methyl, (2) isopropyl, (3) —CONH₂ and (4) cyclopropyl; and

(III) selected from the group consisting of: (1) R⁹ and R¹⁰ being H, and one of R¹¹ and R¹² being selected from alkyl (e.g., methyl or isopropyl), and the other being selected from H or alkyl (e.g., methyl); (2) R⁹ and R¹⁰ being H, and R¹¹ and R¹² being taken together to form a cycloalkyl ring (e.g., cyclopropyl); and (3) R¹¹ and R¹² being H, and one of R⁹ and R¹⁰ being —CONH₂, and the other being H.

When at least one of R⁹, R¹⁰, R¹¹, and R¹² is other than H, compounds of formula 1.0 also include compounds wherein R⁹ and R¹⁰ are H, and R¹¹ and R¹² are the same or different alkyl, (e.g., the same alkyl, and for example said alkyl is methyl.

Compounds of formula 1.0 also include compounds wherein n is 0-4, or 0-2, and preferably 0 or 1.

Compounds of formula 1.0 also include compounds wherein each R³² and R³³ are independently selected from the group consisting of: H, —OR¹⁸, aryl and arylalkyl (e.g., benzyl). Compounds of formula 1.0 also include compounds wherein R³² and R³³ are independently selected from the group consisting of: H, —OH and phenyl, and preferably H.

Compounds of formula 1.0 include, with reference to the C-11 bond, the R- and S-isomers:

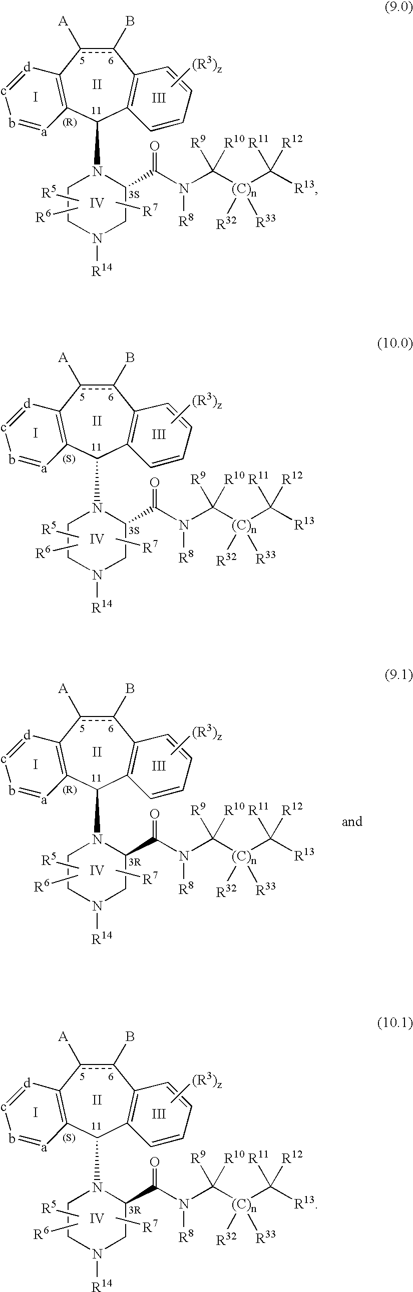

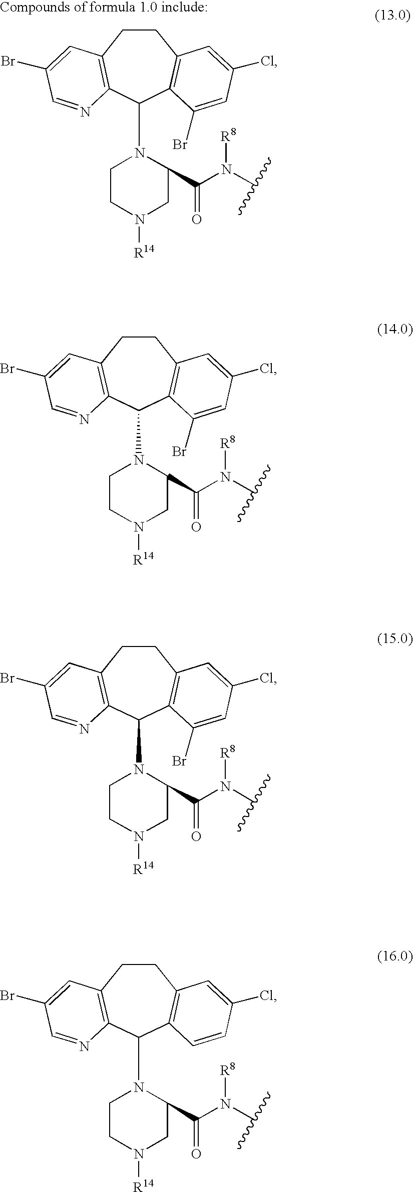

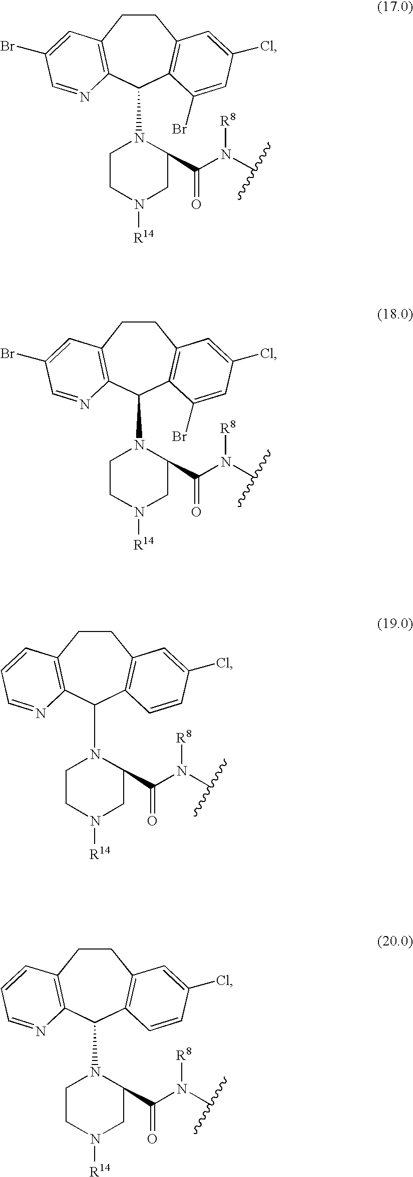

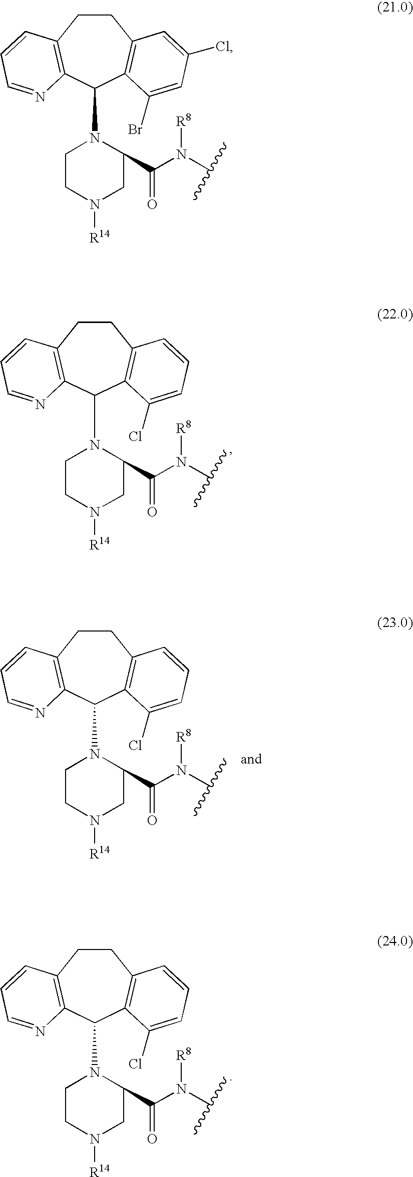

Compounds of the invention also include the 3S counterparts of compounds 13.0 to 20.0, that is compounds whose —C(O)NR⁸ substituent is: 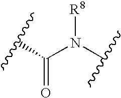
instead of:

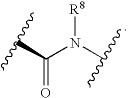

Compounds of the invention also include compounds that have the same structure as compounds 13.0 to 24.0 except that Ring I is a phenyl ring instead of a pyridyl ring.

Compounds of the invention also include compounds that have the same structure as compounds 13.0 to 24.0 except that Ring I is a phenyl ring instead of a pyridyl ring and the —C(O)NR⁸ substituent is 3S: 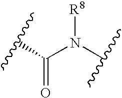
instead of:

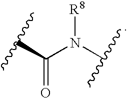

Preferred compounds of formula 1.0 include compounds of the formula: 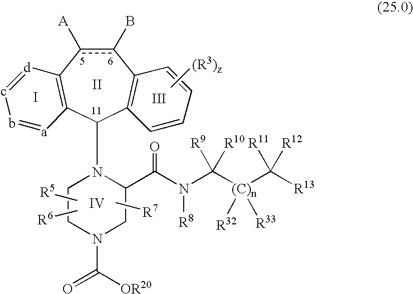
(i.e., wherein R¹⁴ is the carbamate group 5.0) wherein all substituents are as above defined.

A preferred compound of formula 25.0 is: 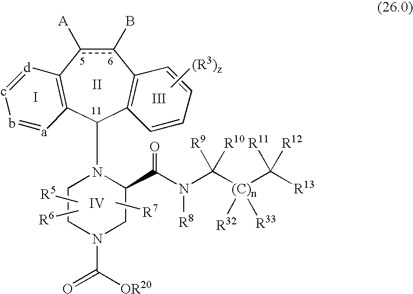
with formula 27.0: 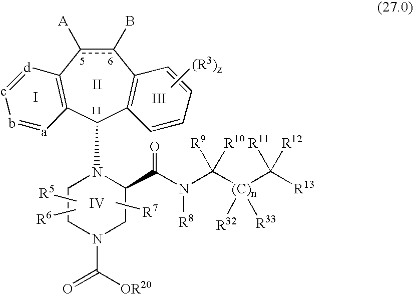
being most preferred (wherein all substituents are as defined above).

Compounds of formula 25.0 include: 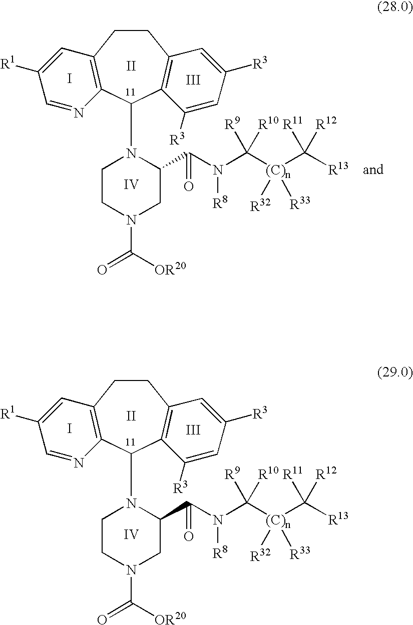
wherein all substituents are as defined above (and each R³ is independently selected).

Preferred compounds of formulas 28.0 and 29.0 are those wherein the R¹ and R³ substituents are selected to produce trihalo, dihalo and monohalo substituted compounds, as described above.

Compounds of formula 29.0 are preferred. Examples of compounds of formula 29.0 include compounds wherein R⁸ is selected from the group consisting of: H, benzyl, 4-fluorobenzyl, 3-pyridylmethyl and cyclopropylmethyl; R²⁰ is selected from the group consisting of: cyclohexyl, i-propyl and t-butyl (and in another example, t-butyl), R¹ is Br or H, R³ at C-8 is Cl, and R³ at C-10 is H. Examples of compound 29.0 also include compounds wherein R⁸ is H, R²⁰ is cyclohexyl, i-propyl or t-butyl (for example, R²⁰ is t-butyl), R¹ is H, R³ is at C-8 is Cl, and R³ at C-10 is H.

Preferred compounds of formula 29.0 include compounds wherein R⁸H; R²⁰ is t-butyl, R¹ is H, R³ at C-8 is Cl, and R³ at C-10 is H.

Representative compounds of this invention include:

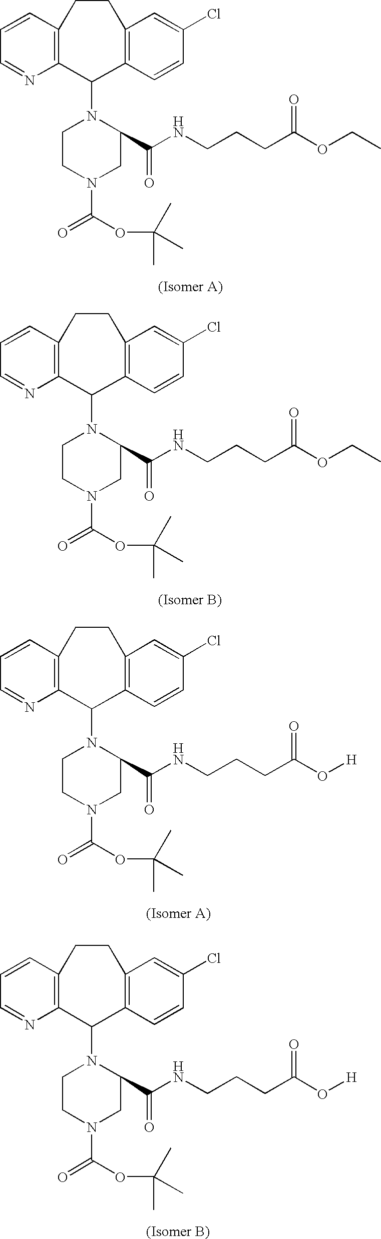

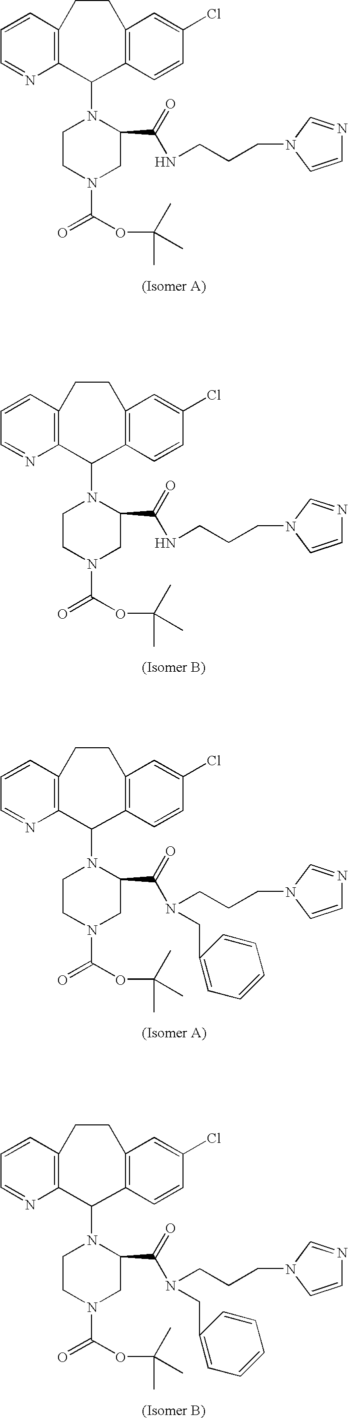

In the compounds above, for the same depicted structure, Isomer A represents one diastereomer, and Isomer B represents another diastereomer.

The compounds of this invention inhibit the activity of farnesyl protein transferase. Thus, this invention provides a method of inhibiting FPT in mammals, especially humans, by the administration of an effective amount (e.g., a therapeutically effective amount) of one or more (e.g., one) compounds of this invention. The administration of the compounds of this invention to patients, to inhibit FPT, is useful in the treatment of cancer.

In any of the methods of treating cancer described herein, unless stated otherwise, the methods can optionally include the administration of an effective amount of one or more (e.g., 1, 2 or 3, or 1 or 2, or 1) chemotherapeutic agents. The chemotherapeutic agents can be administered currently or sequentially with the compounds of this invention.

The methods of treating cancer described herein include methods wherein a combination of drugs (i.e., compounds, or pharmaceutically active ingredients, or pharmaceutical compositions) are used (i.e., the methods of treating cancer of this invention include combination therapies). Those skilled in the art will appreciate that the drugs are generally administered individually as a pharmaceutical composition. The use of a pharmaceutical composition comprising more than one drug is within the scope of this invention.

In any of the methods of treating cancer described herein, unless stated otherwise, the methods can optionally include the administration of an effective amount of radiation therapy. For radiation therapy, □-radiation is preferred.

Examples of cancers which may be treated by the methods of this invention include, but are not limited to: (A) lung cancer (e.g., lung adenocarcinoma and non small cell lung cancer), (B) pancreatic cancers (e.g., pancreatic carcinoma such as, for example, exocrine pancreatic carcinoma), (C) colon cancers (e.g., colorectal carcinomas, such as, for example, colon adenocarcinoma and colon adenoma), (D) myeloid leukemias (for example, acute myelogenous leukemia (AML), CML, and CMML), (E) thyroid cancer, (F) myelodysplastic syndrome (MDS), (G) bladder carcinoma, (H) epidermal carcinoma, (I) melanoma, (J) breast cancer, (K) prostate cancer, (L) head and neck cancers (e.g., squamous cell cancer of the head and neck), (M) ovarian cancer, (N) brain cancers (e.g., gliomas, such as glioma blastoma multiforme), (O) cancers of mesenchymal origin (e.g., fibrosarcomas and rhabdomyosarcomas), (P) sarcomas, (Q) tetracarcinomas, (R) nuroblastomas, (S) kidney carcinomas, (T) hepatomas, (U) non-Hodgkin's lymphoma, (V) multiple myeloma, and (W) anaplastic thyroid carcinoma.

Chemotherapeutic agents (antineoplastic agent) include but are not limited to: microtubule affecting agents, alkylating agents, antimetabolites, natural products and their derivatives, hormones and steroids (including synthetic analogs), and synthetics.

Examples of alkylating agents (including nitrogen mustards, ethylenimine derivatives, alkyl sulfonates, nitrosoureas and triazenes) include: Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Examples of antimetabolites (including folic acid antagonists, pyrimidine analogs, purine analogs and adenosine deaminase inhibitors) include: Methotrexate, 5-Fluorouracil, Floxuridine, Cytarabine, 6-Mercaptopurine, 6-Thioguanine, Fludarabine phosphate, Pentostatine, and Gemcitabine.

Examples of natural products and their derivatives (including vinca alkaloids, antitumor antibiotics, enzymes, lymphokines and epipodophyllotoxins) include: Vinblastine, Vincristine, Vindesine, Bleomycin, Dactinomycin, Daunorubicin, Doxorubicin, Epirubicin, Idarubicin, Paclitaxel (paclitaxel is a microtubule affecting agent and is commercially available as Taxol®), Paclitaxel derivatives (e.g. taxotere), Mithramycin, Deoxyco-formycin, Mitomycin-C, L-Asparaginase, Interferons (especially IFN-a), Etoposide, and Teniposide.

Examples of hormones and steroids (including synthetic analogs) include: 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, and Zoladex.

Examples of synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Examples of other chemotherapeutics include: Navelbene, CPT-11, Anastrazole, Letrazole, Capecitabinbe, Reloxafine, and Droloxafine.

A microtubule affecting agent (e.g., paclitaxel, a paclitaxel derivative or a paclitaxel-like compound), as used herein, is a compound that interferes with cellular mitosis, i.e., having an anti-mitotic effect, by affecting microtubule formation and/or action. Such agents can be, for instance, microtubule stabilizing agents or agents which disrupt microtubule formation.

Microtubule affecting agents, useful in the methods of this invention, are well known to those skilled in the art and include, but are not limited to: Allocoichicine (NSC 406042), Halichondrin B (NSC 609395), Colchicine (NSC 757), Colchicine derivatives (e.g., NSC 33410), Dolastatin 10 (NSC 376128), Maytansine (NSC 153858), Rhizoxin (NSC 332598), Paclitaxel (Taxol®, NSC 125973), Paclitaxel derivatives (e.g., Taxotere, NSC 608832), Thiocolchicine (NSC 361792), Trityl Cysteine (NSC 83265), Vinblastine Sulfate (NSC 49842), Vincristine Sulfate (NSC 67574), Epothilone A, Epothilone, Discodermolide (see Service, (1996) Science, 274:2009), Estramustine, Nocodazole, MAP4, and the like. Examples of such agents are described in, for example, Bulinski (1997) J. Cell Sci. 110:3055-3064, Panda (1997) Proc. Natl. Acad. Sci. USA 94:10560-10564, Muhlradt (1997) Cancer Res. 57:3344-3346, Nicolaou (1997) Nature 387:268-272, Vasquez (1997) Mol. Biol. Cell. 8:973-985, and Panda (1996) J. Biol. Chem. 271:29807-29812.

Chemotherapeutic agents with paclitaxel-like activity include, but are not limited to, paclitaxel and paclitaxel derivatives (paclitaxel-like compounds) and analogues. Paclitaxel and its derivatives (e.g. Taxol and Taxotere) are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

Compounds with possible tubulin polymerization activity can be screened in vitro. For example, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol., 89: 737-758. U.S. Pat. No. 5,569,720 also provides in vitro and in vivo assays for compounds with paclitaxel-like activity.

Thus, in the methods of this invention wherein at least one chemotherapeutic agent is used, examples of said chemotherapeutic agents include those selected from the group consisting of: microtubule affecting agents, alkylating agents, antimetabolites, natural products and their derivatives, hormones and steroids (including synthetic analogs), and synthetics.

In the methods of this invention wherein at least one chemotherapeutic agent is used, examples of said chemotherapeutic agents also include: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

In the methods of this invention wherein at least one chemotherapeutic agent is used, examples of such chemotherapeutic agents include:

(1) taxanes such as paclitaxel (TAXOL®) and/or docetaxel (Taxotere®);

(2) platinum coordinator compounds, such as, for example, carboplatin, cisplatin and oxaliplatin (e.g. Eloxatin);

(3) EGF inhibitors that are antibodies, such as: HER2 antibodies (such as, for example trastuzumab (Herceptin®), Genentech, Inc.), Cetuximab (Erbitux, IMC-C225, ImClone Systems), EMD 72000 (Merck KGaA), anti-EFGRmonoclonal antibody ABX (Abgenix), TheraCIM-h-R³ (Center of Molecular Immunology), monoclonal antibody 425 (Merck KGaA), monoclonal antibody ICR-62 (ICR, Sutton, England); Herzyme (Elan Pharmaceutical Technologies and Ribozyme Pharmaceuticals), PKI 166 (Novartis), EKB 569 (Wyeth-Ayerst), GW 572016 (GlaxoSmithKline), CI 1033 (Pfizer Global Research and Development), trastuzmab-maytansinoid conjugate (Genentech, Inc.), mitumomab (Imclone Systems and Merck KGaA) and Melvax II (Imclone Systems and Merck KgaA);

(4) EGF inhibitors that are small molecules, such as, Tarceva™ (OSI-774, OSI Pharmaceuticals, Inc.), and Iressa (ZD 1839, Astra Zeneca);

(5) VEGF inhibitors that are antibodies such as: bevacizumab (Genentech, Inc.), and IMC-1C11 (ImClone Systems), DC 101 (a KDR VEGF Receptor 2 from ImClone Systems);

(6) VEGF kinase inhibitors that are small molecules such as SU 5416 (from Sugen, Inc), SU 6688 (from Sugen, Inc.), Bay 43-9006 (a dual VEGF and bRAF inhibitor from Bayer Pharmaceuticals and Onyx Pharmaceuticals);

(7) estrogen receptor antagonists or selective estrogen receptor modulators (SERMs), such as tamoxifen, idoxifene, raloxifene, trans-2,3-dihydroraloxifene, levormeloxifene, droloxifene, MDL 103,323, and acolbifene (Schering Corp.);

(8) anti-tumor nucleoside derivatives such as 5-fluorouracil, gemcitabine, capecitabine, cytarabine (Ara-C), fludarabine (F-Ara-A), decitabine, and chlorodeoxyadenosine (Cda, 2-Cda);

(9) epothilones such as BMS-247550 (Bristol-Myers Squibb), and EPO906 (Novartis Pharmaceuticals);

(10) topoisomerase inhibitors such as topotecan (Glaxo SmithKline), and Camptosar (Pharmacia);

(11) vinca alkaloids, such as, navelbine (Anvar and Fabre, France), vincristine and vinblastine;

(12) antibodies that are inhibitors of αVβ3 integrins, such as, LM-609 (see, Clinical Cancer Research, Vol. 6, page 3056-3061, August 2000, the disclosure of which is incorporated herein by reference thereto);

(13) folate antagonists, such as Methotrexate (MTX), and Premetrexed (Alimta);

(14) ribonucleotide reductase inhibitors, such as Hydroxyurea (HU);

(15) anthracyclines, such as Daunorubicin, Doxorubicin (Adriamycin), and Idarubicin;

(16) biologics, such as interferon (e.g., Intron-A and Roferon), pegylated interferon (e.g., Peg-Intron and Pegasys), and Rituximab (Rituxan, antibody used for the treatment of non-Hodgkin's lymphoma);

(17) thalidomide (or related imid);

(18) Bcr/abl kinase inhibitors, such as, for example Gleevec (STI-571), AMN-17, ONO12380, SU11248 (Sunitinib) and BMS-354825

(19) MEK1 and/or MEK2 inhibitors, such as PD0325901 and Arry-142886 (AZD6244);

(20) IGF-1 and IGF-2 inhibitors that are small molecules, such as, for example, NVP-AEW541;

(21) small molecule inhibitors of RAF and BRAF kinases, such as, for example, BAY 43-9006 (Sorafenib);

(22) small molecule inhibitors of cell cycle dependent kinases such as CDK1, CDK2, CDK4 and CDK6, such as, for example, CYC202, BMS387032, and Flavopiridol;

(23) alkylating agents, such as, for example, Temodar® brand of temozolomide;

(24) farnesyl protein transferase inhibitors, such as, for example:
(a) Sarasar® brand of Ionifarnib (i.e., 4-[2-[4-(3,10-dibromo-8-chloro-6,11-dihydro-5H-benzo[5,6]cyclohepta[1,2-b]byridin-11-yl)-1-piperidinyl)-2-oxoethyl]-1-piperidinecarboxamide, see for example, U.S. Pat. No. 5,874,442 issued Feb. 23, 1999, and U.S. Pat. No. 6,632,455 issued Oct. 14, 2003 the disclosures of each being incorporated herein by reference thereto),
(b) compounds of the formula: 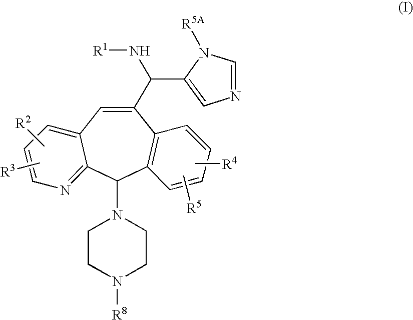
   disclosed in WO 2005/014577 published Feb. 17, 2005, the disclosure of which is incorporated herein by reference thereto, and wherein R¹, R², R³, R⁴, R⁵ and R^{5A} in formula I are as defined in WO 2005/014577.
(c) Zarnestra® brand of tipifarnib (i.e., (R)-6-amino[(4-chlorophenyl)(1-methyl-1H-imidazol-5-yl)methyl]-4-(3-chlorophenyl)-1-methyl-2(1H)-quinolinone, see for example, WO 97/16443 published May 9, 1997 and U.S. Pat. No. 5,968,952 issued Oct. 19, 1999, the disclosures of each being incorporated herein by reference thereto), and
(d) Bristol-Myers Squibb 214662: 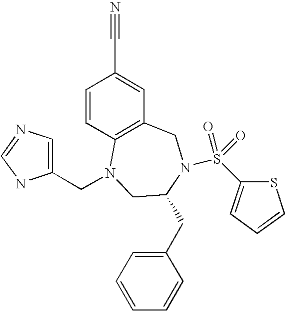
   (see WO97/30992 published Aug. 28, 1997, U.S. Pat. No. 6,011,029 issued Jan. 4, 2000, and U.S. Pat. No. 6,455,523, the disclosures of each being incorporated herein by reference thereto).

Compounds of the formula (see (24)(b) above): 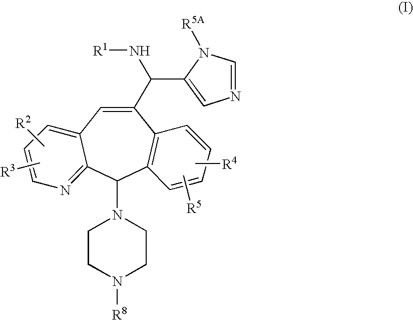
disclosed in WO 2005/014577 published Feb. 17, 2005, have the following definitions of the R¹, R², R³, R⁴, R⁵ and R^{5A} groups: (A) R¹ is selected from the group consisting of: 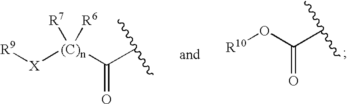
(B) n is 1 to 6; (C) X is selected from the group consisting of O, S, and N; (D) R², R³, R⁴, and R⁵ are independently selected from the group consisting of: H, Br, Cl, and F; (E) R^{5A} is selected from the group consisting of a H, C₁ to C₆ alkyl group, and a C₃ to C₆ cycloalkyl group; (F) R⁶ and R⁷, for each n, are independently selected from the group consisting of: (1) H, (2) C₁ to C₄ alkyl, and (3) a C₃ to C₇ cycloalkyl ring formed by taking R⁶ and R⁷ together with the carbon atom to which they are bonded to; (G) R⁸ is selected from the group consisting of: 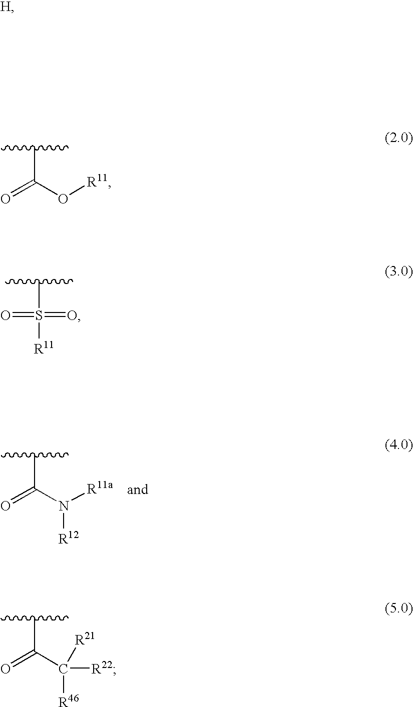
(H) R⁹ is selected from the group consisting of: C₁ to C₆ alkyl group, aryl, heteroaryl, cycloalkyl, heterocycloalkyl, cycloalkylalkyl, heterocycloalkylalkyl, alkenyl, alkynyl, arylalkyl, arylheteroalkyl, cycloalkenyl, heteroalkenyl, heteroalkyl, and heteroalkynyl; (I) or R⁹ is selected from the group consisting of: C₁ to C₆ alkyl group, aryl, heteroaryl, cycloalkyl, heterocycloalkyl, cycloalkylalkyl, heterocycloalkylalkyl, alkenyl, alkynyl, arylalkyl, arylheteroalkyl, cycloalkenyl, heteroalkenyl, heteroalkyl, and heteroalkynyl; wherein (1) said R⁹ aryl, heteroaryl, cycloalkyl, heterocycloalkyl, cycloalkylalkyl, heterocycloalkylalkyl, alkenyl, alkynyl, arylalkyl, arylheteroalkyl, cycloalkenyl, heteroalkenyl, heteroalkyl, and heteroalkynyl groups are substituted with 1 to 3 substituents independently selected from the group consisting of: —OH, halo (e.g., Br, F, or Cl), alkyl (e.g., C₁ to C₆ alkyl), cycloalkyl (e.g., C₃ to C₆, for example cyclopropyl), —NH₂, —NH(C₁ to C₆ alkyl) (e.g., —NHCH₃), —N(C₁ to C₆ alkyl)₂ wherein each alkyl group is independently selected (e.g. —N(CH₃)₂), alkoxy (e.g., methoxy), and —CO₂R¹⁴ wherein R¹⁴ is selected from the group consisting of: H and alkyl (e.g., C₁ to C₆ alkyl, for example methyl and ethyl), provided that the carbon atom, by which said R⁹ group is bonded to the X substituent, is not substituted with a —OH, —NH₂, —NH(C₁ to C₆ alkyl) or —N(C₁ to C₆ alkyl)₂ group; and (2) said R⁹ C₁ to C₆ alkyl group is substituted with 1 to 3 substituents independently selected from the group consisting of: —OH, halo (e.g., Br, F, or Cl), cycloalkyl (e.g., C₃ to C₆, for example cyclopropyl), —NH₂, —NH(C₁ to C₆ alkyl) (e.g., —NHCH₃), —N(C₁ to C₆ alkyl)₂ wherein each alkyl group is independently selected (e.g. —N(CH₃)₂), alkoxy (e.g., methoxy), and —CO₂R¹⁴ wherein R¹⁴ is selected from the group consisting of: H and alkyl (e.g., C₁ to C₆ alkyl, for example methyl and ethyl); provided that the carbon atom, by which said R⁹ group is bonded to the X substituent, is not substituted with a —OH, —NH₂, —NH(C₁ to C₆ alkyl) or —N(C₁ to C₆ alkyl)₂ group; (J) R^{9a} is selected from the group consisting of: alky and arylalkyl; (K) R¹⁰ is selected from the group consisting of: aryl, heteroaryl, cycloalkyl, heterocycloalkyl, cycloalkylalkyl, heterocycloalkylalkyl, alkenyl, alkynyl, arylheteroalkyl, cycloalkenyl, heteroalkenyl, heteroalkyl, and heteroalkynyl; (L) or R¹⁰ is selected from the group consisting of: aryl, heteroaryl, cycloalkyl, heterocycloalkyl, cycloalkylalkyl, heterocycloalkylalkyl, alkenyl, alkynyl, arylheteroalkyl, cycloalkenyl, heteroalkenyl, heteroalkyl, and heteroalkynyl; wherein said R¹⁰ groups are substituted with 1 to 3 substituents independently selected from the group consisting of: —OH, halo (e.g., Br, F, or CI), alkyl (e.g., C₁ to C₆ alkyl), cycloalkyl (e.g., C₃ to C₆, for example cyclopropyl), —NH₂, —NH(C₁ to C₆ alkyl) (e.g., —NHCH₃), —N(C₁ to C₆ alkyl)₂ wherein each alkyl group is independently selected (e.g. —N(CH₃)₂), alkoxy (e.g., methoxy), and —CO₂R¹⁴ wherein R¹⁴ is selected from the group consisting of: H and alkyl (e.g., C₁ to C₆ alkyl, for example methyl and ethyl); (M) R¹¹ is selected from the group consisting of: (1) alkyl (2) substituted alkyl, (3) unsubstituted aryl, (4) substituted aryl, (5) unsubstituted cycloalkyl, (6) substituted cycloalkyl, (7) unsubstituted heteroaryl, (8) substituted heteroaryl, (9) hetero-cycloalkyl, and (10) substituted heterocycloalkyl; wherein said substituted alkyl, substituted cycloalkyl, and substituted heterocycloalkyl R¹¹ groups are substituted with one or more (e.g. 1, 2 or 3) substituents independently selected from the group consisting of: (1) —OH, provided that when there is more than one —OH group then each —OH group is bound to a different carbon atom (i.e., only one —OH group can be bound to a carbon atom), (2) fluoro, and (3) alkyl; and wherein said substituted aryl and substituted heteroaryl R¹¹ groups are substituted with one or more (e.g. 1, 2 or 3) substituents independently selected from the group consisting of: (1) —OH, provided that when there is more than one —OH group then each —OH group is bound to a different carbon atom (i.e., only one —OH group can be bound to a carbon atom), (2) halogen (e.g. Br, Cl or F), and (3) alkyl; (N)R^{11a} is selected from the group consisting of: (1) H, (2) OH, (3) alkyl, (4) substituted alkyl, (5) aryl, (6) substituted aryl, (7) unsubstituted cycloalkyl, (8) substituted cycloalkyl, (9) unsubstituted heteroaryl, (10) substituted heteroaryl, (11) heterocycloalkyl, (12) substituted heterocycloalkyl, and (13) —OR^{9a}; wherein said substituted alkyl, substituted cycloalkyl, and substituted heterocycloalkyl R^{11a} groups are substituted with one or more (e.g. 1, 2 or 3) substituents independently selected from the group consisting of: (1) —OH, provided that when there is more than one —OH group then each —OH group is bound to a different carbon atom (i.e., only one —OH group can be bound to a carbon atom), (2) —CN, (3) —CF₃, (4) fluoro, (5) alkyl, (6) cycloalkyl, (7) heterocycloalkyl, (8) arylalkyl, (9) heteroarylalkyl, (10) alkenyl and (11) heteroalkenyl; and wherein said substituted aryl and substituted heteroaryl R^{11a} groups have one or more (e.g. 1, 2 or 3) substituents independently selected from the group consisting of: (1) —OH, provided that when there is more than one —OH group then each —OH group is bound to a different carbon atom (i.e., only one —OH group can be bound to a carbon atom), (2) —CN, (3) —CF₃, (4) halogen (e.g Br, Cl or F), (5) alkyl, (6) cycloalkyl, (7) heterocycloalkyl, (8) arylalkyl, (9) heteroarylalkyl, (10) alkenyl, and (11) heteroalkenyl; (O)R¹² is selected from the group consisting of: H, alkyl, piperidine Ring V, cycloalkyl, and -alkyl-(piperidine Ring V), wherein piperidine Ring V is 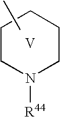
wherein R⁴⁴ is defined below; (P) R²¹, R²² and R⁴⁶ are independently selected from the group consisting of: (1) —H, (2) alkyl (e.g., methyl, ethyl, propyl, butyl or t-butyl), (3) unsubstituted aryl, (e.g. phenyl), (4) substituted aryl substituted with one or more substituents independently selected from the group consisting of: alkyl, halogen, CF₃ and OH, (5) unsubstituted cycloalkyl, (e.g. cyclohexyl), (6) substituted cycloalkyl substituted with one or more substituents independently selected from the group consisting of: alkyl, halogen, CF₃ and OH, (7) heteroaryl of the formula 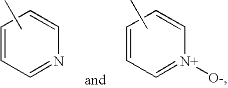
(8) heterocycloalkyl of the formula: 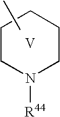
(i.e., piperidine Ring V) wherein R⁴⁴ is selected from the group consisting of: (a) —H, (b) alkyl (e.g., methyl, ethyl, propyl, butyl or t-butyl), (c) alkylcarbonyl (e.g., CH₃C(O)—), (d) alkyloxycarbonyl (e.g., —C(O)O-t-C₄H₉, —C(O)OC₂H₅ and —C(O)OCH₃), (e) haloalkyl (e.g., trifluoromethyl), and (f) —C(O)NH(R⁵¹),
   (9) —NH₂ provided that only one of R²¹, R²², and R⁴⁶ group can be —NH₂ and provided that when one of R², R²², and R⁴⁶ is —NH₂ then the remaining groups are not —OH, (10) —OH provided that only one of R²¹, R²², and R⁴⁶ group can be —OH and provided that when one of R², R²², and R⁴⁶ is —OH then the remaining groups are not —NH₂, and (11) alkyl substituted with one or more substituents (e.g., 1-3, or 1-2, and preferably 1) selected from the group consisting of: —OH and —NH₂ and provided that there is only one —OH or one —NH₂ group on a substituted carbon, or (Q) R²¹ and R²² taken together with the carbon to which they are bound form a cyclic ring selected from the group consisting of: (1) unsubstituted cycloalkyl (e.g., cyclopropyl, cyclobutyl, cyclopentyl, and cyclohexyl), (2) cycloalkyl substituted with one or more substituents independently selected from the group consisting of: alkyl, halogen, CF₃ and OH, (3) unsubstituted cycloalkenyl 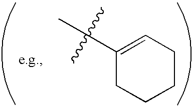
   (4) cycloalkenyl substituted with one or more substituents independently selected from the group consisting of: alkyl, halogen, CF₃ and OH, (5) heterocycloalkyl, e.g., a piperidyl ring of the formula: 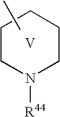
wherein R⁴⁴ is selected from the group consisting of: (a) —H, (b) alkyl (e.g., methyl, ethyl, propyl, butyl or t-butyl), (c) alkylcarbonyl (e.g., CH₃C(O)—), (d) alkyloxy carbonyl (e.g., —C(O)O-t-C₄H₉, —C(O)OC₂H₅, and —C(O)OCH₃), (e) haloalkyl (e.g., trifluoromethyl), and (f) —C(O)NH(R⁵¹),
   (6) unsubstituted aryl (e.g., phenyl), (7) aryl substituted with one or more substituents independently selected from the group consisting of: alkyl (e.g., methyl), halogen (e.g., Cl, Br and F), —CN, —CF₃, OH and alkoxy (e.g., methoxy), and (8) heteroaryl selected from the group consisting of: 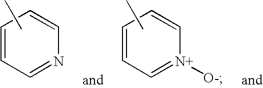
   (R) R⁵¹ is selected from the group consisting of: H and alkyl (e.g., methyl, ethyl, propyl, butyl and t-butyl). For the compounds of formula I, R², R³, R⁴, and R⁵ are preferably independently selected to form an unsubstituted (i.e., R² to R⁵ are H), or a monohalo, dihalo, or trihalo substituted ring system, wherein halo is selected from the group consisting of: Br, Cl and F. Examples of such halo substitutions are: 8-halo (e.g., 8-Cl), 3,8-dihalo (e.g., 3-Br-8-Cl), 3,7,8-trihalo (e.g., 3-Br-7-Br-8-Cl) and 3,8,10-trihalo (e.g., 3-Br-8-Cl-10-Br). A mono halo substituted ring system is preferred, with 8-halo being more preferred, and 8-Cl being most preferred. The compound of formula I is preferably a compound of formula II: 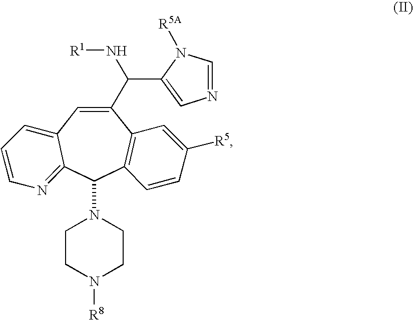
   and more preferably a compound of formula III: 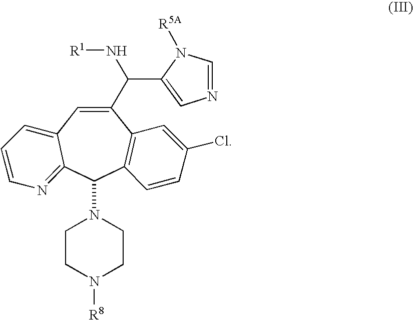
   The compound of formula I is more preferably a compound of formula IIA 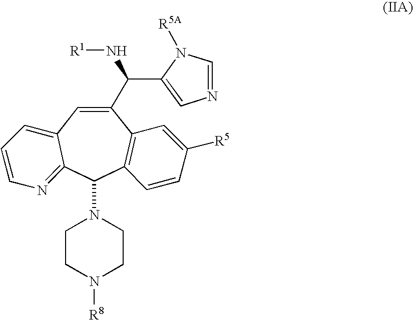
   and even more preferably a compound of formula IIIA 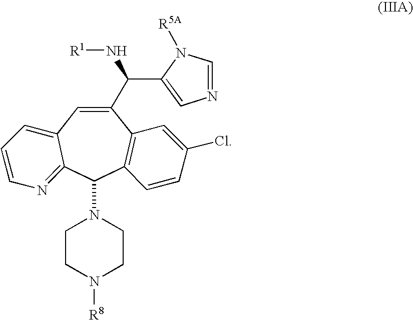
   Compounds of formula I include compounds of formula IV: 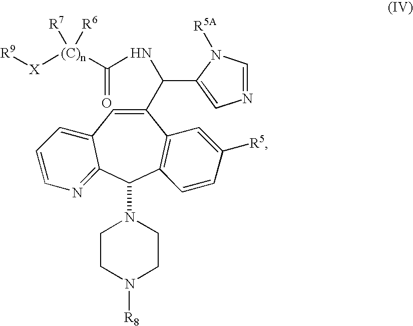
   and preferably a compound of formula V: 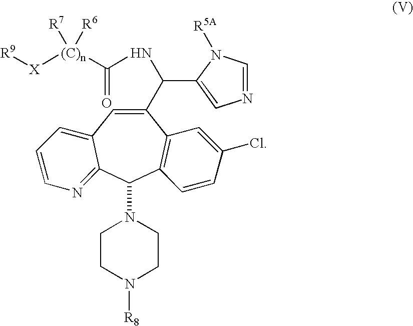
   Compounds of formula I include compounds of formula IVA 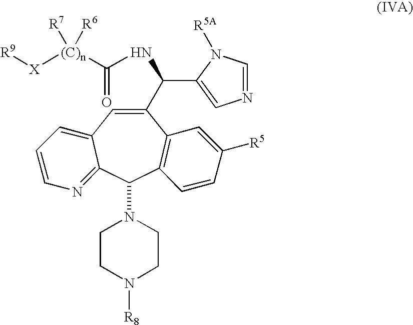
   and preferably compounds of formula VA: 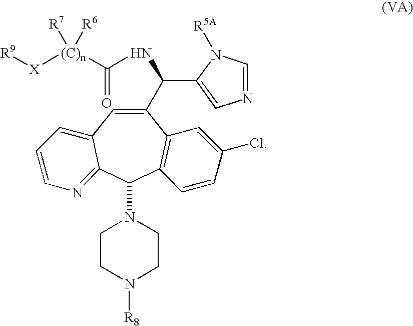
   Compounds of formula I also include compounds of formula VIII: 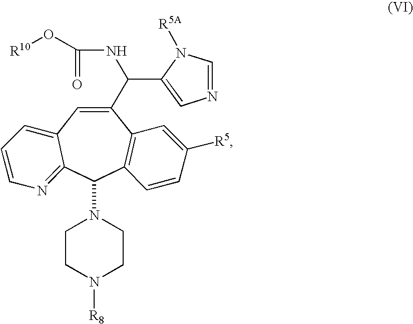
   and preferably compounds of formula VII: 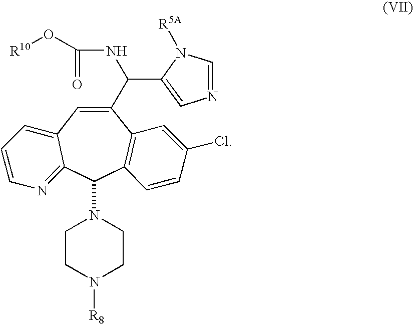
   Compounds of formula I also include compounds of formula VIA: 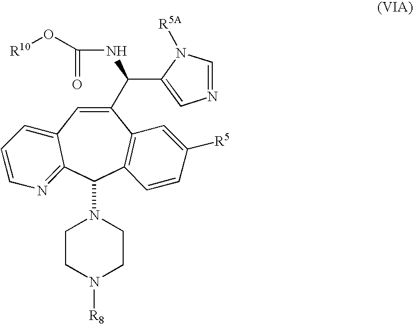
   and preferably compounds of formula VIIA: 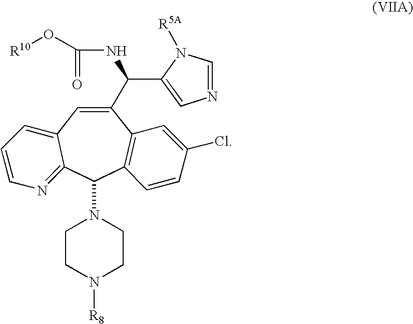
   For compounds of formula I, examples of R^{5A} include, but are not limited to: H, methyl, ethyl, isopropyl and cyclopropyl. For compounds of formula I, R^{5A} is preferably C₁ to C₆ alkyl with methyl being most preferred. For compounds of formula I, X is preferably O. For compounds of formula I, n is preferably 1. For compounds of formula I, R⁶ and R⁷ are preferably independently selected from the group consisting of H, methyl and the cyclopropyl ring formed when R⁶ and R⁷ are taken together with the carbon atom to which they are bonded to. More preferably R⁶ and R⁷ are independently selected from the group consisting of H and methyl. Most preferably R⁶ and R⁷ are H. For compounds of formula I, R⁹ is preferably C₁ to C₆ alkyl, and more preferably methyl. For compounds of formula I, R¹⁰ is preferably selected from the group consisting of: cycloalkyl and cycloalkyl substituted with a C₁ to C₆ alkyl group, more preferably selected from the group consisting of cycloalkyl and cycloalkyl substituted with methyl, most preferably selected from the group consisting of: cyclopropyl and cyclopropyl substituted with a methyl group, and even more preferably R¹⁰ is: 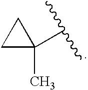
   For compounds of formula I, when R¹ is 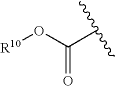
   then R⁸ is preferably 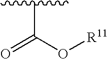
   wherein the R¹¹ substituent is the same as the R¹⁰ substituent. For example, when R¹ is: 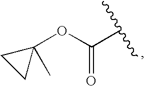
   then R⁸ is preferably 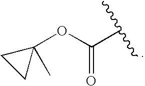
   For compounds of formula I, R⁸ is preferably 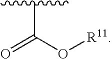
   For compounds of formula I, R⁸ is more preferably 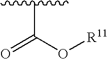
   wherein R¹¹ is selected from the group consisting of: alkyl, unsubstituted cycloalkyl and substituted cycloalkyl. Most preferably, R¹¹ is selected from the group consisting of: alky and substituted cycloalkyl. Even more preferably, R¹¹ is selected from the group consisting of: isopropyl, and cyclopropyl substituted with methyl, i.e., the group 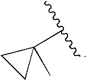
   For compounds of formula I, wherein R¹ is 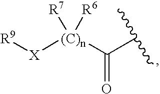
   X is O, n is 1, R⁶ and R⁷ are independently selected from the group consisting of H, methyl and the cyclopropyl ring formed when R⁶ and R⁷ are taken together with the carbon atom to which they are bonded to (wherein preferably R⁶ and R⁷ are independently selected from the group consisting of H and methyl, and more preferably R⁶ and R⁷ are H), and R⁹ is C₁ to C₆ alkyl (preferably methyl), R⁸ is preferably 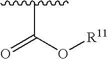
   wherein R¹¹ is preferably alkyl (more preferably isopropyl). For compounds of formula I, wherein R¹ is 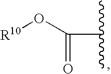
   R¹⁰ is selected from the group consisting of: cycloalkyl and cycloalkyl substituted with a C₁ to C₆ alkyl group (preferably R¹⁰ selected from the group consisting of cycloalkyl and cycloalkyl substituted with methyl, and more preferably selected from the group consisting of: cyclopropyl and cyclopropyl substituted with a methyl group, and most preferably R¹⁰ is: 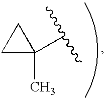
   R⁸ is preferably 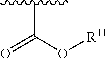
   wherein R¹¹ is selected from the group consisting of: unsubstituted cycloalkyl and substituted cycloalkyl (preferably, R¹¹ is substituted cycloalkyl, and more preferably, R¹¹ is cyclopropyl substituted with methyl, i.e., the group 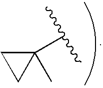
   Examples of the compounds of formula I include, for example, the compounds of formulas 100 to 174 are: 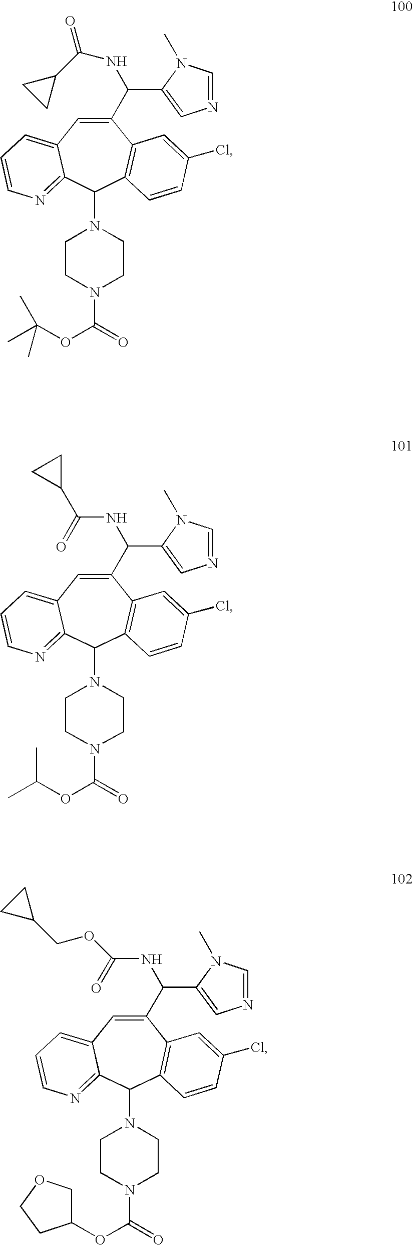 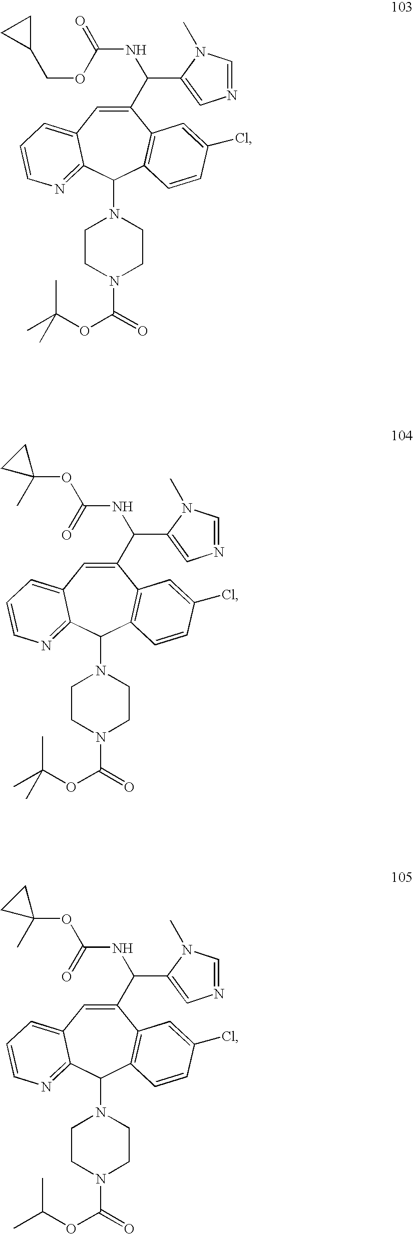 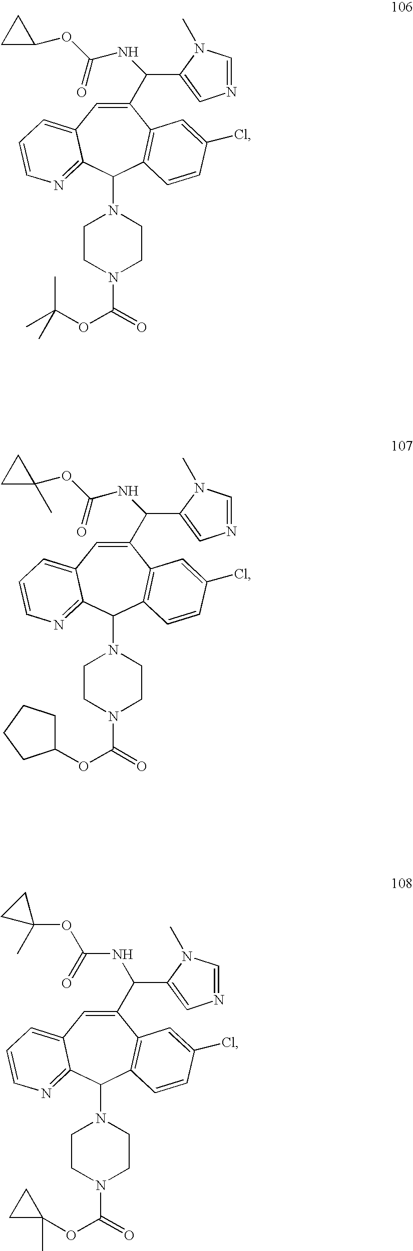 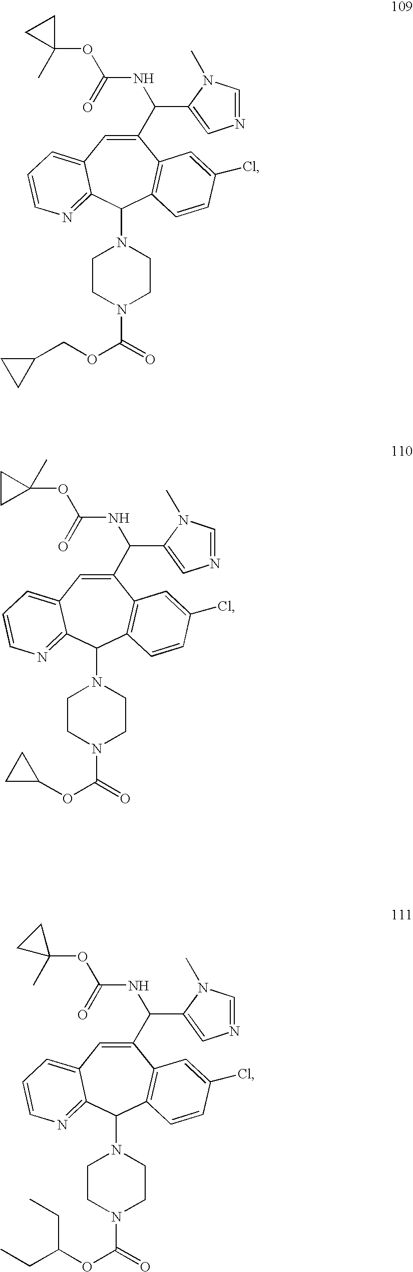 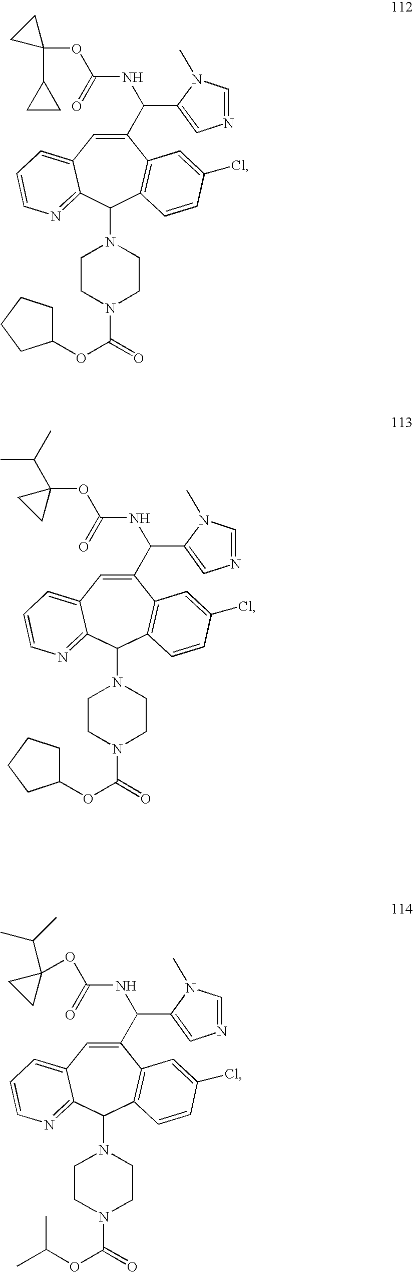 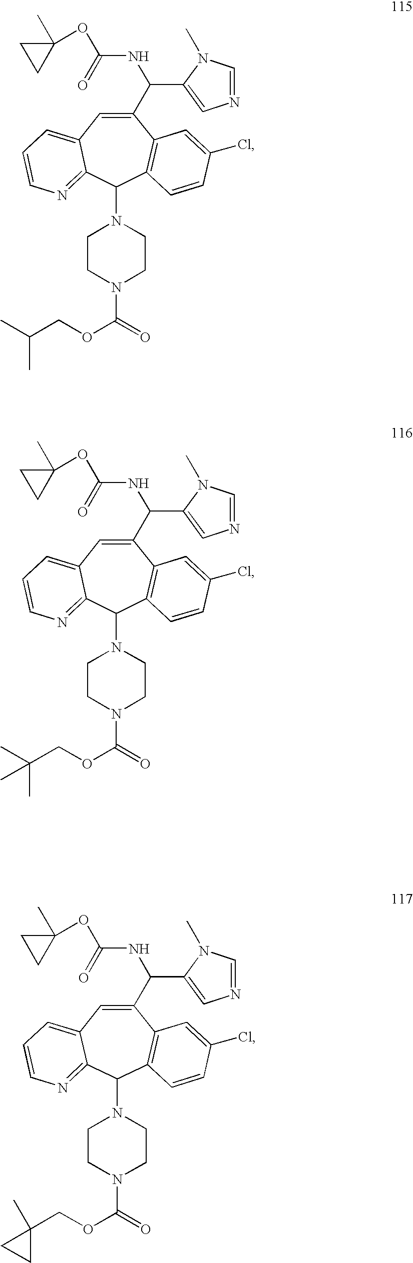 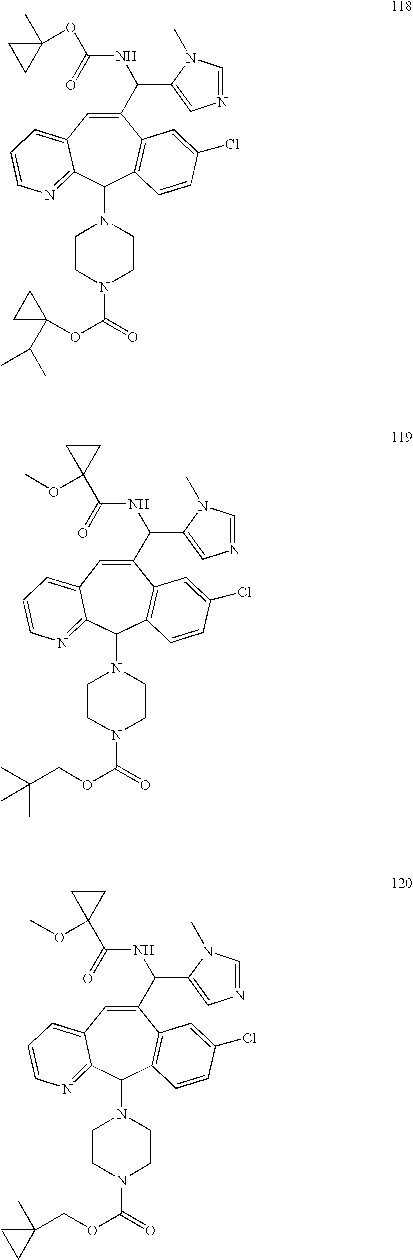 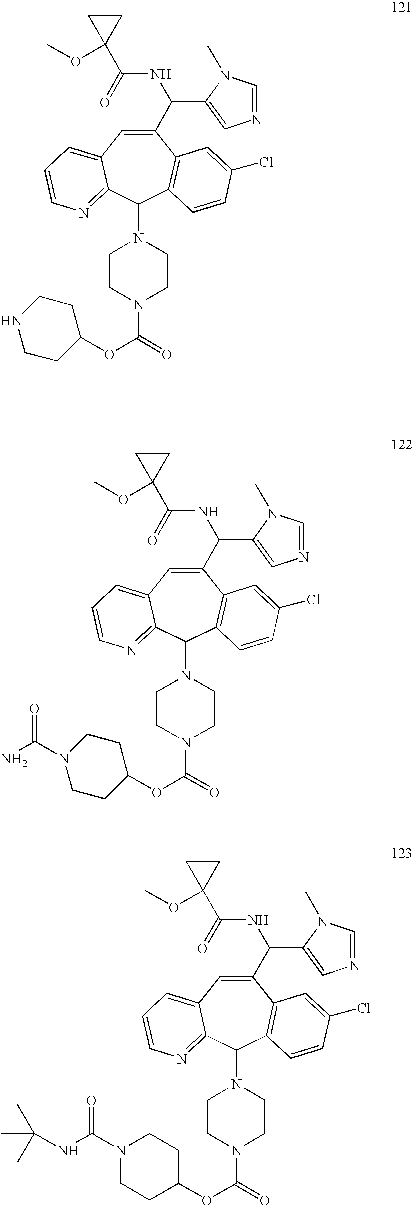 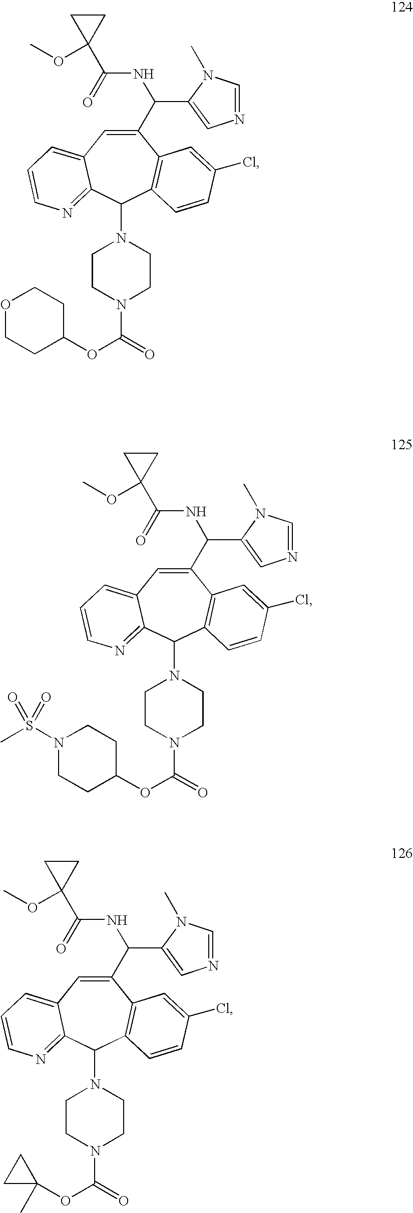 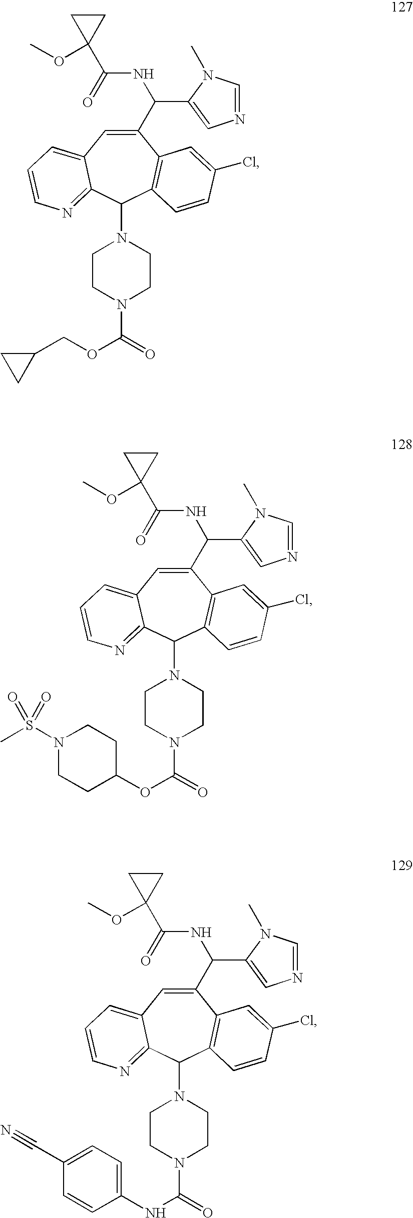 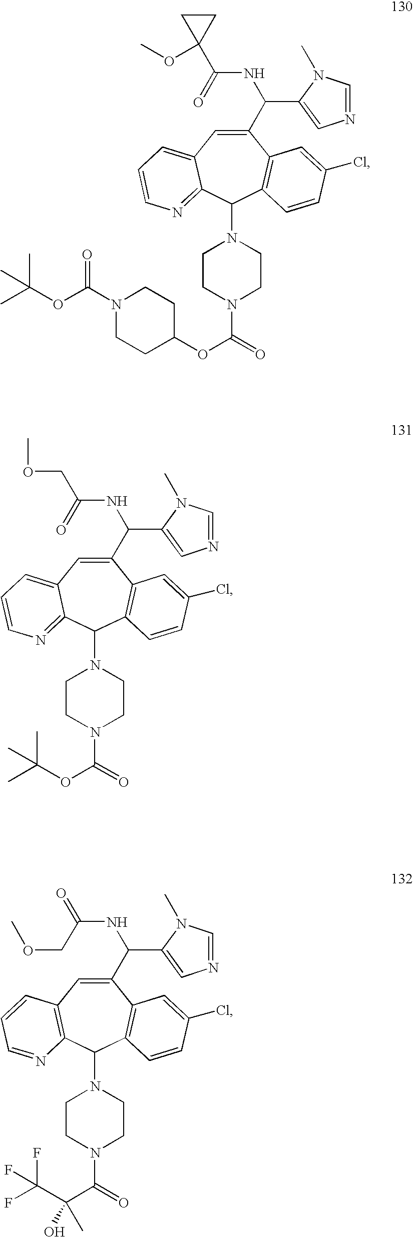 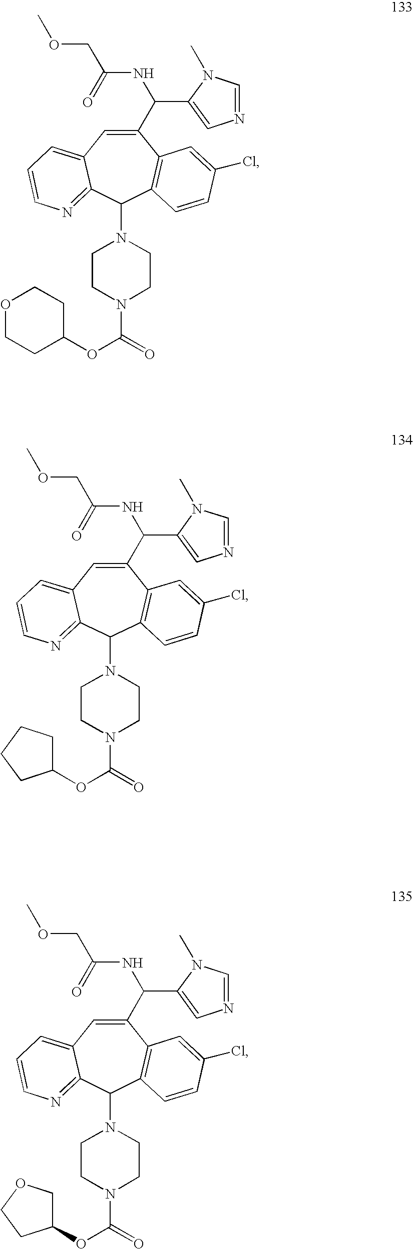 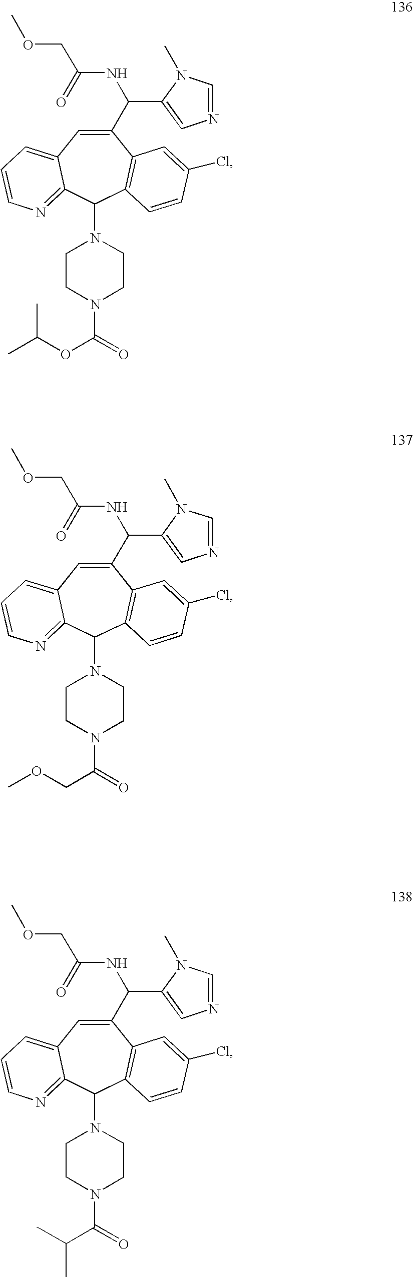 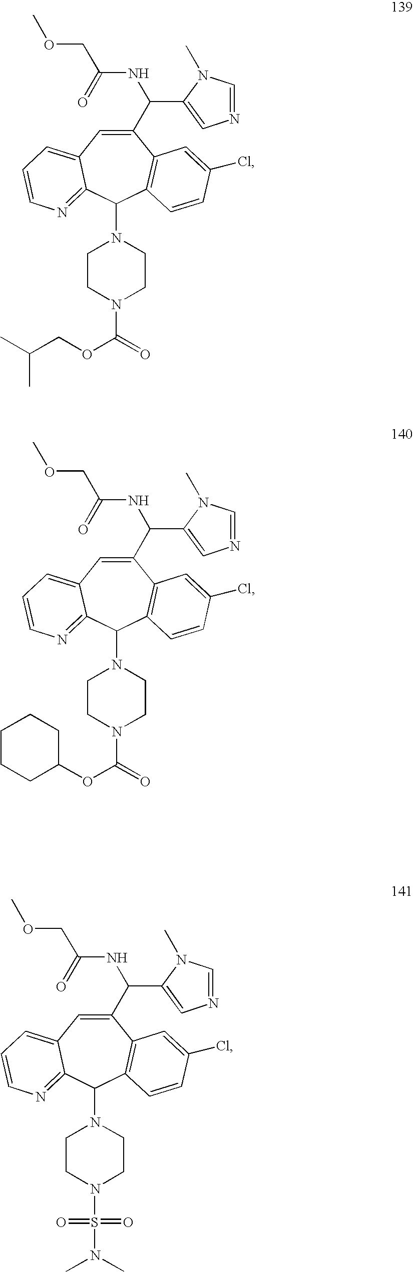 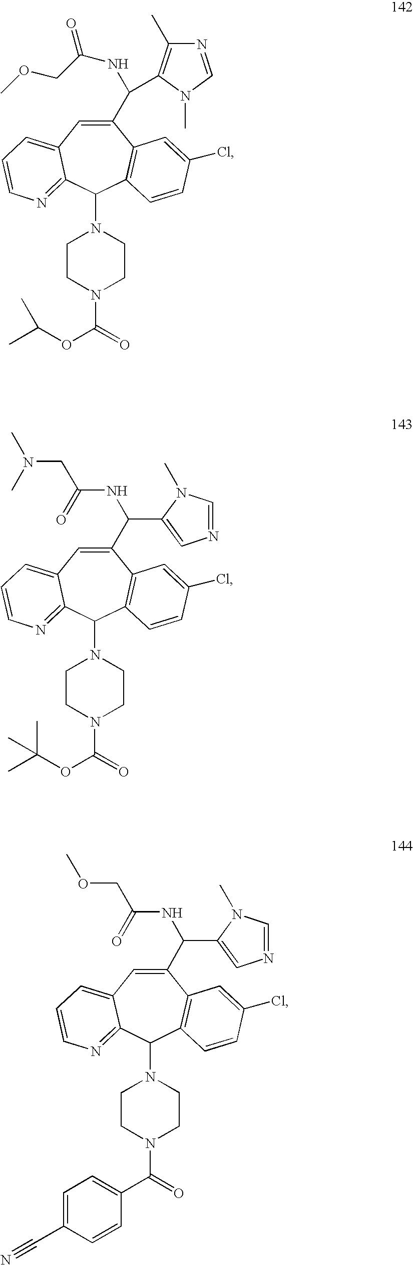 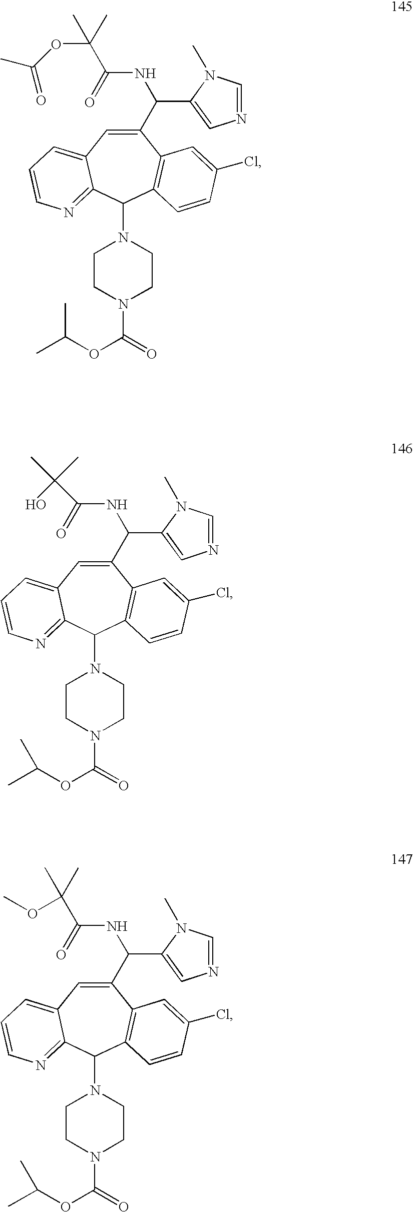 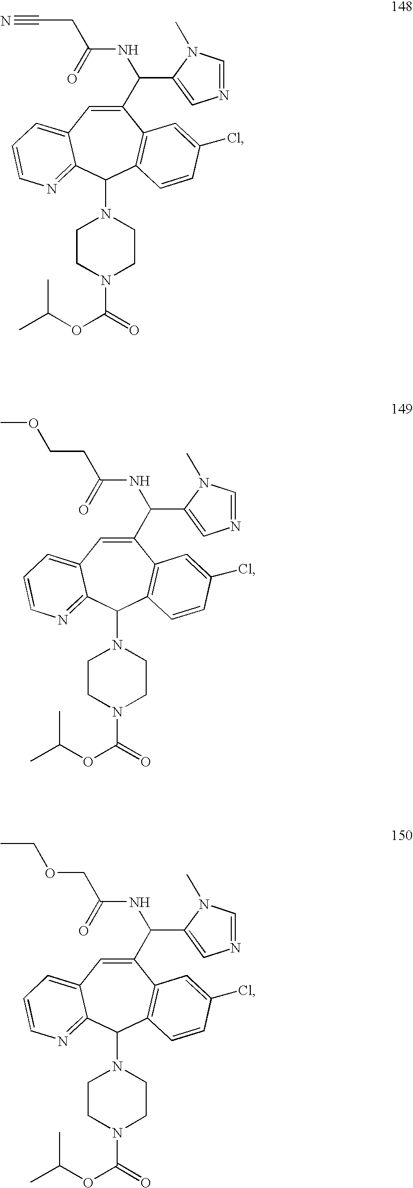 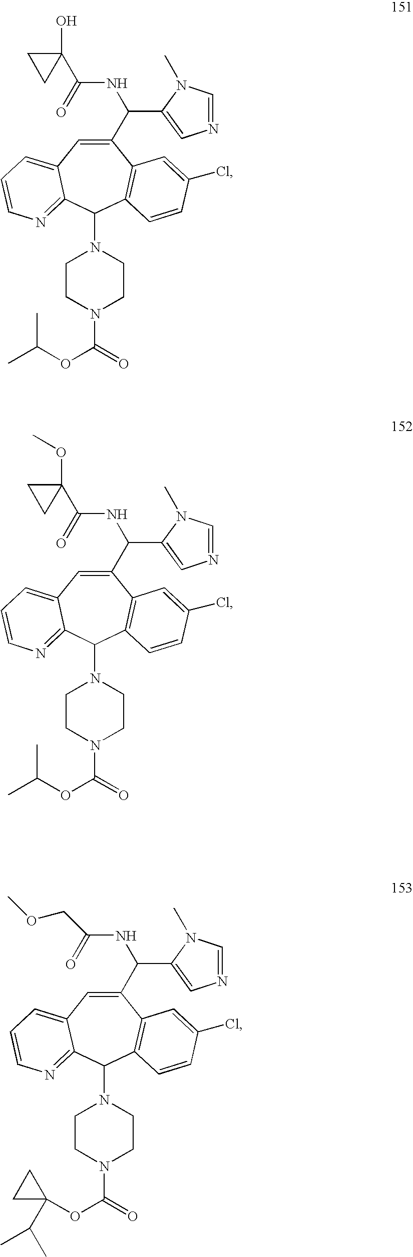 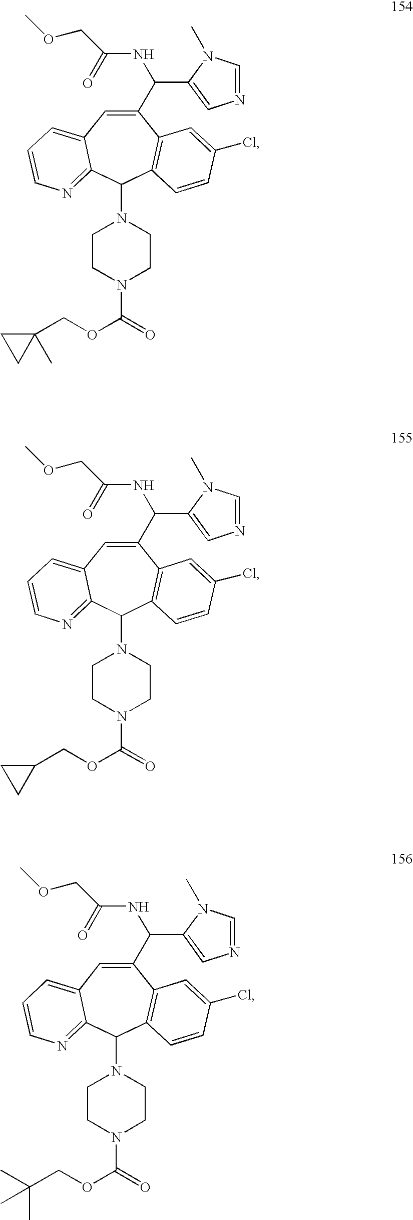 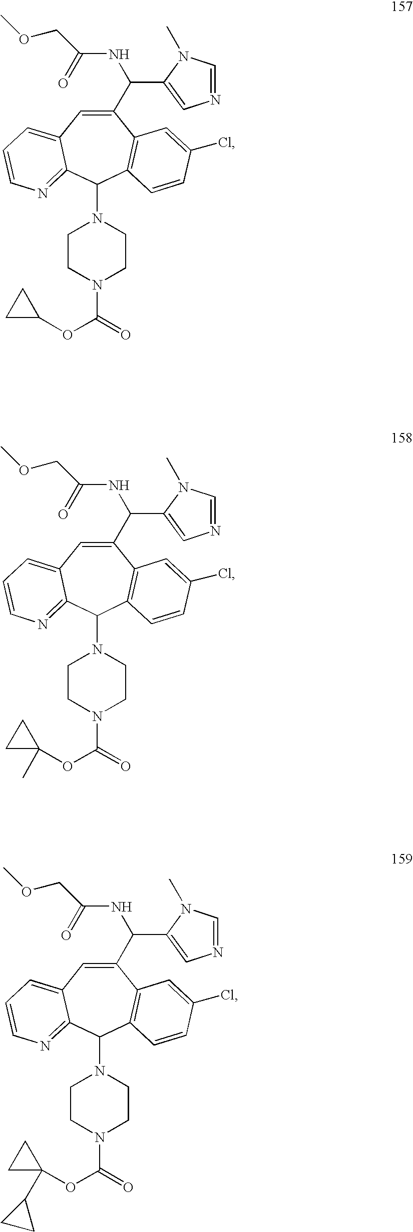 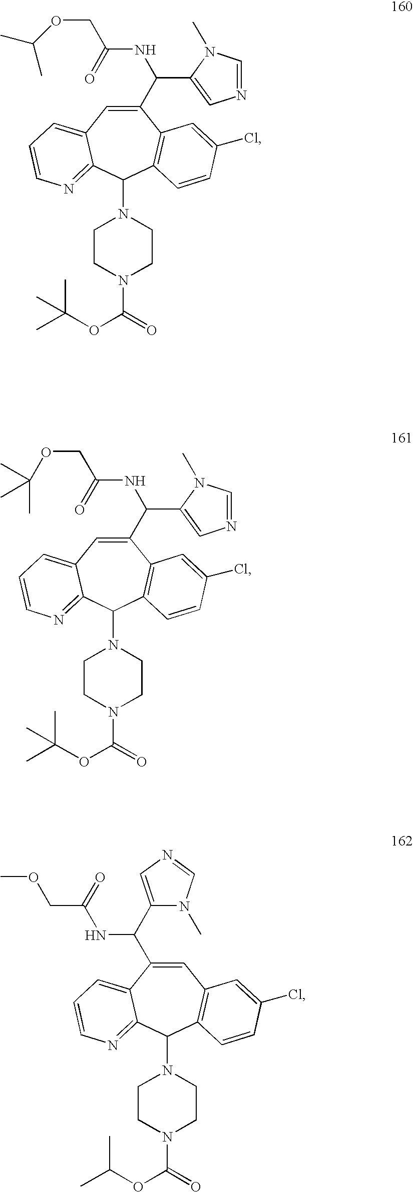 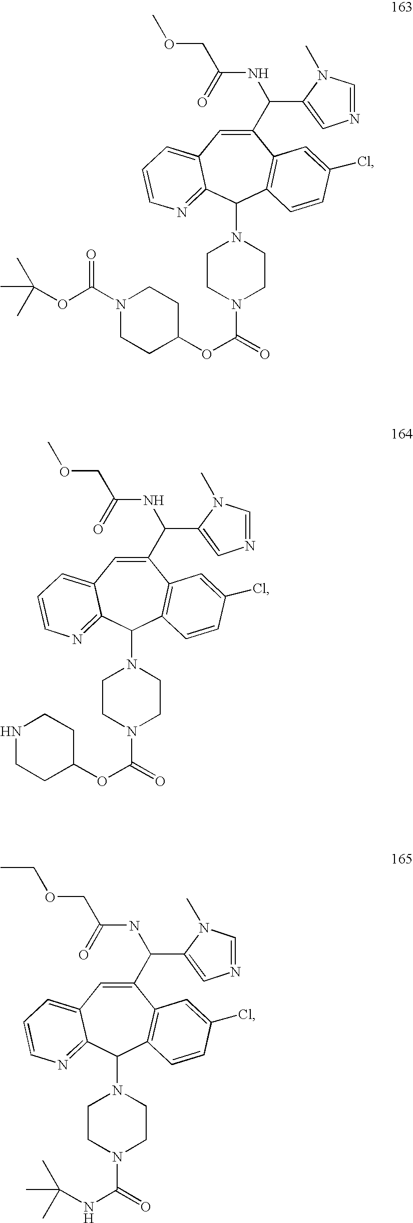 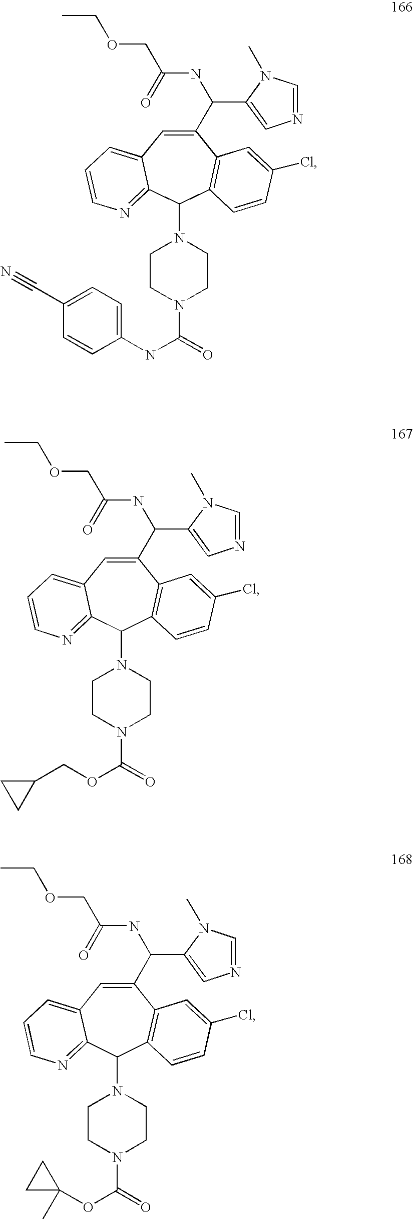 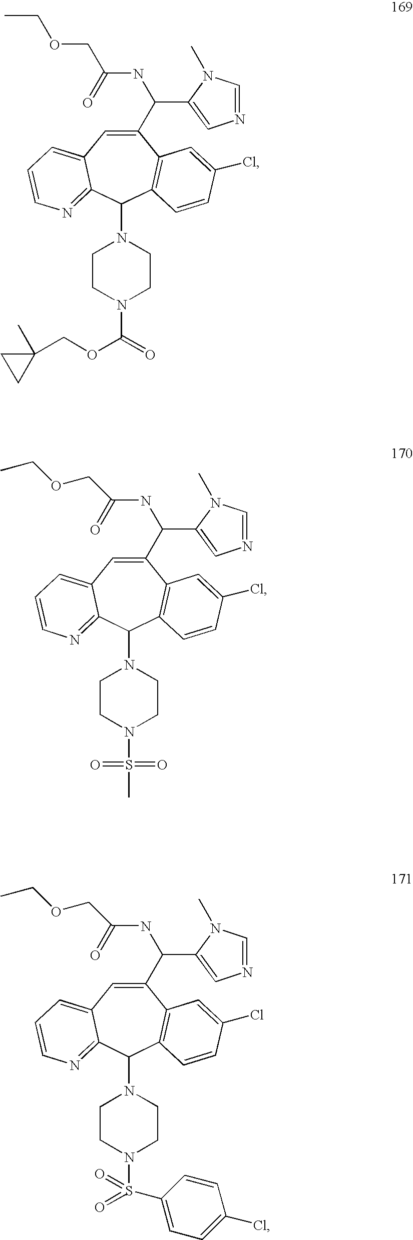 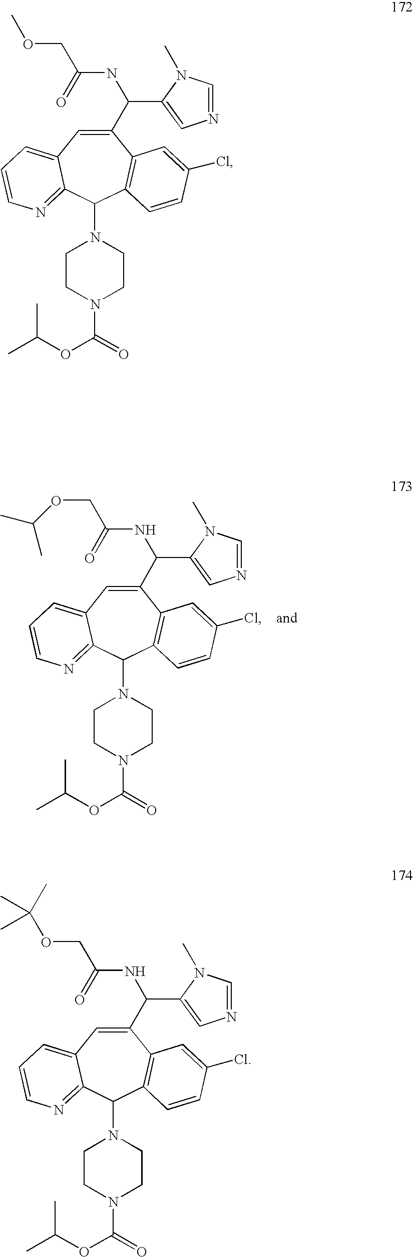
   Representative compounds of compounds 100 to 174 of formula I include, but are not limited to: 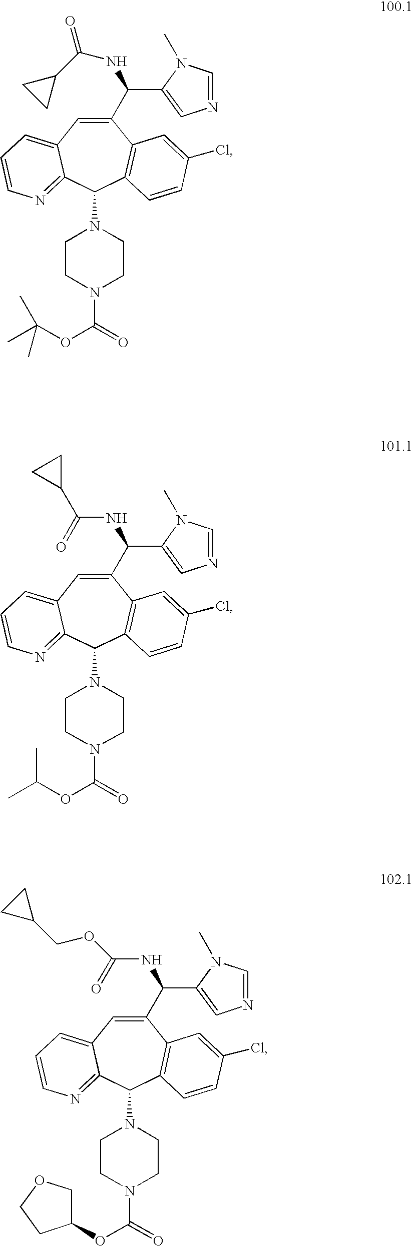 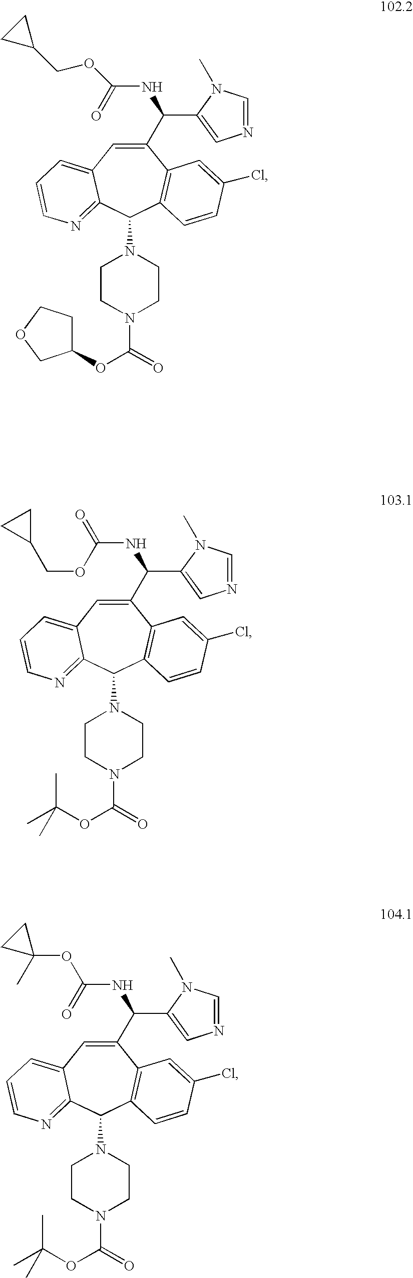 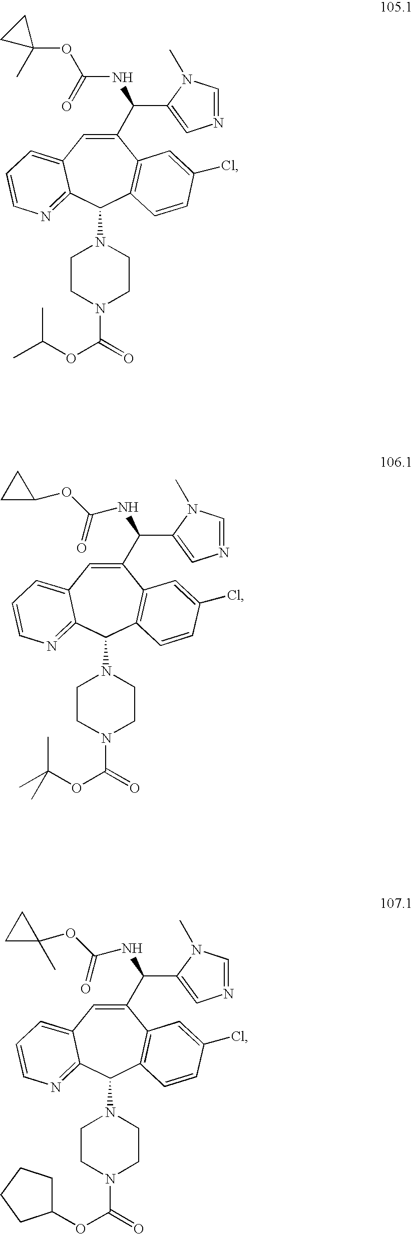 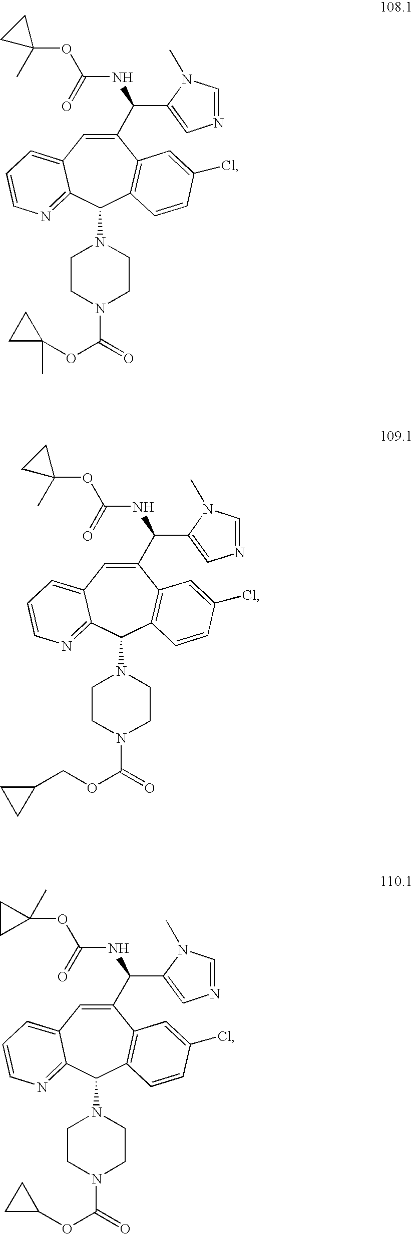 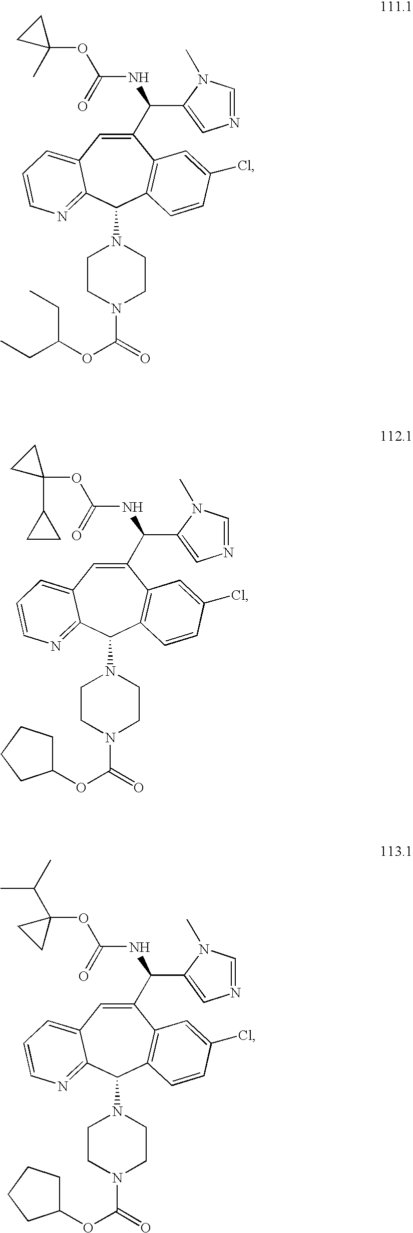 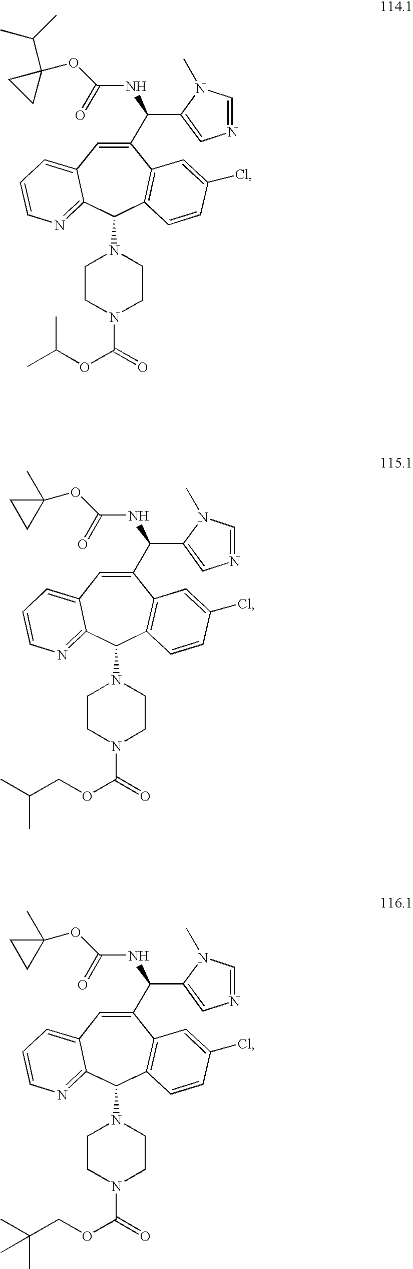 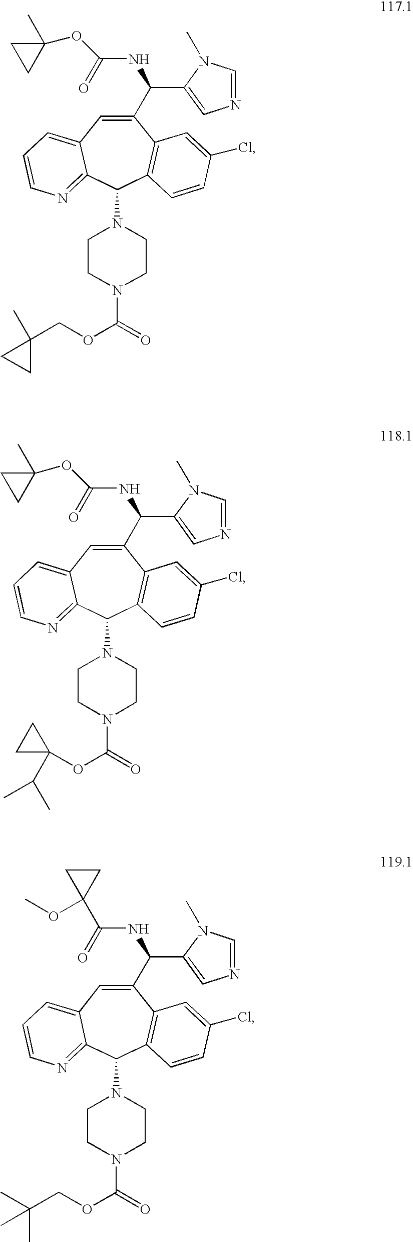 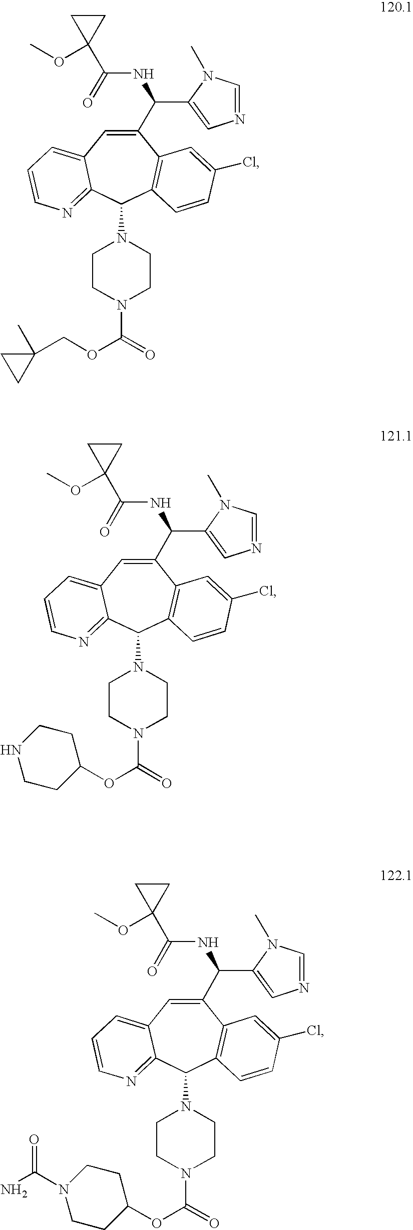 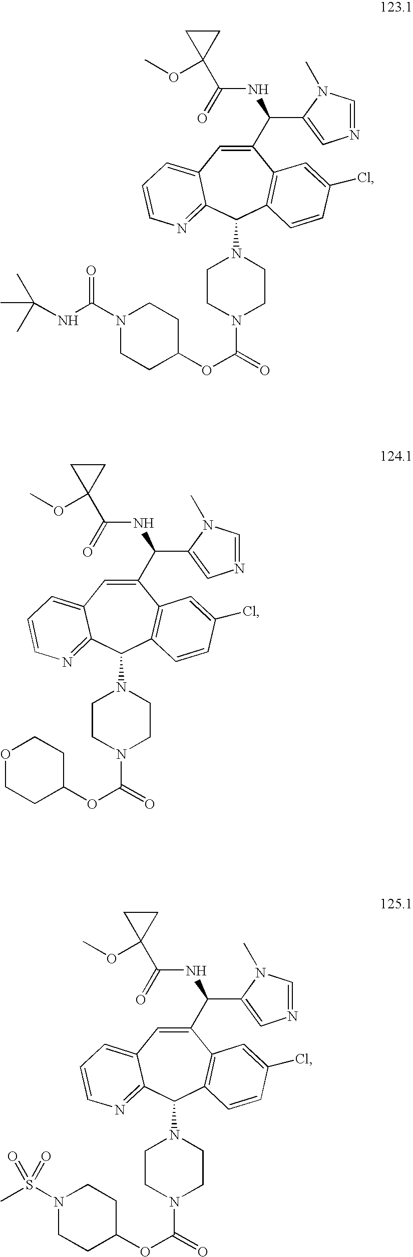 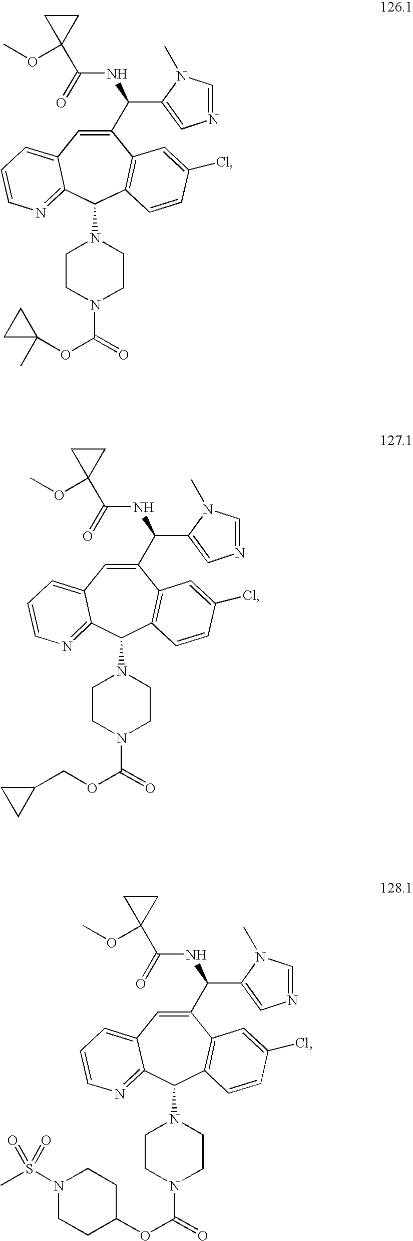 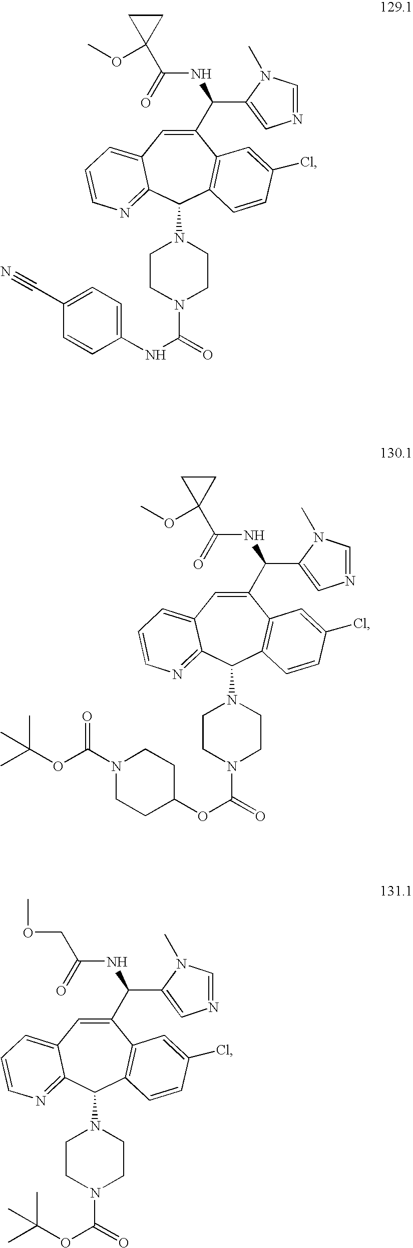 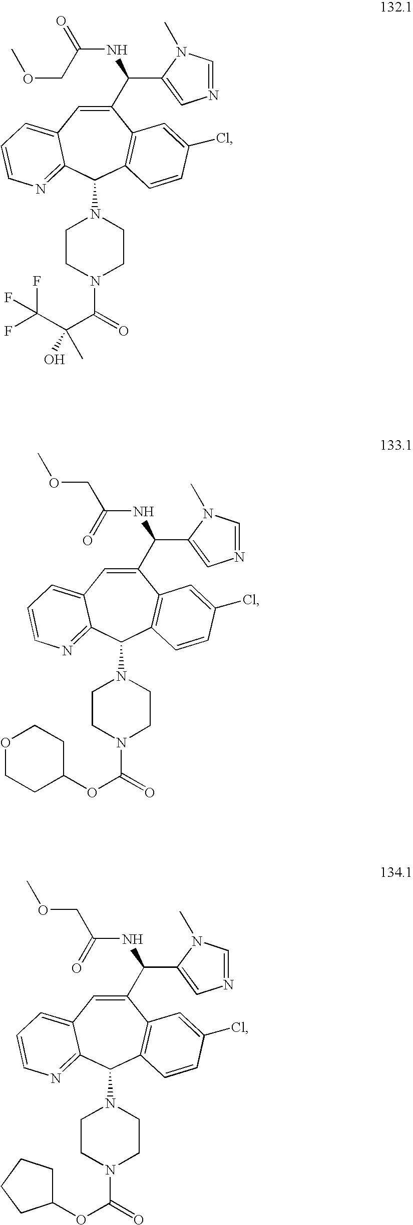 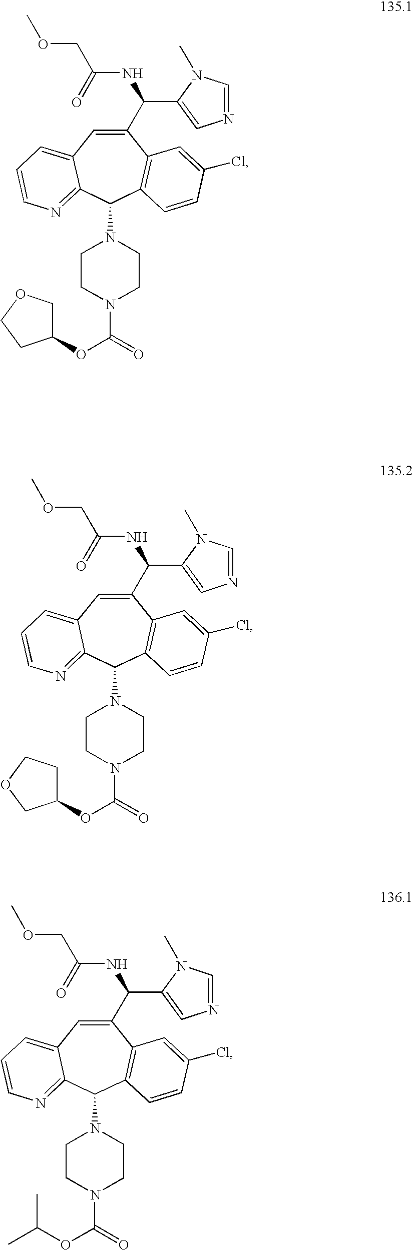 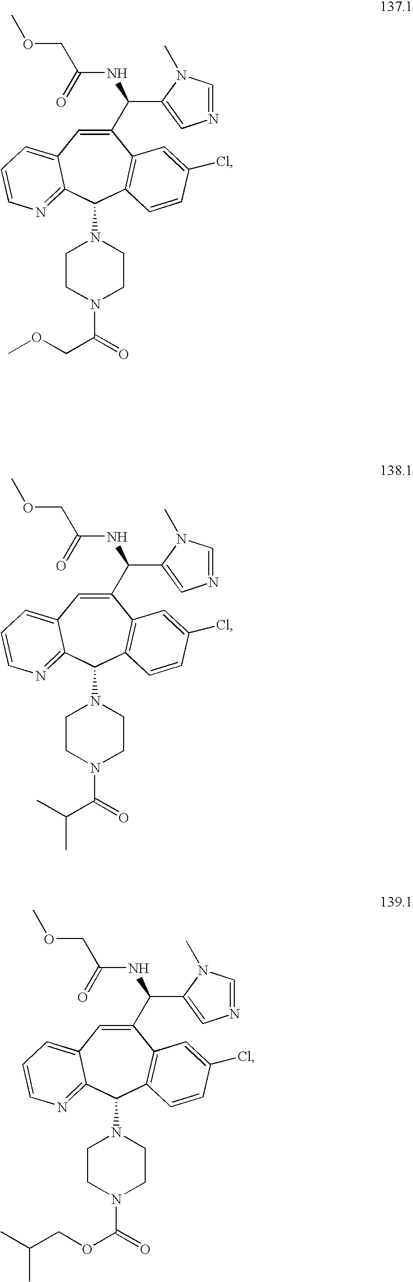 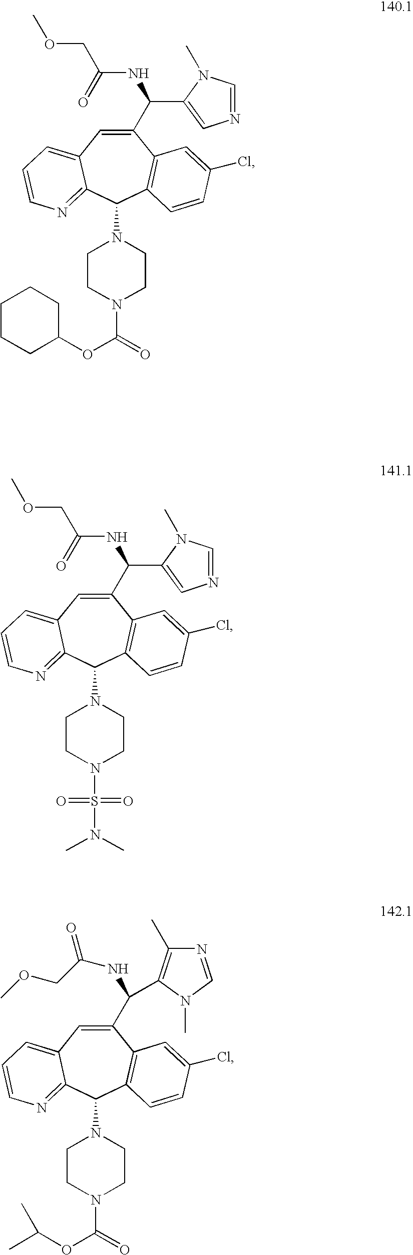 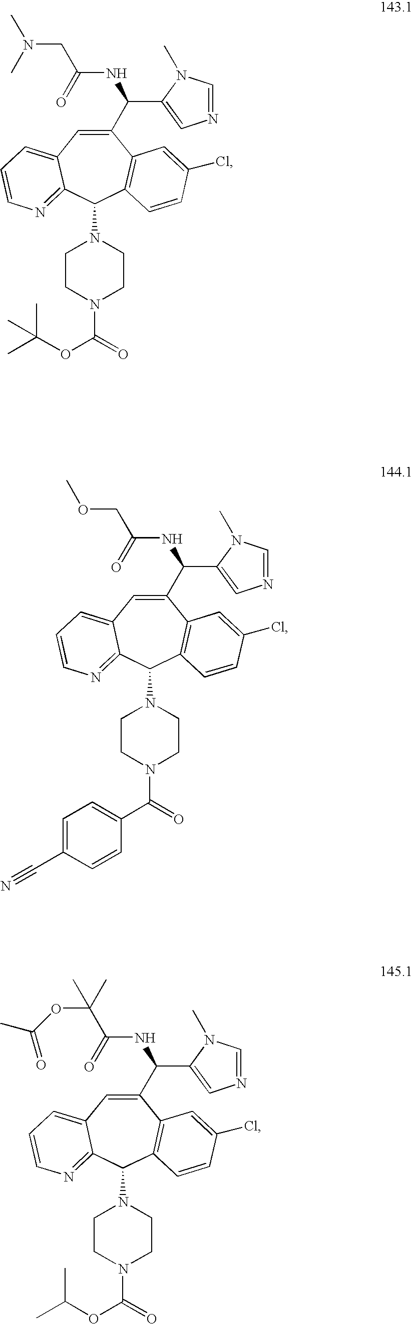 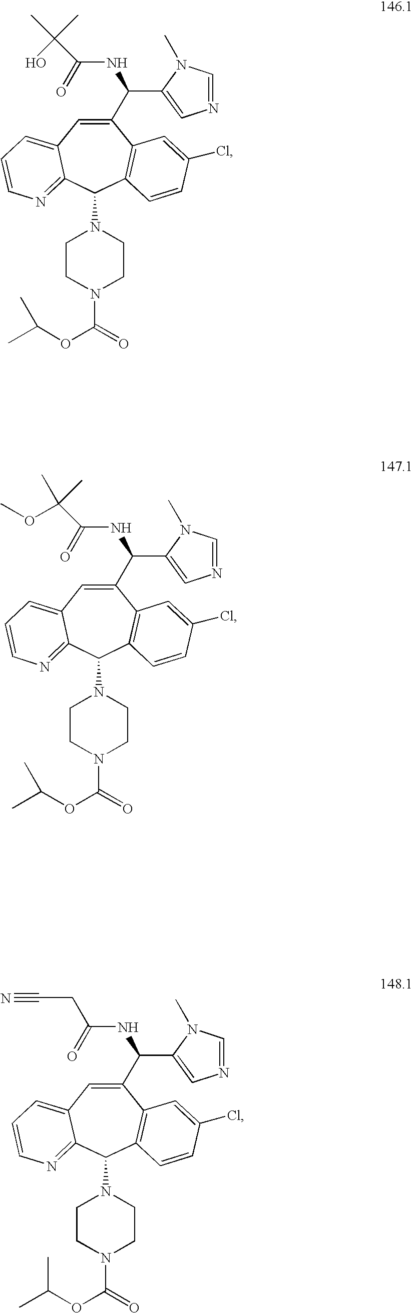 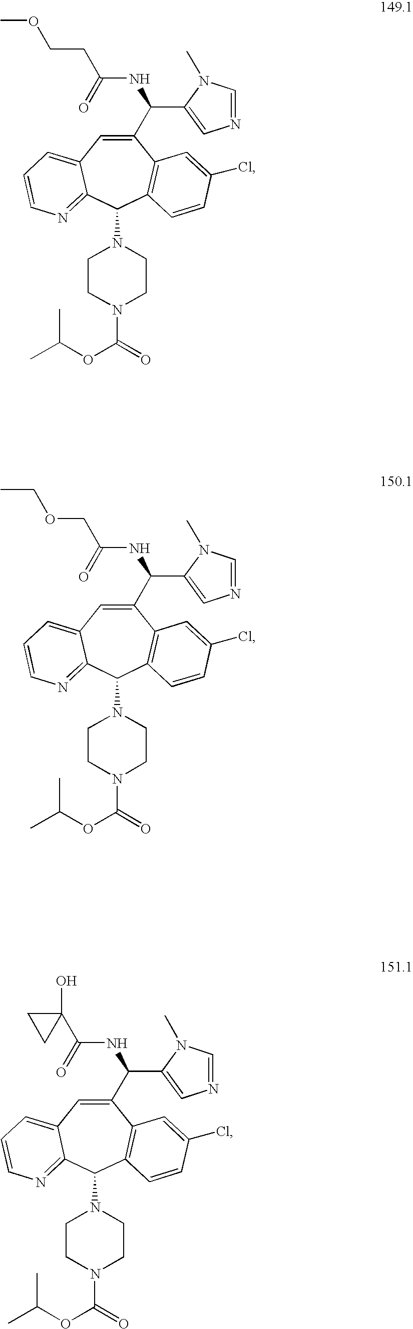 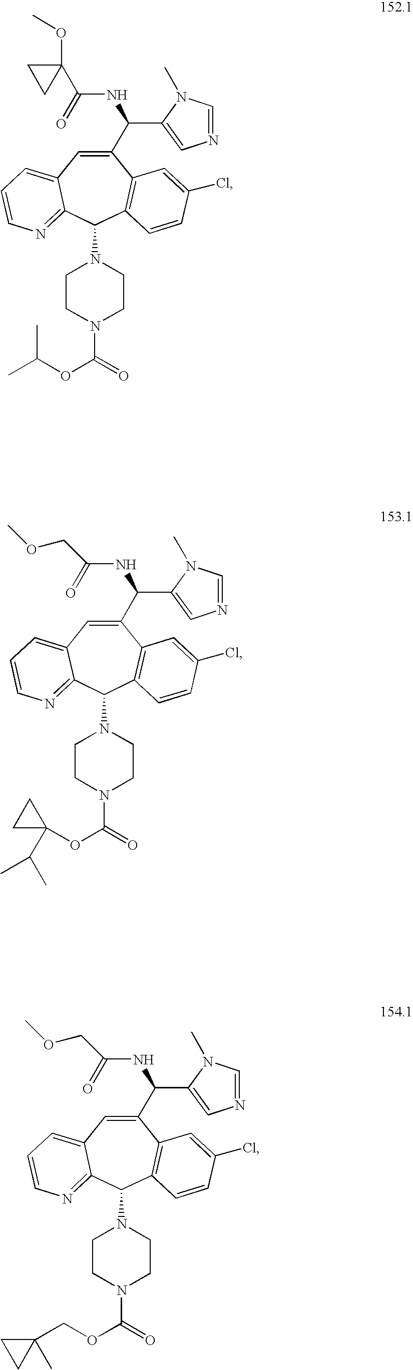 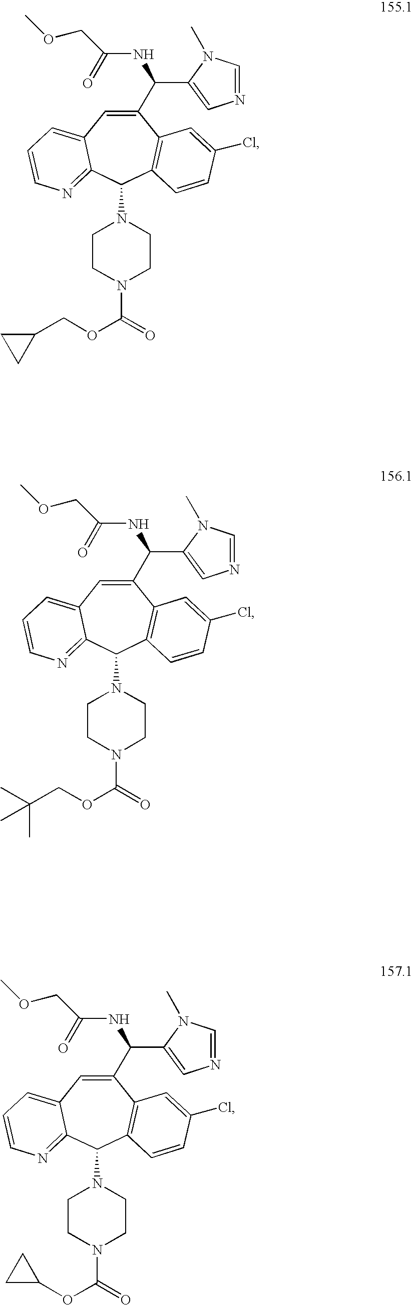 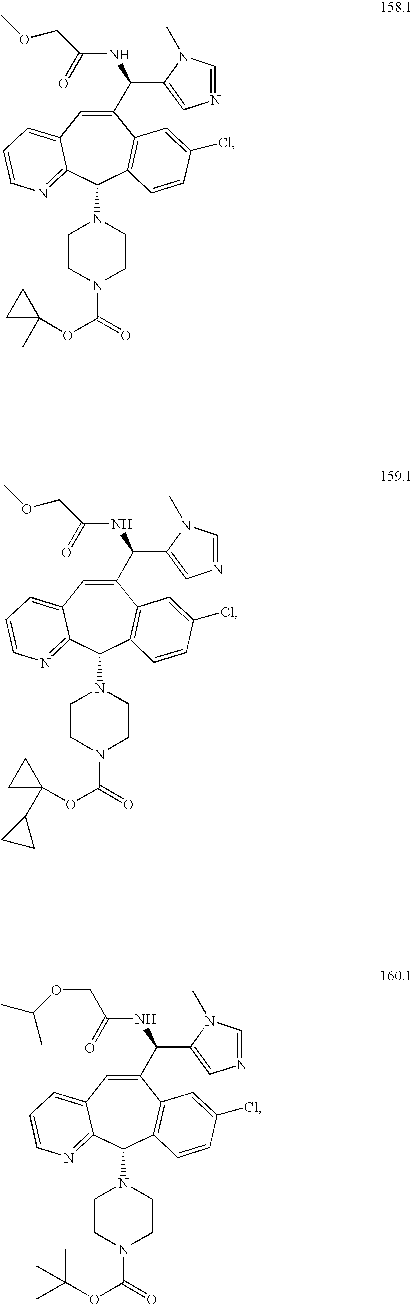 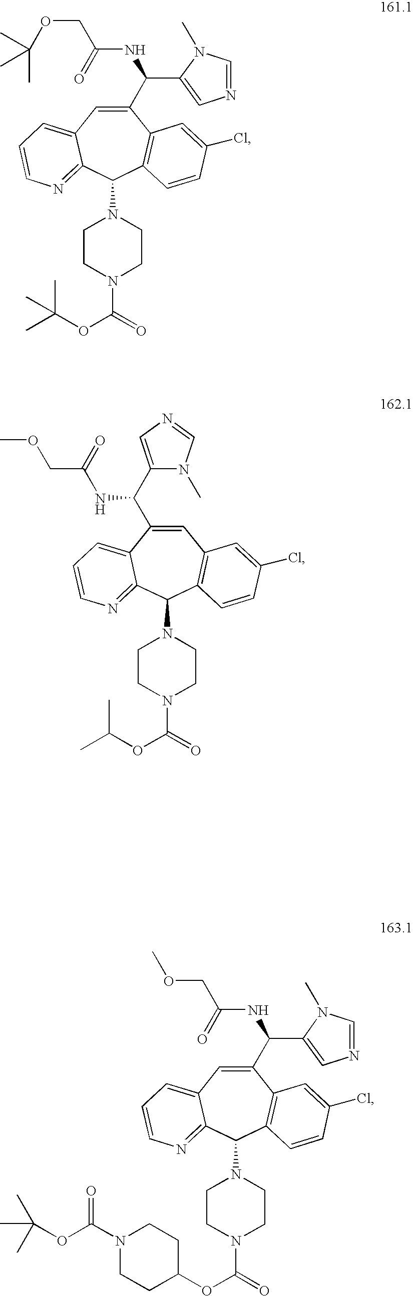 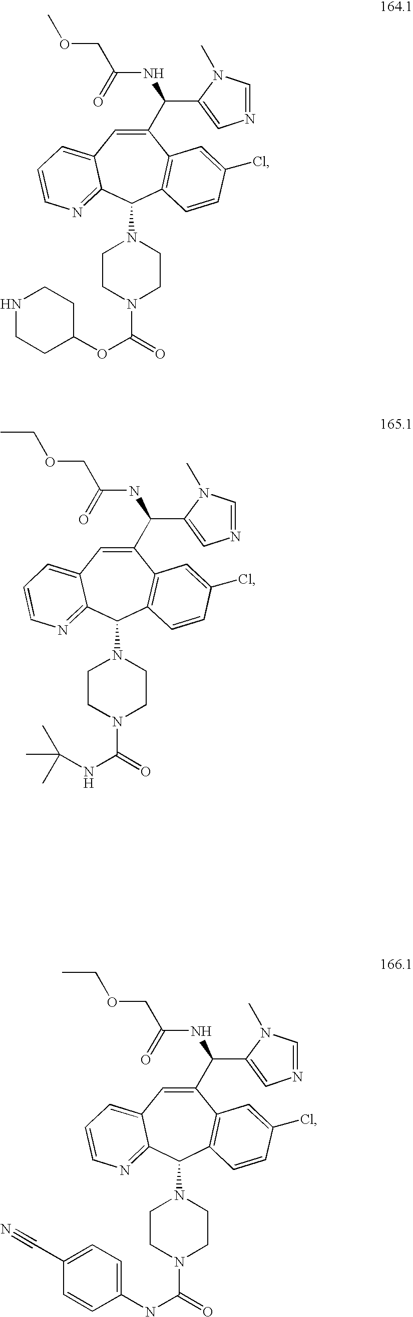 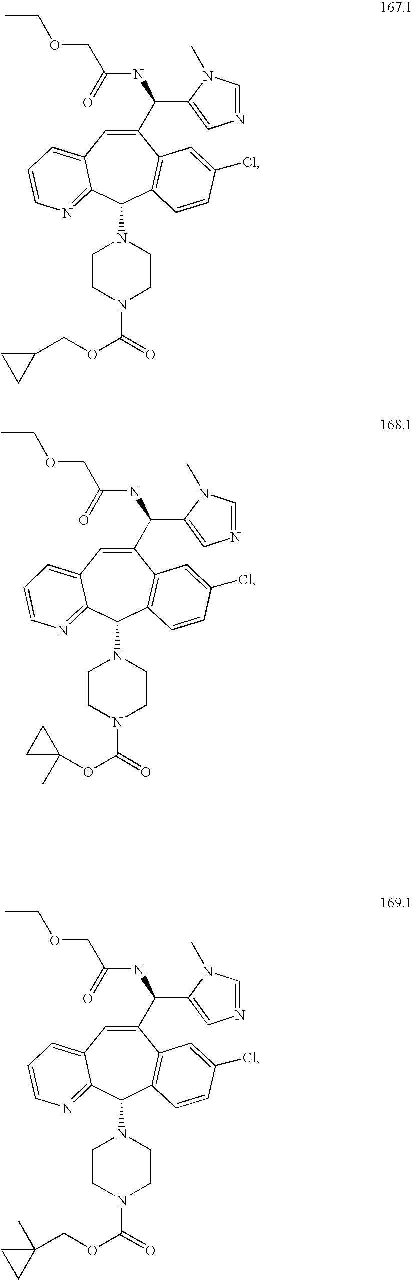 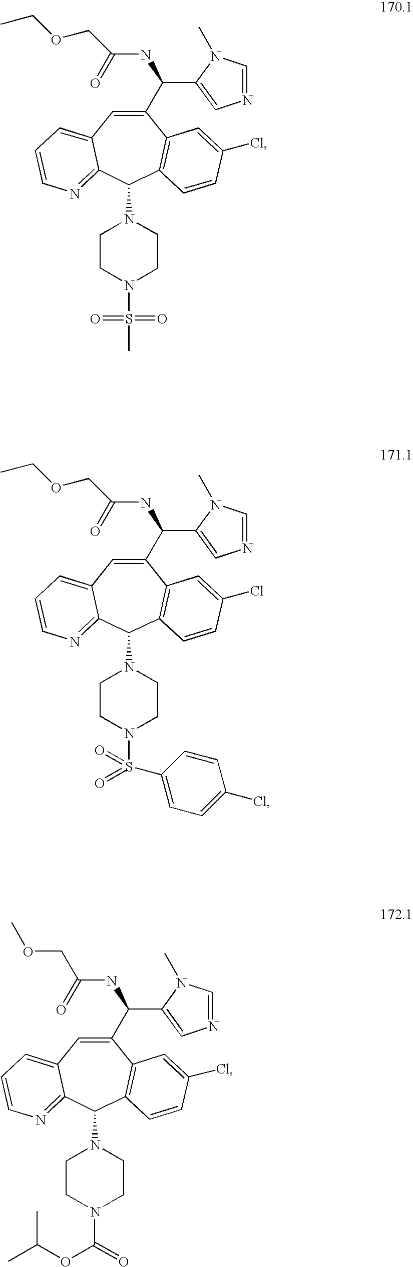 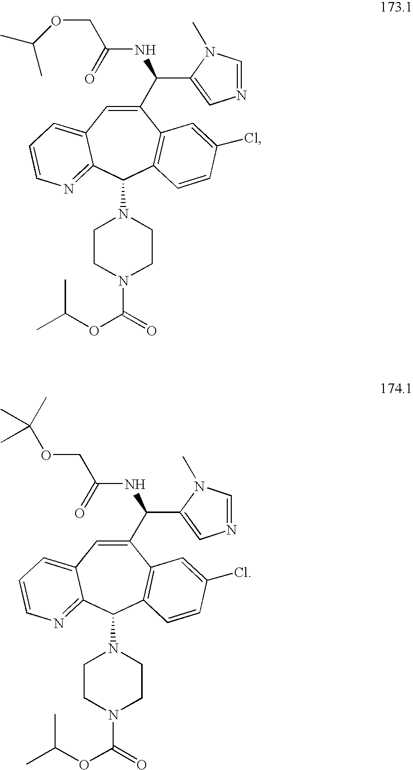
   In one example the compound of formula I is a compound of the formula 101.1. In another example the compound of formula I is a compound of the formula 102.1. In another example the compound of formula I is a compound of the formula 102.2. In another example the compound of formula I is a compound of the formula 105.1. In another example the compound of formula I is a compound of the formula 108.1. In another example the compound of formula I is a compound of the formula 114.1. In another example the compound of formula I is a compound of the formula 118.1. In another example the compound of formula I is a compound of the formula 124.1. In another example the compound of formula I is a compound of the formula 136.1. In another example the compound of formula I is a compound of the formula 139.1. In another example the compound of formula I is a compound of the formula 158.1. In another example the compound of formula I is a compound of the formula 168.1.1.

The Bcr/abl kinase inhibitors, EGF receptor inhibitors, and HER-2 antibodies (EGF receptor inhibitors that are antibodies) described above are also known as signal transduction inhibitors. Therefore, chemotherapeutic agents, as used herein, include signal transduction inhibitors.

Typical signal transduction inhibitors, that are chemotherapeutic agents, include but are not limited to: (i) Bcr/abl kinase inhibitors such as, for example, STI 571 (Gleevec), (ii) Epidermal growth factor (EGF) receptor inhibitor such as, for example, Kinase inhibitors (Iressa, OSI-774) and antibodies (Imclone: C225 [Goldstein et al. (1995), Clin Cancer Res. 1:1311-1318], and Abgenix: ABX-EGF) and (iii) HER-2/neu receptor inhibitors such as, for example, Herceptin®) (trastuzumab).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA), the Physician's Desk Reference, 56^{th} Edition, 2002 (published by Medical Economics company, Inc. Montvale, N.J. 07645-1742), and the Physician's Desk Reference, 57^{th} Edition, 2003 (published by Thompson PDR, Montvale, N.J. 07645-1742); the disclosures of which is incorporated herein by reference thereto.

For example, the compound of formula 1.0 (e.g., a pharmaceutical composition comprising the compound of formula 1.0); can be administered orally (e.g., as a capsule), and the chemotherapeutic agents can be administered intravenously, usually as an IV solution. The use of a pharmaceutical composition comprising more than one drug is within the scope of this invention.

The compound of formula 1.0 and the chemotherapeutic agents are administered in therapeutically effective dosages to obtain clinically acceptable results, e.g., reduction or elimination of symptoms or of the tumor. Thus, the compound of formula 1.0 and chemotherapeutic agents can be administered concurrently or consecutively in a treatment protocol. The administration of the chemotherapeutic agents can be made according to treatment protocols already known in the art.

In general when more than one chemotherapeutic agent is used in the methods of this invention, the chemotherapeutic agents are administered on the same day either concurrently or consecutively in their standard dosage form. For example, the chemotherapeutic agents are usually administered intravenously, preferably by an IV drip using IV solutions well known in the art (e.g., isotonic saline (0.9% NaCl) or dextrose solution (e.g., 5% dextrose)).

When two or more chemotherapeutic agents are used, the chemotherapeutic agents are generally administered on the same day; however, those skilled in the art will appreciate that the chemotherapeutic agents can be administered on different days and in different weeks. The skilled clinician can administer the chemotherapeutic agents according to their recommended dosage schedule from the manufacturer of the agent and can adjust the schedule according to the needs of the patient, e.g., based on the patient's response to the treatment. For example, when gemcitabine is used in combination with a platinum coordinator compound, such as, for example, cisplatin, to treat lung cancer, both the gemcitabine and the cisplatin are given on the same day on day one of the treatment cycle, and then gemcitabine is given alone on day 8 and given alone again on day 15

The compounds of this invention and chemotherapeutic agents can be administered in a treatment protocol that usually lasts one to seven weeks, and is repeated typically from 6 to 12 times. Generally the treatment protocol can last one to four weeks. Treatment protocols of one to three weeks can also be used. A treatment protocol of one to two weeks can also be used. During this treatment protocol or cycle the compounds of this invention can be administered daily while the chemotherapeutic agents can be administered one or more times a week. Generally, a compound of this invention can be administered daily (i.e., once per day), and in one embodiment twice per day, and the chemotherapeutic agent is administered once a week or once every three weeks. For example, the taxanes (e.g., Paclitaxel (e.g., Taxol®) or Docetaxel (e.g., Taxotere®)) can be administered once a week or once every three weeks.

However, those skilled in the art will appreciate that treatment protocols can be varied according to the needs of the patient. Thus, the combination of compounds (drugs) used in the methods of this invention can be administered in variations of the protocols described above. For example, the compounds of this invention can be administered discontinuously rather than continuously during the treatment cycle. Thus, for example, during the treatment cycle the compounds of this invention can be administered daily for a week and then discontinued for a week, with this administration repeating during the treatment cycle. Or the compounds of this invention can be administered daily for two weeks and discontinued for a week, with this administration repeating during the treatment cycle. Thus, the compounds of this invention can be administered daily for one or more weeks during the cycle and discontinued for one or more weeks during the cycle, with this pattern of administration repeating during the treatment cycle. This discontinuous treatment can also be based upon numbers of days rather than a full week. For example, daily dosing for 1 to 6 days, no dosing for 1 to 6 days with this pattern repeating during the treatment protocol. The number of days (or weeks) wherein the compounds of this invention are not dosed do not have to equal the number of days (or weeks) wherein the compounds of this invention are dosed. Usually, if a discontinuous dosing protocol is used, the number of days or weeks that the compounds of this invention are dosed is at least equal or greater than the number of days or weeks that the compounds of this invention are not dosed.

The chemotherapeutic agent could be given by bolus or continuous infusion. The chemotherapeutic agent could be given daily to once every week, or once every two weeks, or once every three weeks, or once every four weeks during the treatment cycle. If administered daily during a treatment cycle, this daily dosing can be discontinuous over the number of weeks of the treatment cycle. For example, dosed for a week (or a number of days), no dosing for a week (or a number of days, with the pattern repeating during the treatment cycle.

The compounds of this invention can be administered orally, preferably as a solid dosage form, and in one embodiment as a capsule, and while the total therapeutically effective daily dose can be administered in one to four, or one to two divided doses per day, generally, the therapeutically effective dose is given once or twice a day, and in one embodiment twice a day. The compounds of this invention can be administered in an amount of about 50 to about 400 mg once per day, and can be administered in an amount of about 50 to about 300 mg once per day. The compounds of this invention are generally administered in an amount of about 50 to about 350 mg twice a day, usually 50 mg to about 200 mg twice a day, and in one embodiment about 75 mg to about 125 mg administered twice a day, and in another embodiment about 100 mg administered twice a day.

If the patient is responding, or is stable, after completion of the therapy cycle, the therapy cycle can be repeated according to the judgment of the skilled clinician. Upon completion of the therapy cycles, the patient can be continued on the compounds of this invention at the same dose that was administered in the treatment protocol, or, if the dose was less than 200 mg twice a day, the dose can be raised to 200 mg twice a day. This maintenance dose can be continued until the patient progresses or can no longer tolerate the dose (in which case the dose can be reduced and the patient can be continued on the reduced dose).

The chemotherapeutic agents, used with the compounds of this invention, are administered in their normally prescribed dosages during the treatment cycle (i.e., the chemotherapeutic agents are administered according to the standard of practice for the administration of these drugs). For example: (a) about 30 to about 300 mg/m² for the taxanes; (b) about 30 to about 100 mg/m² for Cisplatin; (c) AUC of about 2 to about 8 for Carboplatin; (d) about 2 to about 4 mg/m² for EGF inhibitors that are antibodies; (e) about 50 to about 500 mg/m² for EGF inhibitors that are small molecules; (f) about 1 to about 10 mg/m² for VEGF kinase inhibitors that are antibodies; (g) about 50 to about 2400 mg/m² for VEGF inhibitors that are small molecules; (h) about 1 to about 20 mg for SERMs; (i) about 500 to about 1250 mg/m² for the anti-tumor nucleosides 5-Fluorouracil, Gemcitabine and Capecitabine; (j) for the anti-tumor nucleoside Cytarabine (Ara-C) 100-200 mg/m²/day for 7 to 10 days every 3 to 4 weeks, and high doses for refractory leukemia and lymphoma, i.e., 1 to 3 gm/m² for one hour every 12 hours for 4-8 doses every 3 to four weeks; (k) for the anti-tumor nucleoside Fludarabine (F-ara-A) 10-25 mg/m²/day every 3 to 4 weeks; (l) for the anti-tumor nucleoside Decitabine 30 to 75 mg/m² for three days every 6 weeks for a maximum of 8 cycles; (m) for the anti-tumor nucleoside Chlorodeoxyadenosine (CdA, 2-CdA) 0.05-0.1 mg/kg/day as continuous infusion for up to 7 days every 3 to 4 weeks; (n) about 1 to about 100 mg/m² for epothilones; (o) about 1 to about 350 mg/m² for topoisomerase inhibitors; (p) about 1 to about 50 mg/m² for vinca alkaloids; (q) for the folate antagonist Methotrexate (MTX) 20-60 mg/m² by oral, IV or IM every 3 to 4 weeks, the intermediate dose regimen is 80-250 mg/m² IV over 60 minutes every 3 to 4 weeks, and the high dose regimen is 250-1000 mg/m² IV given with leucovorin every 3 to 4 weeks; (r) for the folate antagonist Premetrexed (Alimta) 300-600 mg/m² (10 minutes IV infusion day 1) every 3 weeks; (s) for the ribonucleotide reductase inhibitor Hydroxyurea (HU) 20-50 mg/kg/day (as needed to bring blood cell counts down); (t) the platinum coordinator compound Oxaliplatin (Eloxatin) 50-100 mg/m² every 3 to 4 weeks (preferably used for solid tumors such as non-small cell lung cancer, colorectal cancer and ovarian cancer); (u) for the anthracycline daunorubicin 10-50 mg/m²/day IV for 3-5 days every 3 to 4 weeks; (v) for the anthracycline Doxorubicin (Adriamycin) 50-100 mg/m² IV continuous infusion over 14 days every 3 to 4 weeks, or 10-40 mg/m² IV weekly; (w) for the anthracycline Idarubicin 10-30 mg/m² daily for 1-3 days as a slow IV infusion over 10-20 minutes every 3 to 4 weeks; (x) for the biologic interferon (Intron-A, Roferon) 5 to 20 million IU three times per week; (y) for the biologic pegylated interferon (Peg-intron, Pegasys) 3 to 4 micrograms/kg/day chronic sub cutaneous (until relapse or loss of activity); (z) for the biologic Rituximab (Rituxan) (antibody used for non-Hodgkin's lymphoma) 200-400 mg/m² IV weekly over 4-8 weeks for 6 months; (aa) for the alkylating agent temozolomide 75 mg/m² to 250 mg/m², for example, 150 mg/m², or for example, 200 mg/m², such as 200 mg/m² for 5 days; and (bb) for the MEK1 and/or MEK2 inhibitor PD0325901, 15 mg to 30 mg, for example, 15 mg daily for 21 days every 4 weeks.

Gleevec can be used orally in an amount of about 200 to about 800 mg/day.

Thalidomide (and related imids) can be used orally in amounts of about 200 to about 800 mg/day, and can be continuously dosed or used until releapse or toxicity. See for example Mitsiades et al., “Apoptotic signaling induced by immunomodulatory thalidomide analogs in human multiple myeloma cells; therapeutic implications”, Blood, 99(12):4525-30, Jun. 15, 2002, the disclosure of which is incorporated herein by reference thereto.

The FPT inhibitor Sarasar can be administered orally (e.g., capsule) in amounts of about 50 to about 200 mg given twice a day, or in amounts of about 75 to about 125 mg given twice a day, or in an amount of about 100 mg given twice a day.

For example, Paclitaxel (e.g., Taxol® can be administered once per week in an amount of about 50 to about 100 mg/m² and in another example about 60 to about 80 mg/m². In another example Paclitaxel (e.g., Taxol® can be administered once every three weeks in an amount of about 150 to about 250 mg/m² and in another example about 175 to about 225 mg/m².

In another example, Docetaxel (e.g., Taxotere®) can be administered once per week in an amount of about 10 to about 45 mg/m². In another example Docetaxel (e.g., Taxotere®) can be administered once every three weeks in an amount of about 50 to about 100 mg/m².

In another example Cisplatin can be administered once per week in an amount of about 20 to about 40 mg/m². In another example Cisplatin can be administered once every three weeks in an amount of about 60 to about 100 mg/m².

In another example Carboplatin can be administered once per week in an amount to provide an AUC of about 2 to about 3. In another example Carboplatin can be administered once every three weeks in an amount to provide an AUC of about 5 to about 8.

Thus, in one embodiment directed to the methods of treating cancer using at least one compound of formula 1.0 and at least one chemotherapeutic agent, chemotherapeutic agent is selected from the group consisting of: paclitaxel, docetaxel, carboplatin, cisplatin, gemcitabine, tamoxifen, Herceptin, Cetuximab, Tarceva, Iressa, bevacizumab, navelbine, IMC-1C11, SU5416 and SU6688.

In another embodiment directed to the methods of treating cancer using at least one compound of formula 1.0 and at least one chemotherapeutic agent, the chemotherapeutic agent is selected from the group consisting of: paclitaxel, docetaxel, carboplatin, cisplatin, navelbine, gemcitabine, and Herceptin.

In another embodiment directed to the methods of treating cancer using at least one compound of formula 1.0 and at least one chemotherapeutic agent, the chemotherapeutic agent is selected from the group consisting of: Cyclophasphamide, 5-Fluorouracil, Temozolomide, Vincristine, Cisplatin, Carboplatin, and Gemcitabine.

In another embodiment directed to the methods of treating cancer using at least one compound of formula 1.0 and at least one chemotherapeutic agent, the chemotherapeutic agent is selected from the group consisting of: Gemcitabine, Cisplatin and Carboplatin.

This invention also provides a method of treating cancer in a patient in need of such treatment, said treatment comprising administering to said patient a therapeutically effective amount at least one (e.g., 1, 2 or 3, or 1 or 2, or 1, and usually 1) compound of formula 1.0, and therapeutically effective amounts of at least one (e.g., 1, 2 or 3, or 1 or 2, or 2, or 1) chemotherapeutic agent selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

This invention also provides a method of treating cancer in a patient in need of such treatment, said treatment comprising administering to said patient a therapeutically effective amount at least one (e.g., 1, 2 or 3, or 1 or 2, or 1, and usually 1) compound of formula 1.0, and therapeutically effective amounts of at least two (e.g., 2 or 3, or 2, and usually 2) different antineoplastic agents selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αᵥβ₃ integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

This invention also provides a method of treating cancer in a patient in need of such treatment, said method comprising administering to said patient therapeutically effective amounts at least one (e.g., 1, 2 or 3, or 1 or 2, or 1, and usually 1) compound of formula 1.0, and an antineoplastic agent selected from the group consisting of: (1) EGF inhibitors that are antibodies, (2) EGF inhibitors that are small molecules, (3) VEGF inhibitors that are antibodies, and (4) VEGF inhibitors that are small molecules. Radiation therapy can also be used in conjunction with this above combination therapy, i.e., the above method using a combination of compounds of the invention and antineoplastic agent can also comprise the administration of a therapeutically effect amount of radiation.

This invention also provides a method of treating leukemias (e.g., acute myeloid leukemia (AML), and chronic myeloid leukemia (CML)) in a patient in need of such treatment, said method comprising administering to said patient therapeutically effective amounts at least one (e.g., 1, 2 or 3, or 1 or 2, or 1, and usually 1) compound of formula 1.0, and: (1) Gleevec and interferon to treat CML; (2) Gleevec and pegylated interferon to treat CML; (3) Gleevec to treat CML; (4) an anti-tumor nucleoside derivative (e.g., Ara-C) to treat AML; or (5) an anti-tumor nucleoside derivative (e.g., Ara-C) in combination with an anthracycline to treat AML.

This invention also provides a method of treating non-Hodgkin's lymphoma in a patient in need of such treatment, said method comprising administering therapeutically effective amounts at least one (e.g., 1, 2 or 3, or 1 or 2, or 1, and usually 1) compound of formula 1.0 (for example, as described in any one of Embodiment Nos. 1 to 107) and: (1) a biologic (e.g., Rituxan); (2) a biologic (e.g., Rituxan) and an anti-tumor nucleoside derivative (e.g., Fludarabine); or (3) Genasense (antisense to BCL-2).

This invention also provides a method of treating multiple myeloma in a patient in need of such treatment, said method comprising administering to said patient therapeutically effective amounts of at least one (e.g., 1, 2 or 3, or 1 or 2, or 1, and usually 1) compound of formula 1.0 and: (1) a proteosome inhibitor (e.g., PS-341 from Millenium); or (2) Thalidomide (or related imid).

This invention also provides a method of treating cancer in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, (3) EGF inhibitors that are antibodies, (4) EGF inhibitors that are small molecules, (5) VEGF inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, and (12) antibodies that are inhibitors of αVβ3 integrins.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, (3) EGF inhibitors that are antibodies, (4) EGF inhibitors that are small molecules, (5) VEGF inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, and (12) antibodies that are inhibitors of αVβ3 integrins.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, (3) anti-tumor nucleoside derivatives, (4) topoisomerase inhibitors, and (5) vinca alkaloids.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0, (b) carboplatin, and (c) paclitaxel.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0, (b) cisplatin, and (c) gemcitabine.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0, (b) carboplatin, and (c) gemcitabine.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0, (b) Carboplatin, and (c) Docetaxel.

This invention also provides a method of treating cancer in a patient in need of such treatment, said method comprising administering therapeutically effective amounts of: (a) at least one (e.g., 1, 2 or 3, or 1 or 2, or 1, and usually 1) compound of formula 1.0, and (b) an antineoplastic agent selected from the group consisting of: (1) EGF inhibitors that are antibodies, (2) EGF inhibitors that are small molecules, (3) VEGF inhibitors that are antibodies, (4) VEGF kinase inhibitors that are small molecules.

This invention also provides a method of treating squamous cell cancer of the head and neck, in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, and (2) platinum coordinator compounds.

This invention also provides a method of treating squamous cell cancer of the head and neck, in a patient in need of such treatment, said method comprising administering to said patient therapeutically effective amounts of: (a) at least one (e.g., 1, 2 or 3, or 1 or 2, or 1, and usually 1) compound of formula 1.0, and (b) at least one (e.g., 1, 2 or 3, or 1 or 2, or 2, or 1) antineoplastic agent selected from the group consisting of: (1) taxanes, (2) platinum coordinator compounds, and (3) anti-tumor nucleoside derivatives (e.g., 5-Fluorouracil).

This invention also provides a method of treating CML in a patient in need of such treatment, said method comprising administering therapeutically effective amounts of: (a) at least one (e.g., 1, 2 or 3, or 1 or 2, or 1, and usually 1) compound of formula 1.0, (b) Gleevec, and (c) interferon (e.g., Intron-A).

This invention also provides a method of treating CML in a patient in need of such treatment comprising administering therapeutically effective amounts of: (a) at least one (e.g., 1, 2 or 3, or 1 or 2, or 1, and usually 1) compound of formula 1.0, (b) Gleevec; and (c) pegylated interferon (e.g., Peg-Intron, and Pegasys).

This invention also provides a method of treating CML in a patient in need of such treatment comprising administering therapeutically effective amounts of: (a) at least one (e.g., 1, 2 or 3, or 1 or 2, or 1, and usually 1) compound of formula 1.0 and (b) Gleevec.

This invention also provides a method of treating CMML in a patient in need of such treatment, said method comprising administering to said patient therapeutically effective amounts of at least one (e.g., 1, 2 or 3, or 1 or 2, or 1, and usually 1) compound of formula 1.0.

This invention also provides a method of treating AML in a patient in need of such treatment, said method comprising administering to said patient therapeutically effective amounts of: (a) at least one (e.g., 1, 2 or 3, or 1 or 2, or 1, and usually 1) compound of formula 1.0, and (b) an anti-tumor nucleoside derivative (e.g., Cytarabine (i.e., Ara-C)).

This invention also provides a method of treating AML in a patient in need of such treatment, said method comprising administering to said patient therapeutically effective amounts of: (a) at least one (e.g., 1, 2 or 3, or 1 or 2, or 1, and usually 1) compound of formula 1.0, (b) an anti-tumor nucleoside derivative (e.g., Cytarabine (i.e., Ara-C)), and (c) an anthracycline.

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0, and (b) Rituximab (Rituxan).

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0, (b) Rituximab (Rituxan), and (c) an anti-tumor nucleoside derivative (e.g., Fludarabine (i.e., F-ara-A).

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0, and (b) Genasense (antisense to BCL-2).

This invention also provides a method of treating multiple myeloma in a patient in need of such treatment, said method comprising administering therapeutically effective amounts of: (a) at least one (e.g., 1, 2 or 3, or 1 or 2, or 1, and usually 1) compound of formula 1.0, and (b) a proteosome inhibitor (e.g., PS-341 (Millenium)).

This invention also provides a method of treating multiple myeloma in a patient in need of such treatment, said method comprising administering to said patient therapeutically effective amounts of: (a) at least one (e.g., 1, 2 or 3, or 1 or 2, or 1, and usually 1) compound of formula 1.0, and (b) Thalidomide or related imid.

This invention also provides a method of treating multiple myeloma in a patient in need of such treatment, said method comprising administering therapeutically effective amounts of: (a) at least one (e.g., 1, 2 or 3, or 1 or 2, or 1, and usually 1) compound of formula 1.0, and (b) Thalidomide.

This invention is also directed to the methods of treating cancer described herein, particularly those described above, wherein in addition to the administration of the compound of formula 1.0 and antineoplastic agents, radiation therapy is also administered prior to, during, or after the treatment cycle.

This invention also provides a method for treating cancer (e.g., lung cancer, prostate cancer and myeloid leukemias) in a patient in need of such treatment, said method comprising administering to said patient (1) an effective amount of at least one (e.g., 1, 2 or 3, or 1 or 2, or 1, and usually 1) compound of formula 1.0, in combination with (2) at least one (e.g., 1, 2 or 3, or 1 or 2, or 2, or 1) antineoplastic agent, microtubule affecting agent and/or radiation therapy.

This invention also provides a method of treating cancer in a patient in need of such treatment, said method comprising administering to said patient an effective amount of at least one (e.g., 1, 2 or 3, or 1 or 2, or 1, and usually 1) compound of formula 1.0 in combination with an effective amount of at least one (e.g., 1, 2 or 3, or 1 or 2, or 1, and usually 1) signal transduction inhibitor.

Thus, in one example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once per week in an amount of about 50 to about 100 mg/m², and in another example about 60 to about 80 mg/m², and (3) Carboplatin is administered once per week in an amount to provide an AUC of about 2 to about 3.

In another example (e.g., treating non small cell lung cancer): (1) the compound of formula 1.0 is administered in an amount of about 50 mg to about 200 mg twice a day, and in another example about 75 mg to about 125 mg administered twice a day, and yet in another example about 100 mg administered twice a day, (2) Paclitaxel (e.g., Taxol® is administered once per week in an amount of about 50 to about 100 mg/m², and in another example about 60 to about 80 mg/m², and (3) Cisplatin is administered once per week in an amount of about 20 to about 40 mg/m².

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere®) is administered once per week in an amount of about 10 to about 45 mg/m², and (3) Carboplatin is administered once per week in an amount to provide an AUC of about 2 to about 3.

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere®) is administered once per week in an amount of about 10 to about 45 mg/m², and (3) Cisplatin is administered once per week in an amount of about 20 to about 40 mg/m².

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once every three weeks in an amount of about 150 to about 250 mg/m², and in another example about 175 to about 225 mg/m², and in yet another example 175 mg/m², and (3) Carboplatin is administered once every three weeks in an amount to provide an AUC of about 5 to about 8, and in another example 6.

In another example of treating non small cell lung cancer: (1) the compound of formula 1.0 is administered in an amount of 100 mg administered twice a day, (2) Paclitaxel (e.g., Taxol® is administered once every three weeks in an amount of 175 mg/m², and (3) Carboplatin is administered once every three weeks in an amount to provide an AUC of 6.

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once every three weeks in an amount of about 150 to about 250 mg/m², and in another example about 175 to about 225 mg/m², and (3) Cisplatin is administered once every three weeks in an amount of about 60 to about 100 mg/m².

In another example (e.g., treating non small cell lung cancer): (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere® is administered once every three weeks in an amount of about 50 to about 100 mg/m², and (3) Carboplatin is administered once every three weeks in an amount to provide an AUC of about 5 to about 8.

In another example (e.g., treating non small cell lung cancer): (1) the compound of formula 1.0 is administered in an amount of about 50 mg to about 200 mg twice a day, in another example about 75 mg to about 125 mg administered twice a day, and in yet another example about 100 mg administered twice a day, (2) Docetaxel (e.g., Taxotere® is administered once every three weeks in an amount of about 50 to about 100 mg/m², and (3) Cisplatin is administered once every three weeks in an amount of about 60 to about 100 mg/m².

In another example for treating non small cell lung cancer using the compounds of formula 1.0, Docetaxel and Carboplatin: (1) the compound of formula 1.0 is administered in an amount of about 50 mg to about 200 mg twice a day, and in another example about 75 mg to about 125 mg administered twice a day, and in yet another example about 100 mg administered twice a day, (2) Docetaxel (e.g., Taxotere® is administered once every three weeks in an amount of about 75 mg/m², and (3) Carboplatin is administered once every three weeks in an amount to provide an AUC of about 6.

In another example of the treatments of non-small cell lung cancer described above the Docetaxel (e.g., Taxotere® and Cisplatin, the Docetaxel (e.g., Taxotere® and Carboplatin, the Paclitaxel (e.g., Taxol® and Carboplatin, or the Paclitaxel (e.g., Taxol® and Cisplatin are administered on the same day.

In another example (e.g., CML): (1) the compound of formula 1.0 is administered in an amount of about 100 mg to about 200 mg administered twice a day, (2) Gleevec is administered in an amount of about 400 to about 800 mg/day orally, and (3) interferon (Intron-A) is administered in an amount of about 5 to about 20 million IU three times per week.

In another example (e.g., CML): (1) the compound of formula 1.0 is administered in an amount of about 100 mg to about 200 mg administered twice a day, (2) Gleevec is administered in an amount of about 400 to about 800 mg/day orally, and (3) pegylated interferon (Peg-Intron or Pegasys) is administered in an amount of about 3 to about 6 micrograms/kg/day.

In another example (e.g., non-Hodgkin's lymphoma): (1) the compound of formula 1.0 is administered in an amount of about 50 mg to about 200 mg twice a day, and in another example about 75 mg to about 125 mg administered twice a day, and in yet another example about 100 mg administered twice a day, and (2) Genasense (antisense to BCL-2) is administered as a continuous IV infusion at a dose of about 2 to about 5 mg/kg/day (e.g., 3 mg/kg/day) for 5 to 7 days every 3 to 4 weeks.

In another example (e.g., multiple myeloma): (1) the compound of formula 1.0 is administered in an amount of about 50 mg to about 200 mg twice a day, and in another example about 75 mg to about 125 mg administered twice a day, and in yet another example about 100 mg administered twice a day, and (2) the proteosome inhibitor (e.g., PS-341—Millenium) is administered in an amount of about 1.5 mg/m² twice weekly for two consecutive weeks with a one week rest period.

In another example (e.g., multiple myeloma): (1) the compound of formula 1.0 is administered in an amount of about 50 mg to about 200 mg twice a day, and in another example about 75 mg to about 125 mg administered twice a day, and in yet another example about 100 mg administered twice a day, and (2) the Thalidomide (or related imid) is administered orally in an amount of about 200 to about 800 mg/day, with dosing being continuous until relapse or toxicity.

In one embodiment of the methods of treating cancer of this invention, the chemotherapeutic agents are selected from the group consisting of: paclitaxel, docetaxel, carboplatin, cisplatin, gemcitabine, tamoxifen, Herceptin, Cetuximab, Tarceva, Iressa, bevacizumab, navelbine, IMC-1C11, SU5416 and SU6688.

In another embodiment of the methods of treating cancer of this invention, the chemotherapeutic agents are selected from the group consisting of: paclitaxel, docetaxel, carboplatin, cisplatin, navelbine, gemcitabine, and Herceptin.

Thus, one embodiment of this invention is directed to a method of treating cancer comprising administering to a patient in need of such treatment therapeutically effective amounts of the compound of formula 1.0, a taxane, and a platinum coordination compound.

Another embodiment of this invention is directed to a method of treating cancer comprising administering to a patient in need of such treatment therapeutically effective amounts of the compound of formula 1.0, a taxane, and a platinum coordination compound, wherein said compound of formula 1.0 is administered every day, said taxane is administered once per week per cycle, and said platinum coordinator compound is administered once per week per cycle. In another embodiment the treatment is for one to four weeks per cycle.

Another embodiment of this invention is directed to a method of treating cancer comprising administering to a patient in need of such treatment therapeutically effective amounts of the compound of formula 1.0, a taxane, and a platinum coordination compound, wherein said compound of formula 1.0 is administered every day, said taxane is administered once every three weeks per cycle, and said platinum coordinator compound is administered once every three weeks per cycle. In another embodiment the treatment is for one to three weeks per cycle.

Another embodiment of this invention is directed to a method of treating cancer comprising administering to a patient in need of such treatment therapeutically effective amounts of the compound of formula 1.0, paclitaxel, and carboplatin. In another embodiment, said compound of formula 1.0 is administered every day, said paclitaxel is administered once per week per cycle, and said carboplatin is administered once per week per cycle. In another embodiment the treatment is for one to four weeks per cycle.

Another embodiment of this invention is directed to a method of treating cancer comprising administering to a patient in need of such treatment therapeutically effective amounts of the compound of formula 1.0, paclitaxel, and carboplatin. In another embodiment, said compound of formula 1.0 is administered every day, said paclitaxel is administered once every three weeks per cycle, and said carboplatin is administered once every three weeks per cycle. In another embodiment the treatment is for one to three weeks per cycle.

Another embodiment of this invention is directed to a method for treating non small cell lung cancer in a patient in need of such treatment comprising administering daily a therapeutically effective amount of the compound of formula 1.0, administering a therapeutically effective amount of carboplatin once a week per cycle, and administering a therapeutically effective amount of paclitaxel once a week per cycle, wherein the treatment is given for one to four weeks per cycle. In another embodiment said compound of formula 1.0 is administered twice per day. In another embodiment said carboplatin and said paclitaxel are administered on the same day, and in another embodiment said carboplatin and said paclitaxel are administered consecutively, and in another embodiment said carboplatin is administered after said paclitaxel.

Another embodiment of this invention is directed to a method for treating non small cell lung cancer in a patient in need of such treatment comprising administering daily a therapeutically effective amount of a compound of formula 1.0, administering a therapeutically effective amount of carboplatin once every three weeks per cycle, and administering a therapeutically effective amount of paclitaxel once every three weeks per cycle, wherein the treatment is given for one to three weeks. In another embodiment compound of formula 1.0 is administered twice per day. In another embodiment said carboplatin and said paclitaxel are administered on the same day, and in another embodiment said carboplatin and said paclitaxel are administered consecutively, and in another embodiment said carboplatin is administered after said paclitaxel.

Another embodiment of this invention is directed to a method for treating non small cell lung cancer in a patient in need of such treatment comprising administering about 50 to about 200 mg of a compound of formula 1.0 twice a day, administering carboplatin once per week per cycle in an amount to provide an AUC of about 2 to about 8 (and in another embodiment about 2 to about 3), and administering once per week per cycle about 60 to about 300 mg/m² (and in another embodiment about 50 to 100 mg/m², and in yet another embodiment about 60 to about 80 mg/m²) of paclitaxel, wherein the treatment is given for one to four weeks per cycle. In another embodiment said compound of formula 1.0 is administered in amount of about 75 to about 125 mg twice a day, and in another embodiment about 100 mg twice a day. In another embodiment said carboplatin and said paclitaxel are administered on the same day, and in another embodiment said carboplatin and said paclitaxel are administered consecutively, and in another embodiment said carboplatin is administered after said paclitaxel.

In another embodiment, this invention is directed to a method for treating non small cell lung cancer in a patient in need of such treatment comprising administering about 50 to about 200 mg of a compound of formula 1.0 twice a day, administering carboplatin once every three weeks per cycle in an amount to provide an AUC of about 2 to about 8 (in another embodiment about 5 to about 8, and in another embodiment 6), and administering once every three weeks per cycle about 150 to about 250 mg/m² (and in another embodiment about 175 to about 225 mg/m², and in another embodiment 175 mg/m²) of paclitaxel, wherein the treatment is given for one to three weeks. In another embodiment said compound of formula 1.0 is administered in an amount of about 75 to about 125 mg twice a day, and in another embodiment about 100 mg twice a day. In another embodiment said carboplatin and said paclitaxel are administered on the same day, and in another embodiment said carboplatin and said paclitaxel are administered consecutively, and in another embodiment said carboplatin is administered after said paclitaxel.

Other embodiments of this invention are directed to methods of treating cancer as described in the above embodiments (i.e., the embodiments directed to treating cancer and to treating non small cell lung cancer with a taxane and platinum coordinator compound) except that in place of paclitaxel and carboplatin the taxanes and platinum coordinator compounds used together in the methods are: (1) docetaxel (Taxotere®) and cisplatin; (2) paclitaxel and cisplatin; and (3) docetaxel and carboplatin. In another embodiment of the methods of this invention cisplatin is used in amounts of about 30 to about 100 mg/m². In the another embodiment of the methods of this invention docetaxel is used in amounts of about 30 to about 100 mg/m².

In another embodiment this invention is directed to a method of treating cancer comprising administering to a patient in need of such treatment therapeutically effective amounts of a compound of formula 1.0, a taxane, and an EGF inhibitor that is an antibody. In another embodiment the taxane used is paclitaxel, and the EGF inhibitor is a HER2 antibody (in one embodiment Herceptin) or Cetuximab, and in another embodiment Herceptin is used. The length of treatment, and the amounts and administration of said compound of formula 1.0 and the taxane are as described in the embodiments above. The EGF inhibitor that is an antibody is administered once a week per cycle, and in another embodiment is administered on the same day as the taxane, and in another embodiment is administered consecutively with the taxane. For example, Herceptin is administered in a loading dose of about 3 to about 5 mg/m² (in another embodiment about 4 mg/m²), and then is administered in a maintenance dose of about 2 mg/m² once per week per cycle for the remainder of the treatment cycle (usually the cycle is 1 to 4 weeks). In one embodiment the cancer treated is breast cancer.

In another embodiment this invention is directed to a method of treating cancer comprising administering to a patient in need of such treatment therapeutically effective amounts of: (1) a compound of formula 1.0, (2) a taxane, and (3) an antineoplastic agent selected from the group consisting of: (a) an EGF inhibitor that is a small molecule, (b) a VEGF inhibitor that is an antibody, and (c) a VEGF kinase inhibitor that is a small molecule. In another embodiment, the taxane paclitaxel or docetaxel is used. In another embodiment the antineoplastic agent is selected from the group consisting of: tarceva, Iressa, bevacizumab, SU5416, SU6688 and BAY 43-9006. The length of treatment, and the amounts and administration of said compound of formula 1.0 and the taxane are as described in the embodiments above. The VEGF kinase inhibitor that is an antibody is usually given once per week per cycle. The EGF and VEGF inhibitors that are small molecules are usually given daily per cycle. In another embodiment, the VEGF inhibitor that is an antibody is given on the same day as the taxane, and in another embodiment is administered concurrently with the taxane. In another embodiment, when the EGF inhibitor that is a small molecule or the VEGF inhibitor that is a small molecule is administered on the same day as the taxane, the administration is concurrently with the taxane. The EGF or VEGF kinase inhibitor is generally administered in an amount of about 10 to about 500 mg/m².

In another embodiment this invention is directed to a method of treating cancer comprising administering to a patient in need of such treatment therapeutically effective amounts of a compound of formula 1.0, an anti-tumor nucleoside derivative, and a platinum coordination compound.

Another embodiment of this invention is directed to a method of treating cancer comprising administering to a patient in need of such treatment therapeutically effective amounts of a compound of formula 1.0, an anti-tumor nucleoside derivative, and a platinum coordination compound, wherein said compound of formula 1.0 is administered every day, said anti-tumor nucleoside derivative is administered once per week per cycle, and said platinum coordinator compound is administered once per week per cycle. Although the treatment can be for one to four weeks per cycle, in one embodiment the treatment is for one to seven weeks per cycle.

Another embodiment of this invention is directed to a method of treating cancer comprising administering to a patient in need of such treatment therapeutically effective amounts of a compound of formula 1.0, an anti-tumor nucleoside derivative, and a platinum coordination compound, wherein said compound of formula 1.0 is administered every day, said an anti-tumor nucleoside derivative is administered once per week per cycle, and said platinum coordinator compound is administered once every three weeks per cycle. Although the treatment can be for one to four weeks per cycle, in one embodiment the treatment is for one to seven weeks per cycle.

Another embodiment of this invention is directed to a method of treating cancer comprising administering to a patient in need of such treatment therapeutically effective amounts of a compound of formula 1.0, gemcitabine, and cisplatin. In another embodiment, said compound of formula 1.0 is administered every day, said gemcitabine is administered once per week per cycle, and said cisplatin is administered once per week per cycle. In one embodiment the treatment is for one to seven weeks per cycle.

Another embodiment of this invention is directed to a method of treating cancer comprising administering to a patient in need of such treatment therapeutically effective amounts of a compound of formula 1.0, gemcitabine, and cisplatin. In another embodiment, said compound of formula 1.0 is administered every day, said gemcitabine is administered once per week per cycle, and said cisplatin is administered once every three weeks per cycle. In another embodiment the treatment is for one to seven weeks.

Another embodiment of this invention is directed to a method of treating cancer comprising administering to a patient in need of such treatment therapeutically effective amounts of a compound of formula 1.0, gemcitabine, and carboplatin. In another embodiment said compound of formula 1.0 is administered every day, said gemcitabine is administered once per week per cycle, and said carboplatin is administered once per week per cycle. In another embodiment the treatment is for one to seven weeks per cycle.

Another embodiment of this invention is directed to a method of treating cancer comprising administering to a patient in need of such treatment therapeutically effective amounts of a compound of formula 1.0, gemcitabine, and carboplatin. In another embodiment said compound of formula 1.0 is administered every day, said gemcitabine is administered once per week per cycle, and said carboplatin is administered once every three weeks per cycle. In another embodiment the treatment is for one to seven weeks per cycle.

In the above embodiments using gemcitabine, the compound of formula 1.0 and the platinum coordinator compound are administered as described above for the embodiments using taxanes. Gemcitabine is administered in an amount of about 500 to about 1250 mg/m². In one embodiment the gemcitabine is administered on the same day as the platinum coordinator compound, and in another embodiment consecutively with the platinum coordinator compound, and in another embodiment the gemcitabine is administered after the platinum coordinator compound.

Another embodiment of this invention is directed to a method of treating cancer in a patient in need of such treatment comprising administering to said patient a compound of formula 1.0 and an antineoplastic agent selected from: (1) EGF inhibitors that are antibodies, (2) EGF inhibitors that are small molecules, (3) VEGF inhibitors that are antibodies, and (4) VEGF kinase inhibitors that are small molecules all as described above. The treatment is for one to seven weeks per cycle, and generally for one to four weeks per cycle. The compound of formula 1.0 is administered in the same manner as described above for the other embodiments of this invention. The small molecule antineoplastic agents are usually administered daily, and the antibody antineoplastic agents are usually administered once per week per cycle. In one embodiment the antineoplastic agents are selected from the group consisting of: Herceptin, Cetuximab, Tarceva, Iressa, bevacizumab, IMC-1C11, SU5416, SU6688 and BAY 43-9006.

In the embodiments of this invention wherein a platinum coordinator compound is used as well as at least one other antineoplastic agent, and these drugs are administered consecutively, the platinum coordinator compound is generally administered after the other antineoplastic agents have been administered.

Other embodiments of this invention include the administration of a therapeutically effective amount of radiation to the patient in addition to the administration of a compound of formula 1.0 and antineoplastic agents in the embodiments described above. Radiation is administered according to techniques and protocols well know to those skilled in the art.

Another embodiment of this invention is directed to a pharmaceutical composition comprising at least two different chemotherapeutic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising a compound of formula 1.0 and at least two different antineoplastic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising a compound of formula 1.0 and at least one antineoplastic agent and a pharmaceutically acceptable carrier for intravenous administration. Preferably the pharmaceutically acceptable carrier is an isotonic saline solution (0.9% NaCl) or a dextrose solution (e.g., 5% dextrose).

Other embodiments of this invention are directed to the use of a combination of at least one (e.g., one) compound of formula 1.0 and drugs for the treatment of breast cancer, i.e., this invention is directed to a combination therapy for the treatment of breast cancer. Those skilled in the art will appreciate that the compounds of formula 1.0 and drugs are generally administered as individual pharmaceutical compositions. The use of a pharmaceutical composition comprising more than one drug is within the scope of this invention.

Thus, another embodiment of this invention is directed to a method of treating (or preventing) breast cancer (i.e., postmenopausal and premenopausal breast cancer, e.g., hormone-dependent breast cancer) in a patient in need of such treatment comprising administering to said patient a therapeutically effective amount of at least one (e.g., one) compound of formula 1.0 and a therapeutically effective amount of at least one antihormonal agent selected from the group consisting of: (a) aromatase inhibitors, (b) antiestrogens, and (c) LHRH analogues; and said treatment optionally including the administration of at least one chemotherapeutic agent.

The compound of formula 1.0 is preferably administered orally, and in one embodiment is administered in capsule form.

Examples of aromatase inhibitors include but are not limited to: Anastrozole (e.g., Arimidex), Letrozole (e.g., Femara), Exemestane (Aromasin), Fadrozole and Formestane (e.g., Lentaron).

Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: Goserelin (e.g., Zoladex) and Leuprolide (e.g., Leuprolide Acetate, such as Lupron or Lupron Depot).

Examples of chemotherapeutic agents include but are not limited to: Trastuzumab (e.g., Herceptin), Gefitinib (e.g., Iressa), Erlotinib (e.g., Erlotinib HCl, such as Tarceva), Bevacizumab (e.g., Avastin), Cetuximab (e.g., Erbitux), and Bortezomib (e.g., Velcade).

Preferably, when more than one antihormonal agent is used, each agent is selected from a different category of agent. For example, one agent is an aromatase inhibitor (e.g., Anastrozole, Letrozole, or Exemestane) and one agent is an antiestrogen (e.g., Tamoxifen or Fulvestrant).

Another embodiment of this invention is directed to a method of treating or preventing breast cancer in a patient in need of such treatment wherein said treatment comprises administering a therapeutically effective amount of at least one (e.g., one) compound of formula 1.0 and at least one antihormonal agent selected from the group consisting of: (a) aromatase inhibitors, (b) antiestrogens, and (c) LHRH analogues; and administering an effective amount of at least one chemotherapeutic agent.

Another embodiment of this invention is directed to a method of treating or preventing breast cancer in a patient in need of such treatment wherein said treatment comprises administering a therapeutically effective amount of at least one (e.g., one) compound of formula 1.0 and at least one antihormonal agent selected from the group consisting of: (a) aromatase inhibitors, (b) antiestrogens, and (c) LHRH analogues.

Another embodiment of this invention is directed to a method of treating or preventing breast cancer in a patient in need of such treatment wherein said treatment comprises administering a therapeutically effective amount of at least one (e.g., one) compound of formula 1.0 and at least one antihormonal agent selected from the group consisting of: (a) aromatase inhibitors, and (b) antiestrogens.

Another embodiment of this invention is directed to a method of treating or preventing breast cancer in a patient in need of such treatment wherein said treatment comprises administering a therapeutically effective amount of at least one (e.g., one) compound of formula 1.0, at least one antihormonal agent selected from the group consisting of: (a) aromatase inhibitors and (b) antiestrogens; and at least one chemotherapeutic agent.

Another embodiment of this invention is directed to a method of treating or preventing breast cancer in a patient in need of such treatment wherein said treatment comprises administering a therapeutically effective amount of at least one (e.g., one) compound of formula 1.0 and at least one aromatase inhibitor.

Another embodiment of this invention is directed to a method of treating or preventing breast cancer in a patient in need of such treatment wherein said treatment comprises administering a therapeutically effective amount of at least one (e.g., one) compound of formula 1.0, at least one aromatase inhibitor, and at least one chemotherapeutic agent.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene, and (c) LHRH analogues that are selected from the group consisting of: Goserelin and Leuprolide; and administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene, and (c) LHRH analogues that are selected from the group consisting of: Goserelin and Leuprolide.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, and (b) antiestrogens that are selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene; and administering an effective amount of at least one chemotherapeutic agents are selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0; and (2) at least one aromatase inhibitor selected from the group consisting of Anastrozole, Letrozole, Exemestane, Fadrozole and Formestane.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one aromatase inhibitor that is selected from the group consisting of Anastrozole, Letrozole, Exemestane, Fadrozole and Formestane; and (3) administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one aromatase inhibitor; and (3) at least one LHRH analogue.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one antiestrogen; and (3) at least one LHRH analogue.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one aromatase inhibitor that is selected from the group consisting of Anastrozole, Letrozole, Exemestane, Fadrozole and Formestane; and (3) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of: (1) at least one (e.g., one) compound of formula 1.0; (2) at least one antiestrogen that is selected from the group consisting of: Tamoxifen, Fulvestrant, Raloxifene, and Acolbifene; and (3) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of at least one (e.g., one) compound of formula 1.0 and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0 and Letrazole.

Another embodiment of this invention is directed to a method of treating or preventing breast cancer in a patient in need of such treatment wherein said treatment comprises administering a therapeutically effective amount of at least one (e.g., one) compound of formula 1.0 and Exemestane.

Another embodiment of this invention is directed to a method of treating or preventing breast cancer in a patient in need of such treatment wherein said treatment comprises administering a therapeutically effective amount of at least one (e.g., one) compound of formula 1.0 and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0 and Formestane.

Another embodiment of this invention is directed to a method of treating or preventing breast cancer in a patient in need of such treatment wherein said treatment comprises administering a therapeutically effective amount of at least one (e.g., one) compound of formula 1.0 and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0 and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 and Goserelin.

Another embodiment of this invention is directed to a method of treating or preventing breast cancer in a patient in need of such treatment wherein said treatment comprises administering a therapeutically effective amount of at least one (e.g., one) compound of formula 1.0 and Leuprolide.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Raloxifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Goserelin, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Leuprolein, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Anastrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Letrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Exemestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Fadrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Formestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Goserelin, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Goserelin, and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide, and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Letrozole.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Exemestane.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0, Goserelin and Formestane.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Letrozole.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Exemestane.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0, Leuprolide and Formestane.

Another embodiment of this invention is directed to the treatment or prevention of breast cancer in a patient in need of such treatment, said treatment comprising the administration of a therapeutically effective amount of at least one (e.g., one) compound of formula 1.0 and Anastrozole.

Another embodiment of this invention is directed to the treatment or prevention of breast cancer in a patient in need of such treatment, said treatment comprising the administration of a therapeutically effective amount of at least one (e.g., one) compound of formula 1.0 and Letrozole.

Another embodiment of this invention is directed to the treatment or prevention of breast cancer in a patient in need of such treatment, said treatment comprising the administration of a therapeutically effective amount of at least one (e.g., one) compound of formula 1.0 and Exemestane.

Another embodiment of this invention is directed to the treatment or prevention of breast cancer in a patient in need of such treatment, said treatment comprising the administration of a therapeutically effective amount of at least one (e.g., one) compound of formula 1.0 and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0 and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0, Anastrozole, and Fulvestrant.

Another embodiment of this invention is directed to the treatment or prevention of breast cancer in a patient in need of such treatment, said treatment comprising the administration of a therapeutically effective amount of at least one compound of formula 1.0 (e.g., one), Letrozole, and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0, Exemestane, and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0, Anastrozole, and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0, Letrozole, and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0, Exemestane, and Tamoxifen.

Other embodiments of this invention are directed to any of the above described embodiments for the treatment of Breast Cancer wherein the chemotherapeutic agent is Trastuzumab.

Other embodiments of this invention are directed to any of the above described embodiments for the treatment or prevention of Breast Cancer wherein the method is directed to the treatment of breast cancer.

The compound of formula 1.0, antihormonal agents and chemotherapeutic agents can be administered concurrently or sequentially.

The antihormonal agents and optional chemotherapeutic agents are administered according to their protocols, dosage amounts, and dosage forms that are well know to those skilled in the art (e.g., the Physician's Desk Reference or published literature). For example, for Tamoxifen, Fulvestrant, Raloxifene, Anastrozole, Letrozole, Exemestane, Leuprolide and Goserelin, see the Physician's Desk Reference, 57^{th} Edition, 2003, published by Thomas PDR at Montvale, N.J. 07645-1742, the disclosure of which is incorporated herein by reference thereto.

In general, in the embodiments directed to the methods of treating Breast Cancer: (1) the compound of formula 1.0 can be administered daily (e.g., once per day, and in one embodiment twice a day), (2) the aromatase inhibitors can be administered in accordance with the known protocol for the aromatase inhibitor used (e.g., once per day), (3) the antiestrogens can be administered in accordance with the known protocol for the antiestrogen used (e.g., from once a day to once a month), (4) the LHRH analogue can be administered in accordance with the known protocol for the LHRH analogue used (e.g., once a month to once every three months), and (5) the chemotherapeutic agent can be administered in accordance with the known protocol for the chemotherapeutic agent used (e.g., from once a day to once a week).

Radiation therapy, if administered in the above treatments for breast cancer, is generally administered according to known protocols before administration of the compound of formula 1.0, antihormonal agents and optional chemotherapeutic agents.

Treatment according to the methods of treating breast cancer is continuous (i.e., a continuous dosing schedule is followed). The treatment is continued until there is a complete response, or until the skilled clinician determines that the patient is not benefiting from the treatment (for example, when there is disease progression).

The continuous treatment protocol for breast cancer can be changed to a discontinuous treatment schedule if, in the judgment of the skilled clinician, the patient would benefit from a discontinuous treatment schedule with one or more of the administered drugs. For example, the compound of formula 1.0 can be given using a discontinous treatment schedule while the remaining drugs used in the treatment are given as described herein. An example of a discontinuous treatment protocol for the compound of formula 1.0 is a repeating cycle of three weeks with the compound of formula 1.0 followed by one week without the compound of formula 1.0.

After a complete response is achieved with the breast cancer treatment, maintenance therapy with the compound of formula 1.0 can be continued using the dosing described in the methods of this invention. Maintenance therapy can also include administration of the antihormonal agents using the dosing described in the methods of this invention. Maintenance therapy can just be with the antihormonal agents. For example, after a complete response is achieved, an aromatase inhibitor (e.g., Anastrozole, Letrozole or Exemestane) can be continued for up to five years. Or, for example, an antiestrogen, e.g., Tamoxifen, may be used for up to five years after a complete response is achieved. Or, for example, an antiestrogen (e.g., Tamoxifen) can be used for up to five years after a complete response is achieved followed by the use of an aromatase inhibitor (e.g., Anastrozole, Letrozole or Exemestane) for up to five years.

In the embodiments directed to the treatment of breast cancer described above, the compound of formula 1.0 is administered continuously in a total daily dose of about 100 mg to about 600 mg. Usually this amount is administered in divided doses, and in one embodiment this amount is administered twice a day. In one embodiment the compound of formula 1.0 (for example, as described in any one of Embodiment Nos. 1 to 107) is dosed twice a day in an amount of about 50 mg to about 300 mg per dose. In another embodiment the compound of formula 1.0 is dosed twice a day in an amount of about 100 mg to about 200 mg per dose. Examples include the compound of formula 1.0 being dosed twice a day at 100 mg per dose. Examples also include the compound of formula 1.0 being dosed twice a day at 200 mg per dose.

Anastrozole is administered p.o. and is dosed once a day in amounts of about 0.5 to about 10 mg per dose, and in one embodiment in an amount of about 1.0 mg per dose.

Letrozole is administered p.o. and is dosed once a day in amounts of about 1.0 to about 10 mg per dose, and in one embodiment in an amount of about 2.5 mg per dose.

Exemestane is administered p.o. and is dosed once a day in amounts of about 10 to about 50 mg per dose, and in one embodiment in an amount of about 25 mg per dose.

Fadrozole is administered p.o. and is dosed twice a day in amounts of about 0.5 to about 10 mg per dose, and in one embodiment in an amount of about 2.0 mg per dose.

Formestane is administered i.m. and is dosed once every two weeks in amounts of about 100 to about 500 mg per dose, and in one embodiment in an amount of about 250 mg per dose.

Tamoxifen is administered p.o. and is dosed once a day in amounts of about 10 to about 100 mg per dose, and in one embodiment in an amount of about 20 mg per dose.

Fulvestrant is administered i.m. and is dosed once a month in amounts of about 100 to about 1000 mg per dose, and in one embodiment in an amount of about 250 mg per dose.

Raloxifene is administered p.o. and is dosed once a day in amounts of about 10 to about 120 mg per dose, and in one embodiment in an amount of about 60 mg per dose.

Acolbifene is administered p.o. and is dosed once a day in amounts of about 5 to about 20 mg per dose, and in one embodiment in an amount of about 20 mg per dose.

Goserelin is administered s.c. and is dosed once a month, or once every three months, in amounts of about 2 to about 20 mg per dose, and in one embodiment in an amount of about 3.6 mg per dose when administered once a month, and in another embodiment in an amount of about 10.8 mg per dose when administered once every three months.

Leuprolide is administered s.c. and is dosed once a month, or once every three months, in amounts of about 2 to about 20 mg per dose, and in one embodiment in an amount of about 3.75 mg per dose when administered once a month, and in another embodiment in an amount of about 11.25 mg per dose when administered once every three months.

Trastuzumab is administered by i.v. and is dosed once a week in amounts of about 2 to about 20 mpk per dose, and in one embodiment in an amount of about 2 mpk per dose. Trastuzumab is generally initially administered in a loading dose that is generally twice the dose of the weekly dose. Thus, for example, a 4 mpk loading dose is administered and then dosing is 2 mpk per dose per week.

Gefitinib is administered p.o. and is dosed once a day in amounts of about 100 to about 1000 mg per dose, and in one embodiment in an amount of about 250 mg per dose.

Erlotinib is administered p.o. and is dosed once a day in amounts of about 100 to about 500 mg per dose, and in one embodiment in an amount of about 150 mg per dose.

Bevacizumab is administered i.v. and is dosed once every two weeks in amounts of about 2.5 to about 15 mg per kilogram of body weight per dose, and in one embodiment in an amount of about 10 mg per kilogram per dose.

Cetuximab is administered i.v. and is dosed once a week in amounts of about 200 to about 500 mg per meter squared dose, and in one embodiment in an amount of about 250 mg per meter squared per dose.

Bortezomib is administered i.v. and is dosed twice a week for 2 weeks followed by a 10 day rest period (21 day treatment cycle) for a maximum of 8 treatment cycles in amounts of about 1.0 to about 2.5 mg per meter squared per dose, and in one embodiment in an amount of about 1.3 mg per meter squared per dose.

Thus in one embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Anastrozole p.o. in an amount of about 0.5 to about 10 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Anastrozole in an amount of about 1.0 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Letrozole p.o. in an amount of about 1.0 to about 10 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Letrozole p.o. in an amount of about 2.5 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Exemestane p.o. in an amount of about 10 to about 50 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Exemestane in an amount of about 25 mg per dose wherein each dose is given once a day.

In another embodiment of this invention breast cancer is treated (or prevented) in a patient in need of such treatment wherein said treatment comprises administering to said patient: (1) the compound of formula 1.0 orally in an amount of about 50 mg to about 300 mg per dose wherein each dose is administered twice a day, and (2) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 orally in an amount of about 100 to 200 mg per dose, wherein each dose is administered twice a day, and (2) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, and (2) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, and (2) Tamoxifen p.o. in an amount of about 20 mg per dose wherein each dose is given once a day.

In other embodiments of the invention breast cancer is treated in a patient in need of such treatment wherein said treatment comprises the administration of the compound of formula 1.0, one of the aromatase inhibitors (e.g., Anastrozole, Letrozole, or Exemestane, and in one embodiment Anastrozole), and one of the antiestrogens (e.g., Fulvestrant or Tamoxifen), wherein the compound of formula 1.0, aromatase inhibitor and antiestrogen are administered in the dosages described above.

Thus, for example in another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Anastrozole p.o. in an amount of about 0.5 to about 10 mg per dose wherein each dose is given once a day, and (3) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 p.o in an amount of about 100 to 200 mg per dose, wherein each dose is administered twice a day, (2) Anastrozole p.o. in an amount of about 1.0 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Letrozole p.o in an amount of about 1.0 to about 10 mg per dose wherein each dose is given once a day, and (3) Fulvestrant in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Letrozole p.o. in an amount of about 2.5 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Exemestane p.o. in an amount of about 10 to about 50 mg per dose wherein each dose is given once a day, and (3) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Exemestane p.o. in an amount of about 25 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Anastrozole p.o. in an amount of about 0.5 to about 10 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Anastrozole p.o. in an amount of about 1.0 mg per dose wherein each dose is given once a day, and (3) Tamoxifen p.o. in an amount of about 20 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Letrozole p.o. in an amount of about 1.0 to about 10 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Letrozole p.o. in an amount of about 2.5 mg per dose wherein each dose is given once a day, and (3) Tamoxifen p.o. in an amount of about 20 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 50 mg to about 300 mg per dose wherein each dose is administered twice a day, (2) Exemestane p.o. in an amount of about 10 to about 50 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 p.o. in an amount of about 100 to 200 mg per dose, wherein each dose is administered twice a day, (2) Exemestane p.o. in an amount of about 25 mg per dose wherein each dose is given once a day, and (3) Tamoxifen p.o. in an amount of about 20 mg per dose wherein each dose is given once a day.

Those skilled in the art will appreciate that when other combinations of antihormonal agents are used, the individual antihormonal agent is used in the amounts specified above for that individual antihormonal agent.

Other embodiments of the treatment of Breast Cancer are directed to the methods of treating Breast Cancer described above wherein the compound of formula 1.0 is dosed twice a day in an amount of about 100 mg per dose.

Other embodiments of the treatment of Breast Cancer are directed to the methods of treating Breast Cancer described above wherein the compound of formula 1.0 is dosed twice a day in an amount of about 200 mg per dose.

Other embodiments of the treatment of Breast Cancer are directed to the methods of treating Breast Cancer described above wherein a chemotherapeutic agent is administered in addition to the compound of formula 1.0 and antihormonal agent (or antihormonal agents). In these embodiments the dosage ranges of the compound of formula 1.0 and antihormonal agents are as those described above in the combination therapies, or those described above for the individual compound of formula I and antihormonal agents, and the dosages of the chemotherapeutic agents are those described above for the individual chemotherapeutic agent. The dosages for the chemotherapeutic agents are well known in the art.

Other embodiments of this invention are directed to pharmaceutical compositions comprising the compound of formula 1.0 and at least one antihormonal agent and a pharmaceutically acceptable carrier.

Other embodiments of this invention are directed to pharmaceutical compositions comprising the compound of formula 1.0, at least one antihormonal agent, at least one chemotherapeutic agent, and a pharmaceutically acceptable carrier.

Other embodiments of this invention are directed to pharmaceutical compositions comprising the compound of formula 1.0, at least one chemotherapeutic agent, and a pharmaceutically acceptable carrier.

Those skilled in the art will appreciate that the compounds (drugs) used in the methods of this invention are available to the skilled clinician in pharmaceutical compositions (dosage forms) from the manufacturer and are used in those compositions. So, the recitation of the compound or class of compounds in the above described methods can be replaced with a recitation of a pharmaceutical composition comprising the particular compound or class of compounds. For example, the embodiment directed to a method of treating cancer comprising administering to a patient in need of such treatment therapeutically effective amounts of the compound of formula 1.0, a taxane, and a platinum coordination compound, includes within its scope a method of treating cancer comprising administering to a patient in need of such treatment therapeutically effective amounts of a pharmaceutical composition comprising the compound of formula 1.0, a pharmaceutical composition comprising a taxane, and a pharmaceutical composition comprising a platinum coordination compound.

Those skilled in the art will recognize that the actual dosages and protocols for administration employed in the methods of this invention may be varied according to the judgment of the skilled clinician. The actual dosage employed may be varied depending upon the requirements of the patient and the severity of the condition being treated. Determination of the proper dosage for a particular situation is within the skill of the art. A determination to vary the dosages and protocols for administration may be made after the skilled clinician takes into account such factors as the patient's age, condition and size, as well as the severity of the cancer being treated and the response of the patient to the treatment.

The amount and frequency of administration of the compound of formula 1.0 and the chemotherapeutic agents will be regulated according to the judgment of the attending clinician (physician) considering such factors as age, condition and size of the patient as well as severity of the cancer being treated.

The chemotherapeutic agent can be administered according to therapeutic protocols well known in the art. It will be apparent to those skilled in the art that the administration of the chemotherapeutic agent can be varied depending on the cancer being treated and the known effects of the chemotherapeutic agent on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the cancer to the administered therapeutic agents.

The initial administration can be made according to established protocols known in the art, and then, based upon the observed effects, the dosage, modes of administration and times of administration can be modified by the skilled clinician.

The particular choice of chemotherapeutic agent will depend upon the diagnosis of the attending physicians and their judgement of the condition of the patient and the appropriate treatment protocol.

The determination of the order of administration, and the number of repetitions of administration of the chemotherapeutic agent during a treatment protocol, is well within the knowledge of the skilled physician after evaluation of the cancer being treated and the condition of the patient.

Thus, in accordance with experience and knowledge, the practicing physician can modify each protocol for the administration of an chemotherapeutic agent according to the individual patient's needs, as the treatment proceeds. All such modifications are within the scope of the present invention.

The particular choice of antihormonal agents, optional chemotherapeutic agents and optional radiation will depend upon the diagnosis of the attending physicians and their judgment of the condition of the patient and the appropriate treatment protocol.

The determination of the order of administration, and the number of repetitions of administration of the antihormonal agents, optional chemotherapeutic agents and optional radiation during a treatment protocol, is well within the knowledge of the skilled physician after evaluation of the breast cancer being treated and the condition of the patient.

Thus, in accordance with experience and knowledge, the practicing physician can modify each protocol for the administration of antihormonal agents, optional chemotherapeutic agents and optional radiation according to the individual patient's needs, as the treatment proceeds. All such modifications are within the scope of the present invention.

The attending clinician, in judging whether treatment is effective at the dosage administered, will consider the general well-being of the patient as well as more definite signs such as relief of cancer-related symptoms (e.g., pain, cough (for lung cancer), and shortness of breath (for lung cancer)), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Compounds of the invention may be prepared according to the procedures described in WO 95/10516 published Apr. 20, 1995, WO96/31478 published Oct. 10, 1996, WO 97/23478 published Jul. 3, 1997, U.S. Pat. No. 5,719,148 issued Feb. 17, 1998, and copending application Ser. No. 09/094,687 filed Jun. 15, 1998 (see also WO98/57960 published Dec. 23, 1998); the disclosures of each being incorporated herein by reference thereto; and according to the procedures described below.

Compounds of the invention can be prepared according to the reaction schemes described below.

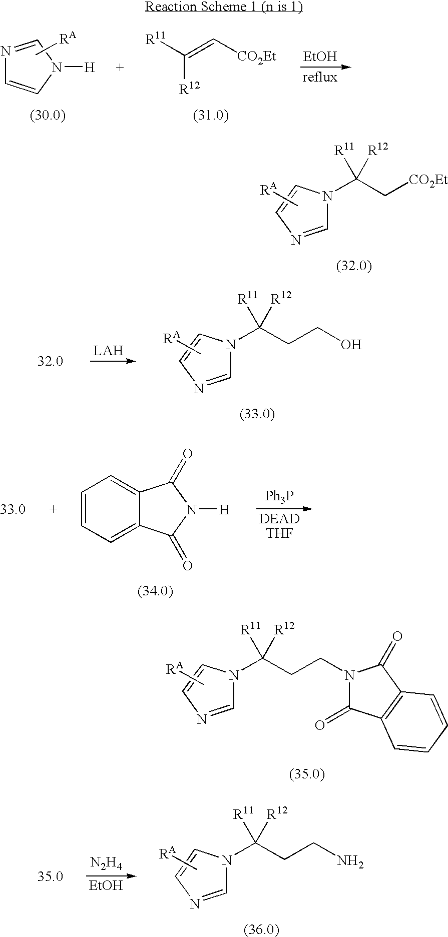

In Scheme 1, R¹¹ and R¹² are preferably methyl when H is bound to the amide nitrogen (i.e., when R⁸ in formula 1.0 is H), and are preferably H when the amide nitrogen is substituted (i.e., R⁸ in formula 1.0 is other than H). Those skilled in the art will appreciate that other acylating agents can be used in place of cyclohexyl isocyanate to obtain compounds having different groups bound to the carbonyl group that is bound to the piperazine nitrogen. Those skilled in the art will also appreciate that other esters can be used in place of compound 31.0 to obtain compounds having different carbon chains between the imidazole ring and the —C(O)NH-group.

In Scheme 1, and the Schemes that follow, Y represents C, N or N⁺O⁻ such that there can only be 0-2 Y substituents that are independently selected from N or N⁺O⁻. R^{A} represents the optional substituents in the imidazole ring that are defined for imidazole ring 4.0 above. R^{B} represents the optional substituents defined above for the aryl or heteroaryl groups for R⁸.

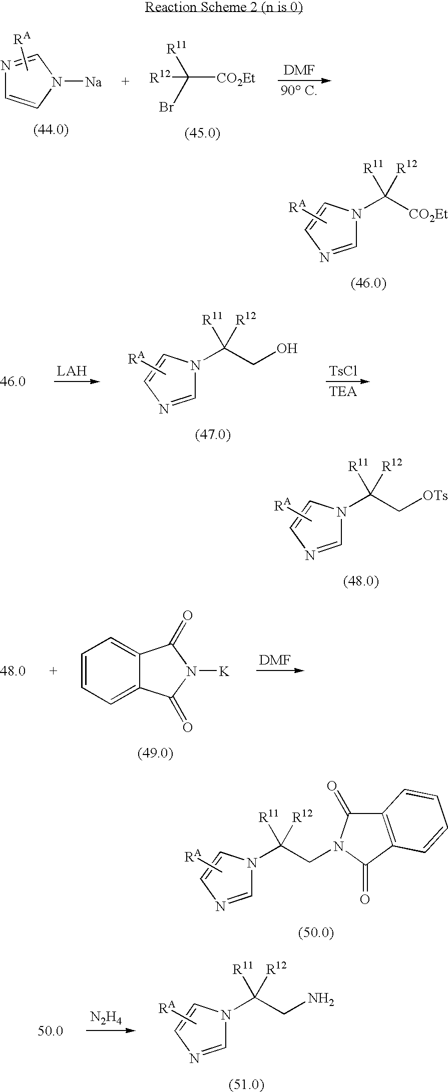

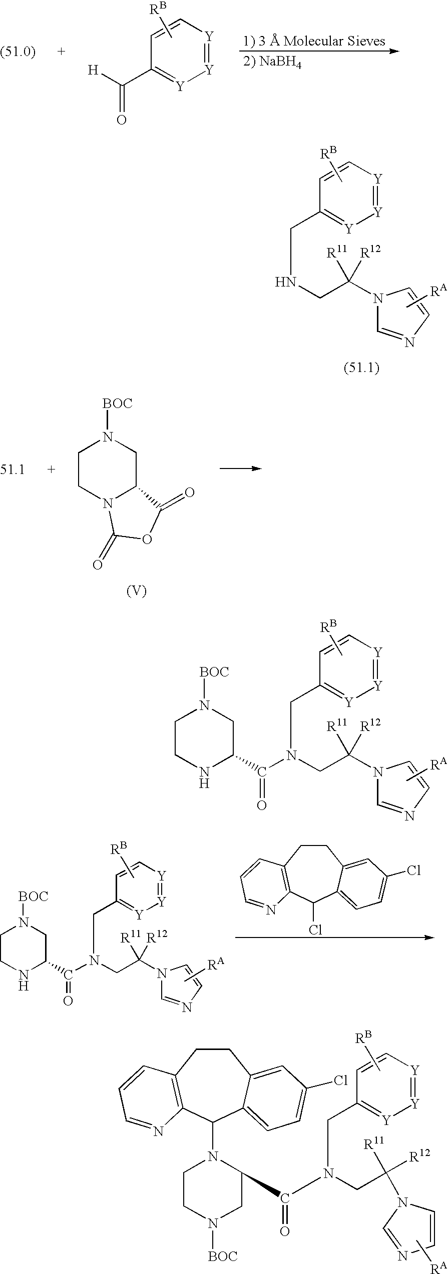

The synthesis of the intermediate amine 51.0 begins with the alkylation of the sodium salt of imidazole (or substituted imidazole) 44.0 with 45.0 at 90° C. Standard LAH reduction of the ester 46.0 gives the alcohol 47.0. Tosylation of 47.0 and displacement of tosylate with potassium phthalimide 49.0 in DMF at 90° C. gives the phthalimido derivative 50.0 which can be readily converted to the amine 51.0 with hydrazine in refluxing EtOH.

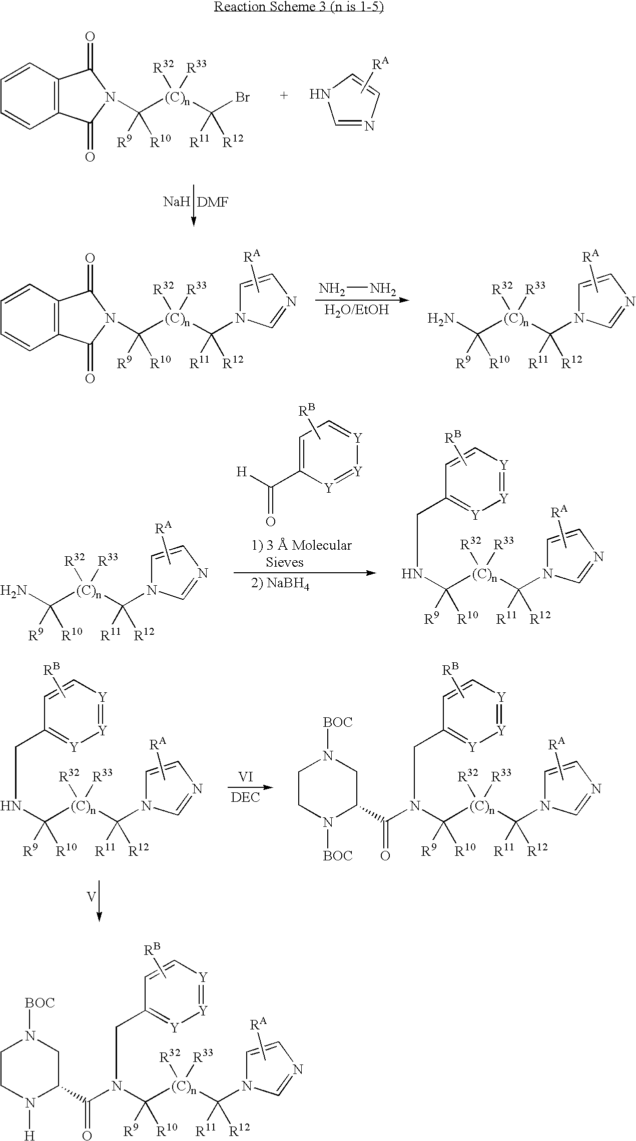

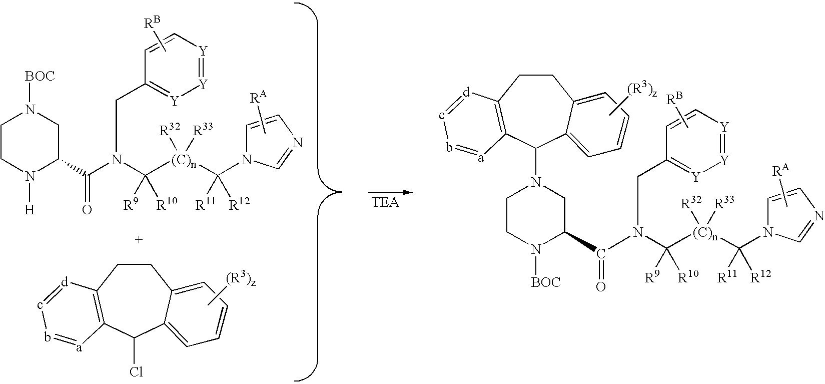

Reactants V and VI are:

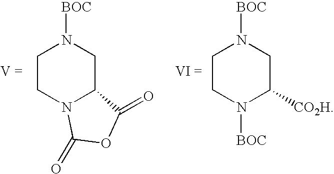

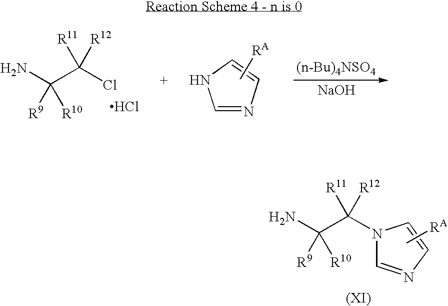

In Scheme 6, the procedure set forth in Scheme 3 is followed, but using 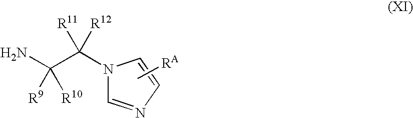
instead of 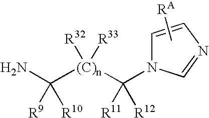
to obtain compounds wherein n is O, Similarly, using 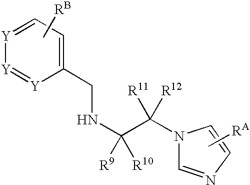
(obtained from XI following the procedures in Scheme 3), instead of 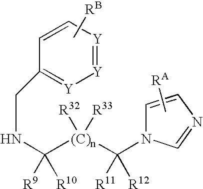
in Scheme 3 produces compounds wherein n is 0.

Those skilled in the art will appreciate that in Schemes 1, 2, 3 and 4, other aldehydes can be used in place of 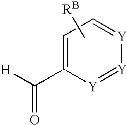
to obtain the other substituents for R⁸ in formula 1.0.

Those skilled in the art will also appreciate that using 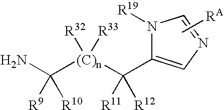
instead of 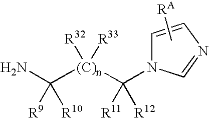
in Scheme 3, and using 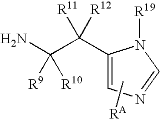
instead of 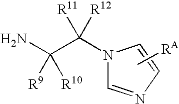
in Scheme 4 will provide the corresponding compounds wherein the imidazole is bound to the alkyl chain by a ring carbon.

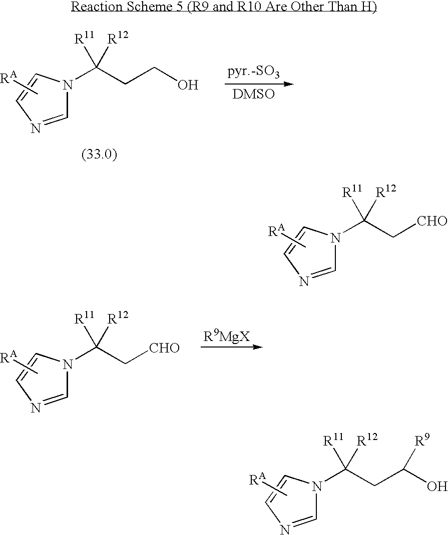

In Scheme 5, the alcohol 33.0 can be oxidized under standard conditions to give the aldehyde. Addition of the corresponding Grignard of R⁹ gives the alcohol which can be carried on to amine as in Scheme 1 or subject to reoxidation to the ketone followed by Grignard addition of R¹⁰. In the case where R⁹=R¹⁰, the ester 32.0 (Scheme 1) can be used as the electrophile with 2 equivalents of the appropriate Grignard reagent being added.

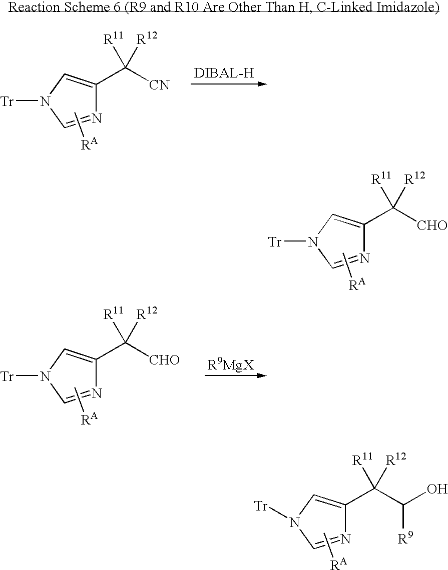

In Scheme 6, the nitrile may be reduced with DIBAL-H to the aldehyde. Similar to the procedure in Scheme 5, the aldehyde can then be treated with the appropriate Grignard reagent to give the alcohol. There can be an additional round of oxidation and Grignard addition to give the R⁹, R¹⁰ disubstituted derivatives with either R⁹=R¹⁰ or R⁹≠R¹⁰. The resulting alcohol may be converted to the amine by the methodology shown in either Schemes 1 or 2.

Compounds useful in this invention are exemplified by the following examples, which examples should not be construed as limiting the scope of the disclosure.

Preparative Examples 1 to 141 are prophetic examples.

### PREPARATIVE EXAMPLE 1

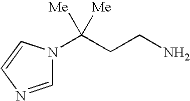 Step A

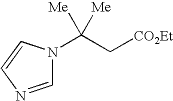

Ethyl 2,2-dimethyl acrylate (50.0 g, 2.0 eq.) is stirred with imidazole (13.28 g, 200 mmol) at 90° C. for 48 hours. The resulting solution is cooled, diluted with water (150 mL) and CH₂Cl₂ (150 mL) and separated. The aqueous layer is washed with CH₂Cl₂ (2×75 mL) and the combined organics are dried over Na₂SO₄ and concentrated in vacuo. The crude mixture is purified by flash chromatography using a 10% MeOH in CH₂Cl₂ solution as eluent to give the pure product.
Step B

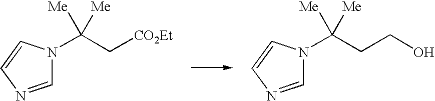

A solution of the title compound from Step A (10.0 g, 50.96 mmol) is treated with LiAlH₄ (51 mL, 1M solution in ether, 1.0 eq.). The reaction mixture is stirred one hour at room temperature before quenching by the dropwise addition of saturated Na₂SO₄ (˜3.0 mL). The resulting slurry is dried with Na₂SO₄ (solid), diluted with EtOAc (100 mL) and filtered through a plug of Celite. The filtrate is concentrated which is used without further purification.
Step C

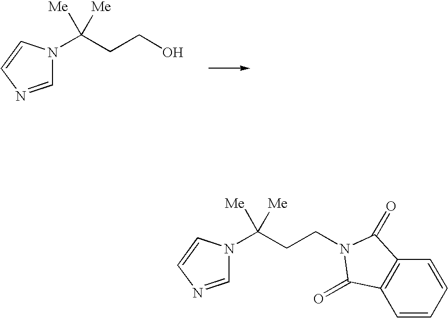

To a solution of the title compound of Step B (6.85 g, 44.42 mmol), phthalimide (7.19 g, 1.1 eq.), and Ph₃P (12.82 g, 1.1 eq.) in THF (200 mL) at 0° C. is added DEAD (7.69 mL, 1.1 eq.) over 10 minutes. The resulting solution is warmed to room temperature and stirred 48 hours. The reaction mixture is concentrated under reduced pressure and the product isolated by crystallization from CH₂Cl₂/Et₂O to give the product
Step D

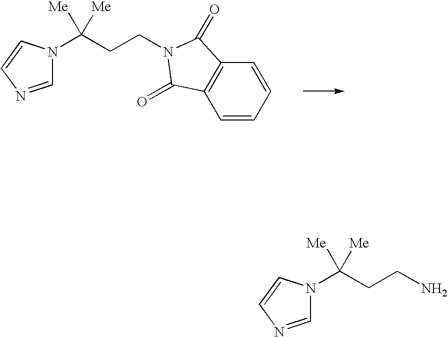

A solution of the title compound from Step C (9.50 g, 33.53 mmol) and N₂H₄ (1.25 mL, 1.2 eq.) in EtOH (100 mL) is heated at reflux 4 hours. The resulting slurry is cooled, filtered, and the filtrate concentrated under reduced pressure. The crude product is purified by flash chromatography using a 15% (10% NH₄OH in MeOH) solution in CH₂Cl₂ as eluent to give then product.

### PREPARATIVE EXAMPLES 2-4

By essentially the same procedure as that set forth in Preparative Example 1, the amines in Column 3 of Table 1 are synthesized from the esters in Column 2. “No.” represents “Preparative Example Number”.

TABLE 1

| No. | ESTER | AMINE |
|---|---|---|
| 2 | 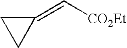 | 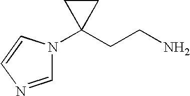 |
| 3 | 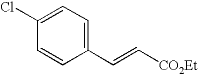 | 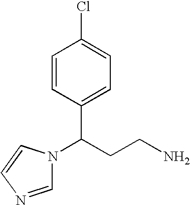 |
| 4 | 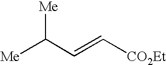 | 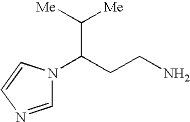 |

### PREPARATIVE EXAMPLE 5

Step A

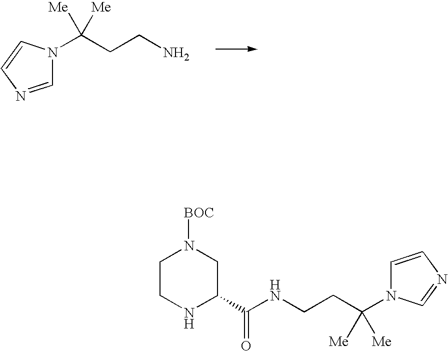

To the title compound from Preparative Example 1, Step D, (0.82 g, 5.35 mmol) in CH₂Cl₂ (10 mL) and TEA (0.75 mL, 1.0 eq) is added piperazine anhydride (1.65 g, 1.2 eq.) (prepare as described in Preparative Example 33) portionwise and the resulting solution is stirred at room temperature. When the reaction is complete (TLC), the solution is concentrated in vacuo and the crude product is purified by flash chromatography using a 10% (10% NH₄OH in MeOH) in CH₂Cl₂ then 20% (10% NH₄OH in MeOH) in CH₂Cl₂ as eluent.
Step B

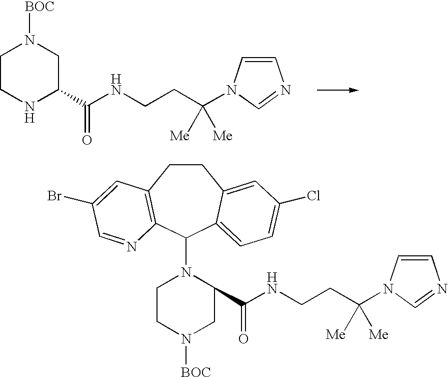

The title compound from Step A is dissolved in CH₂Cl₂ (30 mL) and TEA (7.62 mL, 10 eq.) is added. The reaction mixture is stirred 5 minutes before adding chloride 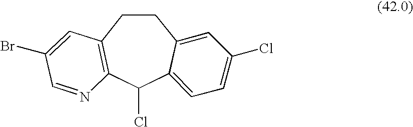
(0.908 g, 0.5 eq.). The resulting solution is stirred at room temperature for 96 hours. The reaction mixture is diluted with water (50 mL), separated and the aqueous layer is extracted with CH₂Cl₂ (2×200 mL). The combined organics are dried over MgSO₄, filtered, and concentrated under reduced pressure. The crude product is purified by flash chromatography using a 5%, 7.5%, and then 10% (10% NH₄OH in MeOH) in CH₂Cl₂ solution as eluent (0.926 g, 30% yield).
Step C

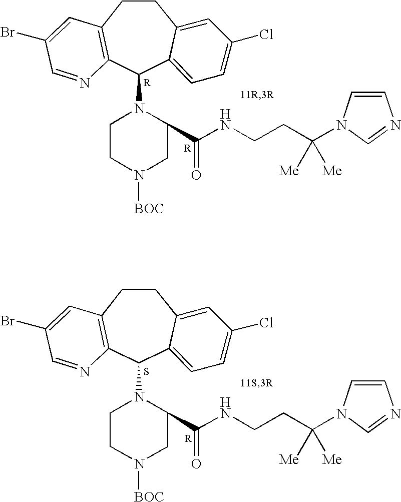

If one were to separate the title compound from Step B using Preparative HPLC using a ChiralPak AD column using a 20% IPA in hexanes with 0.2% diethylamine solution as eluent then one would could the above diastereomers.

### PREPARATIVE EXAMPLE 6

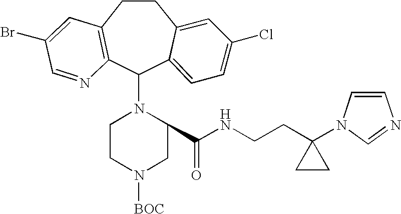

By essentially the same procedure as described in Preparative Example 5, except using the title compound from Preparative Example 2 (Table 1), the title compound is prepared.

If one were to separate the title compound by Preparative HPLC using a CHIRALPAK AD column using a 30% IPA in hexanes containing 0.2% diethylamine solution as eluent, then one could obtain the diastereomers

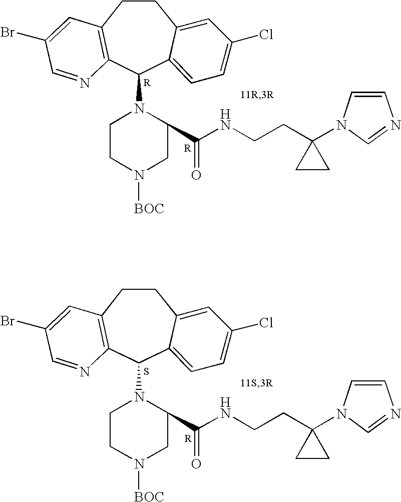

### PREPARATIVE EXAMPLE 7

If one were to follow the procedure set forth in Preparative Example 8, except using the amine 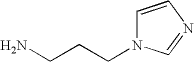
in Step A instead of 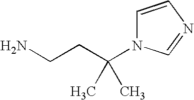
and using the 10-Cl tricycle chloride 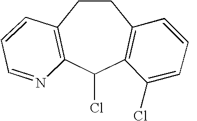
in Step B instead of the 3-Br-8-Cl-tricycle chloride then one could obtain the compounds 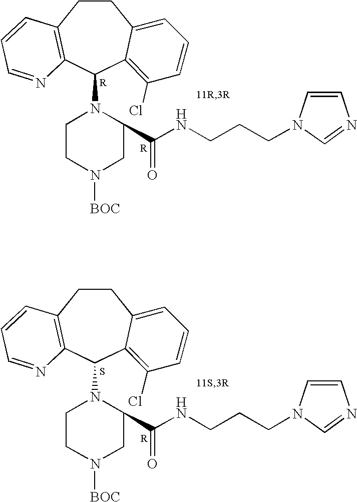
from the compound

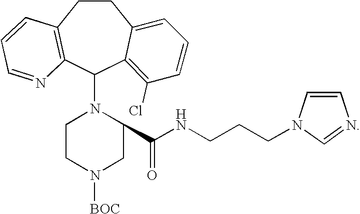

Obtain the 10-Cl tricycle chloride (10,11-diChloro-6,11-dihydro-5H-benzo[5,6]cyclohepta[1,2-B]pyridine) as follows:

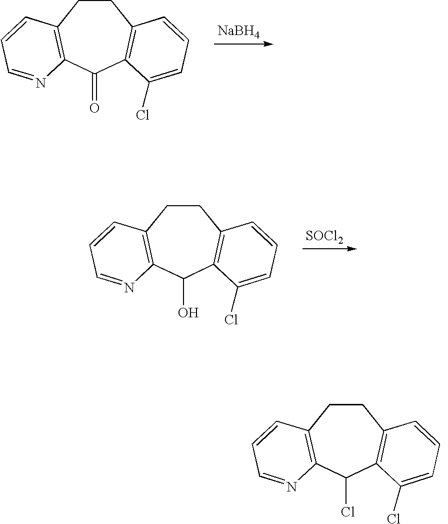

The ketone (starting material) 5,6-dihydro-10-Chloro-11H-benzo[5,6]-cyclohepta[1,2-c]pyridine-11-one, can be prepared following the procedure described by Villani et al., J. Het. Chem. 8, 73-81 (1971). The product is prepared substituting the 10-Chloro for the 10H tricycle and following the procedure described in Preparative Example 169.

### PREPARATIVE EXAMPLE 8

Step A

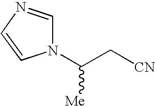

Imidazole (2.73 g, 40.1 mmol) in crotonitrile (10 mL) is heated to reflux overnight. The resulting solution is concentrated in vacuo, the residue diluted with Et₂O (50 mL) and washed with water (2×100 mL) and brine (1×25 mL). The combined organics were dried over Na₂SO₄ and concentrated under reduced pressure. The crude product is purified by flash chromatography using a 15% MeOH in CH₂Cl₂ solution as eluent (2.13 g, 39% yield).
Step B

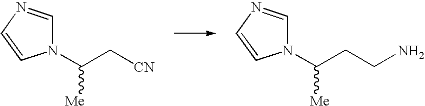

A solution of the title compound from Step A (0.50 g, 0.0037 mmol) in THF (10 mL) is treated with LAH (5.5 mL, 1.0 M in Et₂O, 1.1 eq.). The reaction mixture is stirred at room temperature 3 hours and quenched by the dropwise addition of saturated Na₂SO₄. The resulting slurry is dried by the addition of solid Na₂SO₄ and filtered through a plug of Celite. The filtrate is concentrated under reduced pressure and the crude residue purified by flash chromatography using a 20% (10% NH₄OH in MeOH) solution as eluent (0.03 g, 6% yield).

### PREPARATIVE EXAMPLE 9

Step A

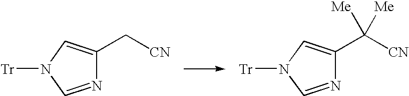

nBuLi (2.5 mL; 2.5M in hexanes; 2.1 eq.) is added to iPr₂NH (0.87 mL, 2.1 eq.) in THF (8.0 mL) at 0° C. The resulting solution is stirred 45 minutes before adding the nitrile (1.0 g, 2.97 mmol) in THF (7.0 mL). The reaction mixture is stirred at 0° C. for 30 minutes before adding MeI (0.37 mL, 2.0 eq.). The resulting solution is warmed to room temperature and stirred one hour. The reaction is quenched by the addition of 1N HCl until acidic, diluted with water (40 mL) and extracted with EtOAc (2×200 mL). The combined organics are dried over Na₂SO₄ and concentrated under reduced pressure. The crude product is purified by flash chromatography using a 40% EtOAc solution in hexanes as eluent.
Step B

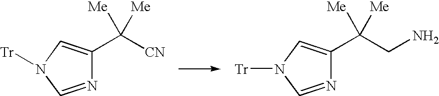

LiAlH₄ (2.7 mL; 1.0 M solution in THF; 1.5 eq.) is added to the title compound from Step A (0.68 g, 1.80 mmol) in THF (5.0 mL). The resulting solution is stirred at room temperature 1.5 hours and quenched by the dropwise addition of saturated Na₂SO₄ (10 mL). The solution is extracted with Et₂O (2×200 mL), the combined organics dried over MgSO₄ and concentrated under reduced pressure (0.6 g, 88% yield).
Step C 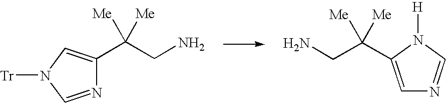
following the same procedure as set forth in Preparative Example 24 Step C, the title compound is prepared.

### PREPARATIVE EXAMPLES 10-14

Following the procedures found in J. Chem. Soc. Perkin 1 (1979), 1341-1344, the following N-substituted histamines are prepared:

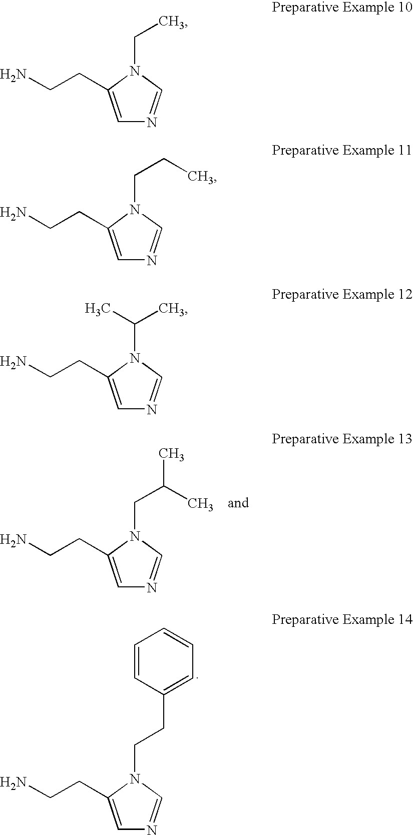

### PREPARATIVE EXAMPLES 15-23

By essentially the same procedure as that set forth in Preparative Example 57, and using the aldehydes and amines set forth in Table 2, one can obtain the intermediate products shown in Table 2.

TABLE 2

| Prep | | | |
|---|---|---|---|
| Ex. | Aldehyde | Amine | Product |
| 15 | 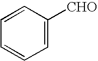 | 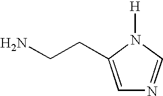 | 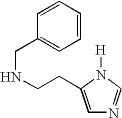 |
| 16 | 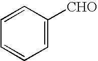 | 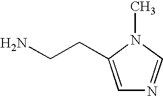 | 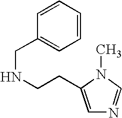 |
| 17 | 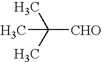 | 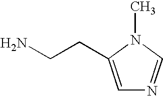 | 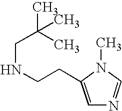 |
| 18 | 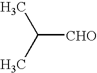 | 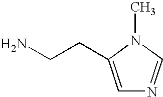 | 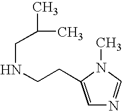 |
| 19 | 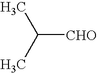 | 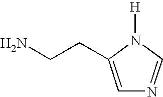 | 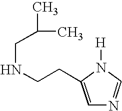 |
| 20 | 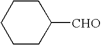 | 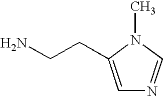 | 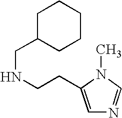 |
| 21 | 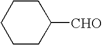 | 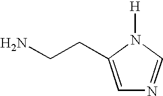 | 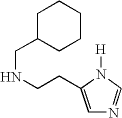 |
| 22 | 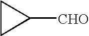 | 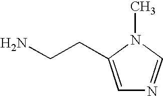 | 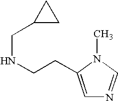 |
| 23 | 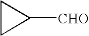 | 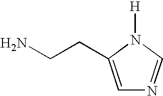 | 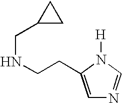 |

The amine products in Table 2 can be reacted with Reactant V (see Scheme 3).

### PREPARATIVE EXAMPLE 24

Step A

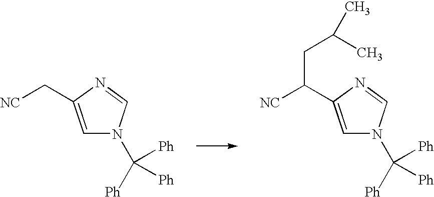

Dissolve the nitrile (1.5 g, 4.29 mmol) in 10 mL of THF and cool to −78° C. under nitrogen. Add 20 mL of a 1.5 M LDA solution (in cyclohexane). Then add dropwise over 2 hr, a solution of 790 mg (4.293 mmol) of 2-methylpropyliodide in 10 mL of THF. Allow to warm to room temperature and stir overnight. Add 10 mL of water followed by 1N HCl until pH of 10-11. Dilute with 100 mL of methylene chloride followed by 20 mL of sat. aqueous Na₂SO₄. Add MgSO₄ until solution is clear. Separate the organic layer and dry over MgSO₄. Concentrate under vacuum and flash chromatograph on silica gel using ethyl acetate-hexane (1-3) to give the product.
Step B

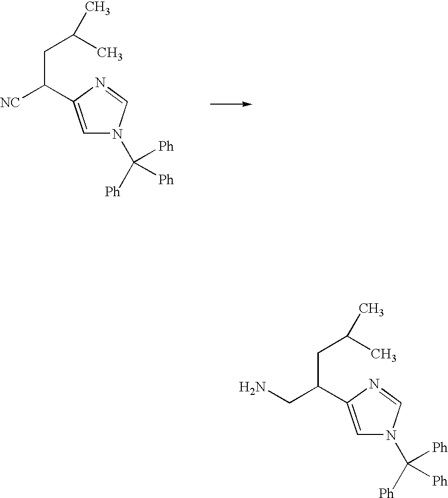

Dissolve the product of Step A (0.5 g, 1.23 mmol) in 10 mL of ethanol saturated with ammonia. Add 8.8 mg (0.017 mmol) of H₂PtCl₆.6H₂O, 1 g of Raney Ni in water and hydrogenate at 54 psi on a Parr shaker over night. Filter through Celite and concentrate under vacuum.
Step C

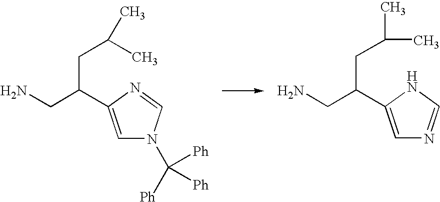

Dissolve the product of Step B (0.165 g, 0.403 mmol) in 4 mL of 2M HCl and 2 mL of methanol. Reflux for 100 min. then concentrate under vacuum. Triturate the residue with ether to give the product hydrochloride.

### PREPARATIVE EXAMPLES 25-28

Following the procedure set forth in Preparative Example 27, but using the indicated alkyl or benzyl halide in place of 2-methyl propyl iodide, the substituted histamines shown are prepared.

### PREPARATIVE EXAMPLE 25

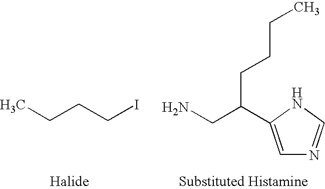

### PREPARATIVE EXAMPLE 26

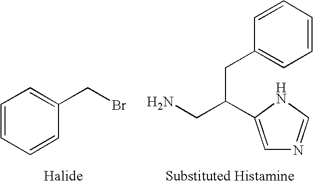

### PREPARATIVE EXAMPLE 27

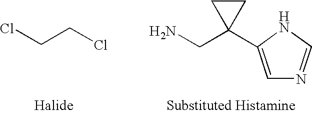

### PREPARATIVE EXAMPLE 28

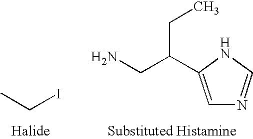

### PREPARATIVE EXAMPLE 29

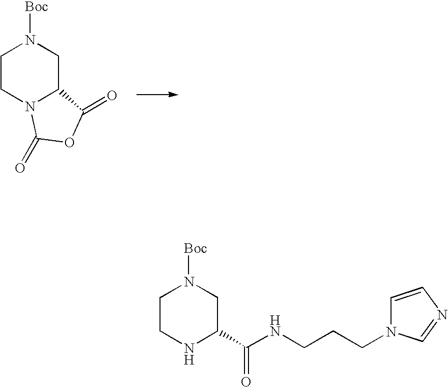

The anhydride (0.5088 g, 1.99 mmoles) (is prepared as described in Preparative Example 33) and 1-(3-aminopropyl)-imidazole (0.260 mL, 2.18 mmoles) are dissolved in anhydrous dichloromethane (10 mL) and the mixture was stirred under argon at 25° C. for 5 min. The mixture is diluted with dichloromethane and extracted with saturated aqueous sodium bicarbonate. The dichloromethane layer is dried (MgSO₄), filtered and evaporated to dryness. The resulting product is chromatographed on a silica gel column using 10% (conc, NH₄OH in methanol)-dichloromethane as the eluent to give the title compound.

### PREPARATIVE EXAMPLE 30

Step A

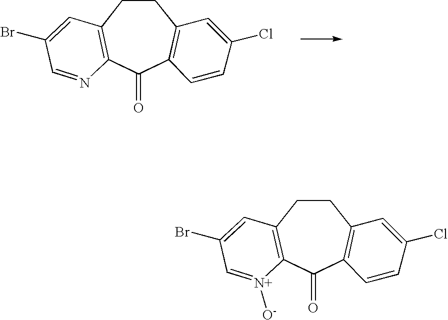

To a solution of 3-bromo-8-chloro-5,6-dihydro-11H-benzo[5,6]cyclohepta[1,2-b]pyridin-11-one (2 g) (6.2 mmoles) in anhydrous dichloromethane (14 ml) at 0° C. and under an argon atmosphere, is added a solution of 3-chloroperbenzoic acid (1.76 g) (10.4 mmoles) in anhydrous dichloromethane (35 ml) dropwise over a period of 30 minutes. The mixture is allowed to warm to room temperature and after 18 h additional 3-chloro-perbenzoic acid (0.88 g) (5.2 mmoles) in anhydrous dichloro-methane (25 ml) is added and the mixture is stirred for a total of 42 h. The mixture is diluted with dichloromethane and washed with 1N NaOH (200 ml). The aqueous layer is extracted with additional dichloromethane (2×200 ml) and the combined organic layers are dried over magnesium sulfate, filtered and evaporated to dryness. The product is chromatographed on silica gel using 0.25%-0.5%-1% (10% conc. NH₄OH in methanol) dichloromethane as the eluant to give the title compound.
Step B

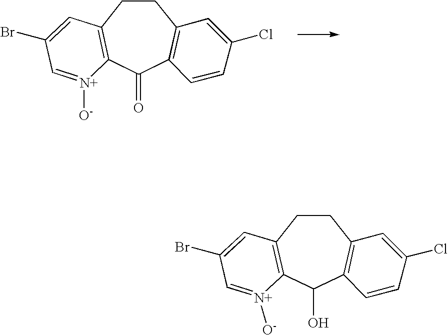

The title compound of Step A (1.3422 g) (3.96 mmoles) is dissolved in methanol (18 ml) and dichloromethane (20 ml) and sodium borohydride (0.219 g) (5.79 mmoles) was added. The mixture is stirred under argon at 0° C. for 1 h and then allowed to warm up to 25° C. over a period of 1 h. The mixture is diluted with dichloromethane (800 ml) and washed with 1N NaOH (150 ml). The aqueous layer is extracted with dichloromethane (2×100 ml) and the combined organic layers are dried over magnesium sulfate, filtered and evaporated to dryness. The product is chromatographed on silica gel using 1% (10% conc. NH₄OH in methanol) dichloro-methane as the eluant to give the title compound.
Step C

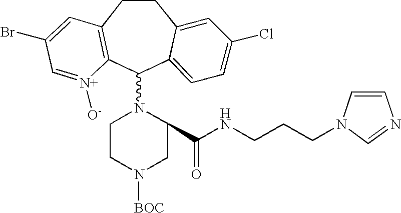

The title compound from Step B (0.552 g, 1.62 mmoles) and triethylamine (1.19 mL, 8.52 mmoles) are dissolved in anhydrous dichloromethane (8.5 mL) and the solution is cooled to 0° C. Methanesulfonyl chloride (0.4 mL, 5.16 mmoles) is added over 30 min and the mixture is stirred at 0° C. for a total of 1.25 h. The solution is evaporated to dryness to give the 11-mesyl derivative which is used without further purification. The latter is dissolved in anhydrous dichloromethane (40 mL) and the solution is stirred at 0° C.

The compound: 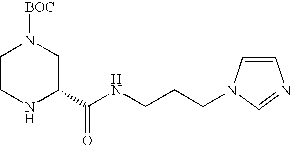
(Preparative Example 29) (0.5 g, 2.11 mmoles) is dissolved in anhydrous dichloromethane (20 mL) and anhydrous DMF (20 mL) is added at 0° C. and the solution is stirred and allowed to warm up to 25° C. over 2 h. The reaction is allowed to proceed at 25° C. for 18 h and is then diluted with dichloromethane and washed with saturated aqueous sodium bicarbonate, dried (MgSO₄), filtered and evaporated to dryness. The product is chromatographed on a silica gel column using 4% (10% conc. NH₄OH in methanol)-dichloro-methane as the eluant to give the title racemic compound.
Step D

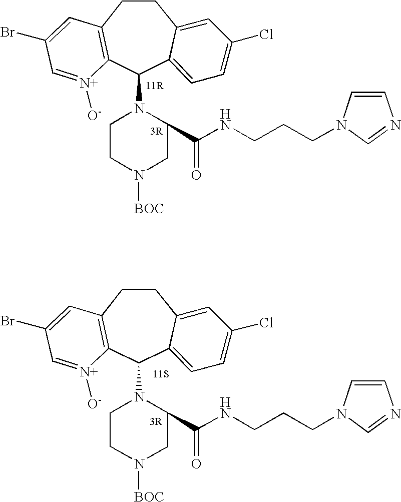

The title racemic compound from Step C above (0.395 g) is subjected to preparative HPLC on a Chiralpak ADO column (50×5 cm) using 65% hexane-35% isopropyl alcohol-0.2% diethylamine as the eluant to give in the order of elution the 11-R(+)-diastereoisomer of the title compound followed by the 11-S(−)-diastereoisomer of the title compound.

### PREPARATIVE EXAMPLE 31

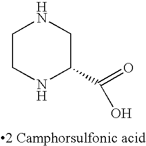

To 2.5 kg of (R)-(−)-camphorsulfonic acid stirring at 60° C. in 1250 ml of distilled water is added a solution of the potassium salt of 2-carboxyl-piperazine (565 gm, 3.35 mol). The mixture is allowed to stir at 95° C. until completely dissolved. The solution is allowed to stand at ambient temperature for 48 hrs. The resulting precipitate is filtered to obtain 1444 gm of damp solid. The solids are then dissolved in 1200 ml of distilled water and heated on a steam bath until all solids dissolved. The hot solution is then set aside to cool slowly for 72 hrs. The crystalline solids are filtered to give 362 gm of the pure 2-R-enantiomeric product.

### PREPARATIVE EXAMPLE 32

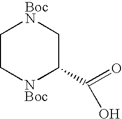

2-R-carboxyl-piperazine-di-(R)-(−)-camphorsulfonic acid (Preparative Example 31) (362 gm, 0.608 mol) is dissolved in 1.4 L of distilled water and 1.4 L of methanol. 75 ml of 50% NaOH is dripped in to the stirred reaction mixture to obtain a ˜pH 9.5 solution. To this solution is added di-tert-butyl-dicarbonate (336 gm, 1.54 mol) as a solid. The pH dropped to ˜7.0. The pH of the reaction mixture is maintained at 9.5 with 50% NaOH (total of 175 ml), and the reaction mixture stirred for 2.5 hours to obtain a precipitate. The reaction mixture is diluted to 9 L with ice/water followed by washing with 2 L of ether. The ether is discarded and the pH of the aqueous layer adjusted to pH 3.0 by the portionwise addition of solid citric acid. The acidified aqueous layer is then extracted with dichloro-methane 3× with 2 L. The organic layers are combined, dried over sodium sulfate, filtered and evaporated to obtain the title compound.

### PREPARATIVE EXAMPLE 33

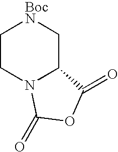

To an ice cold solution N,N-dimethylformamide (49.6 ml) is added, dropwise, thionylchloride (46.7 ml) over a period of 5 minutes in a 5 L round bottom flask under a nitrogen atmosphere. The reaction mixture is allowed to stir for 5 min. and the ice bath is removed and the reaction mixture is allowed to stir at ambient temperature for 30 min. The reaction mixture is cooled again in an ice bath and a solution of N,N-di-tert-butoxycarbonyl-2-R-carboxyl-piperazine (Preparative Example 32) (201.6 gm, 0.61 mmol) in 51.7 ml of pyridine and 1.9 L of acetonitrile is cannulated into the reaction mixture. The reaction mixture is allowed to warm to ambient temperature to obtain a solution. After stirring at ambient temperature for 18 hours, the reaction mixture is filtered and the filtrate is poured into ice water (7 L) and then is extracted with 4×2 L of ethyl acetate, is dried over sodium sulfate, is filtered and is evaporated to dryness under vacuo to obtain the title product.

### PREPARATIVE EXAMPLE 34

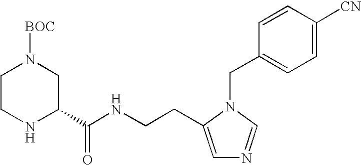

1N-p-Cyanobenzyl histamine (0.34, 1.5 mmol) (is prepared as described in Preparative Example 163) is added to a solution of the Boc-anhydride (Preparative Example 33) (0.38 gm, 1.5 mmol) in 10 ml of dichloromethane and is stirred under a nitrogen. After 1 hr, 0.15 gm more of the Boc-anhydride is added and the reaction monitored for completion by normal phase tlc using 10% methanol/dichloromethane as the eluent. The reaction mixture is poured into brine and is extracted with dichloromethane (3×). The dichloromethane layers are combined, dried over MgSO₄, filtered and evaporated to dryness. The residue is chromatographed on a flash column of silica gel using 5% methanol/dichloromethane to obtain the title compound.

### PREPARATIVE EXAMPLE 35

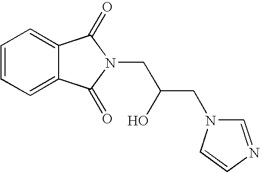

N-(2,3-Epoxypropyl)phthalimide (2.3 gm, 11.3 mmol) is dissolved in N,N-dimethylformamide and imidazole (1.53 gm, 1.5 eq.) is added and the reaction mixture stirred at 90° C. for 5 hours. Brine is added and the product is extracted with ethylacetate to obtain the title product.

### PREPARATIVE EXAMPLE 36

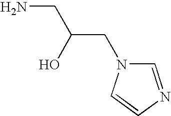

1-Phthalamido-2-hydroxy-3-1-H-imidazole-propane (from Preparative Example 46) (0.6 gm) is dissolved in ethanol and 5 ml of hydrazine hydrate is added. The reaction mixture is refluxed for 3 hours. The reaction mixture is cooled to ambient temperature and the resulting precipitate is filtered. The filtrate is evaporated to dryness to obtain the title product which is used without further purification.

### PREPARATIVE EXAMPLE 37

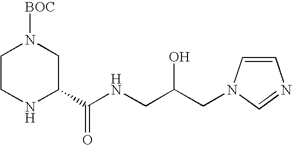

1-Amino-2-hydroxy-3-1-H-imidazole-propane (from Preparative Example 36) (2.2 mmol) is added to a solution of the Boc-anhydride (Preparative Example 33) (0.57 gm, 2.2 mmol) in 10 ml of dichloromethane and is stirred under nitrogen. After 1 hr, 0.15 gm more of the Boc-anhydride is added and the reaction monitored for completion by normal phase tlc using 10% methanol/dichloromethane as the eluent. The reaction mixture is poured into brine and extracted with dichloromethane (3×). The dichloromethane layers is combined, is dried over MgSO₄, is filtered and is evaporated to dryness. The residue is chromatographed on a flash column of silica gel using 5% methanol/dichloromethane to obtain the title compound.

### PREPARATIVE EXAMPLE 38

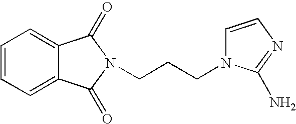

2-Aminoimidazole (8 g, 60 mmol) is dissolved in 200 ml of DMF and cooled in an ice bath. Sodium hydride 60% oil dispersion (2.4 g, 60 mmol) is added portionwise and the reaction mixture is stirred for 1 hour. N-(3-Bromopropyl)-phthalimide (16 g, 74 mmol) is added and the reaction mixture is stirred for ½ hour at 0° C., 1 hour at ambient temperature, and then 1 hour at 85° C. The reaction mixture is then cooled to ambient temperature and is added to brine and is extracted with ethyl acetate to obtain the crude product which was purified by column chromatography using 2% methanol/methylene chloride to obtain the title compound.

### PREPARATIVE EXAMPLE 39

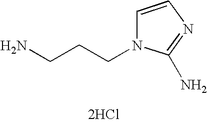

0.5 gm of 1-phthalimidopropyl-2-aminoimidazole (from Preparative Example 38) is refluxed in 20 ml of 6N HCl for 6 hours. The mixture is washed with ethyl acetate and the aqueous layer is evaporated to dryness to obtain the title product.

### PREPARATIVE EXAMPLE 40

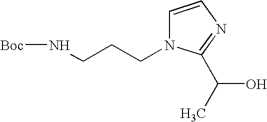

1-tert-Butoxycarbonylaminopropyl-imidazole (0.991 gm, 4.4 mmol) is dissolved in 25 mol of dry THF and cooled to −78° C. A 2.5M solution of n-butyllithium (3.88 ml, 9.68 mmol) in cyclohexanes is added dropwise and the reaction is stirred for ½ hour. Acetaldehyde (0.49 ml, 8.8 mmol) is added and the reaction is stirred for ½ hour. The reaction mixture is allowed to warm to ambient temperature. The reaction is diluted with ethyl acetate and is washed with brine. The ethyl acetate layer is evaporated to obtain a gum which is chromatographed on silica gel to obtain the title product.

### PREPARATIVE EXAMPLE 40

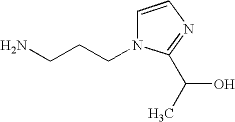

1-tert-Butoxycarbonylaminopropyl-2-hydroxyethyl-imidazole (Preparative Example 40) (0.51 gm) is dissolved in trifluoroacetic acid and is stirred for 3-4 hours. The mixture is evaporated to dryness to obtain the pure TFA salt of the title compound.

### PREPARATIVE EXAMPLE 42

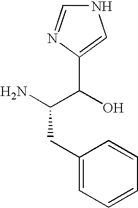

1-N-Trityl-4-iodoimidazole (1.91 gm) is dissolved in 20 ml of dichloromethane and 1.46 ml of ethyl magnesiumbromide is added while stirring. After 15 min. N-Boc-phenylalanine aldehyde (0.5 gm) is added and the reaction mixture is stirred for 18 hours. The reaction mixture is washed with saturated ammonium chloride, dried over magnesium sulfate, and chromatographed on silica gel to obtain the intermediate blocked product. This is then treated with 4M HCl/dioxane for 18 hours. The mixture is evaporated to dryness and is dissolved in distilled water and is washed with ethyl acetate. The aqueous layer is evaporated to obtain pure title product. (MH⁺=218).

### PREPARATIVE EXAMPLE 43

Step A

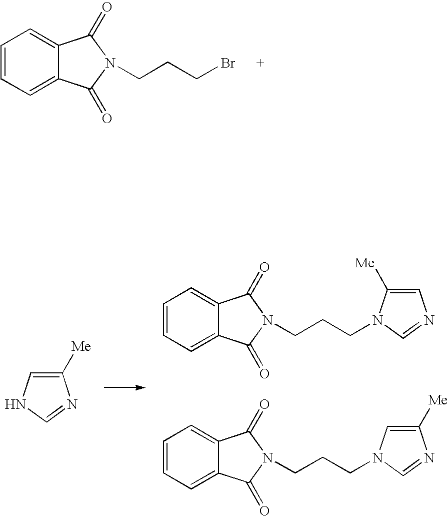

A mixture of N-(3-bromopropyl)phthalimide (12.3 g, 46 mmol), 4-methylimidazole (3.78 g, 46 mmol), sodium hydride (60% in mineral oil, 1.84 g, 46 mmol) and anhydrous DMF (50 mL) is stirred at 25-70° C. under N₂ overnight. The mixture is concentrated in vacuo to give a residue which is diluted with dichloromethane, is filtered, is concentrated in vacuo and is purified by flash column chromatography (silica gel) using 1% MeOH—CH₂Cl₂ saturated with aqueous ammonium hydroxide to give the title compound.
Step B

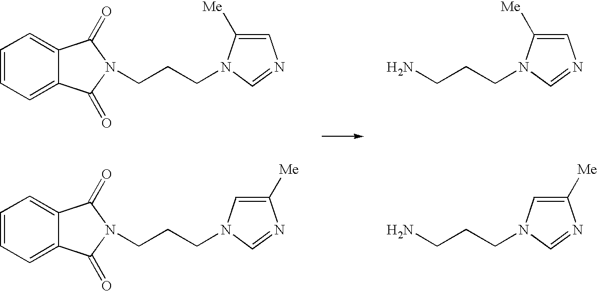

To a solution of the title compound from Step A (8.02 g, 29.8 mmol) which is dissolved in absolute EtOH (150 mL) is added hydrazine-mono hydrate (15 mL) and the mixture is stirred at reflux for 12 h under N₂. The mixture is diluted with dichloromethane, is filtered and is concentrated in vacuo. The residue is purified by flash column chromatography (silica gel) using 5% MeOH—CH₂Cl₂ saturated with aqueous ammonium hydroxide to give the title compound.

### PREPARATIVE EXAMPLES 44-48

Following the procedure set forth in Preparative Example 43, but using the substituted imidazole in Table 3 below instead of 4-methylimidazole in Step A, the amines (Product) listed in Table 3 are prepared.

TABLE 3

| Prep. | | |
|---|---|---|
| Ex. | Imidazole | Product |
| 44 | 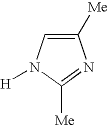 | 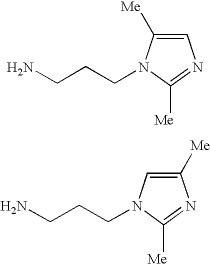 |
| 45 | 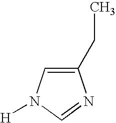 | 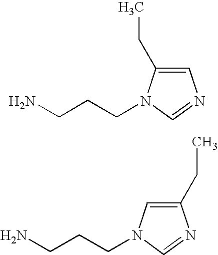 |
| 46 | 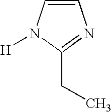 | 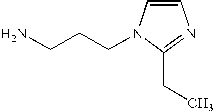 |
| 47 | 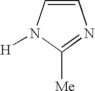 | 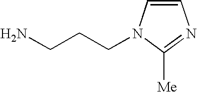 |
| 48 | 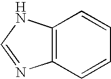 | 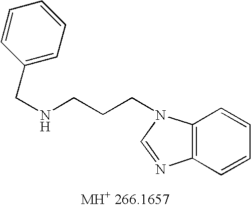 |

### PREPARATIVE EXAMPLE 49

If the procedure set forth in Preparative Example 43 were followed, except the imidazole 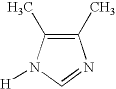
would be used instead of 4-methylimidazole in Step A, the amine 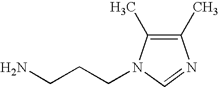
would be obtained.

### PREPARATIVE EXAMPLE 50

If the procedure set forth in Preparative Example 43 were followed, except the imidazole 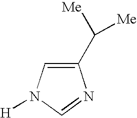
would be used instead of 4-methylimidazole in Step A, the amine 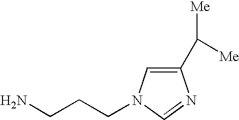
would be obtained.

### PREPARATIVE EXAMPLE 51

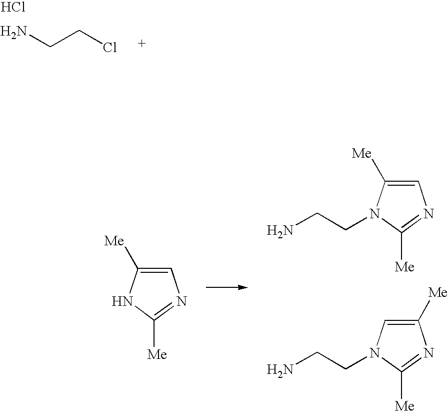

A mixture of 2-chloroethylamine hydrochloride (7.66 g, 66 mmol), 2,4-dimethylimidazole (5.88 g, 61 mmol), tetrabutyl ammonium sulfate (0.83 g, 2.5 mmol), solid NaOH (8.81 g, 220 mmol) and anhydrous acetonitrile (80 mL) is stirred at reflux for 48 h under N₂. The mixture is filtered, concentrated in vacuo and purified by flash column chromatography (silica gel) using 2% MeOH—CH₂Cl₂ saturated with aqueous ammonium hydroxide to give the title compound.

### PREPARATIVE EXAMPLES 52-56

Following the procedure set forth in Preparative Example 68, but using the substituted imidazole or triazole in Table 4 below instead of 2,4-dimethylimidazole, the amines (Product) listed in Table 4 are prepared.

TABLE 4

| Prep. | | |
|---|---|---|
| Ex. | Imidazole | Product |
| 52 | 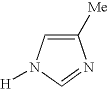 | 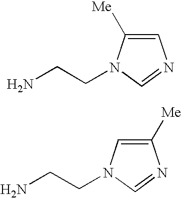 |
| 53 | 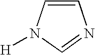 | 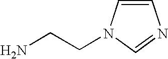 |
| 54 | 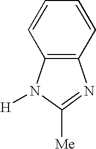 | 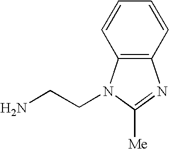 |
| 55 | 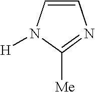 | 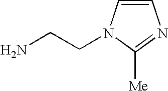 |
| 56 | 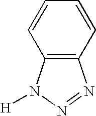 | 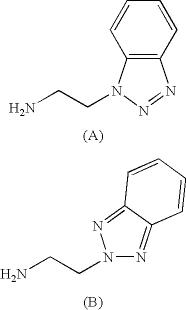 |

### PREPARATIVE EXAMPLE 57

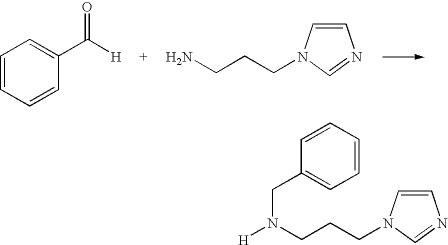

A mixture of 1-(3-aminopropyl)imidazole (37.1 g, 297 mmol), benzaldehyde (30 g, 283 mmol), 3 Å molecular sieves (50 g), sodium acetate (24.1 g, 283 mmol) and anhydrous methanol (700 mL) is stirred at room temperature under N₂ overnight. The mixture is cooled to 0° C. and sodium borohydride (10.9 g, 288 mmol) is added portionwise over 1 hour. The mixture is stirred at room temperature for 3 hours. The mixture is filtered through celite, is washed with methanol, and is concentrated in vacuo to give a residue which is diluted with dichloro-methane and washed with 10% aqueous sodium hydroxide. The organic phases are washed with brine, are dried over anhydrous magnesium sulfate, are filtered and are concentrated in vacuo to give the title compound.

### PREPARATIVE EXAMPLES 58-77

Following the procedure set forth in Preparative Example 57, but using the aldehyde and imidazolylalkyl amine (Imidazole) in Table 5, the amines (Product) in Table 5 are obtained.

TABLE 5

| Prep | | | |
|---|---|---|---|
| Ex. | Aldehyde | Imidazole | Product |
| 58 | 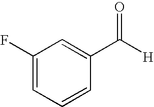 | 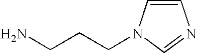 | 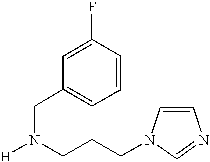 |
| 59 | 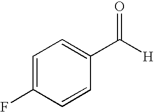 | 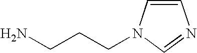 | 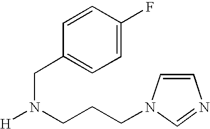 |
| 60 | 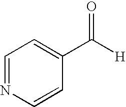 | 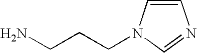 | 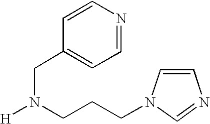 |
| 61 | 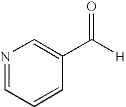 | 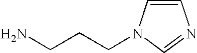 | 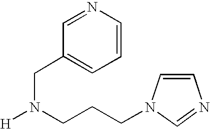 |
| 62 | 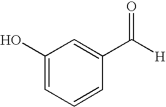 | 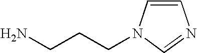 | 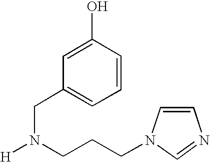 |
| 63 | 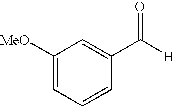 | 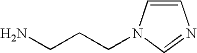 | 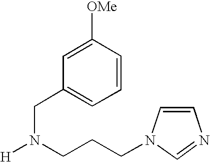 |
| 64 | 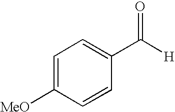 | 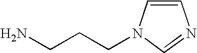 | 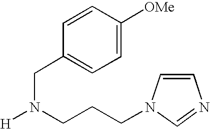 |
| 65 | 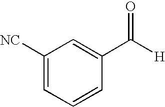 | 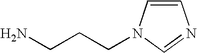 | 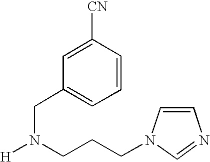 |
| 66 | 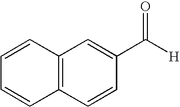 | 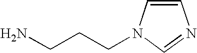 | 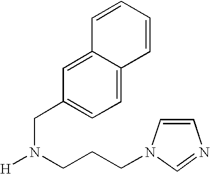 |
| 67 | 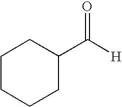 | 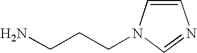 | 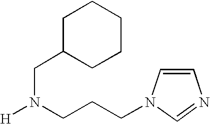 |
| 68 | 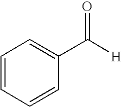 | 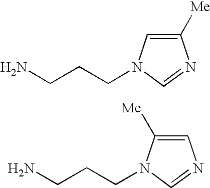 | 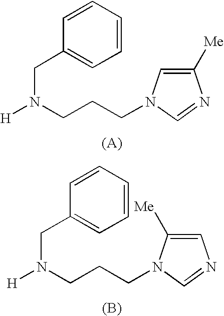 |
| 69 | 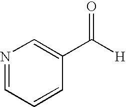 | 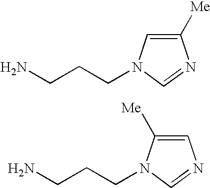 | 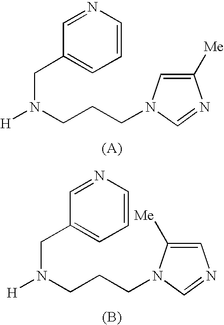 |
| 70 | 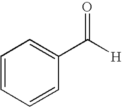 | 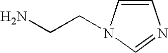 | 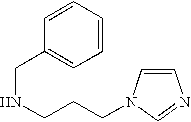 |
| 71 | 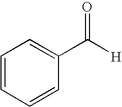 | 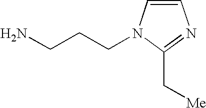 | 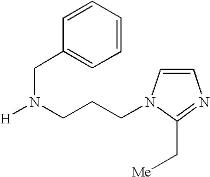 |
| 72 | 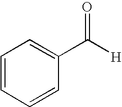 | 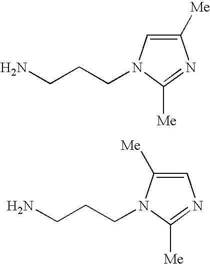 | 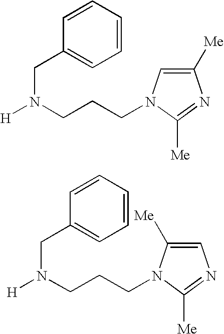 |
| 73 | 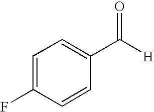 | 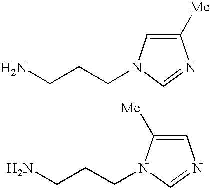 | 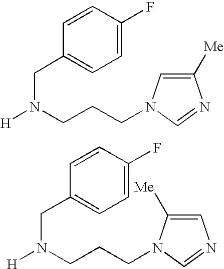 |
| 74 | 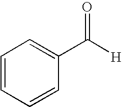 | 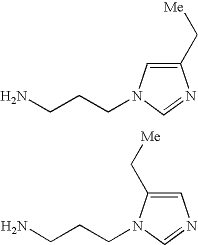 | 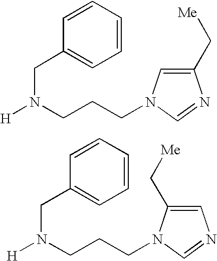 |
| 75 | 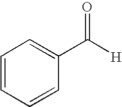 | 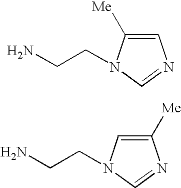 | 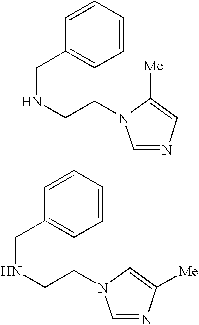 |
| 76 | 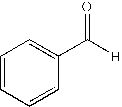 | 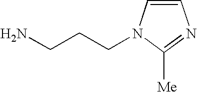 | 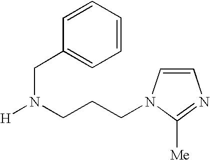 |
| 77 | 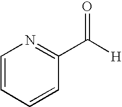 | 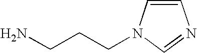 | 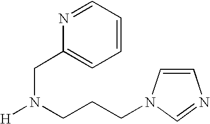 |

### PREPARATIVE EXAMPLE 78

Step A

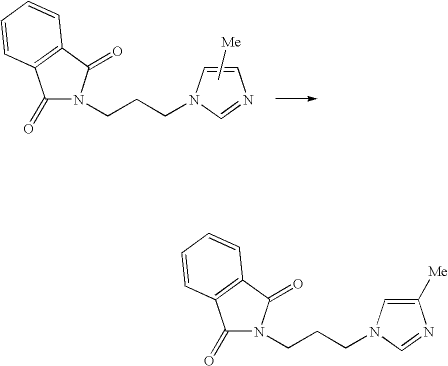

To a CH₂Cl₂ (500 mL) solution of the title compound from Preparative Example 43 Step A (65.7 g) which is cooled to 0° C. is added trityl chloride (27.2 g). The resulting mixture is warmed to and stirred at room temperature for 1.5 hr, then concentrated in vacuo without heating. Purification by flash column chromatography (silica, 1:1 Acetone-EtOAc) affords the pure 4-methyl isomer.
Step B

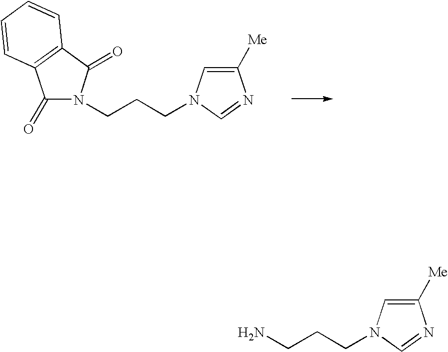

Following essentially the same procedure as that described in Preparative Example 43 Step B except using the pure 4-methylimidazole product from Preparative Example 95.1 Step A (35.02 g), the title compound is afforded.
Step C

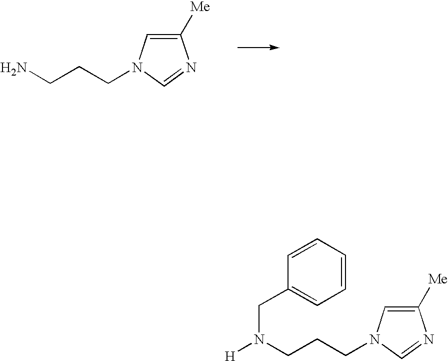

Following essentially the same procedure as that described in Preparative Example 57 except using the pure 4-methylimidazolepropylamine product from Preparative Example 78 Step B above (16.12 g) instead of 1-(3-aminopropyl)-imidazole, the title compound is afforded.

### PREPARATIVE EXAMPLE 79

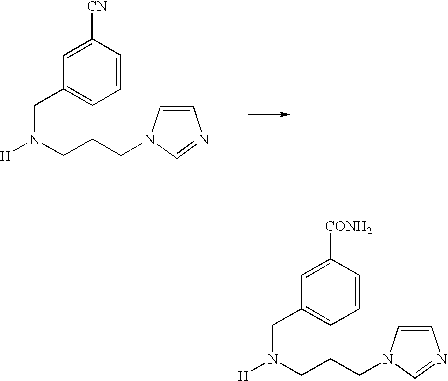

A mixture of the title compound from Preparative Example 65 (0.50 g, 2.1 mmol), absolute EtOH (50 mL), 30% hydrogen peroxide (aq) (0.45 mL, 4.4 mmol) and 1M NaOH (aq) (4.4 mL, 4.4 mmol) is stirred at 50° C. for 12 h. The mixture is concentrated in vacuo and purified by flash column chromatography (silica gel) using 10% MeOH—CH₂Cl₂ saturated with aqueous ammonium hydroxide to give the title compound.

### PREPARATIVE EXAMPLE 80

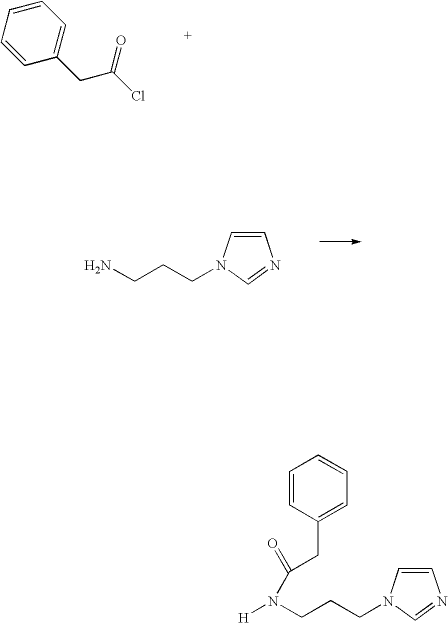

To a cooled (0° C.) solution of 1-(3-aminopropyl)imidazole (Aldrich, 1.9 mL, 16 mmol) and triethylamine (5.6 mL, 40 mmol) which is dissolved in anhydrous CH₂Cl₂ (20 mL) is added phenylacetyl chloride (2.12 mL, 16 mmol). The mixture is warmed to and stirred at room temperature overnight. The mixture is washed with 1N aqueous NaOH, dried over anhydrous MgSO₄ and filtered. The solution is concentrated in vacuo and purified by flash column chromatography (silica gel) using 2% MeOH-98% CH₂Cl₂ saturated with aqueous ammonium hydroxide to give the title compound.

### PREPARATIVE EXAMPLE 81

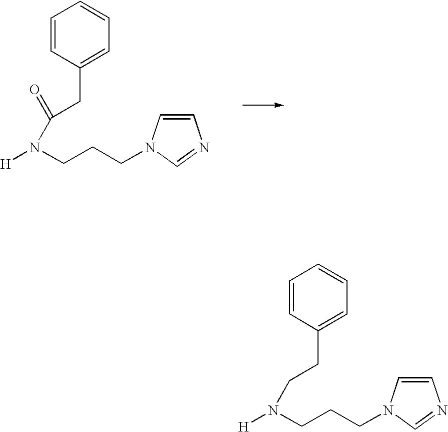

To a refluxing solution of the title compound from Preparative Example 80 (0.51 g, 2.1 mmol) which is dissolved in anhydrous THF (5 mL) is added borane dimethylsulfide complex (6.3 mL, 2M in THF, 13 mmol). After 1 hr, the mixture is cooled to room temperature and stirred overnight. Hydrochloric acid (1N) is added dropwise until the reaction mixture is determined to be acidic (pH paper). The mixture is basified with 1N aqueous NaOH, is extracted with CH₂Cl₂, is dried over anhydrous MgSO₄ and is filtered. The solution is concentrated in vacuo and is purified by flash column chromatography (silica gel) using 2% MeOH-98% CH₂Cl₂ saturated with aqueous ammonium hydroxide to give the title compound.

### PREPARATIVE EXAMPLE 82

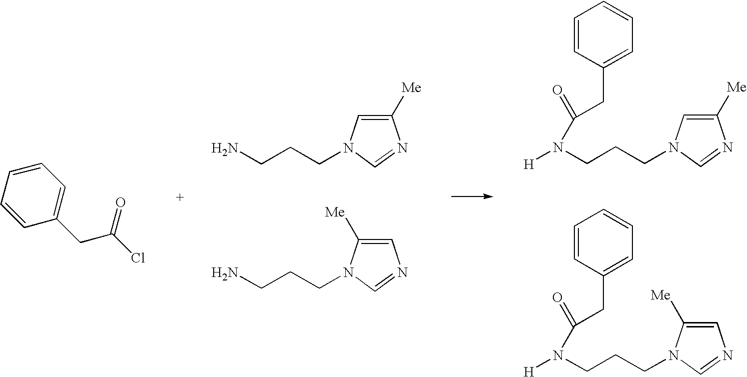

To a cooled (0° C.) solution of the title compound from Preparative Example 43 Step B (0.7 g, 5 mmol) and triethylamine (1.7 mL, 12.5 mmol) which is dissolved in anhydrous CH₂Cl₂ (10 mL) is added phenylacetyl chloride (0.67 mL, 5 mmol). The mixture is warmed to and is stirred at room temperature overnight. The mixture is washed with 1M HCl (aq) and the aqueous phase is basified with 1N aqueous NaOH. This phase is extracted with CH₂Cl₂ and is dried over anhydrous MgSO₄ and is filtered. The solution is concentrated in vacuo to give the title compound.

### PREPARATIVE EXAMPLE 83

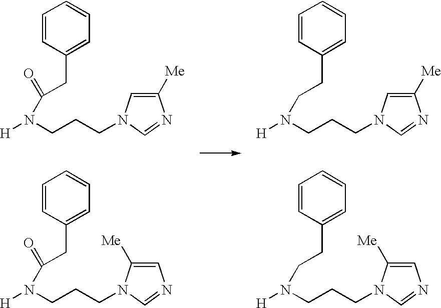

To a refluxing solution of the title compound from Preparative Example 82 (0.66 g, 2.5 mmol) which is dissolved in anhydrous THF (15 mL) is added borane-THF complex (5 mL, 1M in THF, 5 mmol). The mixture is refluxed for 12 h, then cooled to room temperature and concentrated in vacuo. The residue is diluted with 1M HCl and is washed with CH₂Cl₂ then the aqueous phase is basified with 50% aqueous NaOH and is extracted with CH₂Cl₂ and is dried over anhydrous MgSO₄ and is filtered. The solution is concentrated in vacuo and is purified by preparative plate chromatography (silica gel) using 3% MeOH—CH₂Cl₂ saturated with aqueous ammonium hydroxide to give the title compound which is purified by preparative chiral chromatography (Chiralpack AD, 5 cm×50 cm column, flow rate 80 mL/min, 5-8% IPA-Hexane+0.2% diethylamine).

### PREPARATIVE EXAMPLE 84

If the procedure of Preparative Example 82 were followed, but the compound 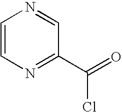
was to be reacted with the title compound from Preparative Example 43 Step B, then the Product 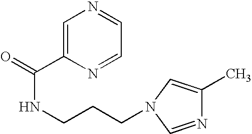
would be obtained.

### PREPARATIVE EXAMPLE 85

If the procedure of Preparative Example 83 were followed, but the Product from Preparative Example 84 was to be used, then the Product 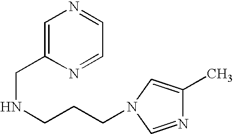
would be obtained.

### PREPARATIVE EXAMPLE 86

Step A

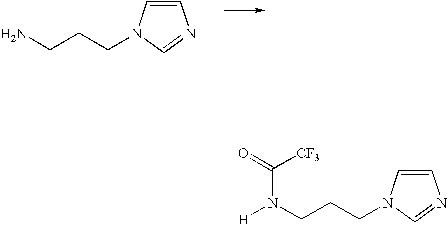

To a cooled (0° C.) solution of 1-(3-aminopropyl)imidazole (10 g, 80 mmol) and triethylamine (17.1 mL, 120 mmol) which is dissolved in anhydrous CH₂Cl₂ (50 mL) is added trifluoroacetic anhydride (12.4 mL, 88 mmol). The mixture is warmed to and is stirred at room temperature overnight. The mixture is washed with water, is dried over anhydrous MgSO₄, is filtered and is concentrated in vacuo to give the title compound.
Step B

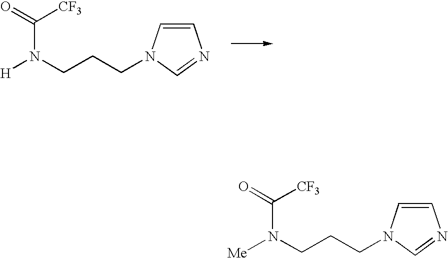

To the title compound from Step A (0.24 g, 1.1 mmol) which is dissolved in anhydrous DMF (10 mL) is added solid sodium hydride (85 mg, 2.1 mmol, 60% dispersion in mineral oil). When gas evolution ceases, methyl iodide (0.1 mL, 1.1 mmol) is added and the mixture is stirred at 70° C. for 40 min. The resulting mixture is cooled to room temperature, is concentrated in vacuo, is diluted with CH₂Cl₂ and is washed with water. The solution is dried over anhydrous MgSO₄, is filtered and is concentrated in vacuo to give an oil (0.28 g). Purification by preparative plate chromatography (silica gel) using 2% MeOH-98% CH₂Cl₂ saturated with aqueous ammonium hydroxide gives the title compound.
Step C

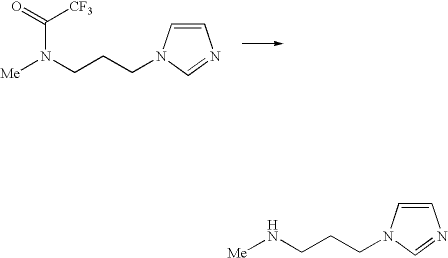

A mixture of the title compound from Step B (74 mg, 0.3 mmol) and 20% KOH in H₂O (0.6 mL) is stirred at room temperature for 15 min. The resulting mixture is concentrated in vacuo and is purified by flash column chromatography (silica gel) using 10% MeOH-90% CH₂Cl₂ saturated with aqueous ammonium hydroxide to give the title compound.

### PREPARATIVE EXAMPLE 87

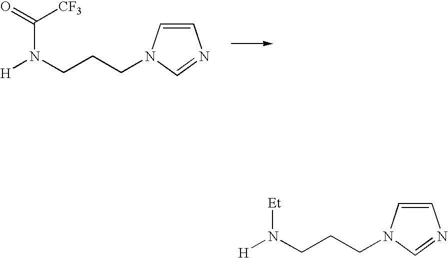

Following a similar procedure as that used for the preparation of the title compounds from Preparative Example 86 Steps B-C, but using ethyl iodide instead of methyl iodide, the ethyl amine is obtained.

### PREPARATIVE EXAMPLE 88

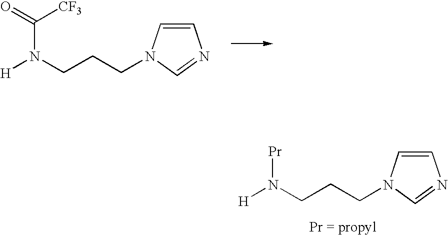

Following a similar procedure as that used for the preparation of the title compounds from Preparative Example 86 Steps B-C, but using propyl iodide instead of methyl iodide, the propyl amine is obtained.

### PREPARATIVE EXAMPLE 89

### Alternative Procedure to Preparative Example 57

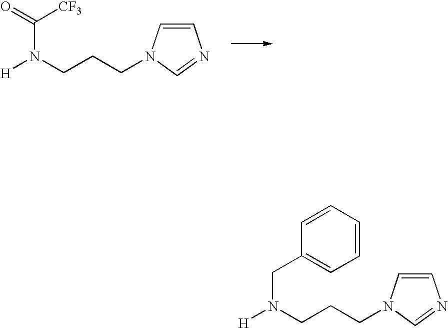

Following a similar procedure as that used for the preparation of the title compounds from Preparative Example 86 Steps B-C, but using benzyl bromide instead of methyl iodide), the benzyl amine is obtained.

### PREPARATIVE EXAMPLE 90

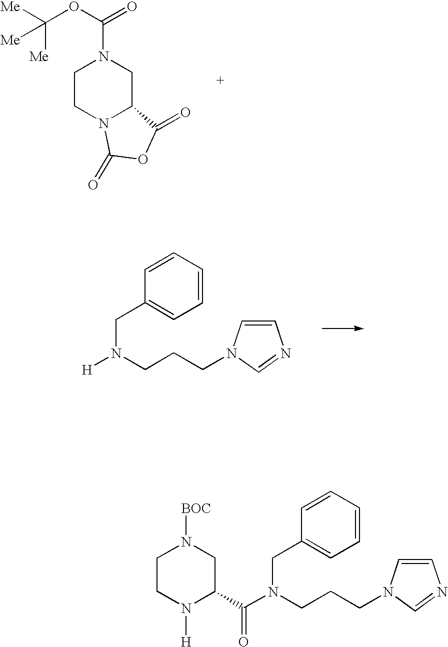

A mixture of the title compound from Preparative Example 57 (1.34 g, 6.2 mmol), the title compound from Preparative Example 33 (1.6 g, 6.2 mmol), triethyl amine (1.3 mL, 9.3 mmol) and anhydrous CH₂Cl₂ (10 mL) is stirred at room temperature for 48 hrs. The resulting mixture is extracted with CH₂Cl₂. The organic phase is dried over anhydrous MgSO₄, is filtered and is concentrated in vacuo to give a residue which is purified by flash column chromatography (silica gel) using 1% MeOH-99% CH₂Cl₂ saturated with aqueous ammonium hydroxide to give the title compound.

### PREPARATIVE EXAMPLE 91

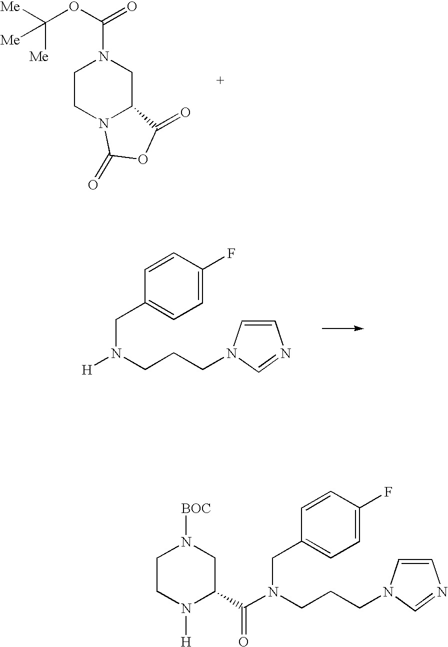

Using the procedure described for Preparative Example 90, but using the title compound from Preparative Example 59, the title compound is prepared.

### PREPARATIVE EXAMPLE 92

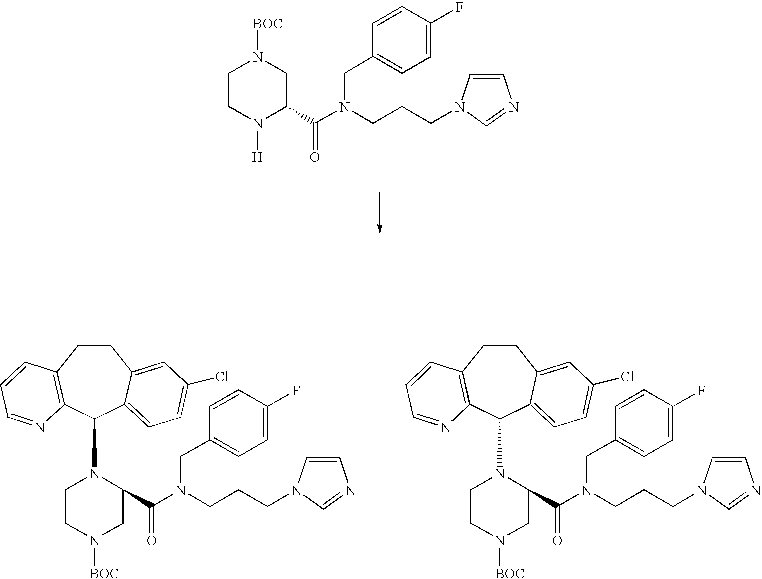

Using the procedure described for Preparative Example 93 (below), but using the title compound from Preparative Example 91 (146 mg, 0.55 mmol), and the 8-Cl-tricyclic chloride (see Preparative Example 7 in WO 95/10516) 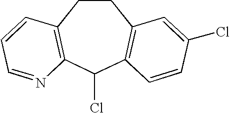
(159 mg, 0.46 mmol), the title compounds are prepared and are separated by preparative plate chromatography (silica gel) using 2% MeOH—CH₂Cl₂ saturated with aqueous ammonium hydroxide.

### PREPARATIVE EXAMPLE 93

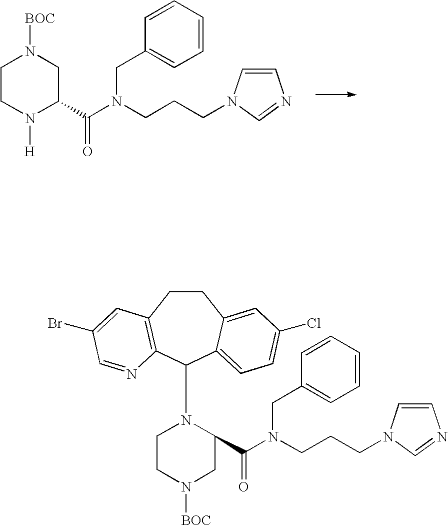

A mixture of the title compound from Preparative Example 90 (510 mg, 1.6 mmol), the tricyclic chloride 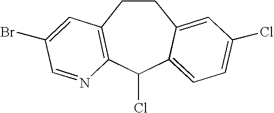
(534 mg, 1.6 mmol), triethylamine (1.1 mL, 7.8 mmol) and CH₂Cl₂ (10 mL) is stirred at room temperature overnight. The reaction mixture is concentrated in vacuo and purified by flash column chromatography (silica gel) using 2% MeOH-98% CH₂Cl₂ saturated with aqueous ammonium hydroxide to give the title compound as a light yellow solid (420 mg, 42%, MH⁺=733). If one were to use preparative chiral chromatography then the diastereomers could be separated.

### PREPARATIVE EXAMPLE 94

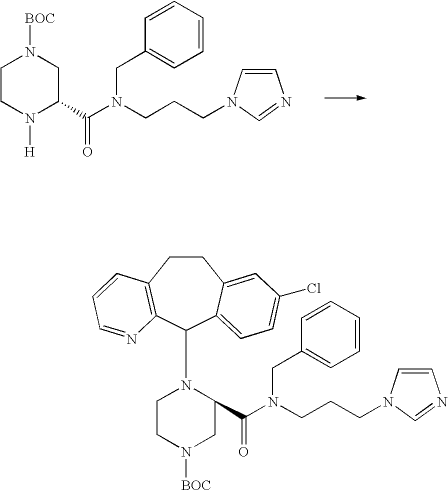

A mixture of the title compound from Preparative Example 90 (1.93 g, 5.9 mmol), the 8-Cl-tricyclic chloride 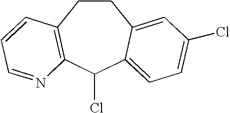
(see Preparative Example 7 in WO95/10516) (1.56 g, 5.9 mmol), triethylamine (4.1 mL, 29.5 mmol) and CH₂Cl₂ (10 mL) is stirred at room temperature for 48 h. The reaction mixture is concentrated in vacuo and is purified by flash column chromatography (silica gel) using 2% MeOH-98% CH₂Cl₂ saturated with aqueous ammonium hydroxide to give the title compound. If one were to use preparative chiral chromatography then the diastereomers could be separated.

### PREPARATIVE EXAMPLE 95

If one were to follow the procedure of Preparative Example 94, using the 10-Cl-tricycle chloride 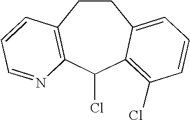
then one would obtain

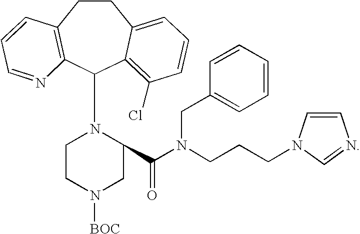

### PREPARATIVE EXAMPLE 96

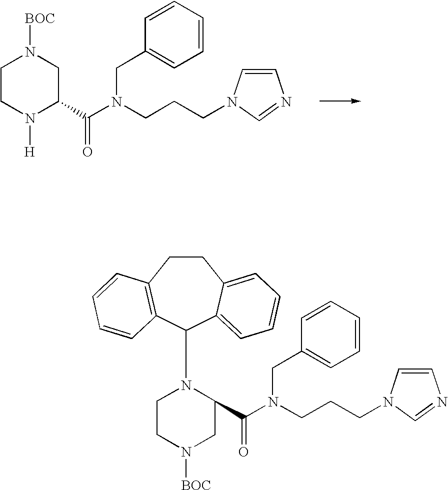

A mixture of the title compound from Preparative Example 90 (200 mg, 0.61 mmol), chlorobenzosuberane (140 mg, 0.61 mmol), triethylamine (0.43 mL, 3.1 mmol) and CH₂Cl₂ (10 mL) is stirred at room temperature overnight. The reaction mixture is concentrated in vacuo and purified by preparative plate chromatography (silica gel) using 2% MeOH—CH₂Cl₂ saturated with aqueous ammonium hydroxide to give the title compound.

### PREPARATIVE EXAMPLE 97

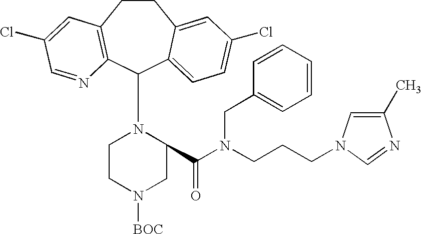

If the procedure of Preparative Example 94 is followed, except the 3,8-dichloro tricyclic compound 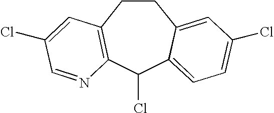
is used instead of the 8-Cl-tricycle chloride, an using an equivalent amount of an amine from Preparative Example 83 instead of the amine shown in Preparative Example 94, the title compound would be obtained.

### PREPARATIVE EXAMPLE 98

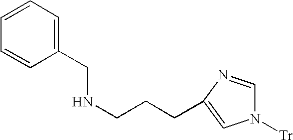 Step A

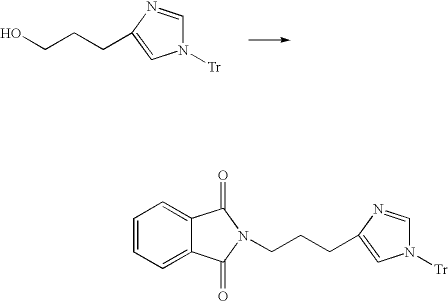

To a stirred solution of 1-(triphenylmethyl-1H-imidazol-4-yl)-3-hydroxypropane (WO 9629315) (5.04 g, 13.68 mmoles), phthalimide (2 g, 13.6 mmoles) and triphenyl phosphine (3.57 g, 13.6 mmoles) in THF (100 mL) at 0° C. is added diethyl azodicarboxylate (2.14 mL, 13.6 mmoles) dropwise. The reaction mixture is stirred for 1 h at 0° C. and then at room temperature for 16 h. Is Filtered to give the title compound.
Step B

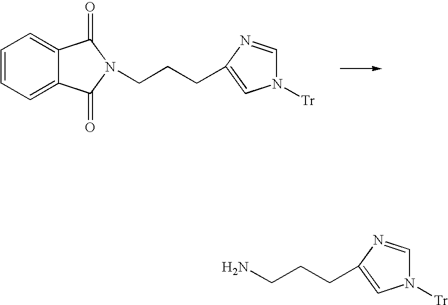

The title compound from Step A (2 g, 4.02 mmoles) and hydrazinehydrate (3.89 mL, 80.39 mmoles) are heated under reflux in ethanol (80 mL) for 16 h. The solids are filtered off and the filtrate is evaporated to give the title compound.
Step C

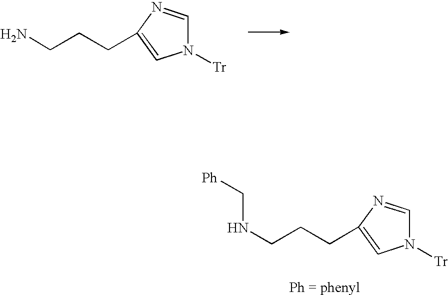

To a stirred solution of the title compound from Step B (1.5 g, 4.08 mmoles) and benzaldehyde (0.433 g, 4.08 mmoles) is added sodium cyanoborohydride (0.256 g, 4.08 mmoles). The pH of the solution is adjusted to ˜4.25 with acetic acid. The reaction mixture is then stirred for 2 h. The pH is then adjusted to 11.5 with 50% NaOH and extracted with ethyl acetate. The ethyl acetate extract is washed with water and brine and dried (MgSO₄), and is evaporated to give a crude residue which is chromatographed on silica gel using 4% (10% conc NH₄OH in methanol)-CH₂Cl₂ as the eluant to give the title compound.

### PREPARATIVE EXAMPLE 99

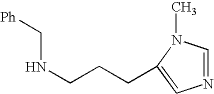 Step A

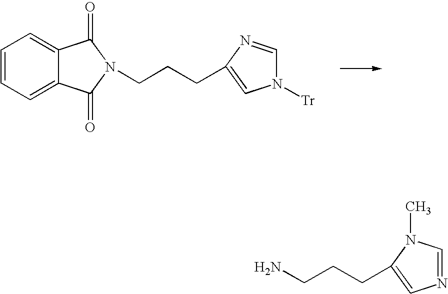

The title compound from Preparative Example 132 Step A (2 g, 4.1 mmoles) in CH₂Cl₂ (20 mL) is treated with methyl iodide (0.75 mL 12.05 mmoles) and stirred for 16 h. Evaporate to dryness to a residue which is then refluxed with 6N HCl (25 mL) for 16 h. Evaporate to dryness, neutralize with aqueous NaHCO₃ and evaporate to dryness again gives solids. Stir with CH₂Cl₂ (100 mL) and MeOH (50 mL) and filter off the solids. The filtrate is evaporated to give the title compound.
Step B

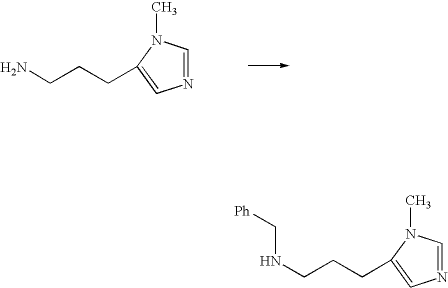

The title compound from Step A (1.97 g 14.14 mmoles), benzaldehyde (1.65 g 15.55 mmoles), sodium acetate (1.1 g, 13.42 mmoles) and 3 Å molecular sieves (2 g) in methanol are stirred for 18 h. To this sodium borohydride (0.519 g 13.72 mmoles) is added and is stirred for 4 h. The solids are filtered off and the filtrate is evaporated to a residue which was chromatographed to give the title compound.

### PREPARATIVE EXAMPLE 100

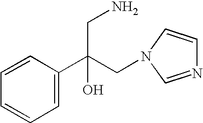 Step A

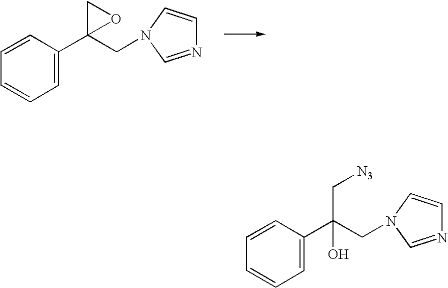

1-(2-Phenyl-2,3-epoxypropyl)-1H-imidazole (GB 2 099818 A) (2.15 g, 10.85 mmoles) and sodium azide (1.41 g, 21.71 mmoles) are heated in DMF (20 mL) at 60° C. for 16 h. Evaporate to dryness and extract with CH₂Cl₂, wash with brine and dry (MgSO₄). Evaporate to give the title compound.
Step B

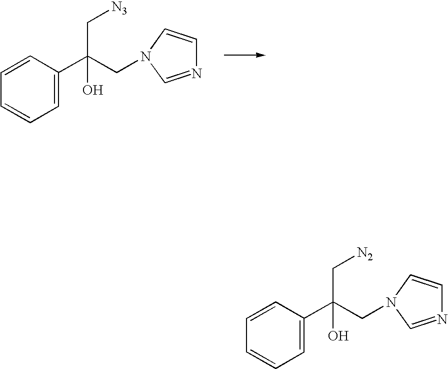

The title compound from Step A (0.8 g, 3.31 mmoles) in ethanol (15 mL) is hydrogenated over 10% Pd on carbon (0.2 g) at 50 psi overnight. The catalyst is filtered off and is evaporated to give the title compound.

### PREPARATIVE EXAMPLE 101

Step A

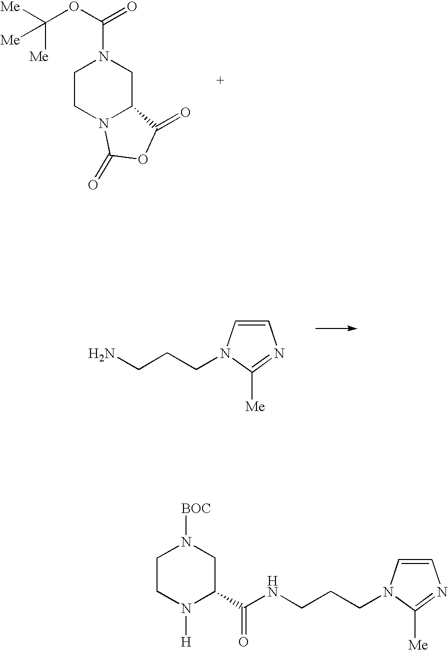

A mixture of the title compound from Preparative Example 47 (1.0 g, 7.2 mmol), the anhydride from Preparative Example 33 (2.2 g, 8.6 mmol), triethyl amine (1.5 mL, 10.8 mmol) and anhydrous CH₂Cl₂ (10 mL) is stirred at room temperature for 12 hrs. The mixture is concentrated in vacuo, is diluted with CH₂Cl₂ and is washed with a saturated aqueous solution of NaHCO₃. The organic phase is dried over anhydrous Na₂SO₄, is filtered and is concentrated in vacuo.
Step B

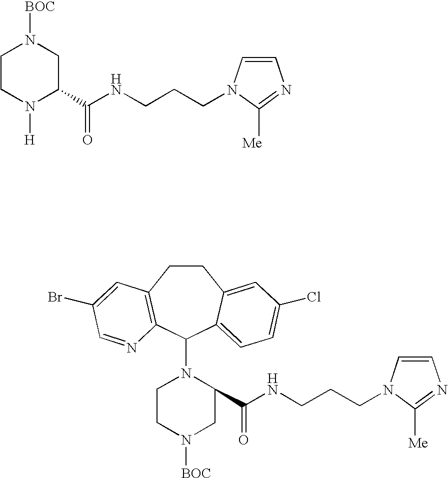

The title compound from Step A above (1.0 g, 7.2 mmol) is dissolved in CH₂Cl₂ (10 mL) and the resulting mixture is stirred for 5 hrs at 25° C. The mixture is concentrated in vacuo, is diluted with CH₂Cl₂ (50 mL) and is combined with the tricyclic chloride 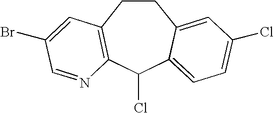
(2.7 g, 7.9 mmol) and triethylamine (5-10 mL) and is stirred at room temperature overnight. The mixture is concentrated in vacuo, is diluted with CH₂Cl₂ and is washed with a saturated aqueous solution of NaHCO₃. The organic phase is dried over anhydrous Na₂SO₄, is filtered, is concentrated in vacuo and is purified by flash column chromatography (silica gel) using 5% MeOH—CH₂Cl₂ saturated with aqueous ammonium hydroxide to give the title compound as a mixture of diastereomers.

### PREPARATIVE EXAMPLE 102

Step A

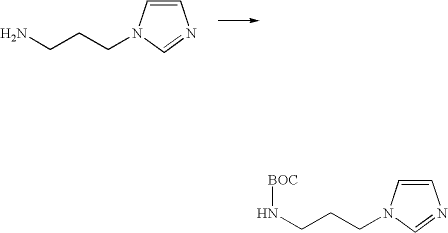

To 3-(1H-imidazol-1-yl)propylamine (20 mL, 167.6 mmol) dissolved in water (200 mL) and MeOH (200 mL) is added 50% NaOH (aq) until pH 9.5. Di-tert-butyldicarbonate (41 g, 187.9 mmol) is added while stirring at room temperature for 4 hrs and while maintaining the pH at 9.5 with 50% NaOH. The mixture is concentrated in vacuo to remove most MeOH, then is extracted with CH₂Cl₂. The organic phase is dried over anhydrous MgSO₄, is filtered and is concentrated in vacuo to give the title compound.
Step B

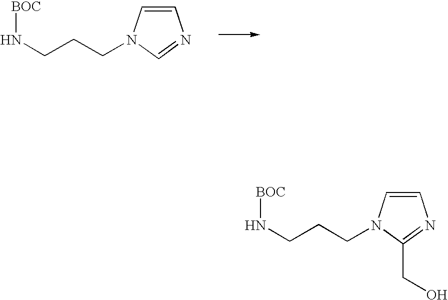

To a solution of the title compound from Step A above (0.50 g, 2.22 mmol) which is dissolved in anhydrous THF (15 ml) and is stirred at −78° C. is added n-butyllithium (2.8 mL, 1.75M in hexane) and the resulting mixture is warmed to and is stirred at −20° C. for 1.5 h. The reaction mixture is recooled to −78° C. and anhydrous DMF (0.35 mL, 4.52 mmol) is added. After warming to and stirring at 25° C. for 2 h, MeOH (2 mL) and NaBH₄ (171 mg, 4.5 mmol) is added and the resulting mixture is stirred for 1 h at 25° C. The mixture is concentrated in vacuo, is diluted with dichloromethane, is washed with water, and the organic phase is dried over anhydrous Na₂SO₄, is filtered, and is concentrated in vacuo. Purification by flash column chromatography (silica gel) using 5-10% MeOH—CH₂Cl₂ saturated with ammonium hydroxide as eluent affords the title compound.
Step C

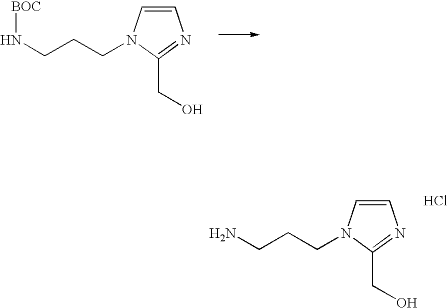

To the title compound from Step B above (0.31 g, 1.2 mmol) is added 4M HCl in dioxane (5 mL) and the mixture is stirred at 25° C. for 12 h. Concentration in vacuo affords a residue which is used directly in Step D.
Step D

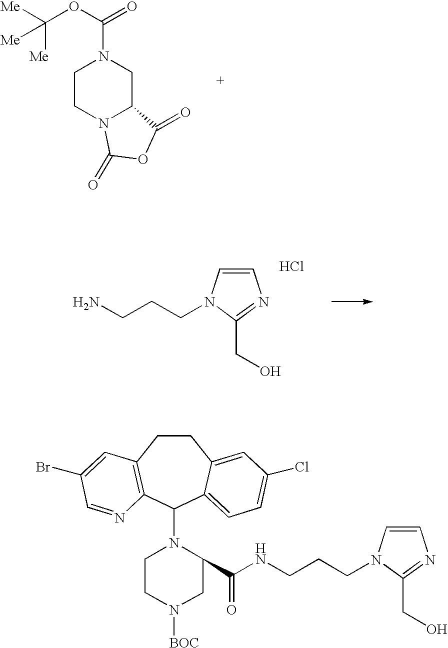

A mixture of the title compound from Step C above, triethylamine (4 mL) and the anhydride from Preparative Example 33 (0.55 g, 2.15 mmol) which is dissolved in anhydrous DMF (10 ml) is stirred at room temperature overnight. The mixture is concentrated in vacuo and is diluted with anhydrous CH₂Cl₂ (5 mL) and DMF (5 mL). The resulting mixture is stirred for 12 hrs at room temperature, then concentrated in vacuo and diluted with anhydrous CH₂Cl₂ (5 mL) and DMF (5 mL). The tricyclic chloride 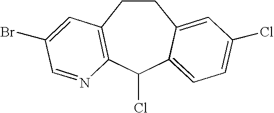
(0.75 g, 2.17 mmol) and triethylamine (3 mL) are added and the mixture is stirred at 25° C. for 48 h. The mixture is concentrated in vacuo, is diluted with CH₂Cl₂ and is washed with a saturated aqueous solution of NaHCO₃. The organic phase is dried over anhydrous Na₂SO₄, is filtered, is concentrated in vacuo and purified by flash column chromatography (silica gel) using 5-10% MeOH—CH₂Cl₂ saturated with aqueous ammonium hydroxide to give the title compound as a mixture of diastereomers.

### PREPARATIVE EXAMPLE 103

Step A

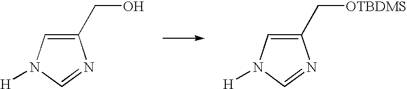

A mixture of 4-hydroxymethylimidazole (2 g, 14.9 mmol), triethylamine (5 mL) and TBDMS-Cl (2.5 g, 16.6 mmol) which is dissolved in anhydrous CH₂Cl₂ (20 ml) is stirred at room temperature overnight. The mixture is filtered, is diluted with anhydrous Et₂O and is refiltered. The filtrate is concentrated in vacuo, is diluted with CH₂Cl₂ and is washed with a saturated aqueous solution of NaHCO₃. The organic phase is dried over anhydrous Na₂SO₄, is filtered and is concentrated in vacuo to give the title compound.
Step B

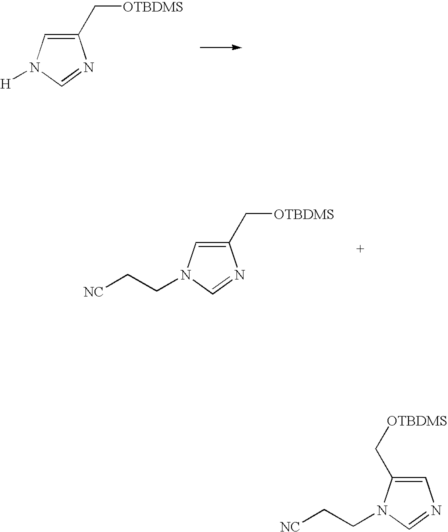

A solution of the title compound from Step A above (2.22 g, 10.5 mmol) which is dissolved in acrylonitrile (10 ml) is stirred at reflux for 48 h. Concentration in vacuo affords the title compound.
Step C

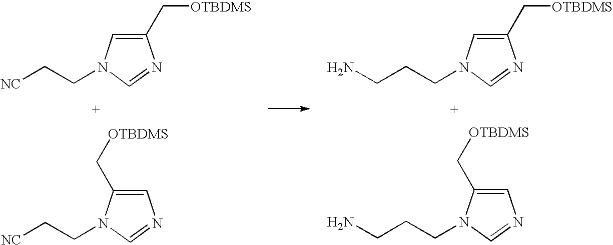

A mixture of the title compound from Step B above (2.08 g, 7.85 mmol), Raney nickel (230 mg), MeOH (20 mL) and NH₄OH (7.5 mL) is stirred in a Parr hydrogenator at room temperature for 48 h. The mixture is filtered through celite, is concentrated in vacuo, is diluted with CH₂Cl₂ and is washed with a saturated aqueous solution of NaHCO₃. The organic phase is dried over anhydrous Na₂SO₄, is filtered, is concentrated in vacuo and is purified by flash column chromatography (silica gel) using 5% MeOH—CH₂Cl₂ saturated with aqueous ammonium hydroxide to give the title compounds.

### PREPARATIVE EXAMPLE 104

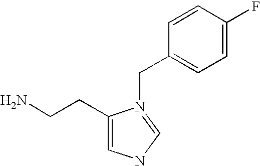

Following the procedure described for Preparative Example 141 except using 4-fluorobenzyl bromide instead of 4-chlorobenzyl chloride in Preparative Example 141 Step C, the title compound is prepared.

### PREPARATIVE EXAMPLE 105

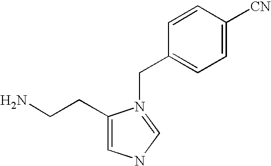

Following the procedure described for Preparative Example 141 except using 4-cyanobenzyl bromide instead of 4-chlorobenzyl chloride in Preparative Example 141 Step C, the title compound is prepared.

### PREPARATIVE EXAMPLE 106

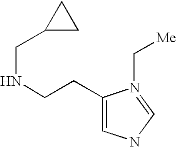

Following the procedure described for Preparative Example 22, except using the title compound from Preparative Example 10 instead of N-1-methyl histamine, the title compound is prepared.

### PREPARATIVE EXAMPLE 107

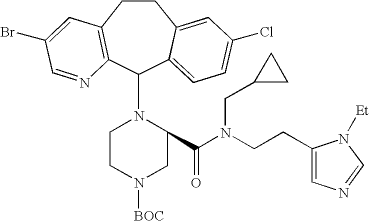

Similarly, using the procedure described for Preparative Example 102 Step D except using the title compound from Preparative Example 106 instead of the title compound from Example 102 Step C, the title compound is prepared as a mixture of diastereomers. The diastereomers could be separated using chromatography in to Diastereomer A and Diastereomer B.

### PREPARATIVE EXAMPLE 109

### 11-Chloro-6,11-dihydro-5H-benzo[5,6]cyclohepta[1,2-B]pyridine

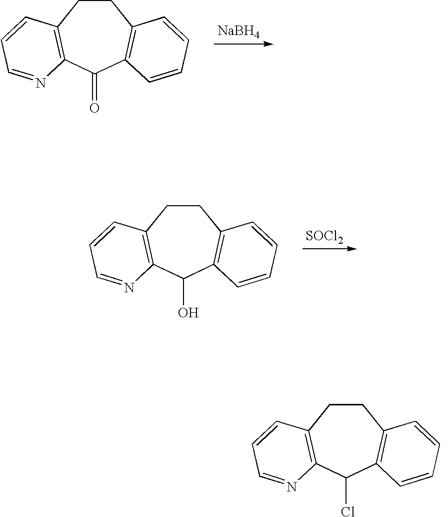

The ketone (starting material) 5,6-dihydro-11H-benzo[5,6]cyclohepta[1,2-c]pyridine-11-one, may be prepared by following the methods described in U.S. Pat. No. 3,419,565.

Sodium borohydride (2 g, 53.3 mmol) is added to a solution of the ketone (3 g, 14.35 mmol) in methanol (50 ml) at 0° C., then stirred for 2 hours at room temperature. The reaction is quenched by addition of ice (10 g) and 2N HCl (10 ml, basified with 2N NaOH (13 ml) and is extracted with MeCl₂ (2×50 ml). The organic layer is separated, dried over MgSO₄, is filtered and solvent is evaporated yielding the alcohol.

Thionyl chloride (3 ml, 41.12 mmol) is added to a solution of the alcohol (2.5 g, 11.84 mmol) in MeCl₂ (50 ml) at room temperature, then is stirred for 1 hour. The solvent is evaporated, water 50 (ml) and 5% NaOH (10 ml) are added. The mixture is extracted with MeCl₂ (100 ml), organic layer is dried over MgSO₄, is filtered, and solvent is evaporated yielding a solid, which is triturated with ether, and filtrate concentrated yielding a solid.

The filtered solid is dried yielding additional material.

### PREPARATIVE EXAMPLE 110

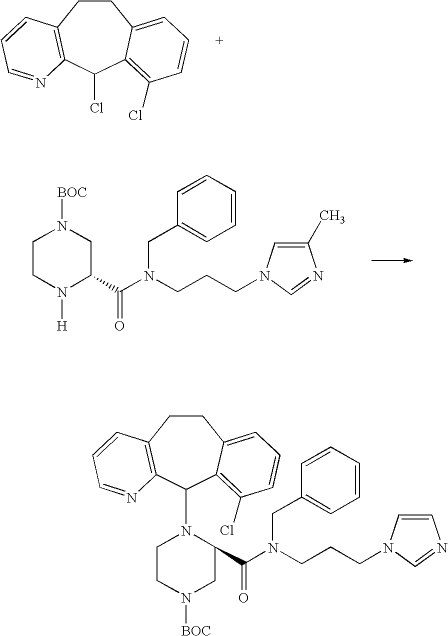

Acetonitrile (5 ml) is added to a mixture of the 10-Chloro tricycle (0.5 g, 1.90 mmol) (Preparative Example 7) and the substituted piperazine (0.78 g, 1.90 mmol). Triethylamine (1 ml, 7.18 mmol) is added, and the mixture stirred overnight at room temperature. Water (50 ml) and 5% NaOH are added and the mixture is extracted with MeCl₂ (2×100 ml). The organic layer is separated, is dried over MgSO₄ and solvent is evaporated yielding desired product as a mixture of 2 diastereomers, which are separated by column chromatography on silica gel, eluting with 5% v/vMeOH/MeCl₂ containing 2% NH₄OH.

### PREPARATIVE EXAMPLE 111

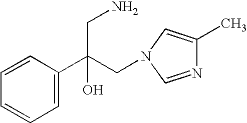 Step A

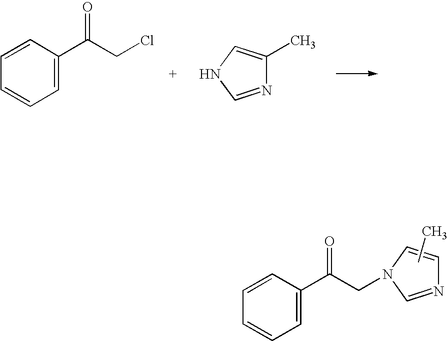

A mixture of 2-chloroacetophenone (25 g, 0.16 moles) and 4-methyl imidazole (66.1 g, 0.8 moles) is heated at 100° C. for 2 h. Cool and the crude product is chromatographed on a silica gel column eluting with CH₂Cl₂/3% CH3OH saturated with aqueous ammonium hydroxide to give a mixture of 4- and 5-methyl 1H-imidazolyl acetophenone.
Step B

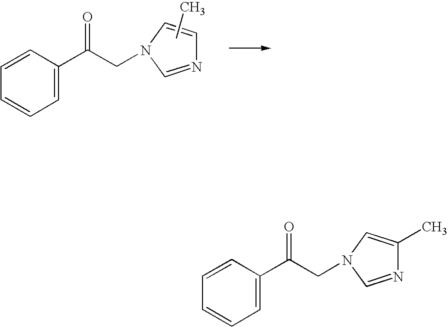

Trityl chloride (7.28 g, 0.26 moles) is added to the product from Step A in CH₂Cl₂ (200 mL) and is stirred overnight at room temperature. The mixture is chromatographed on a silica gel column eluting with ethyl acetate/acetone (3:1) to give 4-methyl-1H-imidazolyl acetophenone.
Step C

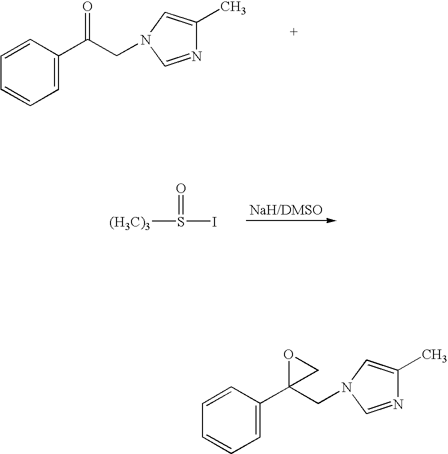

To a mixture of NaH (0.998 g, 24.97 mmoles, and trimethyl sulfoxonium iodide (5.49 g, 24.97 mmoles) in DMSO (50 mL) the product (5 g) from Step B is added and stirred for 1.5 h. Extract the product with ethyl acetate and wash with brine, dry and solvent is evaporated to give 1-(2-phenyl-2,3-epoxypropyl)-1H-4-methyl imidazole.
Step D

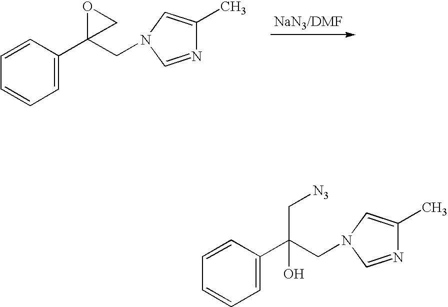

The product from Step C (3.45 g, 16.11 mmoles) and sodium azide (2.093 g, 32.21 mmoles) are heated in DMF (100 mL) at 60° C. for 12 h. Evaporate to dryness and extract with CH₂Cl₂, wash with brine and dry (MgSO₄). Evaporate to give the title compound.
Step E

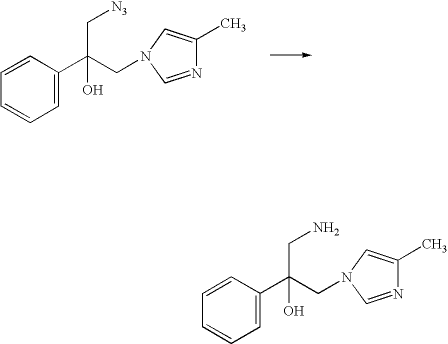

The title compound from Step D in ethanol (80 mL) is hydrogenated over 10% Pd on carbon (1.2 g) at 50 psi overnight. The catalyst is filtered off and evaporated to give the title compound.

### PREPARATIVE EXAMPLES 112-128

Following the procedure set forth in Preparative Example 74 but using the aldehyde and imidazoalkyl amine (Imidazole) in Table 6, the amines (Product) in Table 6 are obtained.

TABLE 6

| Prep | | | |
|---|---|---|---|
| Ex. | Aldehyde | Imidazole | Product |
| 112 | 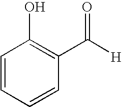 | 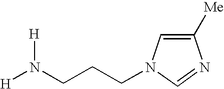 | 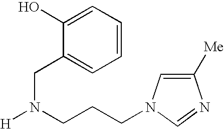 |
| 113 | 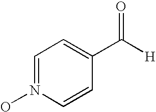 | 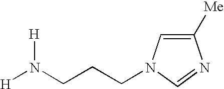 | 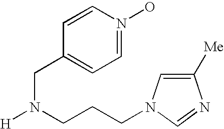 |
| 114 | 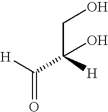 | 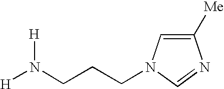 | 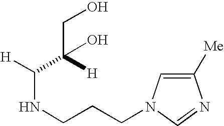 |
| 115 | 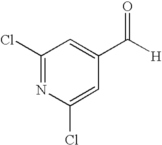 | 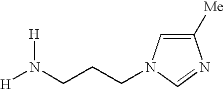 | 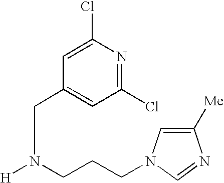 |
| 116 | 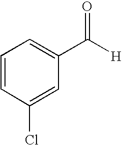 | 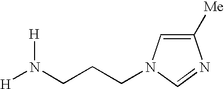 | 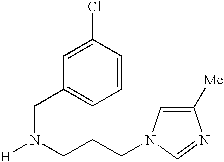 |
| 117 | 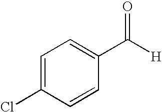 | 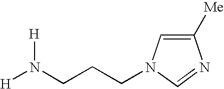 | 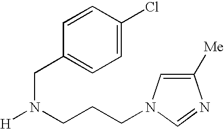 |
| 118 | 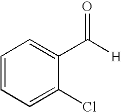 | 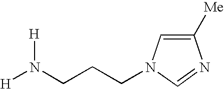 | 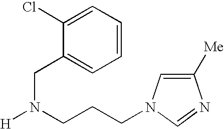 |
| 119 | 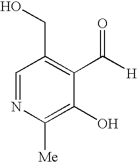 | 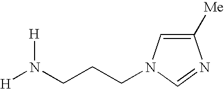 | 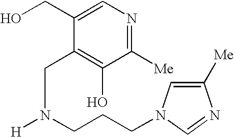 |
| 120 | 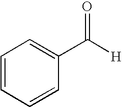 | 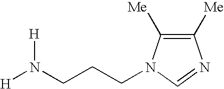 | 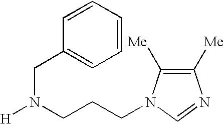 |
| 121 | 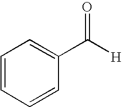 | 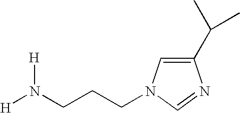 | 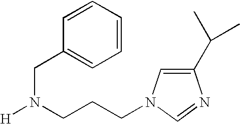 |
| 122 | 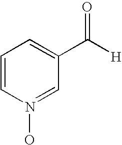 | 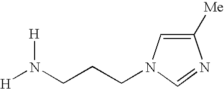 | 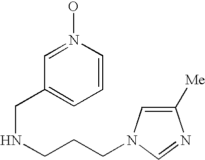 |
| 123 | 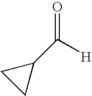 | 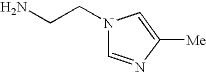 | 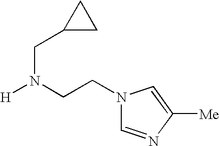 |
| 124 | 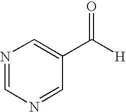 | 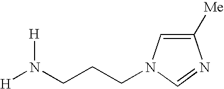 | 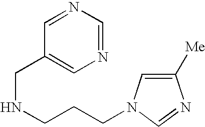 |
| 125 | 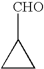 | 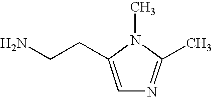 | 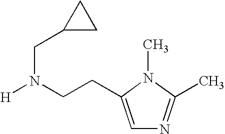 |
| 126 | 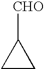 | 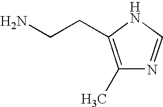 | 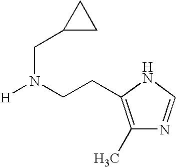 |
| 127 | 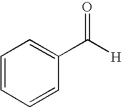 | 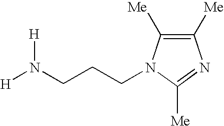 | 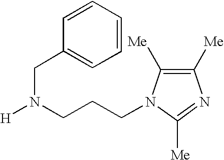 |
| 128 | 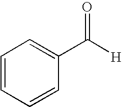 | 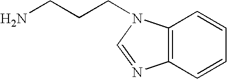 | 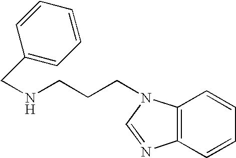 |

### PREPARATIVE EXAMPLE 129

Step A

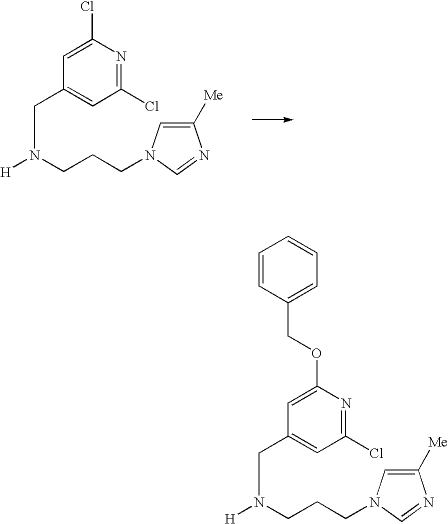

The title compound from Preparative Example 115 (0.9 g), benzyl alcohol (0.68 mL), solid potassium hydroxide (0.66 g), 18-crown-6-ether (80 mg) and anhydrous toluene (20 mL) are stirred at reflux. Purification by preparative plate chromatography (silica, 4% MeOH—CH₂Cl₂, NH₄OH saturated) affords the benzyl ether.
Step B

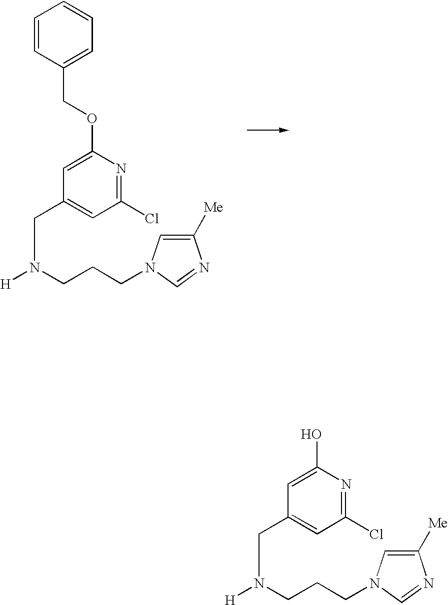

The title compound from Step A above (0.72 g), methanol (60 mL) and 10% palladium on carbon (300 mg) are stirred under 50 psi hydrogen atmosphere for 3 days. Filtration through celite affords a solution which is treated with TEA (3 equiv) and CH₂Cl₂. Filtration and purification by preparative plate chromatography (silica, 5% MeOH—CH₂Cl₂, NH₄OH saturated) affords the title compound.

### PREPARATIVE EXAMPLE 130

If one were to follow a procedure similar to that of Preparative Example 30, except substituting an equivalent amount of 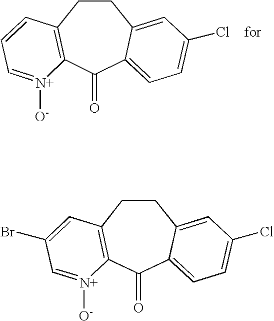
then compounds of the formula 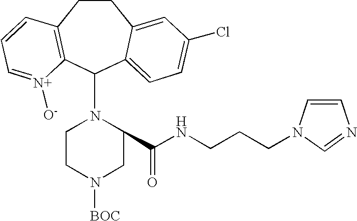
could be obtained.

### PREPARATIVE EXAMPLE 131

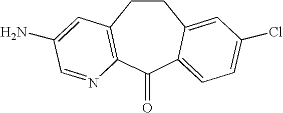

By essentially the same procedure set forth in Njoroge et. al. (J. Med. Chem. (1997), 40, 4290) for the preparation of 3-aminoloratadine only substituting the 3-H ketone (J. Het. Chem (1971) 8, 73) for loratadine, the title compound is prepared.

### PREPARATIVE EXAMPLE 132

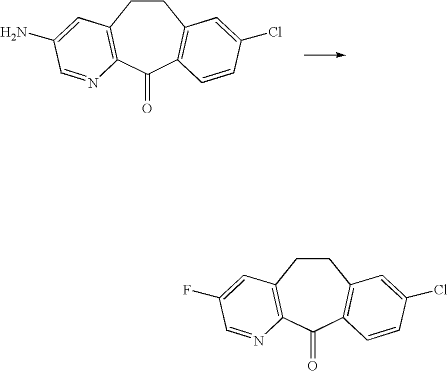

The title compound from Preparative Example 131 (1.62 g, 6.26 mmol) is added portionwise to NO⁺BF4⁻ (0.81 g, 1.1 eq.) in toluene (10 mL) at 0° C. The resulting slurry is stirred at 0° C. for 2.5 hours before warming to room temperature. The reaction mixture is heated at reflux for 2 hours, is cooled, is neutralized with 1N NaOH and is extracted with EtOAc (3×50 mL). The combined organics are washed with 1N HCl (2×25 ml), saturated NaHCO₃ (1×25 mL), and water (1×15 mL), are dried over Na₂SO₄, are filtered, and are concentrated under reduced pressure. The crude product is purified by flash chromatography using a 70:30 hexanes: EtOAc mix as eluent to yield a solid.

### PREPARATIVE EXAMPLE 133

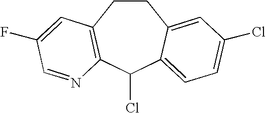

By essentially the same procedure set forth in Preparative Example 109 the title compound is prepared from the ketone of Preparative Example 132 and is used without further purification.

### PREPARATIVE EXAMPLE 134

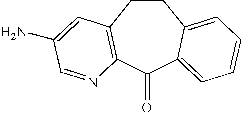

⁺NH₄HCO₂⁻ (2.44 g, 10 eq.) is added to a solution of the title compound from Preparative Example 131 (2.00 g, 7.74 mmol) and 5% Pd/C (0.50 g) in EtOH (100 mL) and the resulting solution is heated to reflux 2 hours. The reaction mixture is cooled, is filtered through a plug of Celite and is concentrated under reduced pressure. The residue is diluted with H₂O (100 mL) and is extracted with CH₂Cl₂ (3×75 mL). The combined organics are dried over Na₂SO₄, are filtered, and are concentrated in vacuo to give a solid which is used without further purification.

### PREPARATIVE EXAMPLE 135

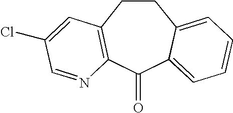

The title compound from Preparative Example 134 (1.22 g, 5.44 mmol) is added portionwise to CuCl₂ (0.88 g, 1.2 eq) and tBuONO (0.98 mL, 1.5 eq) in CH₃CN (25 mL) at 0° C. The resulting solution is warmed to RT and is stirred for 72 hours. The reaction mixture is quenched by the addition of 1M HCl (10 mL), is neutralized with 15% NH₄OH and is extracted with EtOAc (3×100 mL). The combined organics are washed with 15% NH₄OH (1×50 mL), 1M HCl (1×50 mL) and saturated NaHCO₃, are dried over Na₂SO₄, are filtered and are concentrated. The crude product is purified by flash chromatography using a 50:50 EtOAc:hexanes mixture as eluent to give a solid.

### PREPARATIVE EXAMPLE 136

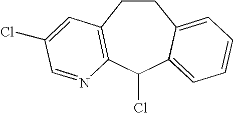

By essentially the same procedure set forth in Preparative Example 109, the title compound is prepared from the ketone of Preparative Example 135 and is used without further purification.

### PREPARATIVE EXAMPLE 137

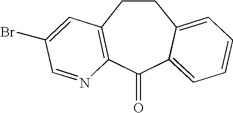

By essentially the same procedure set forth in Preparative Example 135, only substituting CuBr₂ for CuCl₂ the title compound is prepared.

### PREPARATIVE EXAMPLE 138

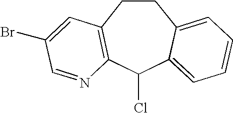

By essentially the same procedure set forth in Preparative Example 109 the title compound is prepared from the ketone of Preparative Example 137 and is used without further purification.

### PREPARATIVE EXAMPLE 139

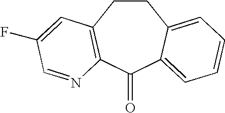

By essentially the same procedure set forth in Preparative Example 132 only substituting the title compound from Preparative Example 134, the title compound can be prepared.

### PREPARATIVE EXAMPLE 140

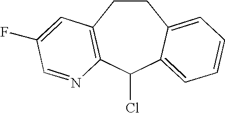

By essentially the same procedure set forth in Preparative Example 109 except starting with the ketone of Preparative Example 139, the title compound can be prepared.

### PREPARATIVE EXAMPLE 141

Step A

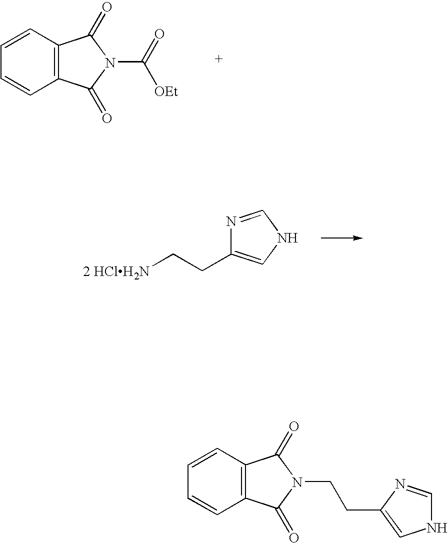

N-Carbethoxyphthalimide (62.8 g, 0.275 mol, 1.1 eq.) is added portionwise over a period of 30 minutes to a stirred solution of histamine dihydrochloride (46.7 g, 0.250 mol, 1.0 eq.) and sodium carbonate (54.3 g, 0.513 mol, 2.05 eq.) in distilled water (1250 ml) at room temperature. The resulting snow-white suspension is stirred vigorously at room temperature for 90 minutes. The solid is filtered off and thoroughly washed with ice-cold distilled water (4×50 ml). The solid is collected and dried under vacuum over P₂O₅ at 60° C. for 12 h to give the title compound.
Step B

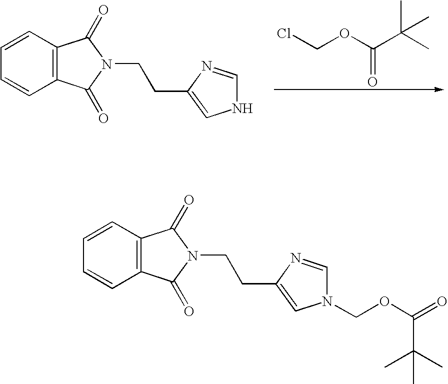

A solution of chloromethyl pivalate (18.5 ml, 0.125 mol, 1.2 eq.) in anhydrous N,N-dimethylformamide (DMF, 100 ml) is added dropwise over a period of one hour to a stirred mixture of Step A above (25.0 g, 0.104 mol, 1.0 eq.) and potassium carbonate (17.2 g, 0.125 mol, 1.2 eq.) in anhydrous DMF (500 ml) at 90° C. under a nitrogen atmosphere. The mixture is stirred at 90° C. for 12 h. The volatiles are removed under vacuum at 50° C. The residue is taken up in brine (100 ml) and extracted with ethyl acetate (4×25 ml). The combined organic extracts are dried over Na₂SO₄, are filtered, and are concentrated under vacuum at 30° C. The residual solid is flash-chromatographed (hexanes:acetone=6:4 v/v) over silica gel to give the title compound.
Step C

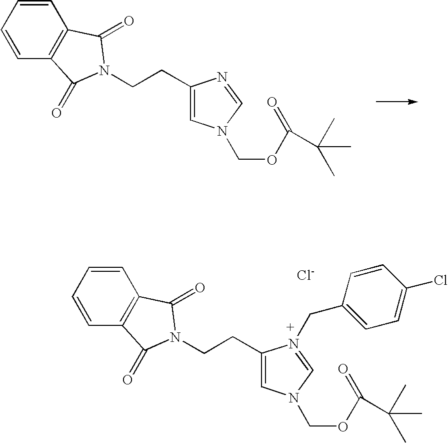

A solution of the title compound from Step B above (5 g, 14.1 mmol) and 4-chlorobenzylchloride (2.5 g, 15.5 mmol) is stirred in anhydrous acetonitrile (60 ml) at reflux under a nitrogen atmosphere for 48 h. The mixture is concentrated in vacuo and is recrystallized from ethyl acetate-hexane to give the title compound as a solid, and the filtrate which is concentrated to give additional product.
Step D

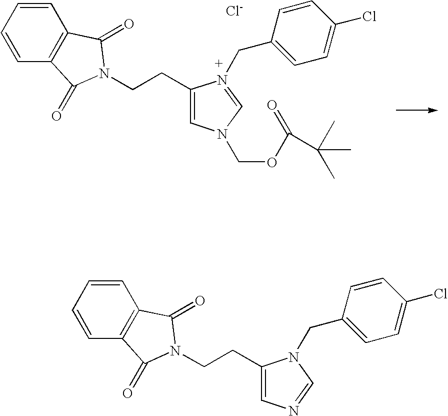

A 7 N solution of ammonia in methanol (10 ml, 0.07 mol) is added slowly to a stirred solution of the title compound from Step C above (3.2 g, 6.6 mmol) is diluted with MeOH (10 mL) at −20° C. The resulting mixture is warmed to room temperature and is stirred for another 12 h, then is concentrated in vacuo and is purified by flash column chromatography (silica gel) using 3% MeOH—CH₂Cl₂ saturated with ammonium hydroxide as eluent to afford the title compound.
Step E

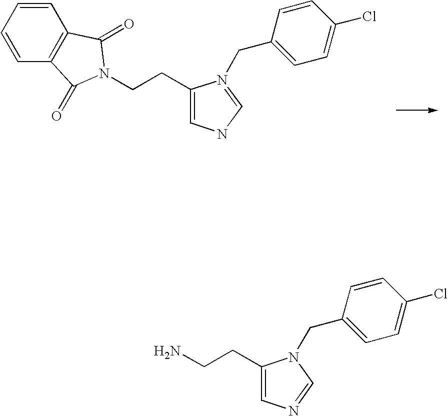

A solution of the title compound from Step D above (1.21 g, 3.3 mmol) and hydrazine monohydrate (1.7 ml, 0.033 mol, 10 eq.) in absolute ethanol (20 ml) is stirred at 50° C. under a nitrogen atmosphere for 20 min. The resulting suspension is diluted with ethanol and dichloromethane and filtered. The filtrate was concentrated in vacuo to afford the title compound.

### EXAMPLE 1

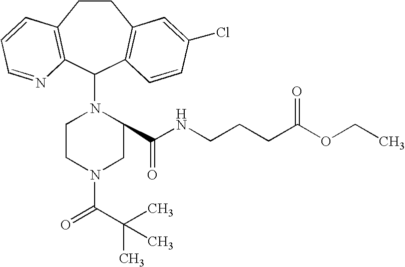 Step 1:

### Preparation of 3R-(3-ethoxycarbonyl-propylcarbamoyl)-piperazine-1-carboxylic acid tert-butyl ester (3)

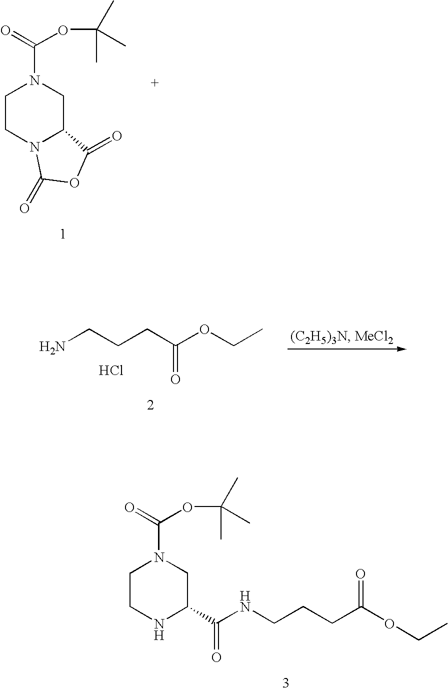

Triethylamine (2 ml; 14.26 mmol) was added to a solution of the anhydride 1 (prepared as described in Preparative Example 44 of U.S. Pat. No. 6,372,747 B1, the disclosure of which is incorporated herein by reference thereto) and ethyl 4-aminobutyrate hydrochloride (1.63 g; 9.76 mmol) in MeCl₂ (20 ml) at 0° C., then stirred at room temperature for 1 hour. The solvent was evaporated yielding compound 3 (3.4 g; 100%) which was used without purification in Step 2.

Step 2

### 4-(8-Chloro-6,11-dihydro-5H-benzo[5,6]cyclohepta[1,2-b]pyridin-11-yl)-3R-(3-ethoxycarbonyl-propylcarbamoyl)-piperazine-1-carboxylic acid tert-butyl ester (5)

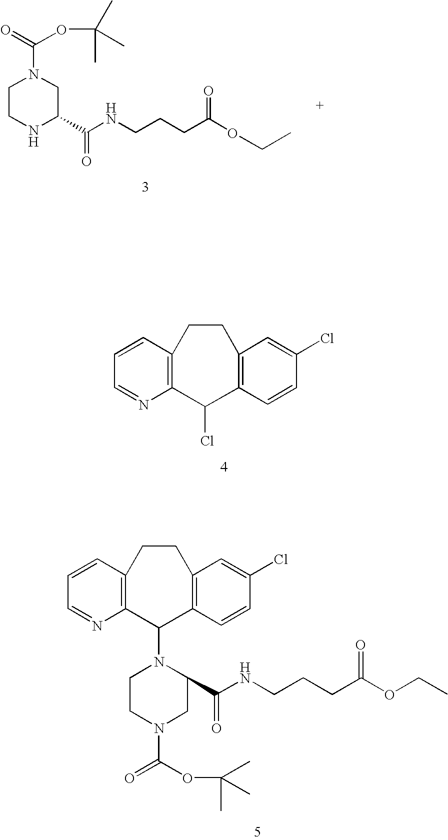

Triethylamine (2 ml; 14.26 mmol) was added to a solution of compound 3 (from Step 1) 3.4 g, 9.91 mmol) and 8,11-Dichloro-6,11-dihydro-5H-benzo[5,6]cyclohepta[1,2-b]pyridine 4 (3.5 g; 13.3 mmol) (prepared as described in U.S. Pat. No. 5,151,423, the disclosure of which is incorporated herein by reference thereto) in acetonitrile (15 ml), then stirred at room temperature overnight. The solvent was evaporated and the residue was extracted with MeCl₂ (100 ml), washed with H₂O (30 ml) then dried over MgSO₄. The solvent was evaporated yielding crude product which was purified on column chromatography eluting with Ethyl acetate:Hexanes v/v 80/20 yielding product as a mixture of 2 diastereomers (4.5 g, 79%).

This product (150 mg, 0.26 mmol) was separated on silica gel Preparative TLC (2000 uM; 20×20 cm-1) into individual diastereomers eluting with 20% acetone in 1:1 MeCl₂:hexanes. The slower eluting diastereomer A 6 (60 mg; 40%) was obtained as a white solid. Mass Spec (High resolution FABS) Calculated for (MH C₃₀H₄₀N₄O₅Cl) 571.2687 Measured 571.2679.

Specific rotation in EtOH [α]_{D}^{20c}=+54.2°

The faster eluting diastereomer B 7 (65 mg:43%) was obtained as a white solid Mass Spec (High resolution FABS) Calculated for (MH C₃₀H₄₀N₄O₅Cl) 571.2687 Measured 571.2679.

Specific rotation in EtOH [α]_{D}^{20c}=+75.8°

### EXAMPLE 2

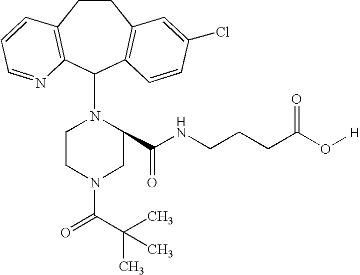 Step 1

### Preparation of 3R-(3-Carboxy-propylcarbamoyl)-4-(8-chloro-6,11-dihydro-5H-benzo[5,6]cyclohepta[1,2-b]pyridin-11-yl)-piperazine-1-carboxylic acid tert-butyl ester (8) Diastereomer A

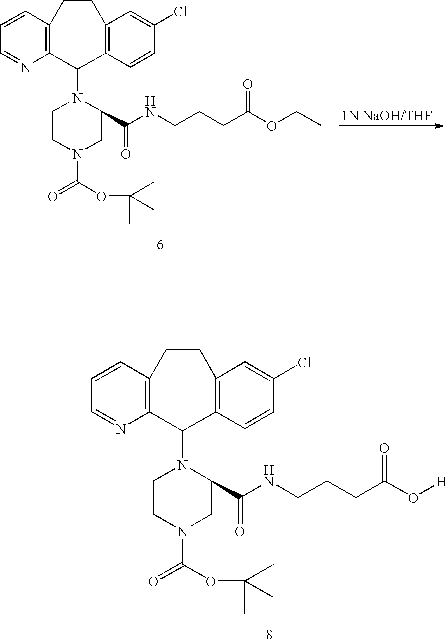

Sodium hydroxide (1 ml; 1N, 1 mmol) was added to a solution of 4-(8-Chloro-6,11-dihydro-5H-benzo[5,6]cyclohepta[1,2-b]pyridin-11-yl)-3-(3-ethoxycarbonyl-propylcarbamoyl)-piperazine-1-carboxylic acid tert-butyl ester 6 (Diastereomer A from Example 1, 20 mg, 0.035 mmol) in THF/MeOH (v/v 1/1 2 ml) then stirred overnight at room temperature. The solvent was evaporated and the residue extracted with ether (5 ml) and H₂O (10 ml). The aqueous layer was separated, added to 10% citric acid (10 ml), then extracted with MeCl₂ (3×25 ml). The organic layers were combined, dried over MgSO₄ filtered and solvent evaporated yielding 8 as a white solid (15 mg; 79%).

Mass Spec (FABS, MH) calculated C₂₈H₃₅ClN₄O₅ (543.068)

Measured 543.1

Step 2

### Preparation of 3R-(3-Carboxy-propylcarbamoyl)-4-(8-chloro-6,11-dihydro-5H-benzo[5,6]cyclohepta[1,2-b]pyridin-11-yl)-piperazine-1-carboxylic acid tert-butyl ester (Diastereomer B, Compound 9)

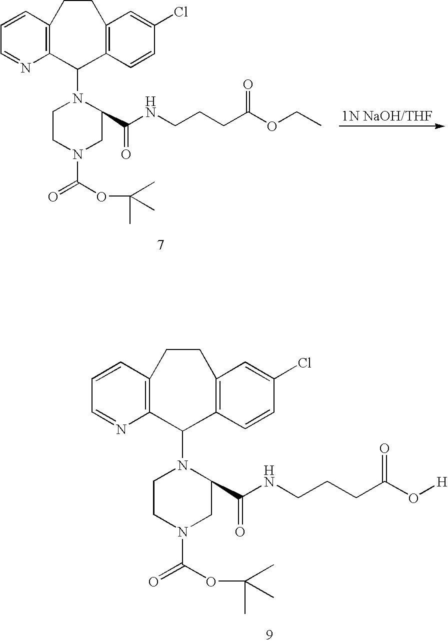

Following a procedure similar to that described in Step 1, but substituting an equivalent amount of compound 7 for compound 6, the title compound 9 was obtained.

Mass Spec (FABS, MH) 543.1

### EXAMPLE 3

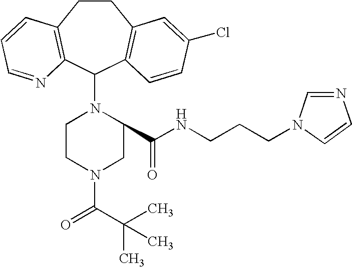 Step 1:

### Preparation of 3-(3-Imidazol-1-yl-propylcarbamoyl)-piperazine-1-carboxylic acid tert-butyl ester (11)

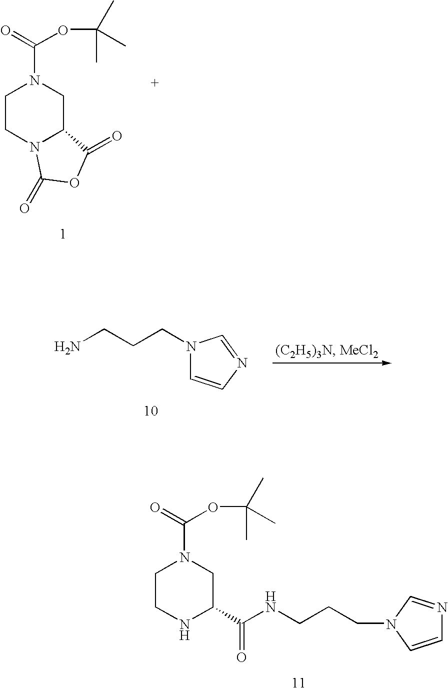

Following a procedure similar to that of Example 1, Step 1, but substituting an equivalent quantity of 1-(3-aminopropyl)-imidazole 10 for ethyl-4-aminobutyrate hydrochloride, the product 11 was obtained.

Step 2

### 4-(8-Chloro-6,11-dihydro-5H-benzo[5,6]cyclohepta[1,2-b]pyridin-11-yl)-3R-(3-imidazol-1-yl-propylcarbamoyl)-piperazine-1-carboxylic acid tert-butyl ester (12)

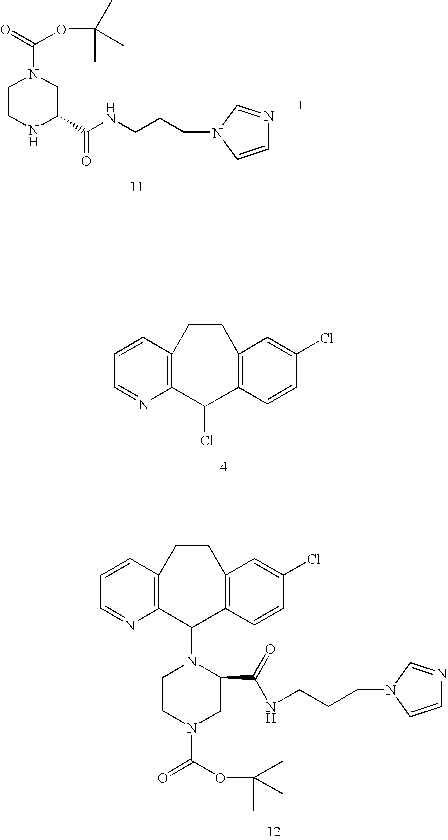

Following a procedure similar to that of Example 1, Step 2, but substituting an equivalent quantity of compound II for compound 3, the crude product was chromatographed on silica gel eluting with 5% MeOH/MeCl₂/NH₄OH yielding 12 as a mixture of 2 diastereomers.

Mass Spec (High resolution; FABS; MH) Calculated MH (565.2694) Observed MH (565.2720).

Compound 12 was separated into single diastereomers on Preparative silica gel (20×20 cm-1) eluting with 5% MeOH/EtOAc/NH₄OH.

Diastereomer A: MH ESMS (565.1). Diastereomer B: MH, ESMS (565.1).

### EXAMPLE 4

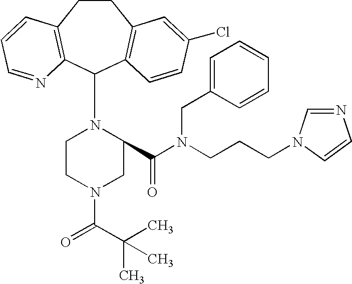 Step 1

### 3-[Benzyl-(3-imidazol-1-yl-propyl)-carbamoyl]-piperazine-1-carboxylic acid tert-butyl ester (14)

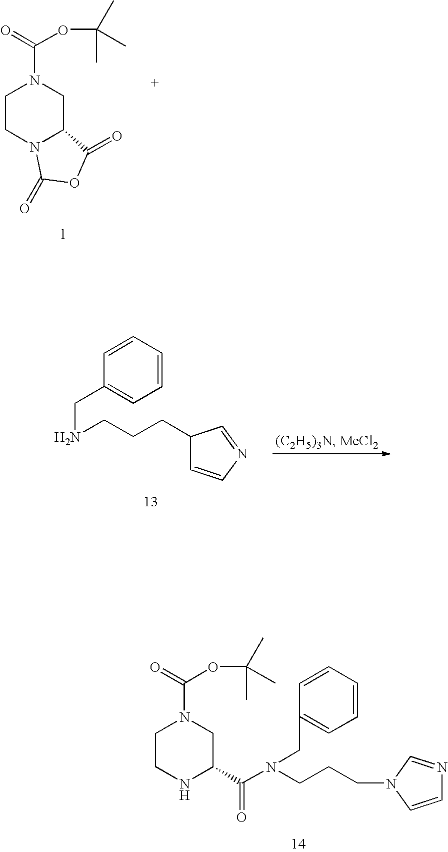

Preparation of starting material 13 (benzyl-[3-(3H-pyrrol-3-yl)-propyl]-amine) is described in Preparative Example 74 of U.S. Pat. No. 6,372,747 B1, the disclosure of which is incorporated herein by reference hereto.

Following a procedure similar to that of Example 1, Step 1, but substituting an equivalent quantity of 13 for ethyl-4-aminobutyrate hydrochloride 2, the title compound 14 was obtained in 72% yield.

Mass Spec (High Resolution: FABS, MH) Calculated: 428.2662 Measured: 428.2674.

Step 2

### 3R-[Benzyl-(3-imidazol-1-yl-propyl)-carbamoyl]-4-(8-chloro-6,11-dihydro-5H-benzo[5,6]cyclohepta[1,2-b]pyridin-11-yl)-piperazine-1-carboxylic acid tert-butyl ester (15)

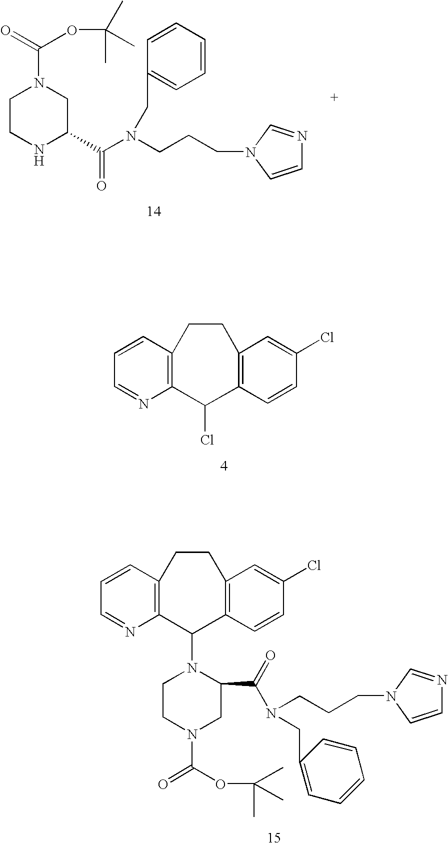

Following a procedure similar to that of Example 1, Step 2, but substituting an equivalent quantity of compound 14 for compound 3, the crude product was chromatographed on silica gel eluting with 5% MeOH/MeCl₂/NH₄OH yielding 15 as a mixture of 2 diastereomers.

Compound 15 was separated into single diastereomers on Preparative silica gel (20×20 cm⁻¹) eluting with 5% MeOH/EtOAc/NH₄OH.

Diastereomer A: High Resolution Mass Spec (FABS MH) Calculated 655.3163 Observed 655.3175.

Diastereomer B: High Resolution Mass Spec (FABS MH) Calculated 655.3163 Observed 655.3185.

Assays

FPT IC₅₀ (inhibition of farnesyl protein transferase, in vitro enzyme assay) was determined, and COS Cell IC₅₀ (Cell-Based Assay) could be determined following the assay procedures described in WO 95/10516, published Apr. 20, 1995. GGPT IC₅₀ (inhibition of geranylgeranyl protein transferase, in vitro enzyme assay), Cell Mat Assay, and anti-tumor activity (in vivo anti-tumor studies) could be determined by the assay procedures described in WO 95/10516. The disclosure of WO 95/10516 is incorporated herein by reference thereto.

Additional assays can be carried out by following essentially the same procedure as described above, but with substitution of alternative indicator tumor cell lines in place of the T24-BAG cells. The assays can be conducted using either DLD-1-BAG human colon carcinoma cells expressing an activated K-ras gene or SW620-BAG human colon carcinoma cells expressing an activated K-ras gene. Using other tumor cell lines known in the art, the activity of the compounds of this invention against other types of cancer cells could be demonstrated.

Soft Agar Assay:

Anchorage-independent growth is a characteristic of tumorigenic cell lines. Human tumor cells can be suspended in growth medium containing 0.3% agarose and an indicated concentration of a farnesyl transferase inhibitor. The solution can be overlayed onto growth medium solidified with 0.6% agarose containing the same concentration of farnesyl transferase inhibitor as the top layer. After the top layer is solidified, plates can be incubated for 10-16 days at 37° C. under 5% CO₂ to allow colony outgrowth. After incubation, the colonies can be stained by overlaying the agar with a solution of MTT (3-[4,5-dimethyl-thiazol-2-yl]-2,5-diphenyltetrazolium bromide, Thiazolyl blue) (1 mg/mL in PBS). Colonies can be counted and the IC₅₀'s can be determined.

The FPT IC₅₀ for the compounds of Examples 1-3 were: (1) 5.2 nM for Diastereomer A (i.e., compound 6) of Example 1, (2) 5.5 nM for Diastereomer B (i.e., compound 7) of Example 1, (3) 5.5 nM for compound 8 of Example 2, and 6.3 nM for compound 9 of Example 2. The diastereomer mixture of Compound 12 in Example 3 had an FPT IC₅₀ of 1.2 nM.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in two to four divided doses.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of the formula: 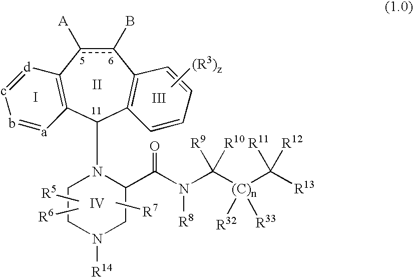
or a pharmaceutically acceptable salt or thererof, wherein:
one of a, b, c and d represents N or N⁺O⁻, and the remaining a, b, c and d groups represent CR¹ wherein each R¹ is independently selected; or
each of a, b, c, and d are CR¹ wherein each R¹ is independently selected;
the dotted line between carbon atoms 5 and 6 represents an optional bond;
when the optional bond is present between C5 and C6, each A and B are each independently selected from the group consisting of: —R¹⁵, halo, —OR¹⁶, —OCO₂R¹⁶ and —OC(O)R¹⁵;
when the optional bond between C5 and C6 is not present, each A and B are each independently selected from the group consisting of: (a) H₂, (b) —(OR¹⁶)₂ wherein each R¹⁶ is independently selected, (c) H and halo, (d) dihalo wherein each halo is independently selected, (e) alkyl and H, (f) (alkyl)₂ wherein each alkyl is independently selected, (g) —H and —OC(O)R¹⁵, (h) H and —OR¹⁵, (i) ═O, (j) aryl and H, (k) ═NOR¹⁵ and (l) —O—(CH₂)ₚ—O— wherein p is 2, 3 or 4;
each R¹ is independently selected from the group consisting of: (a) H, (b) halo, (c) —CF₃, (d) —OR⁵, (e) —COR⁵, (f) —SR⁵, (g) —S(O)ₜR¹⁶ (wherein t is 0, 1 or 2), (h) —N(R⁵)₂, (i) —NO₂, (j) —OC(O)R⁵, (k) —CO₂R⁵, (l) —OCO₂R¹⁶, (m) —CN, (n) —NR¹⁵COOR¹⁶, (o) —SR¹⁶C(O)OR¹⁶, (p) —SR¹⁶N(R¹⁷)₂ (provided that R¹⁶ in —SR¹⁶N(R¹⁷)₂ is not —CH₂—) wherein each R¹⁷ is independently selected from the group consisting of H and —C(O)OR¹⁶, (q) benzotriazol-1-yloxy, (r) tetrazol-5-ylthio, (s) substituted tetrazol-5-ylthio, (t) alkynyl, (u) alkenyl and (v) alkyl, said alkyl or alkenyl group optionally being substituted with halo, —OR¹⁵ or —CO₂R¹⁵;
each R³ is independently selected from the group consisting of: (a) halo, (b) —CF₃, (c) —OR¹⁵, (d) —COR¹⁵, (e) —SR¹⁵, (f) —S(O)ₜR¹⁶ (wherein t is 0, 1 or 2) (g) —N(R¹⁵)₂, (h) —NO₂, (i) —OC(O)R¹⁵, (j) —CO₂R¹⁵, (k) —OCO₂R¹⁶, (l) —CN, (m) —NR¹⁵COOR¹⁶, (n) —SR¹⁶C(O)OR¹⁶, (o) —SR¹⁶N(R¹⁷)₂ (provided that R¹⁶ in —SR¹⁶N(R¹⁷)₂ is not —CH₂—) wherein each R¹⁷ is independently selected from the group consisting of H and —C(O)OR¹⁶, (p) benzotriazol-1-yloxy, (q) tetrazol-5-ylthio, (r) substituted tetrazol-5-ylthio, (s) alkynyl, (t) alkenyl and (u) alkyl, said alkyl or alkenyl group optionally being substituted with halo, —OR¹⁵ or —CO₂R¹⁵; or
two R³ groups taken together with the carbon atoms to which they are bound form a saturated or unsaturated C₅-C₇ ring;
z is 0, 1, 2, or 3;
R⁵, R⁶, and R⁷ are each independently selected from the group consisting of: H, —CF₃, —COR¹⁵, alkyl and aryl, wherein said alkyl or aryl is optionally substituted with —OR¹⁵, —SR¹⁵, —S(O)ₜR¹⁶, —NR¹⁵COOR¹⁶, —N(R¹⁵)₂, —NO₂, —COR¹⁵, —OCOR¹⁵, —OCO₂R¹⁶, —CO₂R¹⁵, and OPO₃R¹⁵, or R⁵ and R⁶ together represent ═O or ═S;
R⁸ is selected from the group consisting of: H, C₃ to C₇ alkyl, aryl, arylalkyl-, heteroaryl, heteroarylalkyl-, cycloalkyl, cycloalkylalkyl-, substituted alkyl, substituted aryl, substituted arylalkyl-, substituted heteroaryl, substituted heteroarylalkyl-, substituted cycloalkyl, substituted cycloalkylalkyl-;
the substituents for the R⁸ substituted groups are independently selected from the group consisting of: alkyl, aryl, arylalkyl-, cycloalkyl, —N(R¹⁸)₂, —OR¹⁸, cycloalkyalkyl-, halo, CN, —C(O)N(R¹⁸)₂, —SO₂N(R¹⁸)₂ and —CO₂R¹⁸; provided that the —OR¹⁸ and —N(R¹⁸)₂ substituents are not bound to the carbon that is bound to the N of the —C(O)NR⁸— moiety;
R⁹ and R¹⁰ are independently selected from the group consisting of: H, alkyl, aryl, arylalkyl-, heteroaryl, heteroarylalkyl-, cycloalkyl or —CON(R¹⁸)₂ (wherein R¹⁸ is as defined above); and the substitutable R⁹ and R¹⁰ groups are optionally substituted with one or more substituents independently selected from the group consisting of: alkyl, cycloalkyl, arylalkyl-, or heterarylalkyl-; or
R⁹ and R¹⁰ together with the carbon atom to which they are bound, form a C₃ to C₆ cycloalkyl ring;
R¹¹ and R¹² are independently selected from the group consisting of: H, alkyl, aryl, arylalkyl-, heteroaryl, heteroarylalkyl-, cycloalkyl, —CON(R¹⁸)₂—OR¹⁸ or —N(R¹⁸)₂; wherein R¹⁸ is as defined above; provided that the —OR¹⁸ and —N(R¹⁸)₂ groups are not bound to a carbon atom that is adjacent to a nitrogen atom; and wherein said substitutable R¹¹ and R¹² groups are optionally substituted with one or more substituents selected from the group consisting of: alkyl, cycloalkyl, arylalkyl-, or heterarylalkyl-; or
R¹¹ and R¹² together with the carbon atom to which they are bound, form a C₃ to C₆ cycloalkyl ring; or
R¹¹ and R¹² taken together with the carbon to which they are bound form a 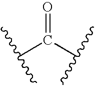
moiety;
R¹³ is selected from the group consisting of: —OR⁴⁰ (wherein R⁴⁰ is an alkyl group), —C(O)OR⁶⁰ and imidazolyl, wherein said imidazolyl is selected from the group consisting of: 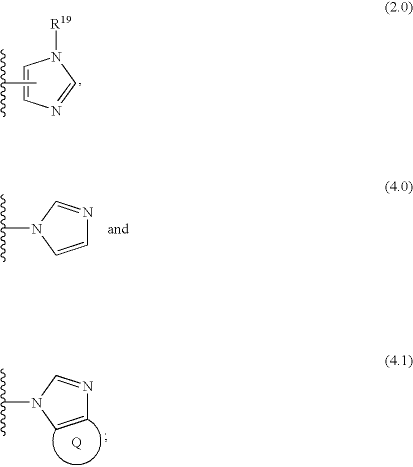
wherein said imidazolyl ring 2.0 is optionally substituted with one or two substituents, and said imidazole ring 4.0 is optionally substituted with 1-3 substituents, and said imidazole ring 4.1 is optionally substituted with one substituent, and wherein said optional substituents for said imidazolyl rings 2.0, 4.0 and 4.1 are bound to the carbon atoms of said imidazolyl rings, and said optional substituents are independently selected from the group consisting of: —NHC(O)R¹⁸, —C(R³⁴)₂OR³⁵, —OR¹⁸, —SR¹⁸, F, Cl, Br, alkyl, aryl, arylalkyl-, cycloalkyl, and —N(R¹⁸)₂ (wherein each R¹⁸ is independently selected);
Q represents an aryl ring, a cycloalkyl ring, or a heteroaryl ring, said Q is optionally substituted with 1 to 4 substituents independently selected from the group consisting of: halo, alkyl, aryl, —OR¹⁸, —N(R¹⁸)₂ (wherein each R¹⁸ is independently selected), —OC(O)R¹⁸, and —C(O)N(R¹⁸)₂ (wherein each R¹⁸ is independently selected);
R¹⁴ is selected from the group consisting of: 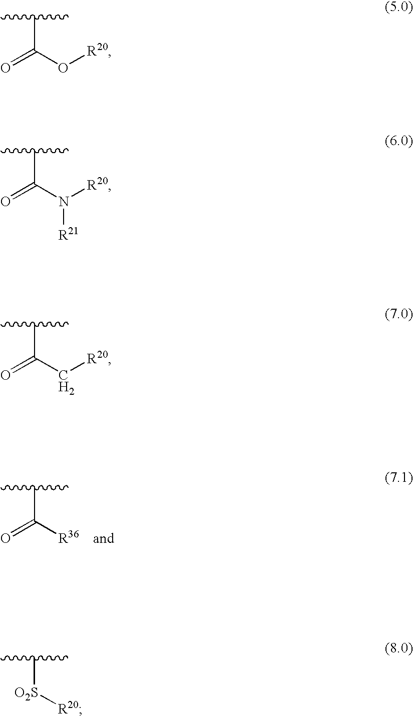
R¹⁵ is selected from the group consisting of: H, alkyl aryl and arylalkyl-;
R¹⁶ is selected from the group consisting of: alkyl and aryl;
each R¹⁸ is independently selected from the group consisting of: H, alkyl, aryl, arylalkyl-, heteroaryl and cycloalkyl;
R¹⁹ is selected from the group consisting of: (1) H, (2) alkyl, (3) aryl, (4) arylalkyl-, (5) substituted arylalkyl-, (6) —C(aryl)₃ and (7) cycloalkyl; and wherein the substituents on said substituted arylalkyl- are selected from the group consisting of: halo and CN;
R²⁰ is selected from the group consisting of: H, alkyl, alkoxy, aryl, arylalkyl-, cycloalkyl, heteroaryl, heteroarylalkyl- and heterocycloalkyl, provided that R²⁰ is not H when R¹⁴ is group 5.0 or 8.0;
when R²⁰ is other than H, then said R²⁰ group is optionally substituted with one or more substituents selected from the group consisting of: halo, alkyl, aryl, —OC(O)R¹⁸, —OR¹⁸ and —N(R¹⁸)₂, wherein each R¹⁸ group is the same or different, provided that said optional substituent is not bound to a carbon atom that is adjacent to an oxygen or nitrogen atom;
R²¹ is selected from the group consisting of: H, alkyl, aryl, arylalkyl-, cycloalkyl, heteroaryl, heteroarylalkyl- and heterocycloalkyl;
when R²¹ is other than H, then said R²¹ group is optionally substituted with one or more substituents selected from the group consisting of: halo, alkyl, aryl, —OR¹⁸ and —N(R¹⁸)₂, wherein each R¹⁸ group is the same or different, provided that said optional substituent is not bound to a carbon atom that is adjacent to an oxygen or nitrogen atom;
n is 0-5;
each R³² and each R³³ for each n, is independently selected from the group consisting of: H, alkyl, aryl, arylalkyl-, heteroaryl, heteroarylalkyl-, cycloalkyl, —CON(R¹⁸)₂, —OR¹⁸ and —N(R¹⁸)₂; and wherein said substitutable R³² and R³³ groups are optionally substituted with one or more substituents selected from the group consisting of: alkyl, cycloalkyl, arylalkyl-, and heterarylalkyl-; or
R³² and R³³ together with the carbon atom to which they are bound, form a C₃ to C₆ cycloalkyl ring;
each R³⁴ is independently selected from the group consisting of: H and alkyl, and R³⁴ is preferably H;
R³⁵ is selected from the group consisting of: H, —C(O)OR²⁰ and —C(O)NHR²⁰;
R³⁶ is selected from the group consisting of: branched alkyl, unbranched alkyl, cycloalkyl, heterocycloalkyl, and aryl; and
R⁶⁰ is selected from the group consisting of: H and alkyl.

2. The compound of claim 1 having the structure:

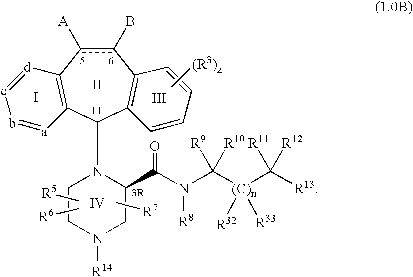

3. The compound of claim 1 having the structure:

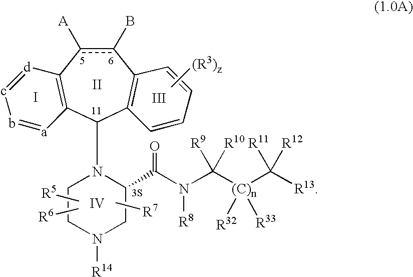

4. The compound of claim 1 wherein R¹ to R⁴ is independently selected from the group consisting of: H, Br or Cl; R⁵ to R⁷ is H; a is N and the remaining b, c and d substituents are carbon; A and B are H₂; and n is 0 or 1.

5. The compound of claim 1 wherein R¹ to R⁴ is independently selected from the group consisting of: H, Br or Cl; R⁵ to R⁷ is H; a is N and the remaining b, c and d substituents are carbon; A and B are H₂; n is 0 or 1; and R¹³ is —C(O)OR⁶⁰ or group 4.0.

6. The compound of claim 1 wherein R¹ to R⁴ is independently selected from H, Br or Cl; R⁵ to R⁷ is H; a, b, c and d are carbon; A and B are H₂; and n is 0 or 1.

7. The compound of claim 1 wherein R⁸ is selected from the group consisting of: H, arylalkyl, substituted arylalkyl, cycloalkylalkyl, substituted cycloalkylalkyl, heteroarylalkyl or substituted heteroarylalkyl.

8. The compound of claim 1 wherein R⁸ is selected from the group consisting of: H and arylalkyl.

9. The compound of claim 1 wherein R⁸ is H.

10. The compound of claim 1 wherein:
(a) R⁹ and R¹⁰ are H;
(b) R¹¹ and R¹²H;
(c) R³² and R³³ are H;
(e) the optional substituents on said R¹³ are independently selected from alkyl.

11. The compound of claim 5 wherein:
(a) R⁹ and R¹⁰ are H;
(b) R¹¹ and R¹²H;
(c) R³² and R³³ are H;
(e) the optional substituents on said R¹³ are independently selected from alkyl.

12. The compound of claim 11 wherein R⁸ is H.

13. The compound of claim 1 wherein R¹⁴ is group 5.0 and R²⁰ is alkyl.

14. The compound of claim 12 wherein R¹⁴ is group 5.0 and R²⁰ is alkyl.

15. The compound of claim 1 wherein R¹⁴ is 7.1 and R³⁶ is alkyl.

16. The compound of claim 12 wherein R¹⁴ is 7.1 and R³⁶ is alkyl.

17. The compound of claim 14 wherein said compound is the 3R isomer.

18. The compound of claim 16 wherein said compound is the 3R isomer.

19. A compound selected from the group consisting of the final compounds of Example 1, 2, 3 and 4.

20. A pharmaceutical composition comprising at least one compound of claim 1, or a pharmaceutically acceptable salt thereof, and a pharmaceutically acceptable carrier.

21. A pharmaceutical composition comprising at least one compound of claim 19, or a pharmaceutically acceptable salt thereof, and a pharmaceutically acceptable carrier.

22. A pharmaceutical composition comprising at least one compound of claim 1 and a pharmaceutically acceptable carrier.

23. A pharmaceutical composition comprising at least one compound of claim 19 and a pharmaceutically acceptable carrier.

24. A method of treating cancer in a patient in need of such treatment, said method comprising administering to said patient an effective amount of at least one compound of claim 1, or a pharmaceutically acceptable salt thereof.

25. A method of treating cancer in a patient in need of such treatment, said method comprising administering to said patient an effective amount of at least one compound of claim 1, or a pharmaceutically acceptable salt thereof, in combination with at least one chemotherapeutic agent.

26. A method of inhibiting farnesyl protein transferase in a patient in need of such treatment, said method comprising administering to said patient an effective amount of at least one compound of claim 1, or a pharmaceutically acceptable salt thereof.

27. A method of treating cancer in a patient in need of such treatment, said method comprising administering to said patient an effective amount of at least one compound of claim 1, or a pharmaceutically acceptable salt thereof, in combination with at least one signal transduction inhibitor.

28. The method of claim 24 wherein said cancer is selected from the group consisting of: lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, nuroblastomas, kidney carcinomas, hepatomas, non-Hodgkin's lymphoma, multiple myeloma, and anaplastic thyroid carcinoma.

29. The method of claim 25 wherein said cancer is selected from the group consisting of: lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, nuroblastomas, kidney carcinomas, hepatomas, non-Hodgkin's lymphoma, multiple myeloma, and anaplastic thyroid carcinoma.

30. The method of claim 24 wherein said cancer is selected from the grop consisting of: melanoma, pancreatic cancer, thyroid cancer, colorectal cancer, lung cancer, breast cancer, and ovarian cancer.

31. The method of claim 25 wherein said cancer is selected from the grop consisting of: melanoma, pancreatic cancer, thyroid cancer, colorectal cancer, lung cancer, breast cancer, and ovarian cancer.

32. A method of treating breast cancer in a patient in need of such treatment, said treatment comprising administering to said patient an effective amount of at least one compound of claim 1 in combination with hormonal therapies.

33. A method of treating breast cancer in a patient in need of such treatment, said treatment comprising administering to said patient an effective amount of at least one compound of claim 1 in combination with hormonal therapies, and in combination with an effective amount of at least one chemotherapeutic agent.

34. A method of preventing breast cancer in a patient in need of such treatment, said treatment comprising administering to said patient an effective amount of at least one compound of claim 1 in combination with hormonal therapies.

35. A method of preventing breast cancer in a patient in need of such treatment, said treatment comprising administering to said patient an effective amount of at least one compound of claim 1 in combination with hormonal therapies, and in combination with an effective amount of at least one chemotherapeutic agent.

36. A method of treating brain cancer in a patient in need of such treatment, said treatment comprising administering to said patient an effective amount of at least one compound of claim 1.

37. A method of treating brain cancer in a patient in need of such treatment, said treatment comprising administering to said patient an effective amount of at least one compound of claim 1 in combination an effective amount of at least one chemotherapeutic agent.

38. The method of claim 37 wherein said chemotherapeutic agent is temozolomide.

39. A method of treating prostate cancer in a patient in need of such treatment, said treatment comprising administering to said patient an effective amount of at least one compound of claim 1.

40. A method of treating prostate cancer in a patient in need of such treatment, said treatment comprising administering to said patient an effective amount of at least one compound of claim 1 in combination an effective amount of at least one chemotherapeutic agent.

41. The method of claim 25 wherein said chemotherapeutic agent is selected from the group consisting of: microtubule affecting agents, alkylating agents, antimetabolites, natural products and their derivatives, hormones and steroids, and synthetics.

42. The method of claim 25 wherein said chemotherapeutic agent is selected from the group consisting of: (1) taxanes, (2) platinum coordinator compounds, (3) epidermal growth factor inhibitors that are antibodies, (4) EGF inhibitors that are small molecules, (5) vascular endolithial growth factor inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha, (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors.

43. A method of treating breast cancer in a patient in need of such treatment comprising administering to said patient a therapeutically effective amount of at least one compound of claim 1 and a therapeutically effective amount of at least one antihormonal agent selected from the group consisting of: (a) aromatase inhibitors, (b) antiestrogens, and (c) LHRH analogues.

44. A method of treating breast cancer in a patient in need of such treatment comprising administering to said patient a therapeutically effective amount of at least one compound of claim 1 and a therapeutically effective amount of at least one antihormonal agent selected from the group consisting of: (a) aromatase inhibitors, (b) antiestrogens, and (c) LHRH analogues; and administering an effective amount of at least one chemotherapeutic agent.

45. A method of treating breast cancer in a patient in need of such treatment said treatment comprising administering to said patient a therapeutically effective amount of at least one compound of claim 1 and at least one antihormonal agent selected from the group consisting of: (a) aromatase inhibitors, and (b) antiestrogens.

46. A method of treating breast cancer in a patient in need of such treatment said treatment comprising administering to said patient a therapeutically effective amount of at least one compound of claim 1, at least one antihormonal agent selected from the group consisting of: (a) aromatase inhibitors and (b) antiestrogens; and at least one chemotherapeutic agent.

47. A method of treating breast cancer in a patient in need of such treatment said treatment comprising administering to said patient a therapeutically effective amount of at least one compound of claim 1 and at least one aromatase inhibitor.

48. A method of treating breast cancer in a patient in need of such treatment said treatment comprising administering to said patient a therapeutically effective amount of at least one compound of claim 1, at least one aromatase inhibitor, and at least one chemotherapeutic agent.

49. A method of treating breast cancer in a patient in need of such treatment said treatment comprising administering to said patient a therapeutically effective amount of: (1) at least one compound of claim 1; and (2) at least one antihormonal agent selected from the group consisting of: (a) aromatase inhibitors selected from the group consisting of Anastrozole, Letrozole, Exemestane, Fadrozole and Formestane, (b) antiestrogens selected from the group consisting of: Tamoxifen, Fulvestrant, Raloxifene, and Acolbifene, and (c) LHRH analogues selected from the group consisting of: Goserelin and Leuprolide; and administering an effective amount of at least one chemotherapeutic agent selected from the group consisting of: Trastuzumab, Gefitinib, Erlotinib, Bevacizumab, Cetuximab, and Bortezomib.

50. A method of treating breast cancer in a patient in need of such treatment said treatment comprising administering to said patient a therapeutically effective amount of: (1) at least one compound of claim 1; and (2) at least one antihormonal agent selected from the group consisting of: (a) aromatase inhibitors selected from the group consisting of Anastrozole, Letrozole, Exemestane, Fadrozole and Formestane, (b) antiestrogens selected from the group consisting of: Tamoxifen, Fulvestrant, Raloxifene, and Acolbifene, and (c) LHRH analogues selected from the group consisting of: Goserelin and Leuprolide.

51. A method of treating breast cancer in a patient in need of such treatment said treatment comprising administering to said patient a therapeutically effective amount of: (1) at least one compound of claim 1; and (2) at least one antihormonal agent selected from the group consisting of: (a) aromatase inhibitors selected from the group consisting of Anastrozole, Letrozole, Exemestane, Fadrozole and Formestane, and (b) antiestrogens selected from the group consisting of: Tamoxifen, Fulvestrant, Raloxifene, and Acolbifene.

52. A method of treating breast cancer in a patient in need of such treatment said treatment comprising administering to said patient a therapeutically effective amount of: (1) at least one compound of claim 1; and (2) at least one antihormonal agent selected from the group consisting of: (a) aromatase inhibitors selected from the group consisting of Anastrozole, Letrozole, Exemestane, Fadrozole and Formestane, (b) antiestrogens selected from the group consisting of: Tamoxifen, Fulvestrant, Raloxifene, and Acolbifene; and administering an effective amount of at least one chemotherapeutic agents are selected from the group consisting of: Trastuzumab, Gefitinib, Erlotinib, Bevacizumab, Cetuximab, and Bortezomib.

53. A method of treating breast cancer in a patient in need of such treatment said treatment comprising administering to said patient a therapeutically effective amount of: (1) at least one compound of claim 1; and (2) at least one aromatase inhibitor selected from the group consisting of Anastrozole, Letrozole, Exemestane, Fadrozole and Formestane.

54. A method of treating breast cancer in a patient in need of such treatment said treatment comprising administering to said patient a therapeutically effective amount of: (1) at least one compound of claim 1; (2) at least one aromatase inhibitor that is selected from the group consisting of Anastrozole, Letrozole, Exemestane, Fadrozole and Formestane; and (3) administering an effective amount of at least one chemotherapeutic agent selected from the group consisting of: Trastuzumab, Gefitinib, Erlotinib, Bevacizumab, Cetuximab, and Bortezomib.

55. A method of treating breast cancer in a patient in need of such treatment said treatment comprising administering to said patient a therapeutically effective amount of: (1) at least one compound of claim 1; (2) at least one aromatase inhibitor; and (3) at least one LHRH analogue.

56. A method of treating breast cancer in a patient in need of such treatment said treatment comprising administering to said patient a therapeutically effective amount of: (1) at least one compound of claim 1; (2) at least one antiestrogen; and (3) at least one LHRH analogue.

57. A method of treating cancer in a patient in need of such treatment, said method comprising administering to said patient an effective amount of at least one compound of claim 19, or a pharmaceutically acceptable salt thereof.

58. A method of treating cancer in a patient in need of such treatment, said method comprising administering to said patient an effective amount of at least one compound of claim 19, or a pharmaceutically acceptable salt thereof, in combination with at least one chemotherapeutic agent.

59. A method of inhibiting farnesyl protein transferase in a patient in need of such treatment, said method comprising administering to said patient an effective amount of at least one compound of claim 19, or a pharmaceutically acceptable salt thereof.

60. A method of treating cancer in a patient in need of such treatment, said method comprising administering to said patient an effective amount of at least one compound of claim 19, or a pharmaceutically acceptable salt thereof, in combination with at least one signal transduction inhibitor.

61. The method of claim 57 wherein said cancer is selected from the grop consisting of: melanoma, pancreatic cancer, thyroid cancer, colorectal cancer, lung cancer, breast cancer, and ovarian cancer.

62. The method of claim 58 wherein said cancer is selected from the grop consisting of: melanoma, pancreatic cancer, thyroid cancer, colorectal cancer, lung cancer, breast cancer, and ovarian cancer.

63. A method of treating breast cancer in a patient in need of such treatment comprising administering to said patient a therapeutically effective amount of at least one compound of claim 19 and a therapeutically effective amount of at least one antihormonal agent selected from the group consisting of: (a) aromatase inhibitors, (b) antiestrogens, and (c) LHRH analogues.

64. A method of treating breast cancer in a patient in need of such treatment comprising administering to said patient a therapeutically effective amount of at least one compound of claim 19 and a therapeutically effective amount of at least one antihormonal agent selected from the group consisting of: (a) aromatase inhibitors, (b) antiestrogens, and (c) LHRH analogues; and administering an effective amount of at least one chemotherapeutic agent.
